(19) United States
(12) Patent Application Publication
(10) Pub. No.: US 2010/0210617 A1
(43) Pub. Date: Aug. 19, 2010

(54) Aryl-Alkylamines And Heteroaryl-Alkylamines As Protein Kinase Inhibitors

(75) Inventors: Steven John Woodhead, Cambridge (GB); Robert Downham, Newmarket (GB); Christopher Hamlett, Cambridge (GB); Steven Howard, Cambridge (GB); Hannah Fiona Sore, Cambridge (GB); Marinus Leendert Verdonk, Burwell (GB); David Winter Walker, Linton (GB); Richard William Arthur Luke, Cheshire (GB)

Correspondence Address: HESLIN ROTHENBERG FARLEY & MESITI PC, 5 COLUMBIA CIRCLE, ALBANY, NY, 12203, US

(73) Assignee: ASTEX THERAPEUTICS LIMITED, Cambridge (GB)

(21) Appl. No.: 11/993,835

(22) PCT Filed: Jun. 21, 2006
(86) PCT No.: PCT/GB06/02287

§ 371 Date: May 5, 2010

Related U.S. Application Data
(60) Provisional application No. 60/692,482, filed on Jun. 21, 2005.
(60) Provisional application No. 60/744,141, filed on Apr. 3, 2006.

(30) Foreign Application Priority Data
Jun. 21, 2005 (GB) 0512642.0

(51) Int. Cl. A61K 31/4155; C07D 231/12; C07D 403/10; C07D 413/10; C07D 413/14; C07D 401/10; A61K 31/415; A61K 31/496; A61K 31/5377; A61K 31/4178; A61K 31/4439; A61K 31/506; A61K 31/497; A61K 31/454; A61P 35/00

(52) U.S. Cl. 514/210.2; 548/377.1; 544/371; 544/140; 544/129; 548/312.4; 546/275.4; 544/333; 546/211; 514/406; 514/254.05; 514/236.5; 514/397; 514/341; 514/256; 514/255.05; 514/326

(57) ABSTRACT

The invention provides a compound of the formula (II): or a salt, solvate, tautomer or N-oxide thereof; wherein n is 0 or 1; one of Y¹ and Y² is CH and the other is selected from CH, CR⁸ and N; q is 0, 1 or 2 provided that q is 0 or 1 when Y¹ or Y² is CR⁸; R¹ aryl or heteroaryl group of 5 to 10 ring members; R^{2a} and R^{3a} each are hydrogen, C₁₋₄ hydrocarbyl or C₁₋₄ acyl wherein the hydrocarbyl and acyl moieties are optionally substituted by fluorine, hydroxy, amino, methylamino, dimethylamino or methoxy; or NR^{2a}R^{3a} forms an imidazole group or a saturated monocyclic 4-7 membered heterocyclic group optionally containing a second heteroatom ring member selected from O and N; R¹⁸ is hydrogen or methyl; R¹⁹ is hydrogen or methyl; R²⁴ is hydrogen or R²⁴, R^{2a} and the intervening nitrogen atom and carbon atoms together form an azetidine, pyrrolidine or piperidine ring; R²⁵ is hydrogen or a C₁₋₄ alkyl group wherein the C₁₋₄ alkyl group is optionally substituted by hydroxy or amino provided that there are at least two carbon atoms between the hydroxy or amino group and the oxygen atom to which R²⁵ is attached; and R4 and R⁵ each are hydrogen or a substituent as defined in the claims

This invention relates to pyrazole-containing aryl- and heteroaryl-alkylamine compounds that inhibit or modulate the activity of protein kinase B (PKB) and protein kinase A (PKA), to the use of the compounds in the treatment or prophylaxis of disease states or conditions mediated by PKB and PKA, and to novel compounds having PKB and PKA inhibitory or modulating activity. Also provided are pharmaceutical compositions containing the compounds and novel chemical intermediates.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, diseases and conditions of the immune system, diseases and conditions of the central nervous system, and angiogenesis.

Apoptosis or programmed cell death is an important physiological process which removes cells no longer required by an organism. The process is important in early embryonic growth and development allowing the non-necrotic controlled breakdown, removal and recovery of cellular components. The removal of cells by apoptosis is also important in the maintenance of chromosomal and genomic integrity of growing cell populations. There are several known checkpoints in the cell growth cycle at which DNA damage and genomic integrity are carefully monitored. The response to the detection of anomalies at such checkpoints is to arrest the growth of such cells and initiate repair processes. If the damage or anomalies cannot be repaired then apoptosis is initiated by the damaged cell in order to prevent the propagation of faults and errors. Cancerous cells consistently contain numerous mutations, errors or rearrangements in their chromosomal DNA. It is widely believed that this occurs in part because the majority of tumours have a defect in one or more of the processes responsible for initiation of the apoptotic process. Normal control mechanisms cannot kill the cancerous cells and the chromosomal or DNA coding errors continue to be propagated. As a consequence restoring these pro-apoptotic signals or suppressing unregulated survival signals is an attractive means of treating cancer.

The signal transduction pathway containing the enzymes phosphatidylinositol 3-kinase (PI3K), PDK1 and PKB amongst others, has long been known to mediate increased resistance to apoptosis or survival responses in many cells. There is a substantial amount of data to indicate that this pathway is an important survival pathway used by many growth factors to suppress apoptosis. The enzyme PI3K is activated by a range of growth and survival factors e.g. EGF, PDGF and through the generation of polyphosphatidylinositols, initiates the activation of the downstream signalling events including the activity of the kinases PDK1 and protein kinase B (PKB) also known as Akt. This is also true in host tissues, e.g. vascular endothelial cells as well as neoplasias. PKB is a protein ser/thr kinase consisting of a kinase domain together with an N-terminal PH domain and C-terminal regulatory domain. The enzyme PKB itself is phosphorylated on Thr 308 by PDK1 and on Ser 473 by an as yet unidentified kinase. Full activation requires phosphorylation at both sites whilst association between PIP3 and the PH domain is required for anchoring of the enzyme to the cytoplasmic face of the lipid membrane providing optimal access to substrates.

Activated PKB in turn phosphorylates a range of substrates contributing to the overall survival response. Whilst we cannot be certain that we understand all of the factors responsible for mediating the PKB dependent survival response, some important actions are believed to be phosphorylation and inactivation of the pro-apoptotic factor BAD and caspase 9, phosphorylation of Forkhead transcription factors e.g. FKHR leading to their exclusion from the nucleus, and activation of the NfkappaB pathway by phosphorylation of upstream kinases in the cascade.

In addition to the anti-apoptotic and pro-survival actions of the PKB pathway, the enzyme also plays an important role in promoting cell proliferation. This action is again likely to be mediated via several actions, some of which are thought to be phosphorylation and inactivation of the cyclin dependent kinase inhibitor of p21^{Cip1/WAF1}, and phosphorylation and activation of mTOR, a kinase controlling several aspects of cell growth.

The phosphatase PTEN which dephosphorylates and inactivates polyphosphatidyl-inositols is a key tumour suppressor protein which normally acts to regulate the PI3K/PKB survival pathway. The significance of the PI3K/PKB pathway in tumourigenesis can be judged from the observation that PTEN is one of the most common targets of mutation in human tumours, with mutations in this phosphatase having been found in ˜50% or more of melanomas (Guldberg et al 1997, Cancer Research 57, 3660-3663) and advanced prostate cancers (Cairns et al 1997 Cancer Research 57, 4997). These observations and others suggest that a wide range of tumour types are dependent on the enhanced PKB activity for growth and survival and would respond therapeutically to appropriate inhibitors of PKB.

There are 3 closely related isoforms of PKB called alpha, beta and gamma, which genetic studies suggest have distinct but overlapping functions. Evidence suggests that they can all independently play a role in cancer. For example PKB beta has been found to be over-expressed or activated in 10-40% of ovarian and pancreatic cancers (Bellacosa et al 1995, Int. J. Cancer 64, 280-285; Cheng et al 1996, PNAS 93, 3636-3641; Yuan et al 2000, Oncogene 19, 2324-2330), PKB alpha is amplified in human gastric, prostate and breast cancer (Staal 1987, PNAS 84, 5034-5037; Sun et al 2001, Am. J. Pathol. 159, 431-437) and increased PKB gamma activity has been observed in steroid independent breast and prostate cell lines (Nakatani et al 1999, J. Biol. Chem. 274, 21528-21532).

The PKB pathway also functions in the growth and survival of normal tissues and may be regulated during normal physiology to control cell and tissue function. Thus disorders associated with undesirable proliferation and survival of normal cells and tissues may also benefit therapeutically from treatment with a PKB inhibitor. Examples of such disorders are disorders of immune cells associated with prolonged expansion and survival of cell population leading to a prolonged or up regulated immune response. For example, T and B lymphocyte response to cognate antigens or growth factors such as interleukin-2 activates the PI3K/PKB pathway and is responsible for maintaining the survival of the antigen specific lymphocyte clones during the immune response. Under conditions in which lymphocytes and other immune cells are responding to inappropriate self or foreign antigens, or in which other abnormalities lead to prolonged activation, the PKB pathway contributes an important survival signal preventing the normal mechanisms by which the immune response is terminated via apoptosis of the activated cell population. There is a considerable amount of evidence demonstrating the expansion of lymphocyte populations responding to self antigens in autoimmune conditions such as multiple sclerosis and arthritis. Expansion of lymphocyte populations responding inappropriately to foreign antigens is a feature of another set of conditions such as allergic responses and asthma. In summary inhibition of PKB could provide a beneficial treatment for immune disorders.

Other examples of inappropriate expansion, growth, proliferation, hyperplasia and survival of normal cells in which PKB may play a role include but are not limited to atherosclerosis, cardiac myopathy and glomerulonephritis.

In addition to the role in cell growth and survival, the PKB pathway functions in the control of glucose metabolism by insulin. Available evidence from mice deficient in the alpha and beta isoforms of PKB suggests that this action is mediated by the beta isoform. As a consequence, modulators of PKB activity may also find utility in diseases in which there is a dysfunction of glucose metabolism and energy storage such as diabetes, metabolic disease and obesity.

Cyclic AMP-dependent protein kinase (PKA) is a serine/threonine protein kinase that phosphorylates a wide range of substrates and is involved in the regulation of many cellular processes including cell growth, cell differentiation, ion-channel conductivity, gene transcription and synaptic release of neurotransmitters. In its inactive form, the PKA holoenzyme is a tetramer comprising two regulatory subunits and two catalytic subunits.

PKA acts as a link between G-protein mediated signal transduction events and the cellular processes that they regulate. Binding of a hormone ligand such as glucagon to a transmembrane receptor activates a receptor-coupled G-protein (GTP-binding and hydrolyzing protein). Upon activation, the alpha subunit of the G protein dissociates and binds to and activates adenylate cyclase, which in turn converts ATP to cyclic-AMP (cAMP). The cAMP thus produced then binds to the regulatory subunits of PKA leading to dissociation of the associated catalytic subunits. The catalytic subunits of PKA, which are inactive when associated with the regulatory sub-units, become active upon dissociation and take part in the phosphorylation of other regulatory proteins.

For example, the catalytic sub-unit of PKA phosphorylates the kinase Phosphorylase Kinase which is involved in the phosphorylation of Phosphorylase, the enzyme responsible for breaking down glycogen to release glucose. PKA is also involved in the regulation of glucose levels by phosphorylating and deactivating glycogen synthase. Thus, modulators of PKA activity (which modulators may increase or decrease PKA activity) may be useful in the treatment or management of diseases in which there is a dysfunction of glucose metabolism and energy storage such as diabetes, metabolic disease and obesity.

PKA has also been established as an acute inhibitor of T cell activation. Anndahl et al, have investigated the possible role of PKA type I in HIV-induced T cell dysfunction on the basis that T cells from HIV-infected patients have increased levels of cAMP and are more sensitive to inhibition by cAMP analogues than are normal T cells. From their studies, they concluded that increased activation of PKA type I may contribute to progressive T cell dysfunction in HIV infection and that PKA type I may therefore be a potential target for immunomodulating therapy.-Aandahl, E. M., Aukrust, P., Sk\dot{a}lhegg, B. S., Müller, F., Frøland, S. S., Hansson, V., Taskén, K. Protein kinase A type I antagonist restores immune responses of T cells from HIV-infected patients. FASEB J. 12, 855-862 (1998).

It has also been recognised that mutations in the regulatory sub-unit of PKA can lead to hyperactivation in endocrine tissue.

Because of the diversity and importance of PKA as a messenger in cell regulation, abnormal responses of cAMP can lead to a variety of human diseases such as irregular cell growth and proliferation (Stratakis, C. A.; Cho-Chung, Y. S.; Protein Kinase A and human diseases. Trends Endrocri. Metab. 2002, 13, 50-52). Over-expression of PKA has been observed in a variety of human cancer cells including those from ovarian, breast and colon patients. Inhibition of PKA would therefore be an approach to treatment of cancer (Li, Q.; Zhu, G-D.; Current Topics in Medicinal Chemistry, 2002, 2, 939-971).

For a review of the role of PKA in human disease, see for example, Protein Kinase A and Human Disease, Edited by Constantine A. Stratakis, Annals of the New York Academy of Sciences, Volume 968, 2002, ISBN 1-57331-412-9.

hERG

In the late 1990s a number of drugs, approved by the US FDA, had to be withdrawn from sale in the US when it was discovered they were implicated in deaths caused by heart malfunction. It was subsequently found that a side effect of these drugs was the development of arrhythmias caused by the blocking of hERG channels in heart cells. The hERG channel is one of a family of potassium ion channels the first member of which was identified in the late 1980s in a mutant Drosophila melanogaster fruitfly (see Jan, L. Y. and Jan, Y. N. (1990). A Superfamily of Ion Channels. Nature, 345(6277):672). The biophysical properties of the hERG potassium ion channel are described in Sanguinetti, M. C., Jiang, C., Curran, M. E., and Keating, M. T. (1995). A Mechanistic Link Between an Inherited and an Acquired Cardiac Arrhythmia: HERG encodes the Ikr potassium channel. Cell, 81:299-307, and Trudeau, M. C., Warmke, J. W., Ganetzky, B., and Robertson, G. A. (1995). HERG, a Human Inward Rectifier in the Voltage-Gated Potassium Channel Family. Science, 269:92-95.

The elimination of hERG blocking activity remains an important consideration in the development of any new drug.

### PRIOR ART

Several classes of compounds have been disclosed as having PKA and PKB inhibitory activity.

For example, a class of isoquinolinyl-sulphonamido-diamines having PKB inhibitory activity is disclosed in WO 01/91754 (Yissum).

WOO/07996 (Chiron) discloses substituted pyrazoles having estrogen receptor agonist activity. The compounds are described as being useful in treating or preventing inter alia estrogen-receptor mediated breast cancer. PKB inhibitory activity is not disclosed.

WO 00/31063 (Searle) discloses substituted pyrazole compounds as p38 kinase inhibitors.

WO 01/32653 (Cephalon) discloses a class of pyrazolone kinase inhibitors. WO 03/059884 (X-Ceptor Therapeutics) discloses N-substituted pyridine compounds as modulators of nuclear receptors.

WO 03/068230 (Pharmacia) discloses substituted pyridones as p38 MAP kinase modulators.

WO 00/66562 (Dr Reddy's Research Foundation) discloses a class of 1-phenyl-substituted pyrazoles for use as anti-inflammatory agents. The 1-phenyl group is substituted by a sulphur-containing substituent as a sulphonamide or sulphonyl group.

WO 2005/061463 (Astex) discloses a class of substituted pyrazole compounds having PKB and PKA inhibitory activity.

### SUMMARY OF THE INVENTION

The invention provides compounds that have protein kinase B (PKB) and protein kinase A (PKA) inhibiting or modulating activity, and which it is envisaged will be useful in preventing or treating disease states or conditions mediated by PKB or PKA.

In a first aspect, the invention provides a compound of the formula (I):

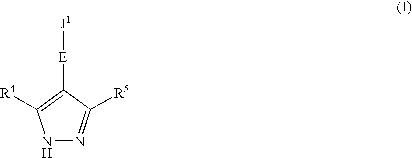

or a salt, solvate, tautomer or N-oxide thereof;
wherein:
E is a monocyclic or bicyclic carbocyclic or heterocyclic group;
R⁴ is selected from hydrogen, halogen, C₁₋₅ saturated hydrocarbyl, C₁₋₅ saturated hydrocarbyloxy, cyano, and CF₃; and
R⁵ is selected from selected from hydrogen, halogen, C₁₋₅ saturated hydrocarbyl, C₁₋₅ saturated hydrocarbyloxy, cyano, CONH₂, CONHR⁹, CF₃, NH₂, NHCOR⁹ or NHCONHR⁹;
R⁹ is a group R^{9a} or (CH₂)R^{9a}, wherein R^{9a} is a monocyclic or bicyclic group which may be carbocyclic or heterocyclic;
   the carbocyclic group or heterocyclic group R^{9a} being optionally-substituted by one or more substituents selected from fluorine, chlorine or a group R¹³;

R¹³ is selected from halogen (other than fluorine or chlorine), hydroxy, trifluoromethyl, cyano, nitro, carboxy, amino, mono- or di-C₁₋₄ hydrocarbylamino; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; provided that R^{b} is other than hydrogen when R^{a} is a bond;
R^{c} is selected from hydrogen and C₁₋₄ hydrocarbyl; and
X¹ is O, S or NR^{c} and X² is ═O, ═S or ═NR^{c}; and
   J¹ is selected from:
   (1) a group of the formula:

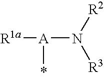

wherein:
the asterisk denotes the point of attachment to the group E;
A is a saturated hydrocarbon linker group containing from 1 to 7 carbon atoms, the linker group having a maximum chain length of 5 atoms extending between R^{1a} and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from oxo, fluorine and hydroxy, provided that the hydroxy group when present is not located at a carbon atom a with respect to the NR²R³ group and provided that the oxo group when present is located at a carbon atom a with respect to the NR²R³ group;
R^{1a} is an aryl or heteroaryl group selected from groups R^{1b}, R^{1c}, R^{1d} and R^{1c};
R^{1b} has the formula:

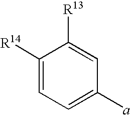

wherein:
“a” denotes the point of attachment to the group A;
R^{1c} is a heteroaryl group selected from pyridine, pyrimidine, pyrazine, pyridone, N-methylpyridone, furan and thiophene, each of which heteroaryl groups is unsubstituted or substituted with one or two substituents selected from chlorine, fluorine, methyl, methoxy and trifluoromethoxy;
R^{1d} has the formula:

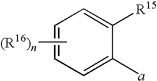

wherein:
“a” denotes the point of attachment to the group A;
R^{1e} has the formula:

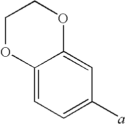

wherein:
“a” denotes the point of attachment to the group A;
R² and R³ are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the linker group A form a saturated monocyclic heterocyclic group having 4-7 ring members and optionally containing a second heteroatom ring member selected from O and N;
or NR²R³ and the carbon atom of linker group A to which it is attached together form a cyano group;
R¹⁴ is selected from hydrogen, fluorine, chlorine, methoxy, cyano, trifluoromethyl, trifluoromethoxy, difluoromethoxy, C₁₋₄ alkyl and cyclopropyl;
R¹⁵ is selected from fluorine, chlorine, methoxy, cyano, trifluoromethyl, trifluoromethoxy, difluoromethoxy, C₁₋₄ alkyl and cyclopropyl; and
R¹⁶ is selected from R¹³ and R¹⁴, and n is 0, 1 or 2, provided that when n is 2, only one group R¹³ may be present; and
   (2) a group of the formula:

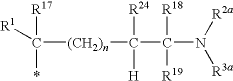

wherein:
the asterisk denotes the point of attachment to the group E;
n is 0 or 1;
R¹ is an aryl or heteroaryl group of 5 to 10 ring members;
R^{2a} and R^{3a} are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy;
or R^{2a} and R^{3a} together with the nitrogen atom to which they are attached form a cyclic group selected from an imidazole group and a saturated monocyclic heterocyclic group having 4-7 ring members and optionally containing a second heteroatom ring member selected from O and N;
R¹⁷ is selected from hydrogen; hydroxy; fluorine; methyl; and a C₁₋₄ alkoxy group wherein the C₁₋₄ alkoxy group is optionally substituted by hydroxy or amino provided that there are at least two carbon atoms between the hydroxy or amino group and the oxygen atom of the C₁₋₄ alkoxy group;
R¹⁸ is selected from hydrogen and methyl;
R¹⁹ is selected from hydrogen and methyl; provided that at least one of R¹⁷, R¹⁸ is R¹⁹ is other than hydrogen; and

R²⁴ is hydrogen or R²⁴, R^{2a} and the intervening nitrogen atom and carbon atoms together form an azetidine, pyrrolidine or piperidine ring;

and
(3) a group of the formula:

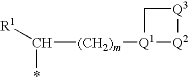

wherein:
the asterisk denotes the point of attachment to the group E;
m is 0 or 1;
Q¹ is CH or N;
Q² is CH₂ or NH;
Q³ is CH₂ or NH, provided that one of Q¹, Q² and Q³ consists of or contains a nitrogen atom and the remainder of Q¹, Q² and Q³ are each CH or CH₂; and
R¹ is as hereinbefore defined;
   and
   (4) a group of the formula:

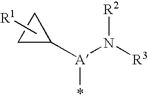

wherein A′ is a saturated hydrocarbon linker group containing from 1 to 5 carbon atoms, the linker group having a maximum chain length of 4 atoms extending between the cyclopropyl group and NR²R³ and a maximum chain length of 4 atoms extending between E and NR²R³;
R¹ is as hereinbefore defined;
R² and R³ are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the group A′ form a saturated monocyclic heterocyclic group having 4-7 ring members and optionally containing a second heteroatom ring member selected from O and N;
or NR²R³ and the carbon atom of group A′ to which it is attached together form a cyano group;
   and
   (5) when (a) one of R^{45aa} is hydrogen and the other is a group R^{45aa} wherein R^{45aa} is C₁₋₄ hydrocarbyl and E is phenyl; or (b) R⁴ and R⁵ are both hydrogen and E is a pyridyl ring in wherein J¹ is attached to the 2-position of the pyridyl ring and the pyrazole group is attached to the 5-position of the pyridyl ring; then J¹ is further selected from:
(5A) a group of the formula:

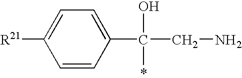

wherein the asterisk indicates the point of attachment to the group E and R²¹ is hydrogen or chlorine; and
(5B) a group of the formula:

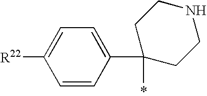

wherein the asterisk indicates the point of attachment to the group E and R²² is chlorine or methoxy provided that when R²² is chlorine, then R^{45aa} is C₂₋₄ hydrocarbyl;
   and
   (6) a group of the formula:

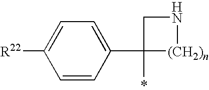

wherein n is 1 or 2 and R²² is chlorine or methoxy; provided that R⁴ and R⁵ are both hydrogen and E is phenyl;
   and
   (7) a group of the formula:

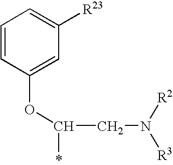

wherein R² and R³ are as defined herein and R²³ is fluorine, chlorine or a group R¹³;
and
(8) a group of the formula:

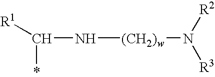

wherein w is 2 or 3 and R¹, R² and R³ are as defined herein;
wherein formula (I) further includes the compounds:
4-(3-chloro-phenyl)-4-[4-(1H-pyrazol-4-yl)-phenyl]-piperidine;
3-(4-chloro-3-fluoro-phenyl)-3-[4-(1H-pyrazol-4-yl)-phenyl]-propylamine; and
{(R)-3-(3,4-dichloro-phenyl)-3-[4-(1H-pyrazol-4-yl)-phenyl]-propylamine;
   and salts, solvates, tautomers and N-oxides thereof;
   but excludes the compounds:
dimethyl-{3-[4-(1H-pyrazol-4-yl)-phenyl]-3-pyridin-2-yl-propyl}-amine;
4-[4-(1H-pyrazol-4-yl)-phenyl]-1,2,3,4,5,6-hexahydro-[4,4′]bipyridinyl;
4-(2-chloro-3-fluoro-phenyl)-4-[4-(1H-pyrazol-4-yl)-phenyl]-piperidine;
4-{4-[2-azetidin-1-yl-1-(4-chloro-phenyl)-ethyl]-phenyl}-1H-pyrazole; and
4-{4-[3-azetidin-1-yl-1-(4-chloro-phenyl)-propyl]-phenyl}-1H-pyrazole.

In a second aspect, the invention provides a compound of the formula (Ia):

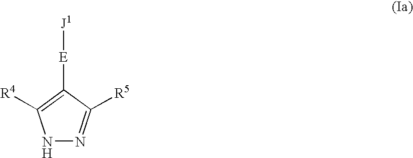

or a salt, solvate, tautomer or N-oxide thereof;
wherein:
E is a monocyclic or bicyclic carbocyclic or heterocyclic group;
R⁴ is selected from hydrogen, halogen, C₁₋₅ saturated hydrocarbyl, C₁₋₅ saturated hydrocarbyloxy, cyano, and CF₃; and
R⁵ is selected from selected from hydrogen, halogen, C₁₋₅ saturated hydrocarbyl, C₁₋₅ saturated hydrocarbyloxy, cyano, CONH₂, CONHR⁹, CF₃, NH₂, NHCOR⁹ or NHCONHR⁹;
R⁹ is a group R^{9a} or (CH₂)R^{9a}, wherein R^{9a} is a monocyclic or bicyclic group which may be carbocyclic or heterocyclic;
   the carbocyclic group or heterocyclic group R^{9a} being optionally substituted by one or more substituents selected from fluorine, chlorine or a group R¹³;
R¹³ is selected from halogen (other than fluorine or chlorine), hydroxy, trifluoromethyl, cyano, nitro, carboxy, amino, mono- or di-C₁₋₄ hydrocarbylamino; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; provided that R^{b} is other than hydrogen when R^{a} is a bond;
R^{c}C is selected from hydrogen and C₁₋₄ hydrocarbyl; and
X¹ is O, S or NR^{c} and X² is ═O, ═S or ═NR^{c}; and
   J¹ is selected from:
   (1) a group of the formula:

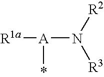

wherein:
the asterisk denotes the point of attachment to the group E;
A is a saturated hydrocarbon linker group containing from 1 to 7 carbon atoms, the linker group having a maximum chain length of 5 atoms extending between R^{1a} and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from oxo, fluorine and hydroxy, provided that the hydroxy group when present is not located at a carbon atom a with respect to the NR²R³ group and provided that the oxo group when present is located at a carbon atom a with respect to the NR²R³ group;
R^{1a} is an aryl or heteroaryl group selected from groups R^{1b}, R^{1c}, R^{1d} and R^{1e};
R^{1b} has the formula:

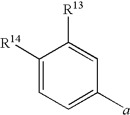

wherein:
“a” denotes the point of attachment to the group A;
R^{1c} is a heteroaryl group selected from pyridine, pyrimidine, pyrazine, pyridone, N-methylpyridone, furan and thiophene, each of which heteroaryl groups is unsubstituted or substituted with one or two substituents selected from chlorine, fluorine, methyl, methoxy and trifluoromethoxy;
R^{1d} has the formula:

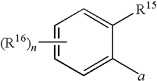

wherein:
“a” denotes the point of attachment to the group A;
R^{1e} has the formula:

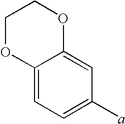

wherein:
“a” denotes the point of attachment to the group A
R² and R³ are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the linker group A form a saturated monocyclic heterocyclic group having 4-7 ring members and optionally containing a second heteroatom ring member selected from O and N;
or NR²R³ and the carbon atom of linker group A to which it is attached together form a cyano group;
R¹⁴ is selected from hydrogen, fluorine, chlorine, methoxy, cyano, trifluoromethyl, trifluoromethoxy, difluoromethoxy, C₁₋₄ alkyl and cyclopropyl;
R¹⁵ is selected from fluorine, chlorine, methoxy, cyano, trifluoromethyl, trifluoromethoxy, difluoromethoxy, C₁₋₄ alkyl and cyclopropyl; and
R¹⁶ is selected from R¹³ and R¹⁴, and n is 0, 1 or 2, provided that when n is 2, only one group R¹³ may be present;
   and
   (2) a group of the formula:

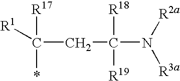

wherein:
the asterisk denotes the point of attachment to the group E;
R¹ is an aryl or heteroaryl group of 5 to 10 ring members;
R^{2a} and R^{3a} are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy;
or R^{2a} and R^{3a} together with the nitrogen atom to which they are attached form a cyclic group selected from an imidazole group and a saturated monocyclic heterocyclic group having 4-7 ring members and optionally containing a second heteroatom ring member selected from O and N;
R¹⁷ is selected from hydrogen, hydroxy, fluorine and methyl;
R¹⁸ is selected from hydrogen and methyl;
R¹⁹ is selected from hydrogen and methyl; provided that at least one of R¹⁷, R¹⁸ and R¹⁹ is other than hydrogen;
   and
   (3) a group of the formula:

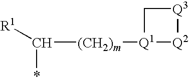

wherein:
the asterisk denotes the point of attachment to the group E;
m is 0 or 1;
Q¹ is CH or N;
Q² is CH₂ or NH;
Q³ is CH₂ or NH, provided that one of Q¹, Q² and Q³ consists of or contains a nitrogen atom and the remainder of Q¹, Q² and Q³ are each CH or CH₂; and
R¹ is as hereinbefore defined;
   and
   (4) a group of the formula:

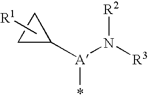

wherein A′ is a saturated hydrocarbon linker group containing from 1 to 5 carbon atoms, the linker group having a maximum chain length of 4 atoms extending between the cyclopropyl group and NR²R³ and a maximum chain length of 4 atoms extending between E and NR²R³;
R¹ is as hereinbefore defined;
R² and R³ are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the group A′ form a saturated monocyclic heterocyclic group having 4-7 ring members and optionally containing a second heteroatom ring member selected from O and N;
or NR²R³ and the carbon atom of group A′ to which it is attached together form a cyano group;
   and
   (5) when (a) R⁴ is hydrogen and R⁵ is a group R^{5aa} wherein R^{5aa is C}₁₋₄ hydrocarbyl and E is phenyl; or (b) R⁴ and R⁵ are both hydrogen and E is a pyridyl ring in wherein J¹ is attached to the 2-position of the pyridyl ring and the pyrazole group is attached to the 5-position of the pyridyl ring; then J¹ is further selected from:
(5A) a group of the formula:

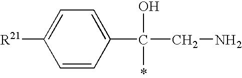

wherein the asterisk indicates the point of attachment to the group E and R²¹ is hydrogen or chlorine; and
(5B) a group of the formula:

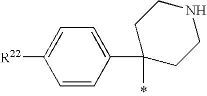

wherein the asterisk indicates the point of attachment to the group E and R²² is chlorine or methoxy provided that when R²² is chlorine, then R^{5aa} is C₂₋₄ hydrocarbyl;
   and
   (6) a group of the formula:

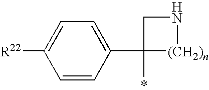

wherein n is 1 or 2 and R²² is chlorine or methoxy; provided that R⁴ and R⁵ are both hydrogen and E is phenyl;
and
   (7) a group of the formula:

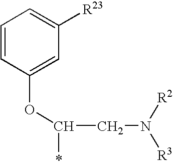

wherein R² and R³ are as defined herein and R²³ is fluorine, chlorine or a group R¹³;
wherein formula (I) further includes the compounds:
4-(3-chloro-phenyl)-4-[4-(1H-pyrazol-4-yl)-phenyl]-piperidine;
3-(4-chloro-3-fluoro-phenyl)-3-[4-(1H-pyrazol-4-yl)-phenyl]-propylamine; and
{(R)-3-(3,4-dichloro-phenyl)-3-[4-(1H-pyrazol-4-yl)-phenyl]-propylamine;
   and salts, solvates, tautomers and N-oxides thereof;
   but excludes the compounds:
dimethyl-{3-[4-(1H-pyrazol-4-yl)-phenyl]-3-pyridin-2-yl-propyl}-amine;
4-[4-(1H-pyrazol-4-yl)-phenyl]-1,2,3,4,5,6-hexahydro-[4,4]bipyridinyl;
4-(2-chloro-3-fluoro-phenyl)-4-[4-(1H-pyrazol-4-yl)-phenyl]-piperidine;
4-{-4-[2-azetidin-1-yl-1-(4-chloro-phenyl)-ethyl]-phenyl}-1H-pyrazole; and
4-{4-[3-azetidin-1-yl-1-(4-chloro-phenyl)-propyl]-phenyl}-1H-pyrazole.

In another aspect, the invention provides a compound of the formula (Ib):

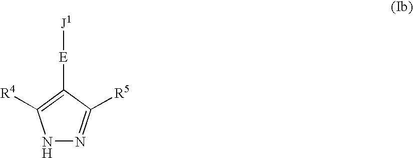

or a salt, solvate, tautomer or N-oxide thereof;
wherein:
E is a monocyclic or bicyclic carbocyclic or heterocyclic group;
R⁴ is selected from hydrogen, halogen, C₁₋₅ saturated hydrocarbyl, C₁₋₅ saturated hydrocarbyloxy, cyano, and CF₃; and
R⁵ is selected from selected from hydrogen, halogen, C₁₋₅ saturated hydrocarbyl, C₁₋₅ saturated hydrocarbyloxy, cyano, CONH₂, CONHR⁹, CF₃, NH₂, NHCOR⁹ or NHCONHR⁹;
R⁹ is a group R^{9a} or (CH₂)R^{9a}, wherein R^{9a} is a monocyclic or bicyclic group which may be carbocyclic or heterocyclic;
   the carbocyclic group or heterocyclic group R^{9a} being optionally substituted by one or more substituents selected from fluorine, chlorine or a group R¹³;
R¹³ is selected from halogen (other than fluorine or chlorine), hydroxy, trifluoromethyl, cyano, nitro, carboxy, amino, mono- or di-C₁₋₄ hydrocarbylamino; a group R^{a}—R^{b} wherein R^{a} is a bond, O, CO, X¹C(X²), C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; provided that R^{b} is other than hydrogen when R^{a} is a bond;
R^{c} is selected from hydrogen and C₁₋₄ hydrocarbyl; and
X¹ is O, S or Me and X² is ═O, ═S or ═NR^{c}; and
   J¹ is selected from:
   (1) a group of the formula:

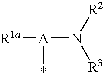

wherein:
the asterisk denotes the point of attachment to the group E;
A is a saturated hydrocarbon linker group containing from 1 to 7 carbon atoms, the linker group having a maximum chain length of 5 atoms extending between R^{1a} and NR²R³ and a maximum chain length of 4 atoms extending between E and NR²R³, wherein one of the carbon atoms in the linker group may optionally be replaced by an oxygen or nitrogen atom; and wherein the carbon atoms of the linker group A may optionally bear one or more substituents selected from oxo, fluorine and hydroxy, provided that the hydroxy group when present is not located at a carbon atom a with respect to the NR²R³ group and provided that the oxo group when present is located at a carbon atom a with respect to the NR²R³ group;
R^{1a} is an aryl or heteroaryl group selected from groups R^{1b}, R^{1c} and R^{1d};
R^{1b} has the formula:

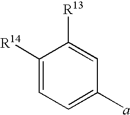

wherein:
“a” denotes the point of attachment to the group A;
R^{1c} is a heteroaryl group selected from pyridine, pyrimidine, pyrazine, pyridone, N-methylpyridone, furan and thiophene, each of which heteroaryl groups is unsubstituted or substituted with one or two substituents selected from chlorine, fluorine, methyl, methoxy and trifluoromethoxy;
R^{1d} has the formula:

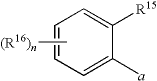

wherein:
“a” denotes the point of attachment to the group A;
R² and R³ are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the linker group A form a saturated monocyclic heterocyclic group having 4-7 ring members and optionally containing a second heteroatom ring member selected from O and N;
or NR²R³ and the carbon atom of linker group A to which it is attached together form a cyano group;
R¹⁴ is selected from hydrogen, fluorine, chlorine, methoxy, cyano, trifluoromethyl, trifluoromethoxy, difluoromethoxy, C₁₋₄ alkyl and cyclopropyl;
R¹⁵ is selected from fluorine, chlorine, methoxy, cyano, trifluoromethyl, trifluoromethoxy, difluoromethoxy, C₁₋₄ alkyl and cyclopropyl; and
R¹⁶ is selected from R¹³ and R¹⁴, and n is 0, 1 or 2, provided that when n is 2, only one group R¹³ may be present;
   and
   (2) a group of the formula:

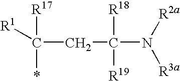

wherein:
the asterisk denotes the point of attachment to the group E;
R¹ is an aryl or heteroaryl group;
R^{2a} and R^{3a} are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy;
or R^{2a} and R^{3a} together with the nitrogen atom to which they are attached form a cyclic group selected from an imidazole group and a saturated monocyclic heterocyclic group having 4-7 ring members and optionally containing a second heteroatom ring member selected from O and N;
R¹⁷ is selected from hydrogen, hydroxy, fluorine and methyl;
R¹⁸ is selected from hydrogen and methyl;
R¹⁹ is selected from hydrogen and methyl; provided that at least one of R¹⁷, R¹⁸ and R¹⁹ is other than hydrogen;
   and
   (3) a group of the formula:

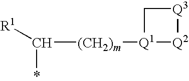

wherein:
the asterisk denotes the point of attachment to the group E;
m is 0 or 1;
Q¹ is CH or N;
Q² is CH₂ or NH;
Q³ is CH₂ or NH, provided that only one of Q¹, Q² and Q³ consists of or contains a nitrogen atom; and

R¹ is as hereinbefore defined;

but excluding the compounds:
dimethyl-{3-[4-(1H-pyrazol-4-yl)-phenyl]-3-pyridin-2-yl-propyl}-amine;
4-[4-(1H-pyrazol-4-yl)-phenyl]-1,2,3,4,5,6-hexahydro-[4,4′]bipyridinyl;
4-(2-chloro-3-fluoro-phenyl)-4-[4-(1H-pyrazol-4-yl)-phenyl]-piperidine;
4-{-4-[2-azetidin-1-yl-1-(4-chloro-phenyl)-ethyl]-phenyl}-1H-pyrazole; and
4-{-4-[3-azetidin-1-yl-1-(4-chloro-phenyl)-propyl]-phenyl}-1H-pyrazole.

The invention further provides:
A compound per se of the formula (II), (IIa), (IIb), (IIc), (III), (IIIA), (IV), (V), (VI), (VII), (VIIa), (VIII), (IX) or any other sub-group or embodiment of the formula (I) as defined herein.
A compound of the formula (I), (Ia), (Ib), (II), (IIa), (IIb), (IIc), (III), (IIIa), (IV), (V), (VI), (VII), (VIIa), (VIII), (IX) or any sub-group thereof as defined herein for use in the prophylaxis or treatment of a disease state or condition mediated by protein kinase B.
The use of a compound of (I), (Ia), (Ib), (II), (IIa), (IIb), (IIc), (III), (IIIa), (IV), (V), (VI), (VII), (VIIa), (VIII), (IX) or any sub-group thereof as defined herein for the manufacture of a medicament for the prophylaxis or treatment of a disease state or condition mediated by protein kinase B.
A method for the prophylaxis or treatment of a disease state or condition mediated by protein kinase B, which method comprises administering to a subject in need thereof a compound of the (I), (Ia), (Ib), (II), (IIa), (IIb), (IIe), (III), (IIIa), (IV), (V), (VI), (VII), (VIIa), (VIII), (IX) or any sub-group thereof as defined herein.
A compound of the formula (I), (Ia), (Ib), (II), (IIa), (IIb), (IIc), (III), (IIIa), (IV), (V), (VI), (VII), (VIIa), (VIII), (IX) or any sub-group thereof as defined herein for use in treating a disease or condition comprising or arising from abnormal cell growth or abnormally arrested cell death in a mammal.
The use of a compound of (I), (Ia), (Ib), (II), (IIa), (IIb), (IIc), (III), (IIIa), (IV), (V), (VI), (VII), (VIIa), (VIII), (IX) or any sub-group thereof as defined herein for the manufacture of a medicament for treating a disease or condition comprising or arising from abnormal cell growth or abnormally arrested cell death in a mammal.
A method for treating a disease or condition comprising or arising from abnormal cell growth or abnormally arrested cell death in a mammal, the method comprising administering to the mammal a compound of the (I), (Ia), (Ib), (II), (IIa), (IIb), (IIc), (III), (IIIa), (IV), (V), (VI), (VII), (VIIa), (VIII), (IX) or any sub-group thereof as defined herein in an amount effective to inhibit protein kinase B activity.
A method of inhibiting protein kinase B, which method comprises contacting the kinase with a kinase-inhibiting compound of the formula (I), (Ia), (Ib), (II), (IIa), (IIb), (IIc), (III), (IIIa), (IV), (V), (VI), (VII), (VIIa), (VIII), (IX) or any sub-group thereof as defined herein.
A method of modulating a cellular process (for example cell division) by inhibiting the activity of a protein kinase B using a compound of the formula (I), (Ia), (Ib), (II), (IIa), (IIb), (IIc), (III), (IIIa), (IV), (V), (VI), (VII), (VIIa), (VIII), (IX) or any sub-group thereof as defined herein.
A compound of the formula (I), (Ia), (Ib), (II), (IIb), (IIc), (III), (IIIa), (IV), (V), (VI), (VII), (VIIa), (VIII), (IX) or any sub-group or embodiment thereof as defined herein for use in the prophylaxis or treatment of a disease state or condition mediated by protein kinase A.
The use of a compound of the formula (I), (Ia), (Ib), (II), (IIa), (IIb), (IIc), (III), (IIIa), (IV), (V), (VI), (VII), (VIIa), (VIII), (IX) or any sub-group or embodiment thereof as defined herein for the manufacture of a medicament for the prophylaxis or treatment of a disease state or condition mediated by protein kinase A.
A method for the prophylaxis or treatment of a disease state or condition mediated by protein kinase A, which method comprises administering to a subject in need thereof a compound of the formula (I), (Ia), (Ib), (II), (IIa), (IIb), (IIc), (III), (IIIa), (IV), (V), (VI), (VII), (VIIa), (VIII), (IX) or any sub-group or embodiment thereof as defined herein.
A method for treating a disease or condition comprising or arising from abnormal cell growth or abnormally arrested cell death in a mammal, the method comprising administering to the mammal a compound of the formula (I), (Ia), (Ib), (II), (IIa), (IIb), (IIc), (III), (IIIa), (IV), (V), (VI), (VII), (VIIa), (VIII), (IX) or any sub-group or embodiment thereof as defined herein in an amount effective to inhibit protein kinase A activity.
A method of inhibiting protein kinase A, which method comprises contacting the kinase with a kinase-inhibiting compound of the formula (I), (Ia), (Ib), (II), (IIa), (IIb), (IIc), (III), (IIIa), (IV), (V), (VI), (VII), (VIIa), (VIII), (IX) or any sub-group or embodiment thereof as defined herein.
A method of modulating a cellular process (for example cell division) by inhibiting the activity of a protein kinase A using a compound of the formula (I), (Ia), (Ib), (II), (IIa), (IIb), (IIc), (III), (IIIa), (IV), (V), (VI), (VII), (VIIa), (VIII), (IX) or any sub-group or embodiment thereof as defined herein.
The use of a compound of the formula (I), (Ia), (Ib), (II), (IIa), (III), (IIIa), (IV), (V), (VI), (VII), (VIIa), (VIII), (IX) or any sub-group thereof as defined herein for the manufacture of a medicament for the prophylaxis or treatment of a disease state or condition arising from abnormal cell growth or abnormally arrested cell death.
A method for treating a disease or condition comprising or arising from abnormal cell growth in a mammal, which method comprises administering to the mammal a compound of the formula (I), (Ia), (Ib), (II), (IIa), (IIb), (IIc), (III), (IIIa), (IV), (V), (VI), (VII), (VIIa), (VIII), (IX) or any sub-group thereof as defined herein in an amount effective in inhibiting abnormal cell growth or abnormally arrested cell death.
A method for alleviating or reducing the incidence of a disease or condition comprising or arising from abnormal cell growth or abnormally arrested cell death in a mammal, which method comprises administering to the mammal a compound of the formula (I), (Ia), (Ib), (II), (IIa), (IIb), (IIc), (III), (IIIa), (IV), (V), (VI), (VII), (VIIa), (VIII), (IX) or any sub-group thereof as defined herein in an amount effective in inhibiting abnormal cell growth.
A pharmaceutical composition comprising a novel compound of the formula (I), (Ia), (Ib), (II), (IIa), (IIb), (IIc), (III), (IIIa), (IV), (V), (VI), (VII), (VIIa), (VIII), (IX) or any sub-group thereof as defined herein and a pharmaceutically acceptable carrier.
A compound of the formula (I), (Ia), (Ib), (II), (IIa), (IIb), (IIc), (III), (IIIa), (IV), (V), (VI), (VII), (VIIa), (VIIa), (VIII), (IX) or any sub-group thereof as defined herein for use in medicine.
A compound of the formula (I), (Ia), (Ib), (II), (IIa), (IIb), (IIc), (III), (IIIa), (IV), (V), (VI), (VII), (VIIa), (VIII), (IX) or any sub-group thereof as defined herein for the prophylaxis or treatment of any one of the disease states or conditions disclosed herein.
The use of a compound of the formula (I), (Ia), (Ib), (II), (IIa), (IIc), (III), (IIIa), (IV), (V), (VI), (VII), (VIIa), (VIII), (IX) or any sub-group thereof as defined herein for the manufacture of a medicament for the prophylaxis or treatment of any one of the disease states or conditions disclosed herein.
A method for the treatment or prophylaxis of any one of the disease states or conditions disclosed herein, which method comprises administering to a patient (e.g. a patient in need thereof) a compound (e.g. a therapeutically effective amount) of the formula (I), (Ia), (Ib), (II), (IIa), (IIb), (IIc), (III), (IIIa), (IV), (V), (VI), (VII), (VIIa), (VIII), (IX) or any sub-group thereof as defined herein.
A method for alleviating or reducing the incidence of a disease state or condition disclosed herein, which method comprises administering to a patient (e.g. a patient in need thereof) a compound (e.g. a therapeutically effective amount) of the formula (I), (Ia), (Ib), (II), (IIa), (IIb), (IIc), (III), (IIIa), (IV), (V), (VI), (VII), (VIIa), (VIII), (IX) or any sub-group thereof as defined herein.
A method for the diagnosis and treatment of a disease state or condition mediated by protein kinase B, which method comprises (i) screening a patient to determine whether a disease or condition from which the patient is or may be suffering is one which would be susceptible to treatment with a compound having activity against protein kinase B; and (ii) where it is indicated that the disease or condition from which the patient is thus susceptible, thereafter administering to the patient a compound of the formula (I), (Ia), (Ib), (II), (IIa), (IIb), (IIc), (III), (IIIa), (IV), (V), (VI), (VII), (VIIa), (VIII), (IX) or any sub-group thereof as defined herein.
A compound of the formula (I), (Ia), (Ib), (II), (IIa), (IIb), (IIc), (III), (IIIa), (IV), (V), (VI), (VII), (VIIa), (VIII), (IX) or any sub-group thereof as defined herein for the treatment or prophylaxis of a disease state or condition in a patient who has been screened and has been determined as suffering from, or being at risk of suffering from, a disease or condition which would be susceptible to treatment with a compound having activity against protein kinase B.
The use of a compound of the formula (I), (Ia), (Ib), (II), (IIa), (IIb), (IIc), (III), (IIIa), (IV), (V), (VI), (VII), (VIIa), (VIII), (IX) or any sub-group thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against protein kinase B.
A method for the diagnosis and treatment of a disease state or condition mediated by protein kinase A, which method comprises (i) screening a patient to determine whether a disease or condition from which the patient is or may be suffering is one which would be susceptible to treatment with a compound having activity against protein kinase A; and (ii) where it is indicated that the disease or condition from which the patient is thus susceptible, thereafter administering to the patient a compound of the formula (I), (Ia), (Ib), (II), (IIa), (IIb), (IIc), (III), (IIIa), (IV), (V), (VI), (VII), (VIIa), (VIII), (IX) or any sub-group or embodiment thereof as defined herein.
A compound of the formula (I), (Ia), (Ib), (II), (IIa), (IIb), (IIc), (III), (IIIa), (IV), (V), (VI), (VII), (VIIa), (VIII), (IX) or any sub-group or embodiment thereof as defined herein for the treatment or prophylaxis of a disease state or condition in a patient who has been screened and has been determined as suffering from, or being at risk of suffering from, a disease or condition which would be susceptible to treatment with a compound having activity against protein kinase A.
The use of a compound of the formula (I), (Ia), (Ib), (II), (IIa), (IIb), (IIc), (III), (IIIa), (IV), (V), (VI), (VII), (VIIa), (VIII), (IX) or any sub-group or embodiment thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against protein kinase A.

### GENERAL PREFERENCES AND DEFINITIONS

In this specification, the structural drawings have been prepared using the ISIS Draw Program.

In certain cases, hydrogen atoms may not be shown but are merely implied. Thus, for example, some amino and hydroxy groups may appear simply as:
—N—, —N or —O

Where such apparently incomplete structures are shown, they are to be interpreted as though the hydrogen atoms are present, unless the context requires otherwise.

The following general preferences and definitions shall apply to each of the moieties A, E, G and the R groups and any sub-definition, sub-group or embodiment thereof, unless the context indicates otherwise.

Any references to Formula (I) herein shall be taken also to refer to formulae (Ia), (Ib), (II), (IIa), (IIb), (IIc), (III), (IIIa), (IV), (V), (VI), (VII), (VIIa), (VIII), (IX) and any other sub-group of compounds within formula (I) unless the context requires otherwise.

In this specification, the use of a letter following the number in an “R number” (“R group”) indicates that the group or substituent in question is a subset of the group or substituent defined by the “R number” without the letter following the number. For example, in formula (I), the R groups R^{1a}, R^{1b}, R^{1c} and R^{1d} are all subsets of R¹. Similarly, the R groups R^{2a} and R^{3a} are subsets of R² and R³ respectively. Unless the context indicates otherwise, references herein to preferences, embodiments and examples of a group (an “R group”) or substituent defined by an “R number” apply also to the aforementioned subsets thereof.

References to “carbocyclic” and “heterocyclic” groups as used herein shall, unless the context indicates otherwise, include both aromatic and non-aromatic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, and preferably 5 or 6 ring members. Examples of bicyclic groups are those containing 8, 9, 10, 11 and 12 ring members, and more usually 9 or 10 ring members.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or by a non-aromatic ring. The aryl or heteroaryl groups can be monocyclic or bicyclic groups and can be unsubstituted or substituted with one or more substituents, for example one or more groups R¹⁰ as defined herein.

The term non-aromatic group embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C═C or N═C bond. The term “fully saturated” refers to rings where there are no multiple bonds between ring atoms. Saturated carbocyclic groups include cycloalkyl groups as defined below. Partially saturated carbocyclic groups include cycloalkenyl groups as defined below, for example cyclopentenyl, cycloheptenyl and cyclooctenyl.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a 5- or 6-membered ring containing 1, 2 or 3 ring heteroatoms;
e) a pyrazole ring fused to a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered ring containing 1, 2 or 3 ring heteroatoms; and
o) a cyclopentyl ring fused to a 5- or 6-membered ring containing 1, 2 or 3 ring heteroatoms.

Examples of bicyclic heteroaryl groups containing a six membered ring fused to a five membered ring include but are not limited to benzfuran, benzthiophene, benzimidazole, benzoxazole, benzisoxazole, benzthiazole, benzisothiazole, isobenzofuran, indole, isoindole, indolizine, indoline, isoindoline, purine (e.g., adenine, guanine), indazole, benzodioxole and pyrazolopyridine groups.

Examples of bicyclic heteroaryl groups containing two fused six membered rings include but are not limited to quinoline, isoquinoline, chroman, thiochroman, chromene, isochromene, isochroman, benzodioxan, quinolizine, benzoxazine, benzodiazine, pyridopyridine, quinoxaline, quinazoline, cinnoline, phthalazine, naphthyridine and pteridine groups.

Examples of polycyclic aryl and heteroaryl groups containing an aromatic ring and a non-aromatic ring include tetrahydronaphthalene, tetrahydroisoquinoline, tetrahydroquinoline, dihydrobenzothiene, dihydrobenzofuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphthyl, indenyl, and tetrahydronaphthyl groups.

Examples of non-aromatic heterocyclic groups are groups having from 3 to 12 ring members, more usually 5 to 10 ring members. Such groups can be monocyclic or bicyclic, for example, and typically have from 1 to 5 heteroatom ring members (more usually 1, 2, 3 or 4 heteroatom ring members), usually selected from nitrogen, oxygen and sulphur.

The heterocylic groups can contain, for example, cyclic ether moieties (e.g as in tetrahydrofuran and dioxane), cyclic thioether moieties (e.g. as in tetrahydrothiophene and dithiane), cyclic amine moieties (e.g. as in pyrrolidine), cyclic sulphones (e.g. as in sulpholane and sulpholene), cyclic sulphoxides, cyclic sulphonamides and combinations thereof (e.g. thiomorpholine). Other examples of non-aromatic heterocyclic groups include cyclic amide moieties (e.g. as in pyrrolidone) and cyclic ester moieties (e.g. as in butyrolactone).

Examples of monocyclic non-aromatic heterocyclic groups include 5-, 6- and 7-membered monocyclic heterocyclic groups. Particular examples include morpholine, thiomorpholine and its S-oxide and S,S-dioxide (particularly thiomorpholine), piperidine (e.g. 1-piperidinyl, 2-piperidinyl 3-piperidinyl and 4-piperidinyl), N-alkyl piperidines such as N-methyl piperidine, piperidone, pyrrolidine (e.g. 1-pyrrolidinyl, 2-pyrrolidinyl and 3-pyrrolidinyl), pyrrolidone, azetidine, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazone, piperazine, and N-alkyl piperazines such as N-methyl piperazine, N-ethyl piperazine and N-isopropylpiperazine.

One sub-group of monocyclic non-aromatic heterocyclic groups includes morpholine, piperidine (e.g. 1-piperidinyl, 2-piperidinyl 3-piperidinyl and 4-piperidinyl), piperidone, pyrrolidine (e.g. 1-pyrrolidinyl, 2-pyrrolidinyl and 3-pyrrolidinyl), pyrrolidone, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazone, piperazine, and N-alkyl piperazines such as N-methyl piperazine. In general, preferred non-aromatic heterocyclic groups include piperidine, pyrrolidine, azetidine, morpholine, piperazine and N-alkyl piperazines. A further particular example of a non-aromatic heterocyclic group, which also forms part of the above group of preferred non-aromatic heterocyclic groups, is azetidine.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Each of the definitions of carbocyclic and heterocyclic groups in this specification may optionally exclude any one or any combination of two or more of the following moieties:
substituted or unsubstituted pyridone rings;
substituted or unsubstituted pyrrolo[1,2-a]pyrimid-4-ones;
substituted or unsubstituted pyrazolones.

Where reference is made herein to carbocyclic and heterocyclic groups, the carbocyclic or heterocyclic ring can, unless the context indicates otherwise, be unsubstituted or substituted by one or more substituent groups R¹⁰ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{o} is selected from hydrogen and C₁₋₄ hydrocarbyl; and
X¹ is O, S or NR^{c} and X² is ═O, ═S or ═NR^{c}.

Where the substituent group R¹⁰ comprises or includes a carbocyclic or heterocyclic group, the said carbocyclic or heterocyclic group may be unsubstituted or may itself be substituted with one or more further substituent groups R¹⁰. In one sub-group of compounds of the formula (I), such further substituent groups R¹⁰ may include carbocyclic or heterocyclic groups, which are typically not themselves further substituted. In another sub-group of compounds of the formula (I), the said further substituents do not include carbocyclic or heterocyclic groups but are otherwise selected from the groups listed above in the definition of R¹⁰.

The substituents R¹⁰ may be selected such that they contain no more than 20 non-hydrogen atoms, for example, no more than 15 non-hydrogen atoms, e.g. no more than 12, or 10, or 9, or 8, or 7, or 6, or 5 non-hydrogen atoms.

Where the carbocyclic and heterocyclic groups have a pair of substituents on adjacent ring atoms, the two substituents may be linked so as to form a cyclic group. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group. Examples of such linked substituent groups include:

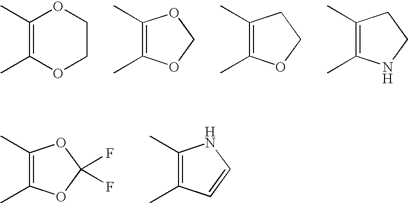

Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone, except where otherwise stated. In certain cases, as defined herein, one or more of the carbon atoms making up the carbon backbone may be replaced by a specified atom or group of atoms. Examples of hydrocarbyl groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or, where stated, can be substituted by one or more substituents as defined herein. The examples and preferences expressed below apply to each of the hydrocarbyl substituent groups or hydrocarbyl-containing substituent groups referred to in the various definitions of substituents for compounds of the formula (I) unless the context indicates otherwise.

Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups), specific examples being any individual value or combination of values selected from C₁, C₂, C₃, C₄, C₅, C₆, C₇ and C₈ hydrocarbyl groups.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl (vinyl), 1-propenyl, 2-propenyl (allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8.carbon atoms, and particular examples are C₃₋₆ cycloalkenyl groups.

Examples of alkynyl groups include, but are not limited to, ethynyl and 2-propynyl (propargyl) groups. Within the sub-set of alkynyl groups having 2 to 8 carbon atoms, particular examples are C₂₋₆ alkynyl groups, such as C₂₋₄ alkynyl groups.

Examples of carbocyclic aryl groups include substituted and unsubstituted phenyl, naphthyl, indane and indene groups.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups.

The terms C₁₋₈ hydrocarbyl as used herein encompasses alkyl, alkenyl, alkynyl, cycloalkyl, cycloalkenyl, phenyl, benzyl and phenylethyl groups wherein the preferences for and examples of each of the aforesaid groups are as defined above. Within this definition, particular hydrocarbyl groups are alkyl, cycloalkyl, phenyl, benzyl and phenylethyl (e.g. 1-phenylethyl or 2-phenylethyl) groups, one subset of hydrocarbyl groups consisting of alkyl and cycloalkyl groups and in particular C₁₋₄ alkyl and cycloalkyl groups such as methyl, ethyl, n-propyl, isopropyl, n-butyl, isobutyl, tert-butyl, cyclopropyl and cyclobutyl.

The term C₁₋₄ hydrocarbyl as used herein encompasses alkyl, alkenyl, alkynyl, cycloalkyl and cycloalkenyl groups wherein the preferences for and examples of the aforesaid groups are as defined above. Within this definition, particular C₁₋₄ hydrocarbyl groups are alkyl and cycloalkyl groups, such as methyl, ethyl, n-propyl, isopropyl, n-butyl, isobutyl, tert-butyl, cyclopropyl and cyclobutyl.

The term C₁₋₅ saturated hydrocarbyl as used herein encompasses alkyl and cycloalkyl groups having 1 to 5 carbon atoms. Within this definition, particular C₁₋₅ saturated hydrocarbyl groups are methyl, ethyl, n-propyl, isopropyl, n-butyl, isobutyl, tert-butyl, cyclopropyl and cyclobutyl.

When present, and where stated, a hydrocarbyl group can be optionally substituted by one or more substituents selected from hydroxy, oxo, alkoxy, carboxy, halogen, cyano, nitro, amino, mono- or di-C₁₋₄ hydrocarbylamino, and monocyclic or bicyclic carbocyclic and heterocyclic groups having from 3 to 12 (typically 3 to 10 and more usually 5 to 10) ring members. Preferred substituents include halogen such as fluorine. Thus, for example, the substituted hydrocarbyl group can be a partially fluorinated or perfluorinated group such as difluoromethyl or difluoromethyl. In one embodiment preferred substituents include monocyclic carbocyclic and heterocyclic groups having 3-7 ring members.

Where present and where stated, one or more carbon atoms of a hydrocarbyl group may optionally be replaced by O, S, SO, SO₂, NR^{c}, X¹C(X²), C(X²)X¹ or X¹C(X²)X¹ (or a sub-group thereof) wherein X¹ and X² are as hereinbefore defined, provided that at least one carbon atom of the hydrocarbyl group remains. For example, 1, 2, 3 or 4 carbon atoms of the hydrocarbyl group may be replaced by one of the atoms or groups listed, and the replacing atoms or groups may be the same or different. In general, the number of linear or backbone carbon atoms replaced will correspond to the number of linear or backbone atoms in the group replacing them. Examples of groups in which one or more carbon atom of the hydrocarbyl group have been replaced by a replacement atom or group as defined above include ethers and thioethers (C replaced by O or S), amides, esters, thioamides and thioesters (C—C replaced by X¹C(X²) or C(X²)X¹), sulphones and sulphoxides (C replaced by SO or SO₂), amines (C replaced by NR^{c}). Further examples include ureas, carbonates and carbamates (C—C—C replaced by X¹C(X²)X¹).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members.

The definition “R^{a}—R^{b}” as used herein, either with regard to substituents present on a carbocyclic or heterocyclic moiety, or with regard to other substituents present at other locations on the compounds of the formula (I), includes inter alia compounds wherein IV is selected from a bond, O, CO, OC(O), SC(O), NR^{c}C(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}C(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O)OC(S)NR^{c}, SC(S) NR^{c}C(S)NR^{c}, OC(NR^{c})NR^{c}, SC(NR^{c})NR^{c}, NR^{c}C(NR^{c}NR^{c}, S, SO, SO₂NR^{c}, SO₂NR^{c} and NR^{c}SO₂ wherein R¹ is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

When R^{a} is O and R^{b} is a C₁₋₄ hydrocarbyl group, R^{a} and R^{b} together form a hydrocarbyloxy group. Preferred hydrocarbyloxy groups include saturated hydrocarbyloxy such as alkoxy (e.g. C₁₋₆ alkoxy, more usually C₁₋₄ alkoxy such as ethoxy and methoxy, particularly methoxy), cycloalkoxy (e.g. C₃₋₆ cycloalkoxy such as cyclopropyloxy, cyclobutyloxy, cyclopentyloxy and cyclohexyloxy) and cycloalkyalkoxy (e.g. C₃₋₆ cycloalkyl-C₁₋₂ alkoxy such as cyclopropylmethoxy).

The hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), alkoxy (e.g. as in methoxyethoxy), hydroxy-C₁₋₂ alkyl (as in hydroxyethoxyethoxy) or a cyclic group (e.g. a cycloalkyl group or non-aromatic heterocyclic group as hereinbefore defined). Examples of alkoxy groups bearing a non-aromatic heterocyclic group as a substituent are those in which the heterocyclic group is a saturated cyclic amine such as morpholine, piperidine, pyrrolidine, piperazine, C₁₋₄-alkyl-piperazines, C₃₋₄-cycloalkyl-piperazines, tetrahydropyran or tetrahydrofuran and the alkoxy group is a C₁₋₄ alkoxy group, more typically a C₁₋₃ alkoxy group such as methoxy, ethoxy or n-propoxy.

Alkoxy groups may be substituted by, for example, a monocyclic group such as pyrrolidine, piperidine, morpholine and piperazine and N-substituted derivatives thereof such as N-benzyl, N—C₁₋₄ acyl and N—C₁₋₄ alkoxycarbonyl. Particular examples include pyrrolidinoethoxy, piperidinoethoxy and piperazinoethoxy.

When R^{a} is a bond and R^{b} is a C₁₋₈ hydrocarbyl group, examples of hydrocarbyl groups R^{a}—R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine (particular examples including bromoethyl, chloroethyl, difluoromethyl, 2,2,2-trifluoroethyl and perfluoroalkyl groups such as trifluoromethyl), or hydroxy (e.g. hydroxymethyl and hydroxyethyl), C₁₋₈ acyloxy (e.g. acetoxymethyl and benzyloxymethyl), amino and mono- and dialkylamino (e.g. aminoethyl, methylaminoethyl, dimethylaminomethyl, dimethylaminoethyl and tert-butylaminomethyl), alkoxy (e.g. C₁₋₂ alkoxy such as methoxy—as in methoxyethyl), and cyclic groups such as cycloalkyl groups, aryl groups, heteroaryl groups and non-aromatic heterocyclic groups as hereinbefore defined).

Particular examples of alkyl groups substituted by a cyclic group are those wherein the cyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkyl group is a C₁₋₄ alkyl group, more typically a C₁₋₃ alkyl group such as methyl, ethyl or n-propyl. Specific examples of alkyl groups substituted by a cyclic group include pyrrolidinomethyl, pyrrolidinopropyl, morpholinomethyl, morpholinoethyl, morpholinopropyl, piperidinylmethyl, piperazinomethyl and N-substituted forms thereof as defined herein.

Particular examples of alkyl groups substituted by aryl groups and heteroaryl groups include benzyl, phenethyl and pyridylmethyl groups.

When R^{a} is SO₂NR^{c}, R^{b} can be, for example, hydrogen or an optionally substituted C₁₋₈ hydrocarbyl group, or a carbocyclic or heterocyclic group. Examples of R^{a}—R^{b} where R^{a} is SO₂NR^{c} include aminosulphonyl, C₁₋₄ alkylaminosulphonyl and di-C₁₋₄ alkylaminosulphonyl groups, and sulphonamides formed from a cyclic amino group such as piperidine, morpholine, pyrrolidine, or an optionally N-substituted piperazine such as N-methyl piperazine.

Examples of groups R^{a}—R^{b} where R^{a} is SO₂ include alkylsulphonyl, heteroarylsulphonyl and mylsulphonyl groups, particularly monocyclic aryl and heteroaryl sulphonyl groups. Particular examples include methylsulphonyl, phenylsulphonyl and toluenesulphonyl.

When R^{a} is NR^{c}, R^{b} can be, for example, hydrogen or an optionally substituted C₁₋₈ hydrocarbyl group, or a carbocyclic or heterocyclic group. Examples of R^{a}—R^{b} where R^{a} is NR^{c} include amino, C₁₋₄ alkylamino (e.g. methylamino, ethylamino, propylamino, isopropylamino, tert-butylamino), di-C₁₋₄ alkylamino (e.g. dimethylamino and diethylamino) and cycloalkylamino (e.g. cyclopropylamino, cyclopentylamino and cyclohexylamino).

### SPECIFIC EMBODIMENTS AND PREFERENCES

### R⁴

In formula (I), R⁴ is selected from hydrogen, halogen, C₁₋₅ saturated hydrocarbyl, C₁₋₄ saturated hydrocarbyloxy, cyano, and CF₃.

Typically, R⁴ is selected from hydrogen, halogen, C₁₋₅ saturated hydrocarbyl, cyano and CF₃.

More typically, R⁴ is selected from hydrogen, methyl, ethyl, isopropyl, cyclopropyl and CF₃.

Preferred values for R⁴ include hydrogen and methyl. In a particular embodiment, R⁴ is hydrogen.

### R⁵

In formula (I), R⁵ is selected from hydrogen, halogen, C₁₋₅ saturated hydrocarbyl, C₁₋₅ saturated hydrocarbyloxy, cyano, CONH₂, CONHR⁹, CF₃, NH₂, NHCOR⁹ and NHCONHR⁹; NHCONHR⁹ where R⁹ is a group R^{9a} or (CH₂)R⁹⁸, wherein R^{9a} is an optionally substituted monocyclic or bicyclic group which may be carbocyclic or heterocyclic.

Examples of carbocyclic and heterocyclic groups are set out above in the General Preferences and Definitions section.

Typically the carbocyclic and heterocyclic groups are monocyclic.

Preferably the carbocyclic and heterocyclic groups are aromatic.

Particular examples of the group R⁹ are optionally substituted phenyl or benzyl.

Preferably, R⁵ is selected from selected from hydrogen, halogen, C₁₋₅ saturated hydrocarbyl, cyano, CONH₂, CONHR⁹, CF₃, NH₂, NHCOR⁹ and NHCONHR⁹ where R⁹ is optionally substituted phenyl or benzyl.

More preferably, R⁵ is selected from selected from hydrogen, halogen, C₁₋₅ saturated hydrocarbyl, cyano, CF₃, NH₂, NHCOR⁹ and NHCONHR⁹ where R⁹ is optionally substituted phenyl or benzyl.

The group R⁹ is typically unsubstituted phenyl or benzyl, or phenyl or benzyl substituted by 1,2 or 3 substituents selected from halogen; hydroxy; trifluoromethyl; cyano; carboxy; C₁₋₄alkoxycarbonyl; C₁₋₄ acyloxy; amino; mono- or di-C₁₋₄ alkylamino; C₁₋₄ alkyl optionally substituted by halogen, hydroxy or C₁₋₂ alkoxy; C₁₋₄ alkoxy optionally substituted by halogen, hydroxy or C₁₋₂ alkoxy; phenyl, five and six membered heteroaryl groups containing up to 3 heteroatoms selected from O, N and S; and saturated carbocyclic and heterocyclic groups containing up to 2 heteroatoms selected from O, S and N.

Particular examples of the moiety R⁵ include hydrogen, fluorine, chlorine, bromine, methyl, ethyl, hydroxyethyl, methoxymethyl, cyano, CF₃, NH₂, NHCOR^{9b} and NHCONHR^{9b} where R^{9b} is phenyl or benzyl optionally substituted by hydroxy, C₁₋₄ acyloxy, fluorine, chlorine, bromine, trifluoromethyl, cyano, C₁₋₄ hydrocarbyloxy (e.g. alkoxy) and C₁₋₄ hydrocarbyl (e.g. alkyl) optionally substituted by C₁₋₂ alkoxy or hydroxy.

In one preferred embodiment, R⁵ is selected from hydrogen, halogen, C₁₋₅ saturated hydrocarbyl, cyano and CF₃.

Preferred examples of R⁵ include hydrogen, methyl, ethyl, isopropyl, cyclopropyl, trifluoromethyl and cyano.

In one embodiment, R⁵ is hydrogen, methyl or cyano. In another embodiment, R⁵ is hydrogen or methyl.

### The Group “E”

In formula (I), E is a monocyclic or bicyclic carbocyclic or heterocyclic group and can be selected from the groups set out above in the section headed General Preferences and Definitions.

Preferred groups E are monocyclic and bicyclic aryl and heteroaryl groups and, in particular, groups containing a six membered aromatic or heteroaromatic ring such as a phenyl, pyridine, pyrazine, pyridazine or pyrimidine ring, more particularly a phenyl, pyridine, pyrazine or pyrimidine ring, and more preferably a pyridine or phenyl ring.

Examples of bicyclic groups include benzo-fused and pyrido-fused groups wherein the group A and the pyrazole ring are both attached to the benzo- or pyrido-moiety.

In one embodiment, E is a monocyclic group.

Particular examples of monocyclic groups include monocyclic aryl and heteroaryl groups such as phenyl, thiophene, furan, pyrimidine, pyrazine and pyridine, phenyl being presently preferred.

One subset of monocyclic aryl and heteroaryl groups comprises phenyl, thiophene, furan, pyrimidine and pyridine.

Examples of non-aromatic monocyclic groups include cycloalkanes such as cyclohexane and cyclopentane, and nitrogen-containing rings such as piperazine and piperazone.

It is preferred that the group A and the pyrazole group are not attached to adjacent ring members of the group E. For example, the pyrazole group can be attached to the group E in a meta or para relative orientation. Examples of such groups E include 1,4-phenylene, 1,3-phenylene, 2,5-pyridylene and 2,4-pyridylene, 1,4-piperazinyl, and 1,4-piperazonyl. Further examples include 1,3-disubstituted five membered rings .

The groups E can be unsubstituted or can have up to 4 substituents R⁸ which may be selected from the group R¹⁰ as hereinbefore defined. More typically however, the substituents R⁸ are selected from hydroxy; oxo (when E is non-aromatic); halogen (e.g. chlorine and bromine; or fluorine); trifluoromethyl; cyano; C₁₋₄ hydrocarbyloxy optionally substituted by C₁₋₂ alkoxy or hydroxy; and C₁₋₄ hydrocarbyl optionally substituted by C₁₋₂ alkoxy or hydroxy.

Preferably there are 0-3 substituents, more preferably 0-2 substituents, for example 0 or 1 substituent. In one embodiment, the group E is unsubstituted.

E may be other than:
a substituted pyridone group;
a substituted thiazole group;
a substituted or unsubstituted pyrazole or pyrazolone group;
a substituted or unsubstituted bicyclic fused pyrazole group;
a phenyl ring fused to a thiophene ring or a six membered nitrogen-containing heteroaryl ring fused to a thiophene ring;
a substituted or unsubstituted piperazine group;

The group E can be an aryl or heteroaryl group having five or six members and containing up to three heteroatoms selected from O, N and S, the group E being represented by the, formula:

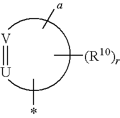

where * denotes the point of attachment to the pyrazole group, and “a” denotes the attachment of the group A;
r is 0, 1 or 2;
U is selected from N and CR^{12a}; and
V is selected from N and CR^{12b}; where R^{12a} and R^{12b} are the same or different and each is hydrogen or a substituent containing up to ten atoms selected from C, N, O, F, Cl and S provided that the total number of non-hydrogen atoms present in R^{12a} and R^{12b} together does not exceed ten;
or R^{12a} and R^{12b} together with the carbon atoms to which they are attached form an unsubstituted five or six membered saturated or unsaturated ring containing up to two heteroatoms selected from O and N; and
R¹⁰ is as hereinbefore defined.

In one preferred group of compounds, E is a group:

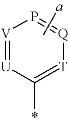

where * denotes the point of attachment to the pyrazole group, and “a” denotes the attachment of the group A;
P, Q and T are the same or different and are selected from N, CH and NCR¹⁰, provided that the group A is attached to a carbon atom; and U, V and R¹⁰ are as hereinbefore defined.

Examples of R^{12a} and R^{12b} include hydrogen and substituent groups R¹⁰ as hereinbefore defined having no more than ten non-hydrogen atoms. Particular examples of R^{12a} and R^{12b} include methyl, ethyl, propyl, isopropyl, cyclopropyl, cyclobutyl, cyclopentyl, fluorine, chlorine, methoxy, trifluoromethyl, hydroxymethyl, hydroxyethyl, methoxymethyl, difluoromethoxy, trifluoromethoxy, 2,2,2-trifluoroethyl, cyano, amino, methylamino, dimethylamino, CONH₂, CO₂Et, CO₂H, acetamido, azetidinyl, pyrrolidino, piperidine, piperazino, morpholino, methylsulphonyl, aminosulphonyl, mesylamino and trifluoroacetamido.

Preferably, when U is CR^{12a} and/or V is CR^{12b} the atoms or groups in R^{12a} and R^{12b} that are directly attached to the carbon atom ring members C are selected from H, O (e.g. as in methoxy), NH (e.g. as in amino and methylamino) and CH₂ (e.g. as in methyl and ethyl).

Particular examples of the linker group E, together with their points of attachment to the group A (*) and the pyrazole ring (*) are shown in Table 1 below.

TABLE 1

| | |
|---|---|
| 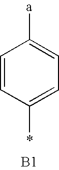 | 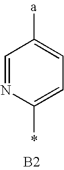 |
| 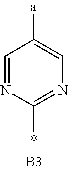 | 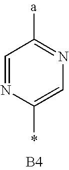 |
| 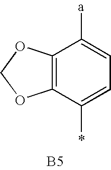 | 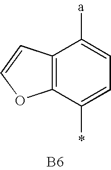 |
| 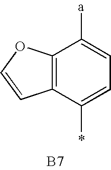 | 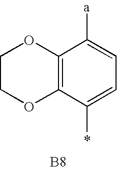 |
| 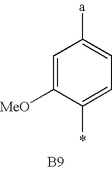 | 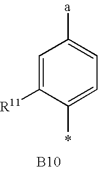 |
| 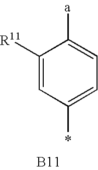 | 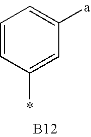 |
| 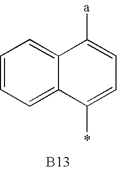 | 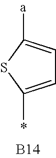 |
| 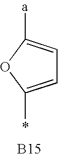 | 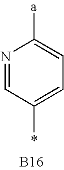 |

In the table, the substituent group R¹¹ is selected from methyl, chlorine, fluorine and trifluoromethyl.

Preferred groups E are groups B1, B2, B11 and B16, particularly B1.

The following optional exclusions may apply to the definition of E in formula (I) and any sub-groups or sub-definitions thereof as defined herein:
E may be other than a phenyl group having a sulphur atom attached to the position para with respect to the pyrazole group.
E may be other than a substituted or unsubstituted benzimidazole, benzoxazole or benzthiazole group.

### The Group J¹

In one embodiment, the group J¹ is:

(1) a group having the formula:

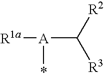

wherein the asterisk denotes the point of attachment to the group E; A is as defined herein; and R^{1a} is an aryl or heteroaryl group selected from groups R^{1b}, R^{1c} and R^{1d}.

### The Group “A”

In formula (I), A is a saturated hydrocarbon linker group containing from 1 to 7 carbon atoms, the linker group having a maximum chain length of 5 atoms extending between R^{1a} and NR²R³ and a maximum chain length of 4 atoms extending between E and NR²R³. Within these constraints, the moieties E and R^{1a} can each be attached at any location on the group A.

The term “maximum chain length” as used herein refers to the number of atoms lying directly between the two moieties in question, and does not take into account any branching in the chain or any hydrogen atoms that may be present. For example, in the structure A shown below:

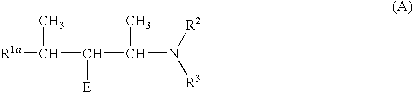

the chain length between R^{1a} and NR²R³ is 3 atoms whereas the chain length between E and NR²R³ is 2 atoms.

In general it is presently preferred that the linker group has a maximum chain length of 3 atoms (for example 1 or 2 atoms).

In one embodiment, the linker group has a chain length of 1 atom extending between R^{1a} and NR²R³.

In another embodiment, the linker group has a chain length of 2 atoms extending between R^{1a} and NR²R³.

In a further embodiment, the linker group has a chain length of 3 atoms extending between R^{1a} and NR²R³.

It is preferred that the linker group has a maximum chain length of 3 atoms extending between E and NR²R³.

In one particularly preferred group of compounds, the linker group has a chain length of 2 or 3 atoms extending between R^{1a} and NR²R³ and a chain length of 2 or 3 atoms extending between E and NR²R³.

One of the carbon atoms in the linker group may optionally be replaced by an oxygen or nitrogen atom.

When present, the nitrogen atom may be linked directly to the group E.

In one embodiment, the carbon atom to which the group R^{1a} is attached is replaced by an oxygen atom.

In another embodiment, R^{1a} and E are attached to the same carbon atom of the linker group, and a carbon atom in the chain extending between E and NR²R³ is replaced by an oxygen atom.

When a nitrogen atom or oxygen atom are present, it is preferred that the nitrogen or oxygen atom and the NR²R³ group are spaced apart by at least two intervening carbon atoms.

In one particular group of compounds within formula (I), the linker atom linked directly to the group E is a carbon atom and the linker group A has an all-carbon skeleton.

The carbon atoms of the linker group A may optionally bear one or more substituents selected from oxo, fluorine and hydroxy, provided that the hydroxy group is not located at a carbon atom a with respect to the NR²R³ group, and provided also that the oxo group is located at a carbon atom a with respect to the NR²R³ group. Typically, the hydroxy group, if present, is located at a position β with respect to the NR²R³ group. In general, no more than one hydroxy group will be present. Where fluorine is present, it may be present as a single fluorine substituent or may be present in a difluoromethylene or trifluoromethyl group, for example. In one embodiment, a fluorine atom is located at a position β with respect to the NR²R³ group.

It will be appreciated that that when an oxo group is present at the carbon atom adjacent the NR²R³ group, the compound of the formula (I) will be an amide.

In one embodiment of the invention, no fluorine atoms are present in the linker group A.

In another embodiment of the invention, no hydroxy groups are present in the linker group A.

In a further embodiment, no oxo group is present in the linker group A.

In one group of compounds of the formula (I) neither hydroxy groups nor fluorine atoms are present in the linker group A, e.g. the linker group A is unsubstituted.

Preferably, when a carbon atom in the linker group A is replaced by a nitrogen atom, the group A bears no more than one hydroxy substituent and more preferably bears no hydroxy substituents.

When there is a chain length of four atoms between E and NR²R³, it is preferred that the linker group A contains no nitrogen atoms and more preferably has an all carbon skeleton.

In order to modify the susceptibility of the compounds to metabolic degradation in vivo, the linker group A can have a branched configuration at the carbon atom attached to the NR²R³ group. For example, the carbon atom attached to the NR²R³ group can be attached to a pair of gem-dimethyl groups.

In one particular group of compounds of the formula (I), the portion R^{1a}-A-NR²R³ of the compound is represented by the formula R^{1a}-(G)ₖ-(CH₂)ₘ—W—O_{b}—(CH₂)ₙ—(CR⁶R⁷)ₚ—NR²R³ wherein G is NH, NMe or O; W is attached to the group E and is selected from (CH₂)ⱼ—CR²⁰, (CH₂)ⱼ—N and (NH)ⱼ—CH; b is 0 or 1, j is 0 or 1, k is 0 or l, m is 0 or 1, n is 0, 1, 2, or 3 and p is 0 or 1; the sum of b and k is 0 or 1; the sum of j, k, m, n and p does not exceed 4; R⁶ and R⁷ are the same or different and are selected from methyl and ethyl, or CR⁶R⁷ forms a cyclopropyl group; and R²⁰ is selected from hydrogen, methyl, hydroxy and fluorine;

In another sub-group of compounds of the formula (I), the portion R^{1a}-A-NR²R³ of the compound is represented by the formula R^{1a}-(G)ₖ(CH₂)ₘ—X—(CH₂)ₙ—(CR⁶R⁷)ₚ—NR²R³ wherein G is NH, NMe or O; X is attached to the group E and is selected from (CH₂)ⱼ—CH, (CH₂)ⱼ—N and (NH)ⱼ—CH; j is 0 or 1, k is 0 or l, m is 0 or 1, n is 0, 1, 2, or 3 and p is 0 or 1, and the sum of j, k, m, n and p does not exceed 4; and R⁶ and R⁷ are the same or different and are selected from methyl and ethyl, or CR⁶R⁷ forms a cyclopropyl group.

A particular group CR⁶R⁷ is C(CH₃)₂.

Preferably X is (CH₂); —CH.

Particular configurations where the portion R^{1a}-A-NR²R³ of the compound is represented by the formula R^{1a}-(G)ₖ—(CH₂)ₘ—X(CH₂).(CR⁶R⁷)ₚ—NR²R³ are those wherein:
k is 0, m is 0 or 1, n is 0,1, 2 or 3 and p is 0.
k is 0, m is 0 or 1, n is 0, 1 or 2 and p is 1.
X is (CH₂)ⱼ—CH, k is 1, m is 0, n is 0, 1, 2 or 3 and p is 0.
X is (CH₂)ⱼ—CH, k is 1, m is 0, n is 0, 1 or 2 and p is 1.
X is (CH₂)ⱼ—CH, is O, k is 1, m is 0,n is 0, 1, 2 or 3 and p is 0.

Particular configurations wherein the portion R^{1a}-A-NR²R³ of the compound is represented by the formula R^{1a}-(G)ₖ-(CH₂)ₘ—W—O_{b}—(CH₂)ₙ—(CR⁶R⁷)ₚ—NR²R³ are those wherein:
k is 0, m is 0, W is (CH₂)ⱼ—CR²⁰, j is 0, R²⁰ is hydrogen, b is 1, n is 2 and p is 0.
k is 0, m is 0, W is (CH₂)ⱼ—CR²⁰, j is 0, R²⁰ is hydroxy, b is 0, n is 1 and p is 0.
k is 0, m is 0, W is (CH₂)ⱼ—CR²⁰, j is 0, R²⁰ is methyl, b is 0, n is 1 and p is 0.
k is 0, m is 0, W is (CH₂)ⱼ—CR²⁰, j is 0, R²⁰ is fluorine, b is 0, n is 1 and p is 0.

In one preferred configuration, the portion R^{1a}-A-NR²R³ of the compound is represented by the formula R^{1a}—X—(CH₂)ₙ—NR²R³ wherein X is attached to the group E and is a group CH, and n is 2.

Particular examples of the linker group A, together with their points of attachment to the groups R^{1a}, E and NR²R³, are shown in Table 2 below.

TABLE 2

| | |
|---|---|
| 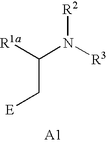 | 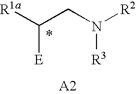 |
| 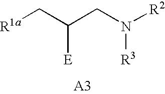 | 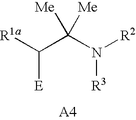 |
| 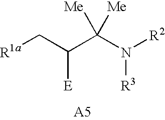 | 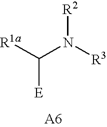 |
| 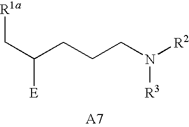 | 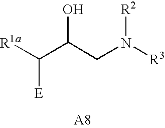 |
| 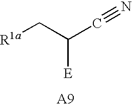 | 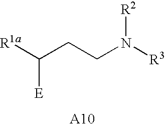 |
| 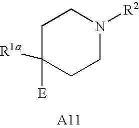 | 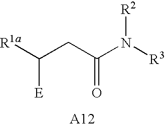 |
| 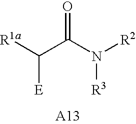 | 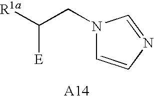 |
| 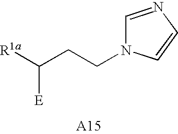 | 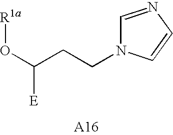 |
| 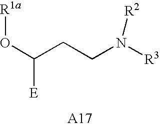 | 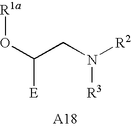 |
| 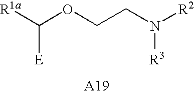 | 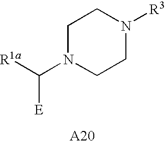 |
| 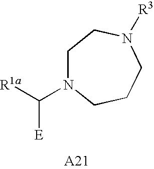 | 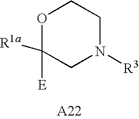 |
| 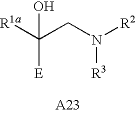 | 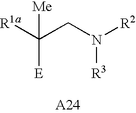 |
| 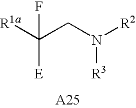 | 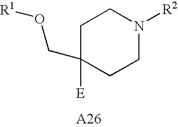 |
| 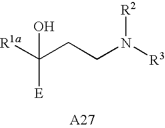 | 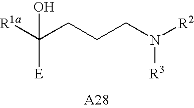 |
| 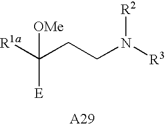 | 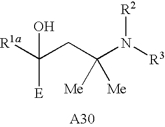 |
| 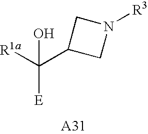 | 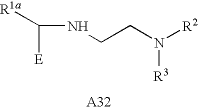 |

One set of groups consists of groups A1 to A25.

Another set of groups consists of groups A1 to A27

A further set of groups consists of groups A1 to A32

Currently preferred groups include A1, A2, A3, A6, A10, All, A22, A23 and A27.

One particular set of groups includes A1, A2, A3, A10 and A11.

A further particular set of groups includes A2 and A11.

Another particular set of groups includes A6, A22 and A23.

A further set of groups includes A1, A2 and A3.

One particularly preferred group is A27.

In group A2, the asterisk designates a chiral centre. In one embodiment, the compounds have the R configuration at this chiral centre. In another embodiment, the compounds have the S configuration at this chiral centre.

### R² and R³

In one group of compounds of the formula (I), R² and R³ are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy.

When the hydrocarbyl moiety is substituted by a hydroxy, amino, methylamino, dimethylamino or methoxy group, typically there are at least two carbon atoms between the substituent and the nitrogen atom of the group NR²R³. Particular examples of substituted hydrocarbyl groups are hydroxyethyl and hydroxypropyl.

In another group of compounds of the invention, R² and R³ are independently selected from hydrogen, C₁₋₄ hydrocarbyl and C₁₋₄ acyl.

Typically the hydrocarbyl group, whether substituted or unsubstituted, is an alkyl group, more usually a C₁, C₂ or C₃ alkyl group, and preferably a methyl group. In one particular sub-group of compounds, R² and R³ are independently selected from hydrogen and methyl and hence NR²R³ can be an amino, methylamino or dimethylamino group. In one particular embodiment, NR²R³ can be an amino group. In another particular embodiment, NR²R³ can be a methylamino group.

In an alternative embodiment, the C₁₋₄ hydrocarbyl group can be a cyclopropyl, cyclopropylmethyl or cyclobutyl group.

In another group of compounds, R² and R³ together with the nitrogen atom to which they are attached form a cyclic group selected from an imidazole group and a saturated monocyclic heterocyclic group having 4-7 ring members and optionally containing a second heteroatom ring member selected from O and N.

In a further group of compounds, R² and R³ together with the nitrogen atom to which they are attached form a saturated monocyclic heterocyclic group having 4-7 ring members and optionally containing a second heteroatom ring member selected from O and N.

The saturated monocyclic heterocyclic group can be unsubstituted or substituted by one or more substituents R¹⁰ as defined above in the General Preferences and Definitions section of this application. Typically, however, any substituents on the heterocyclic group will be relatively small substituents such as C₁₋₄ hydrocarbyl (e.g. methyl, ethyl, n-propyl, i-propyl, cyclopropyl, n-butyl, sec-butyl and tent-butyl), fluorine, chlorine, hydroxy, amino, methylamino, ethylamino and dimethylamino. Particular substituents are methyl groups.

The saturated monocyclic ring can be an azacycloalkyl group such as an azetidine, pyrrolidine, piperidine or azepane ring, and such rings are typically unsubstituted.

Alternatively, the saturated monocyclic ring can contain an additional heteroatom selected from O and N, and examples of such groups include morpholine and piperazine. Where an additional N atom is present in the ring, this can form part of an NH group or an N—C₁₋₄alkyl group such as an N-methyl, N-ethyl, N-propyl or N-isopropyl group.

Where NR²R³ forms an imidazole group, the imidazole group can be unsubstituted or substituted, for example by one or more relatively small substituents such as C₁₋₄ hydrocarbyl (e.g. methyl, ethyl, propyl, cyclopropyl and butyl), fluorine, chlorine, hydroxy, amino, methylamino, ethylamino and dimethylamino. Particular substituents are methyl groups.

In a further group of compounds, one of R² and R³ together with the nitrogen atom to which they are attached and one or more atoms from the linker group A form a saturated monocyclic heterocyclic group having 4-7 ring members and optionally containing a second heteroatom ring member selected from O and N.

Examples of such compounds include compounds wherein NR²R³ and A form a unit of the formula:

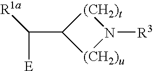

where t and u are each 0, 1, 2 or 3 provided that the sum oft and u falls within the range of 2 to 4.

Further examples of such compounds include compounds wherein NR²R³ and A form a cyclic group of the formula:

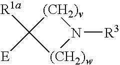

where v and w are each 0, 1, 2 or 3 provided that the sum of v and w falls within the range of 2 to 5. Particular examples of cyclic compounds are those in which v and w are both 2.

Further examples of such compounds include compounds wherein NR²R³ and A form a cyclic group of the formula:

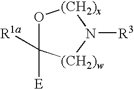

where x and w are each 0, 1, 2 or 3 provided that the sum of x and w falls within the range of 2 to 4. Particular examples of cyclic compounds are those in which x is 2 and w is 1.

In formula (I), embodiment (1), R^{1a} is an aryl or heteroaryl group selected from groups R^{1b}, R^{1c} and R^{1d}.

### R^{1b}

The group R^{1b} is represented by the formula:

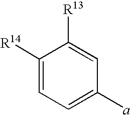

wherein “a” denotes the point of attachment to the group A.

### R¹³

The group R¹³ is selected from halogen (other than fluorine or chlorine), hydroxy, trifluoromethyl, cyano, nitro, carboxy, amino, mono- or di-C₁₋₄ hydrocarbylamino; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; provided that R^{b} is other than hydrogen when R^{a} is a bond;
R^{c} is selected from hydrogen and C₁₋₄ hydrocarbyl; and
X¹ is O, S or NR^{c} and X² is ═O, ═S or ═NR^{c}.

In one sub-group of compounds, leis selected from halogen (other than fluorine or chlorine), hydroxy, amino and a group R^{a}—R^{b} where R^{a} is selected from a bond, O, CO, C(O)O, C(O)NR^{c}, NR^{c}C(O), NR^{c}C(O)O, NR^{c}, SO, SO₂, SOW, and SO₂NR^{c}; and R^{b} is selected from hydrogen; carbocyclic and heterocyclic groups having 5 or 6 ring members; and C₁₋₈ hydrocarbyl (e.g. C₁₋₈ alkyl or C₃₋₇ cycloalkyl) optionally substituted by one or more substituents selected from hydroxy, oxo, amino, mono- or di-C₁₋₄ hydrocarbylamino, carboxy, and carbocyclic and heterocyclic groups having from 3 to 7 ring members, and wherein one or more of the carbon atoms of the C₁₋₈ hydrocarbyl group may optionally be replaced by O, S, C(O)O, C(O)NR^{o} or NR^{c}; provided that R^{b} is other than hydrogen when R^{a} is a bond.

In another sub-group of compounds, R¹³ may be selected from halogen (other than fluorine or chlorine), OH, NH₂, CH₂OH, CH₂NH₂, O—C₁₋₆-alkyl, NH—C₁₋₆ alkyl, aryl, heteroaryl, C₃₋₇ cycloalkyl, heterocyclyl, O-heteroaryl, O—C₃₋₇ cycloalkyl, O-heterocycloalkyl, C(═O)C₁₋₆ alkyl, C(═O)OC₁₋₆ alkyl, C(═O)NH₂, C(═O)NHC₁₋₆ alkyl, C(═O)N(C₁₋₈ alkyl)₂, NH(C₁₋₆ alkyl), N(C₁₋₆ alkyl)₂, NC(═O)C₁₋₆ alkyl, C₆ aryl, OC₆ aryl, C(═O)C₆aryl, C(═O)OC₆aryl, C(═O)NH₂, C(═O)NHC₆aryl, C(═O)N(C₆aryl)₂, NH(C₆ aryl), N(C₆ aryl)₂, NC(═O)C₆ aryl, C₅₋₆ heterocyclyl, OC₅₋₆heterocyclyl, C(═O)C₅₋₆heterocyclyl, C(═O)OC₅₋₆ heterocyclyl, C(═O)NHC₅₋₆heterocyclyl, C(═O)N(C₅₋₆ heterocyclyl)₂, NH(C₅₋₆ heterocyclyl), N(C₅₋₆heterocyclyl)₂, NC(═O)C₅₋₆ heterocyclyl, C(═O)NHC₁₋₆ alkyl, C₃₋₅ aryl, S(═O)C₁₋₆ alkyl, S(═O)N—C₁₋₆ alkyl and SO₂N—C₁₋₆ alkyl; and a group [sol], CH₂CH₂[sol], CH₂[sol] or OCH₂CH₂[sol] where [sol] is selected from the following groups

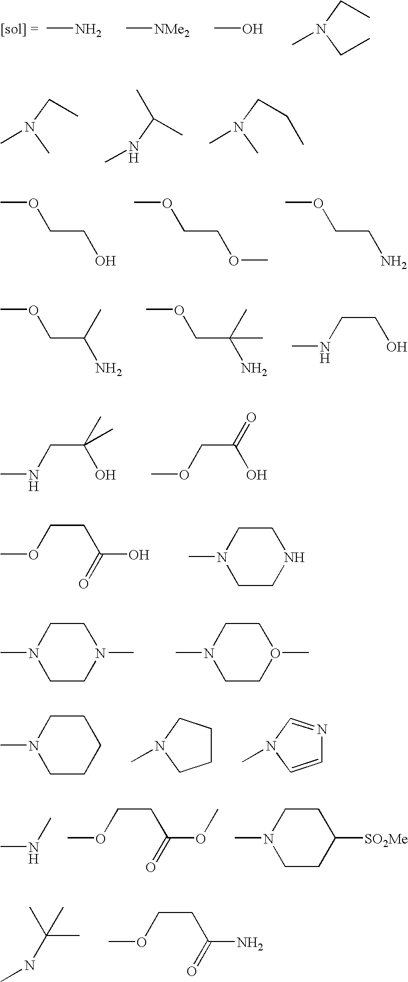

More particularly, R¹³ may be selected from halogen (other than fluorine or chlorine), OH, NH₂, CH₂OH, CH₂NH₂, NH—C₁₋₆ alkyl, aryl, heteroaryl, C₃₋₇ cycloalkyl, heterocyclyl, O-heteroaryl, O—C₃₋₇ cycloalkyl, O-heterocycloalkyl, C(═O)C₁₋₆ alkyl, C(═O)OC₁₋₆ alkyl, C(O)NH₂, C(═O)NHC₁₋₆ alkyl, NH(C₁₋₆ alkyl), NC(D)C₁₋₆ alkyl, C₆ aryl, OC₆ aryl, C(═O)C₆ aryl, C(═O)OC₆aryl, C(═O)NH₂, C(═O)NHC₆ aryl, NH(C₆ aryl), NC(═O)C₆ aryl, C₅₋₆ heterocyclyl, OC₅₋₆ heterocyclyl, C(═O)C₅₋₆ heterocyclyl, C(═O)OC₅₋₆ heterocyclyl, C(═O)NHC₅₋₆ heterocyclyl, NH(C₅₋₄ heterocyclyl), NC(═O)C₅₋₆ heterocyclyl, C(═O)NHC₁₋₆ alkyl, C₅₋₆ aryl, S(═O)C₁₋₆ alkyl, S(═O)N—C₁₋₆ alkyl and SO₂N—C₁₋₆ alkyl; and a group [sol], CH₂CH₂[sol], CH₂[sol] or OCH₂CH₂[sol] where [sol] is as defined above.

In another sub-group of compounds, R¹³ is selected from a group [sol], CH₂[sol], CH₂CH₂[sol], CH₂CH₂CH₂[sol], OCH₂CH₂[sol] or OCH₂CH₂CH₂[sol] where [sol] is selected from the following groups

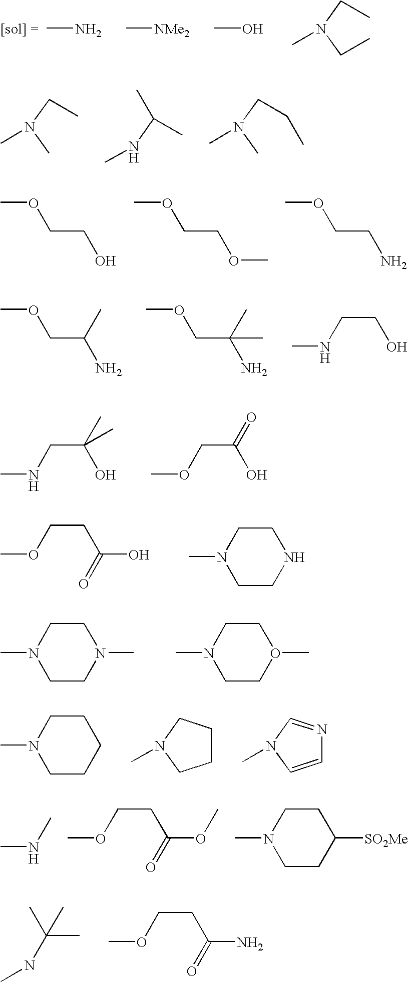

Particular examples of the group R¹³ are optionally substituted monocyclic 5 and 6 membered heterocyclic groups containing 1 or 2 heteroatoms selected from O, N and S.

Preferred heterocyclic groups are groups containing at least one nitrogen atom ring member and such groups include piperidine, piperazine, N-methyl piperazine, morpholine, pyrrolidine and imidazole each optionally substituted by one or more substituents selected from methyl, hydroxy, C═O (in the case of non-aromatic rings).

Alternatively, R¹³ can be a heteroaryl ring, for example a monocyclic heteroaryl ring containing one or two nitrogen atoms such as a pyridine, pyrimidine, pyridazine or pyrazine ring, a particular example being a pyridine ring.

The heterocyclic groups may be linked to the benzene ring through a carbon atom or through a nitrogen atom. In the case of non-aromatic groups such as morpholine, particular heterocyclic groups are those that are linked to the benzene ring via a nitrogen atom.

### R¹⁴

R¹⁴ is selected from hydrogen, fluorine, chlorine, methoxy, cyano, trifluoromethyl, trifluoromethoxy, difluoromethoxy, C₁₋₄ alkyl and cyclopropyl.

More typically, R¹⁴ is selected from hydrogen, fluorine and chlorine, and preferably is hydrogen or chlorine.

In one embodiment, R¹⁴ is hydrogen.

In another embodiment, R¹⁴ is chlorine.

### R^{1c}

R^{1c} is a heteroaryl group selected from pyridine, pyrimidine, pyrazine, pyridone, N-methylpyridone, furan and thiophene, each of which heteroaryl groups is unsubstituted or substituted with one or two substituents selected from chlorine, fluorine, methyl, methoxy and trifluoromethoxy.

Preferred groups include pyridyl and pyrimidinyl groups with pyridine being particularly preferred. The pyridyl group may be a 2-pyridyl, 3-pyridyl or 4-pyridyl group.

In one embodiment, the heteroaryl group is unsubstituted.

In another embodiment, the heteroaryl group bears one or two substituents.

For six membered heterocycles, it is preferred that the heteroaryl group is:

(i) mono-substituted at a position meta or para relative to its point of attachment to the group A; or
(ii) disubstituted at positions meta and para relative to its point of attachment to the group A; or
(iii) disubstituted at positions ortho and para relative to its point of attachment to the group A.

For five membered heterocycles, it is preferred that the heteroaryl group is monosubstituted at a position 2 atoms removed from the point of attachment to the group A.

Examples of substitution patterns for the heteroaryl group are as follows:

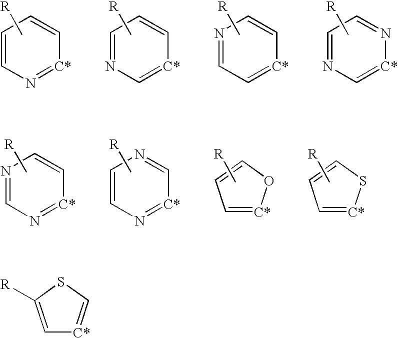

where R═Cl, F, Me, OMe or OCF₃ and the point of attachment to the group A is denoted by an asterisk *.

### R^{1d}

The group R^{1d} is represented by the formula:

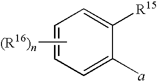

wherein:
“a” denotes the point of attachment to the group A;
R¹⁵ is selected from fluorine, chlorine, methoxy, cyano, trifluoromethyl, trifluoromethoxy, difluoromethoxy, C₁₋₄ alkyl and cyclopropyl; and
R¹⁶ is selected from R¹³ and R¹⁴, and n is 0, 1 or 2, provided that when n is 2, only one group R¹³ may be present.

Preferably, n is 0 or 1.

Preferably and more preferably is selected from hydrogen, fluorine and chlorine. In one embodiment R¹⁶ is selected from hydrogen and chlorine. In another embodiment, R¹⁶ is hydrogen. In a further embodiment, R¹⁶ is chlorine.

In another embodiment, J¹ is:

(2) a group of the formula:

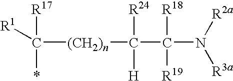

wherein:
the asterisk denotes the point of attachment to the group E;
n is 0 or 1;
R¹ is an aryl or heteroaryl group having 5 to 10 ring members;
R^{2a} and R^{3a} are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy;
or R^{2a} and R^{3a} together with the nitrogen atom to which they are attached form a cyclic group selected from an imidazole group and a saturated monocyclic heterocyclic group having 4-7 ring members and optionally containing a second heteroatom ring member selected from O and N;
R¹⁷ is selected from hydrogen; hydroxy; fluorine; methyl; and a C₁₋₄ alkoxy group wherein the C₁₋₄ alkoxy group is optionally substituted by hydroxy or amino provided that there are at least two carbon atoms between the hydroxy or amino group and the oxygen atom of the C₁₋₄ alkoxy group;
R¹⁸ is selected from hydrogen and methyl;
R¹⁹ is selected from hydrogen and methyl; provided that at least one of R¹⁷, R¹⁸ and R¹⁹ is other than hydrogen; and
R²⁴ is hydrogen or R²⁴, R^{2a} and the intervening nitrogen atom and carbon atoms together form an azetidine, pyrrolidine or piperidine ring.

In one embodiment, R¹⁷ is selected from hydroxy and a C₁₋₄ alkoxy group wherein the C₁₋₄ alkoxy group is optionally substituted by hydroxy or amino provided that there are at least two carbon atoms between the hydroxy or amino group and the oxygen atom of the C₁₋₄ alkoxy group.

Within this embodiment, R¹⁷ is more particularly selected from hydroxy, methoxy, 2-hydroxyethoxy and 2-aminoethoxy. More preferably, R¹⁷ is selected from hydroxy and methoxy. In one group of compounds, R¹⁷ is hydroxy. In another group of compounds, R¹⁷ is methoxy.

In another embodiment, R¹⁷ is selected from hydrogen, fluorine and methyl.

In the group (CH₂)ₙ, n can be 0 or 1. In one group of compounds, n is 0. In another group of compounds, n is 1.

R²⁴ can be hydrogen or R²⁴, R^{2a} and the intervening nitrogen atom and carbon atoms together form an azetidine, pyrrolidine or piperidine ring.

In one group of compounds, R²⁴ is hydrogen.

In another embodiment, R²⁴, R^{2a} and the intervening nitrogen atom and carbon atoms together form an azetidine, pyrrolidine or piperidine ring, and more particularly an azetidine ring. In this group of compounds, R¹⁸ and R¹⁹ typically are both hydrogen.

When R²⁴, R^{2a} and the intervening nitrogen atom and carbon atoms together form an azetidine, pyrrolidine or piperidine ring, R^{3a} may be hydrogen or optionally substituted C₁₋₄ hydrocarbyl or optionally substituted C₁₋₄ acyl as hereinbefore defined. More typically however, R^{3a} is hydrogen or a C₁₋₄ alkyl group such as methyl, and most preferably is hydrogen.

Particular groups of the formula:

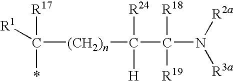

are those identified as A27, A28, A29, A30 and A31 (and particularly A27) in Table 2 above, but wherein the R-group R^{1a} is replaced by R¹.

In a further embodiment, R¹⁷ is selected from hydrogen, hydroxy, fluorine and methyl, n is 0 and R²⁴ is hydrogen; i.e. the group J¹ has the formula:

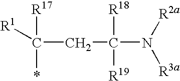

wherein:
the asterisk denotes the point of attachment to the group E;
R¹ is an aryl or heteroaryl group having 5 to 10 ring members;

R^{2a} and R^{3a} are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy;
or R^{2a} and R^{3a} together with the nitrogen atom to which they are attached form a cyclic group selected from an imidazole group and a saturated monocyclic heterocyclic group having 4-7 ring members and optionally containing a second heteroatom ring member selected from O and N;
R¹⁷ is selected from hydrogen, hydroxy, fluorine and methyl;
R¹⁸ is selected from hydrogen and methyl;
R¹⁹ is selected from hydrogen and methyl; provided that at least one of R¹⁷, R¹⁸ and R¹⁹ is other than hydrogen.

Within this embodiment, R^{2a} and R^{3a} are subsets of the groups R² and R³ defined above and, where the context permits, the preferences, subsets, subgroups, embodiments and examples set out herein for R² and R³ apply also to R^{2a} and R^{3a}.

In one sub-group of compounds, R¹⁸ and R¹⁹ are both hydrogen and R¹⁷ is selected from hydroxy, fluorine and methyl.

In another sub-group of compounds, R¹⁷ is hydrogen and R¹⁸ and R¹⁹ are both methyl.

In a further sub-group of compounds, R¹⁷ is hydroxy and R¹⁸ and R¹⁹ are both hydrogen.

In a further embodiment, J¹ is:

(3) a group of the formula:

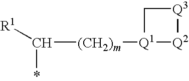

wherein:
the asterisk denotes the point of attachment to the group E;
m is 0 or 1;
Q¹ is CH or N;
Q² is CH₂ or NH;
Q³ is CH₂ or NH, provided that only one of Q¹, Q² and Q³ consists of or contains a nitrogen atom.

In one sub-group of compounds, m is 0.

In another sub-group of compounds, m is 1.

Within this embodiment, particular forms of the moiety:

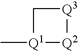

are the groups:

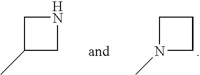

In a further embodiment, J¹ is:

(4) a group of the formula:

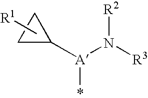

wherein A′ is a saturated hydrocarbon linker group containing from 1 to 5 carbon atoms, the linker group having a maximum chain length of 4 atoms extending between the cyclopropyl group and NR²R³ and a maximum chain length of 4 atoms extending between E and NR²R³;
R¹ is as hereinbefore defined;
R² and R³ are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the group A′ form a saturated monocyclic heterocyclic group having 4-7 ring members and optionally containing a second heteroatom ring member selected from O and N;
or NR²R³ and the carbon atom of group A′ to which it is attached together form a cyano group.

Within this embodiment, R¹ and A′ can be attached to the same carbon atom on the cyclopropane group or to adjacent carbon atoms on the cyclopropane group. Preferably, however, R¹ and A′ are attached to adjacent carbon atoms on the cyclopropane group.

### R¹

In embodiments (2), (3), (4) and (8), the group R¹ is an aryl or heteroaryl group having 5 to 10 ring members and may be selected from the list of such groups set out in the section headed General Preferences and Definitions.

R¹ can be monocyclic or bicyclic.

In one embodiment, R¹ is monocyclic. Particular examples of monocyclic aryl and heteroaryl groups are six membered aryl and heteroaryl groups containing up to 2 nitrogen ring members, and five membered heteroaryl groups containing up to 3 heteroatom ring members selected from O, S and N.

In another embodiment, R¹ is bicyclic.

Examples of bicyclic heteroaryl groups are groups containing a six membered ring fused to a five membered ring or a six membered ring fused to another six membered ring.

The heteroaryl groups typically have up to three heteroatom ring members selected from nitrogen, oxygen and sulphur, and more usually have up to two heteroatom ring members. For example, the heteroaryl groups may contain a single heteroatom ring member selected from O, N and S; or two heteroatom ring members of which one is N and the other is selected from O, N and S.

Particular examples of bicyclic heteroaryl groups include but are not limited to benzofuran, benzothiophene, benzimidazole, benzoxazole, benzisoxazole, benzothiazole, benzisothiazole, isobenzofuran, indole, isoindole, indolizine, indoline, isoindoline, indazole, benzodioxole, chroman, thiochroman, chromene, isochromene, chroman, isochroman, benzodioxan, quinolizine, benzoxazine, benzodiazine, pyridopyridine, quinoxaline, quinazoline, tetrahydronaphthalene, tetrahydroisoquinoline, tetrahydroquinoline, dihydrobenzothiene, dihydrobenzofuran, 2,3-dihydro-benzo[1,4]dioxine, benzo[1,3]dioxole, 4,5,6,7-tetrahydrobenzofuran, indoline and indane groups.

One particular set of aryl and heteroaryl groups consists of phenyl, naphthyl, thienyl, furan, pyrimidine, pyridine, benzimidazole, benzoxazole, benzisoxazole, benzothiazole and benzisothiazole,

Another particular set of aryl and heteroaryl groups consists of benzimidazole, phenyl, naphthyl, thienyl, furan, pyrimidine and pyridine.

A further set of aryl and heteroaryl groups consists of phenyl, naphthyl, thienyl, furan, pyrimidine and pyridine, with phenyl being presently preferred.

The group R¹ can be unsubstituted or substituted by up to 5 substituents, and examples of substituents are those listed in group R¹⁰ above.

One group of substituents includes hydroxy; C₁₋₄ acyloxy; fluorine; chlorine; bromine; trifluoromethyl; cyano; CONH₂; nitro; C₁₋₄ hydrocarbyloxy and C₁₋₄ hydrocarbyl each optionally substituted by C₁₋₂ alkoxy, carboxy or hydroxy; C₁₋₄ acylamino; benzoylamino; pyrrolidino; piperidino; morpholino; piperazine; N-methylpiperazino; pyrrolidinocarbonyl; piperidinocarbonyl; morpholinocarbonyl; piperazinocarbonyl; five and six membered heteroaryl and heteroaryloxy groups containing one or two heteroatoms selected from N, O and S; phenyl; phenyl-C₁₋₄ alkyl; phenyl-C₁₋₄ alkoxy; heteroaryl-C₁₋₄ alkyl; heteroaryl-C₁₋₄ alkoxy and phenoxy, wherein the heteroaryl, heteroaryloxy, phenyl, phenyl-C₁₋₄ alkyl, phenyl-C₁₋₄ alkoxy, heteroaryl-C₁₋₄ alkyl, heteroaryl-C₁₋₄ alkoxy and phenoxy groups are each optionally substituted with 1, 2 or 3 substituents selected from C₁₋₂ acyloxy, fluorine, chlorine, bromine, trifluoromethyl, cyano, CONH₂, C₁₋₂ hydrocarbyloxy and C₁₋₂ hydrocarbyl each optionally substituted by methoxy or hydroxy.

Particular substituents include hydroxy; Cᵢ₄ acyloxy; fluorine; chlorine; bromine; trifluoromethyl; cyano; CONH₂; nitro; C₁₋₄ hydrocarbyloxy and C₁₋₄ hydrocarbyl each optionally substituted by C₁₋₂ alkoxy, carboxy or hydroxy; C₁₋₄ acylamino; benzoylamino; pyrrolidinocarbonyl; piperidinocarbonyl; morpholinocarbonyl; piperazinocarbonyl; five and six membered heteroaryl and heteroaryloxy groups containing one or two heteroatoms selected from N, O and S; phenyl; phenyl-C₁₋₄ alkyl; phenyl-C₁₋₄ alkoxy; heteroaryl-C₁₋₄ alkyl; heteroaryl-C₁₋₄ alkoxy and phenoxy, wherein the heteroaryl, heteroaryloxy, phenyl, phenyl-C₁₋₄ alkyl, phenyl-C₁₋₄ alkoxy, heteroaryl-C₁₋₄ alkyl, heteroaryl-C₁₋₄ alkoxy and phenoxy groups are each optionally substituted with 1, 2 or 3 substituents selected from C₁₋₂ acyloxy, fluorine, chlorine, bromine, trifluoromethyl, cyano, CONH₂, C₁₋₂ hydrocarbyloxy and C₁₋₂ hydrocarbyl each optionally substituted by methoxy or hydroxy.

A further sub-group of substituents includes hydroxy; acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy; C₁₋₄ acylamino; benzoylamino; piperidino; morpholino; piperazine; N-methylpiperazino; pyrrolidinocarbonyl; piperidinocarbonyl; morpholinocarbonyl; piperazinocarbonyl; five and six membered heteroaryl groups containing one or two heteroatoms selected from N, O and S, the heteroaryl groups being optionally substituted by one or more C₁₋₄ alkyl substituents; phenyl; pyridyl; and phenoxy wherein the phenyl, pyridyl and phenoxy groups are each optionally substituted with 1, 2 or 3 substituents selected from C₁₋₂ acyloxy, fluorine, chlorine, bromine, trifluoromethyl, cyano, C₁₋₂ hydrocarbyloxy and C₁₋₂ hydrocarbyl each optionally substituted by methoxy or hydroxy.

Another group of substituents includes hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy; C₁₋₄ acylamino; benzoylamino; pyrrolidinocarbonyl; piperidinocarbonyl; morpholinocarbonyl; piperazinocarbonyl; five and six membered heteroaryl groups containing one or two heteroatoms selected from N, O and S, the heteroaryl groups being optionally substituted by one or more C₁₋₄ alkyl substituents; phenyl; pyridyl; and phenoxy wherein the phenyl, pyridyl and phenoxy groups are each optionally substituted with 1, 2 or 3 substituents selected from C₁₋₂ acyloxy, fluorine, chlorine, bromine, trifluoromethyl, cyano, hydrocarbyloxy and C₁₋₂ hydrocarbyl each optionally substituted by methoxy or hydroxy.

In one sub-group of compounds, the substituents for R¹ are chosen from hydroxy; C₁₋₄ acyloxy; fluorine; chlorine; bromine; trifluoromethyl; cyano; piperidino; morpholino; piperazino; N-methylpiperazino; and C₁₋₄ hydrocarbyloxy and C₁₋₄ hydrocarbyl each optionally substituted by C₁₋₂ alkoxy or hydroxy.

In another sub-group of compounds, the substituents for R¹ are chosen from hydroxy; Cₘ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy.

Although up to 5 substituents may be present, more typically there are 0, 1, 2, 3 or 4 substituents, preferably 0, 1, 2 or 3, and more preferably 0, 1 or 2.

In one embodiment, the group R¹ is unsubstituted or substituted by up to 5 substituents selected from hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy.

In a further embodiment, the group R¹ can have one or two substituents selected from hydroxy, fluorine, chlorine, cyano, phenyloxy, pyrazinyloxy, benzyloxy, methyl and methoxy.

In another embodiment, the group R¹ can have one or two substituents selected from fluorine, chlorine, trifluoromethyl, methyl and methoxy.

When R¹ is a phenyl group, particular examples of substituent combinations include mono-chlorophenyl and dichlorophenyl.

Further examples of substituent combinations include those wherein R¹ is hydroxyphenyl, fluorochlorophenyl, cyanophenyl, methoxyphenyl, methoxy-chlorophenyl, fluorophenyl, difluorophenyl, phenoxyphenyl, pyrazinyloxyphenyl or benzyloxyphenyl.

When R¹ is a six membered aryl or heteroaryl group, a substituent may advantageously be present at the para position on the six-membered ring. Where a substituent is present at the para position, it is preferably larger in size than a fluorine atom.

In another embodiment, when (a) R⁴ is hydrogen and R⁵ is a group R^{5aa} wherein R^{5aa} is C₁₋₄ hydrocarbyl and E is phenyl; or (b) R⁴ and R⁵ are both hydrogen and E is a pyridyl ring in wherein J¹ is attached to the 2-position of the pyridyl ring and the pyrazole group is attached to the 5-position of the pyridyl ring; J¹ is

(5A) a group of the formula:

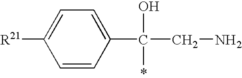

wherein the asterisk indicates the point of attachment to the group E and R²¹ is hydrogen or chlorine.
In a further embodiment, when R⁴ is hydrogen and R⁵ is a group R^{5aa} wherein R^{5aa} is C₁₋₄ hydrocarbyl and E is phenyl; J¹ is
(5B) a group of the formula:

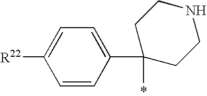

wherein the asterisk indicates the point of attachment to the group E and R²² is chlorine or methoxy provided that when R²² is chlorine, then R^{5aa} is C₂₋₄ hydrocarbyl.

In another embodiment, when R⁴ and R⁵ are both hydrogen and E is phenyl; then J¹ is:

(6) a group of the formula:

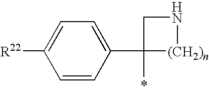

wherein n is 1 or 2 and R²² is chlorine or methoxy.

In another embodiment, J¹ is

(7) a group of the formula:

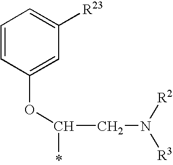

wherein R² and R³ are as defined herein and R²³ is fluorine, chlorine or a group R¹³.

In one sub-group of compounds within embodiment (7), R⁴ and R⁵ are both hydrogen and E is phenyl.

Particular and preferred groups R¹³ are as hereinbefore defined.

In a further embodiment, J¹ is

(8) a group of the formula:

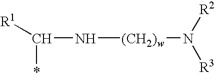

wherein w is 2 or 3 and R¹, R² and R³ are as defined herein;

Within this embodiment, w is typically 2.

### Particular and Preferred Subgroups

One sub-group of compounds of the formula (I) has the general formula (II):

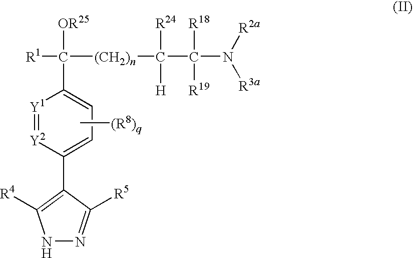

or a salt, solvate, tautomer or N-oxide thereof, wherein
n is 0 or 1;
one of Y¹ and Y² is CH and the other is selected from CH, CR⁸ and N;
q is 0, 1 or 2 provided that q is 0 or 1 when Y¹ or Y² is CR⁸;
R¹ is an aryl or heteroaryl group;
R^{2a} and R^{3a} are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy;
or R^{2a} and R^{3a} together with the nitrogen atom to which they are attached form a cyclic group selected from an imidazole group and a saturated monocyclic heterocyclic group having 4-7 ring members and optionally containing a second heteroatom ring member selected from O and N;
R⁸ is selected from hydroxy; halogen; trifluoromethyl; cyano; C₁₋₄ hydrocarbyloxy optionally substituted by C₁₋₂ alkoxy or hydroxy; and C₁₋₄ hydrocarbyl optionally substituted by C₁₋₂ alkoxy or hydroxy;
R¹⁸ is selected from hydrogen and methyl;
R¹⁹ is selected from hydrogen and methyl;
R²⁴ is hydrogen or R²⁴, R^{2a} and the intervening nitrogen atom and carbon atoms together form an azetidine, pyrrolidine or piperidine ring; and
R²⁵ is selected from hydrogen or a C₁₋₄ alkyl group wherein the C₁₋₄ alkyl group is optionally substituted by hydroxy or amino provided that there are at least two carbon atoms between the hydroxy or amino group and the oxygen atom to which R²⁵ is attached; and
R⁴ and R⁵ are each as hereinbefore defined.

In one embodiment of formula (II), Y¹ is CH. Within this embodiment, Y² can also be CH, or Y² can be CR⁸ or N.

Particular embodiments are those wherein:
Y¹ is CH and Y² is CH;
Y¹ is CH and Y² is N;
Y¹ is CH and Y² is CR⁸ such as CF;
Y² is CH and Y¹ is CR⁸ such as C-Me or CF; and
Y² is CH and Y¹ is N.

Within formula (II), the moiety:

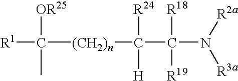

can be, for example, selected from groups D1 to D5:

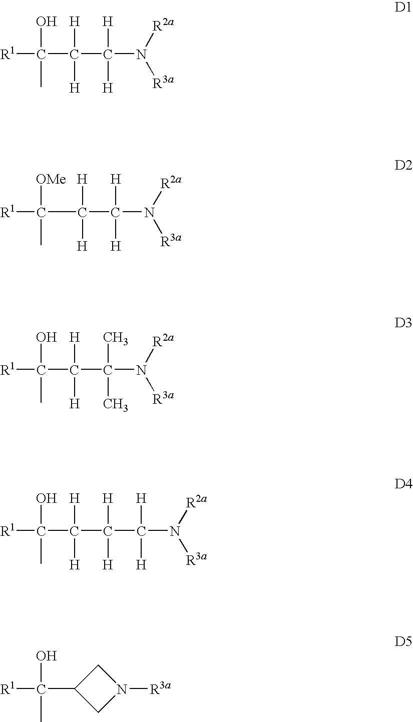

Another sub-group of compounds has the general formula (IIa):

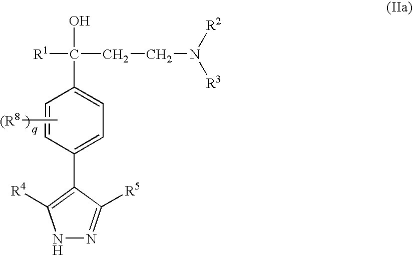

and salts, solvates, tautomers and N-oxides thereof;
wherein R¹, R², R³, R⁴, R⁵, and R⁸ are each as hereinbefore defined, and q is 0, 1 or 2 (preferably 0 or 1).

Within formulae (II) and (IIa), R⁸ may be selected, for example, from hydroxy; halogen (e.g. fluorine, chlorine and bromine); trifluoromethyl; cyano; C₁₋₄ alkoxy optionally substituted by C₁₋₂ alkoxy or hydroxy; and C₁₋₄ alkyl optionally substituted by C₁₋₂ alkoxy or hydroxy.

Typically, R⁸ is absent or is selected from hydroxy, fluorine, chlorine, trifluoromethyl, methyl, ethyl and hydroxymethyl.

Alternatively, R⁸ may be absent (q=0) or selected from methyl, fluorine, chlorine, methoxy, trifluoromethyl and cyano.

In one sub-group of compounds, R⁸ is absent (q=0) or is selected from methyl and fluorine, In another sub-group of compounds, R⁸ is absent (q=0) or is a fluorine atom.

Within formulae (II) and (IIa), R⁴ and R⁵ are preferably each selected from hydrogen and C₁₋₄ hydrocarbyl, more preferably saturated C₁₋₄ hydrocarbyl. In one sub-group of compounds, R⁴ is hydrogen and R⁵ is hydrogen or saturated C₁₋₄ hydrocarbyl.

Examples of saturated C₁₋₄ hydrocarbyl groups are C₁₋₄ alkyl groups, C₃₋₄ cycloalkyl and cyclopropylmethyl groups. Particular examples of saturated C₁₋₄ hydrocarbyl groups are methyl, ethyl, propyl, isopropyl, cyclopropyl, butyl, isobutyl, tert-butyl and cyclopropylmethyl.

In one sub-group of compounds within formula (II), R⁴ and R⁵ are both hydrogen.

In one sub-group of compounds within formula (IIa), R⁴ and R⁵ are both hydrogen.

R² and R³, and R^{2a} and R^{3a}, may be as hereinbefore defined but preferably are independently selected from hydrogen and saturated C₁₋₄ hydrocarbyl wherein the saturated hydrocarbyl moiety are optionally substituted by one or more substituents selected from fluorine, hydroxy, amino, methylamino, dimethylamino and methoxy; or R² and R³ together with the nitrogen atom to which they are attached form a cyclic group selected from an imidazole group and a saturated monocyclic heterocyclic group having 4-7 ring members and optionally containing a second heteroatom ring member selected from O and N.

More preferably, R² and R³, and R^{2a} and R^{3a}, are independently selected from hydrogen and saturated C₁₋₄ hydrocarbyl, for example C₁₋₄ alkyl such as methyl and ethyl. In one preferred group of compounds, R² and R³ are both hydrogen.

Within formulae (II) and (IIa), the group R¹ can be an aryl or heteroaryl group and may be selected from the list of such groups set out in the section headed General Preferences and Definitions.

R¹ can be monocyclic or bicyclic and, in one preferred embodiment, is monocyclic. Examples of such groups include phenyl, naphthyl, thienyl, furan, pyrimidine and pyridine, with phenyl being presently preferred.

The group R¹ can be unsubstituted or substituted by up to 5 substituents, and examples of substituents are those listed in group R¹⁰ above.

Particular substituents include hydroxy; C₁₋₄ acyloxy; fluorine; chlorine; bromine; trifluoromethyl; cyano; CONH₂; nitro; C₁₋₄ hydrocarbyloxy and C₁₋₄ hydrocarbyl each optionally substituted by C₁₋₂ alkoxy, carboxy or hydroxy; C₁₋₄ acylamino; benzoylamino; pyrrolidinocarbonyl; piperidinocarbonyl; morpholinocarbonyl; piperazinocarbonyl; five and six membered heteroaryl and heteroaryloxy groups containing one or two heteroatoms selected from N, O and S; phenyl; phenyl-C₁₋₄ alkyl; phenyl-C₁₋₄ alkoxy; heteroaryl-C₁₋₄ alkyl; heteroaryl-C₁₋₄ alkoxy and phenoxy, wherein the heteroaryl, heteroaryloxy, phenyl, phenyl-C₁₋₄ alkyl, phenyl-C₁₋₄ alkoxy, heteroaryl-C₁₋₄ alkyl, heteroaryl-C₁₋₄ alkoxy and phenoxy groups are each optionally substituted with 1, 2 or 3 substituents selected from C₁₋₂ acyloxy, fluorine, chlorine, bromine, trifluoromethyl, cyano, CONH₂, C₁₋₂ hydrocarbyloxy and C₁₋₂ hydrocarbyl each optionally substituted by methoxy or hydroxy.

More particular substituents include hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy; C₁₋₄ acylamino; benzoylamino; pyrrolidinocarbonyl; piperidinocarbonyl; morpholinocarbonyl; piperazinocarbonyl; five and six membered heteroaryl groups containing one or two heteroatoms selected from N, O and S, the heteroaryl groups being optionally substituted by one or more C₁₋₄ alkyl substituents; phenyl; pyridyl; and phenoxy wherein the phenyl, pyridyl and phenoxy groups are each optionally substituted with 1, 2 or 3 substituents selected from C₁₋₂ acyloxy, fluorine, chlorine, bromine, trifluoromethyl, cyano, C₁₋₂ hydrocarbyloxy and C₁₋₂ hydrocarbyl each optionally substituted by methoxy or hydroxy.

In one sub-group of compounds, the substituents for R¹ are chosen from hydroxy; C₁₋₄ acyloxy; fluorine; chlorine; bromine; trifluoromethyl; cyano; saturated C₁₋₄ hydrocarbyloxy and saturated C₁₋₄ hydrocarbyl each optionally substituted by C₁₋₂ alkoxy or hydroxy.

Although up to 5 substituents may be present, more typically there are 0, 1, 2, 3 or 4 substituents, preferably 0, 1, 2 or 3, and more preferably 0, 1 or 2.

In one embodiment, the group R¹ is unsubstituted or substituted by up to 5 (e.g. 1, 2, 3, 4, or 5) substituents selected from hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy.

In a further embodiment, the group R¹ can have one or two substituents selected from hydroxy, fluorine, chlorine, cyano, phenyloxy, pyrazinyloxy, benzyloxy, methyl and methoxy.

In another embodiment, the group R¹ can have one or two substituents selected from fluorine, chlorine, trifluoromethyl, methyl and methoxy.

When R¹ is a phenyl group, particular examples of substituent combinations include mono-chlorophenyl (e.g. 4-chlorophenyl or 3-chlorophenyl), trifluoromethylphenyl (e.g. 4-trifluoromethylphenyl), trifluoromethoxyphenyl (e.g. 4-trifluoro-methoxyphenyl), tert-butylphenyl (e.g. 4-tert-butylphenyl), dichlorophenyl (e.g. 3,4-dichlorophenyl), fluoro-chlorophenyl (e.g. 2-fluoro-4-chlorophenyl and 3-fluoro-4-chlorophenyl), and 4-chloro-3-(4-morpholinyl)-phenyl groups.

In one sub-group of compounds, when R¹ is a phenyl group, particular examples of substituent combinations include mono-chlorophenyl (e.g. 4-chlorophenyl), dichlorophenyl (e.g. 3,4-dichlorophenyl) and fluoro-chlorophenyl (e.g. 3-fluoro-4-chlorophenyl) groups.

When R¹ is a heteroaryl group, examples include unsubstituted and substituted pyridine, furan and thiophene groups and more particularly pyridine and thiophene groups. Particular examples are pyridine and thiophene groups that bear a substituent selected from fluorine, chlorine, C₁₋₄ alkyl (e.g. tert-butyl), C₁₋₄ alkoxy (e.g. methoxy), trifluoromethyl, trifluoromethoxy, difluoromethoxy and 5-6 membered saturated heterocyclic rings containing a nitrogen ring member and optionally a second heteroatom ring member selected from O and N.

More particular examples of heteroaryl groups R¹ are 5-chloro-2-thienyl and 5-chloro-2-pyridyl groups.

A further sub-group of compounds within formula (II) is represented by the formula (IIb):

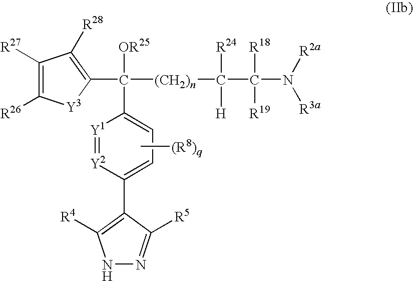

or a salt, solvate, tautomer or N-oxide thereof; wherein
n is 0 or 1;
one of Y¹ and Y² is CH and the other is selected from CH and N;
Y³ is CH═CH, CH═N or S;
q is 0 or 1;
R^{2a} is hydrogen or methyl;
R^{3a} is hydrogen or methyl;
R⁸ is fluorine or methyl;
R¹⁸ is hydrogen or methyl;
R¹⁹ is hydrogen or methyl;
R²⁴K is hydrogen or R²⁴, R^{2a} and the intervening nitrogen atom and carbon atoms together form an azetidine, pyrrolidine or piperidine ring; and
R²⁵ is hydrogen or methyl provided that there are at least two carbon atoms between the hydroxy or amino group and the oxygen atom to which R²⁵ is attached;
one of R⁴ and R⁵ is hydrogen and the other is hydrogen, methyl or trifluoromethyl;
R²⁶ is hydrogen, chlorine, fluorine, trifluoromethyl, difluoromethoxy, trifluoromethoxy, C₁₋₄ alkyl or C₁₋₃ alkoxy;
R²⁷ is hydrogen, chlorine, fluorine, trifluoromethyl, difluoromethoxy, trifluoromethoxy, C₁₋₄ alkyl, C₁₋₃ alkoxy, morpholinyl, pyrrolidinyl, piperidinyl or piperazinyl; and
R²⁸ is hydrogen or fluorine; provided that no more than 2 of R²⁶, R²⁷ and R²⁸ are other than hydrogen.

In compounds of the formulae (II), (IIa) and (IIb), the carbon atoms shown with the asterisk in the moieties:

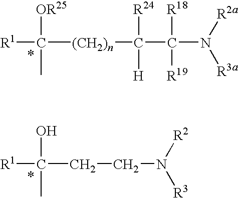

are chiral centres and the compounds can exist as R and S isomeric forms with regard to these chiral centres. In one embodiment, the compounds of formulae (II), (IIa) and (IIb) can have the S configuration about the chiral centres shown.

A preferred sub-group of compounds within formula (IIb) can be represented by the formula (IIc):

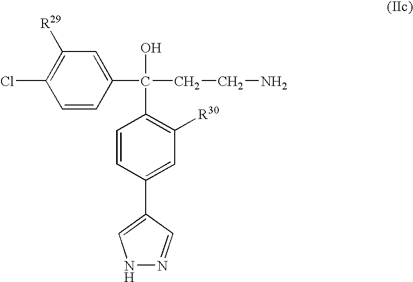

and salts, solvates and tautomers thereof;
wherein R²⁹ is hydrogen, chlorine or fluorine and R³⁰ is hydrogen or fluorine.

Within formula (IIc), a particularly preferred compound is the compound wherein R²⁹ is hydrogen and R³⁰ is hydrogen

Another sub-group of compounds of formula (I) can be represented by formula (III):

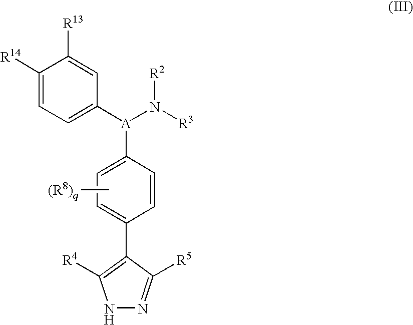

and salts, solvates, tautomers and N-oxides thereof;
wherein A, R², R³, R⁴, R⁵, R⁸, R¹³, R¹⁴ and q are as defined herein.

Examples of the moiety A are the groups illustrated in Table 2 but wherein the group R^{1a} in the structures shown in the Table is replaced by the moiety:

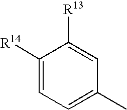

Particular preferences for and examples of R⁴, R⁵, R⁸ and q are as set out above in respect of formula (II). In one sub-group of compounds, R⁴ is hydrogen, R⁵ is hydrogen, C₁₋₄ alkyl (e.g. methyl), C₃₋₄ cycloalkyl or cyclopropylmethyl; and q is 0 (R⁸ is absent).

The group R¹³ is preferably selected from hydroxy, trifluoromethyl, cyano, amino, mono- or di-C₁₋₄ hydrocarbylamino; a group R^{a}—R^{b} wherein R^{a} is a bond, O, CO, S, SO, SO₂ or NR^{c}; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂ or NR^{c}; provided that R^{b} is other than hydrogen when R^{a} is a bond; and R¹ is selected from hydrogen and C₁₋₄ hydrocarbyl.

In one particular sub-group of compounds, R¹³ is a monocyclic or bicyclic carbocyclic or heterocyclic group having from 3 to 12 ring members, and examples of such groups are as set out above.

In one particular embodiment, R¹³ is a benzoxazole group, e.g. a 2-benzoxazole group.

In formula (III), R¹⁴ is typically selected from hydrogen, fluorine, chlorine, methoxy, cyano, trifluoromethyl, trifluoromethoxy, difluoromethoxy, methyl and ethyl.

In one particular embodiment, R¹⁴ is hydrogen.

One sub-group of compounds within formula (III) can be represented by the formula (IIIa):

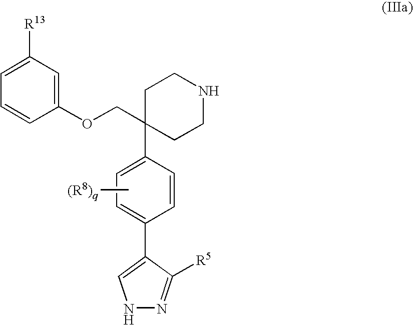

and salts, solvates, tautomers and N-oxides thereof;
wherein R⁵, R⁸, R¹³ and q are as defined above in relation to formula (III).

Particular compounds within formula (IIIa) are those in which R⁵ is hydrogen or C₁₋₄ alkyl (e.g. methyl), q is 0 and R¹³ is a monocyclic or bicyclic carbocyclic or heterocyclic group having from 3 to 12 ring members, for example a benzoxazole group, e.g. a 2-benzoxazole group.

A further sub-group of compounds of the formula (I) has the general formula (IV):

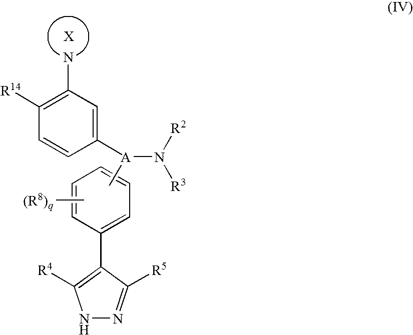

and salts, solvates, tautomers and N-oxides thereof;
wherein the group A is attached to the meta or para position of the benzene ring, the ring X is a 5 or 6 membered monocyclic heterocyclic group containing 1 or 2 nitrogen ring members and up to 1 oxygen ring member wherein a nitrogen ring member of the monocyclic heterocyclic group is attached to the benzene ring; q is 0-4; R², R³, R⁴, R⁵ and R¹⁴ are as defined herein in respect of formula (I) and sub-groups, examples and preferences thereof; and R⁸ is a substituent group as hereinbefore defined. In formula (II), q is preferably 0, 1 or 2, more preferably 0 or 1 and most preferably 0. Preferably the group A is attached to the para position of the benzene ring.

Within formula (III), R¹⁴ is preferably selected from fluorine, chlorine and hydrogen and most preferably is hydrogen.

The heterocyclic group X is preferably selected from morpholine, piperidine, pyrrolidine, azetidine, piperazine, imidazole each optionally substituted by up to 3 methyl groups.

Particular heterocyclic groups are morpholine and N-methylpiperazine.

Examples of the moiety A are the groups illustrated in Table 2 but wherein the group R^{1a} in the structures shown in the Table is replaced by the moiety:

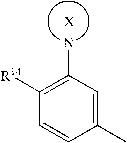

Another sub-group of compounds of the formula (I) is represented by the formula (V):

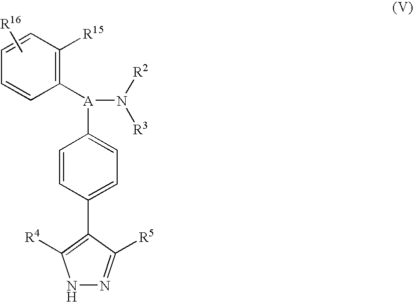

and salts, solvates, tautomers and N-oxides thereof;
where A and R² to R⁵, R¹⁵ and R¹⁶ are as defined herein.

Examples of the moiety A are the groups illustrated in Table 2 but wherein the group R^{1a} in the structures shown in the Table is replaced by the moiety:

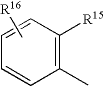

A further sub-group of compounds can be represented by the formula (VI):

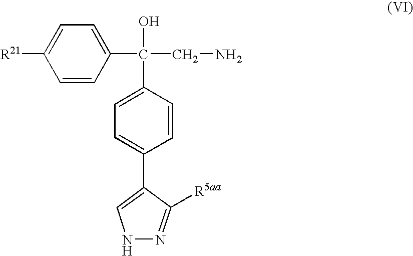

and salts, solvates, tautomers and N-oxides thereof;
wherein R²¹ is as defined herein; R^{5aa} is C₁₋₄ hydrocarbyl, preferably saturated hydrocarbyl.

Another sub-group of compounds can be represented by the formula (VII):

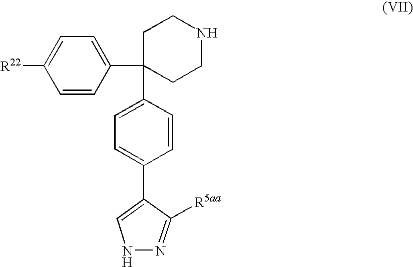

and salts, solvates, tautomers and N-oxides thereof;
wherein R^{5aa} is C₁₋₄ hydrocarbyl; and R^{5aa} is chlorine or methoxy; provided that when R²² is chlorine, then R^{5aa} is C₂₋₄ hydrocarbyl.

Examples of the C₁₋₄ hydrocarbyl group R^{5aa} in formulae (V) and (VI) are C₁₋₄ alkyl groups (e.g. C₂₋₄ alkyl groups) and C₃₋₄ cycloalkyl groups. Particular examples are methyl, ethyl, propyl, isopropyl, cyclopropyl, butyl, isobutyl, tert-butyl and cyclopropylmethyl. Preferably R^{5aa} is selected from (i) methyl, ethyl, isopropyl and cyclopropyl; or (ii) ethyl, isopropyl and cyclopropyl.

A further sub-group of compounds can be represented by the formula (VIIa):

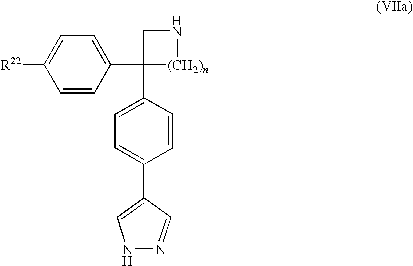

and salts, solvates, tautomers and N-oxides thereof
wherein n is 1 or 2 and R²² is as denied herein.

In one embodiment, n is 1.

In another embodiment, n is 2.

Another group of compounds within formula (I) is represented by the formula (VIII):

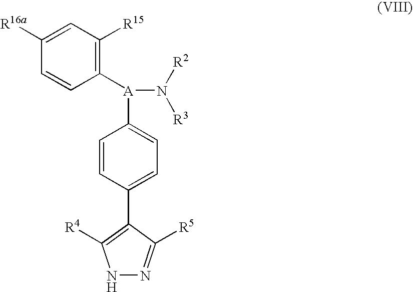

and salts, solvates, tautomers and N-oxides thereof;
wherein R^{16a} is hydrogen or chlorine and R¹⁵ is selected from fluorine, chlorine, methoxy, cyano, trifluoromethyl, trifluoromethoxy, difluoromethoxy, C₁₋₄ alkyl and cyclopropyl, or a subgroup thereof as defined herein.

Examples of the moiety A are the groups illustrated in Table 2 but wherein the group R^{1a} in the structures shown in the Table is replaced by the moiety:

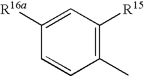

A further group of compounds within formula (I) is represented by formula (IX):

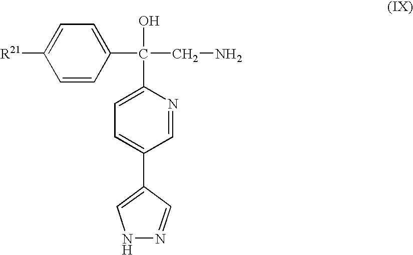

and salts, solvates, tautomers and N-oxides thereof;
wherein R²¹ is as defined herein.

In a further aspect, the invention provides a compound selected from:
3-(4-chloro-3-fluoro-phenyl)-3-[4-(1H-pyrazol-4-yl)-phenyl]-propylamine; and
{(R)-3-(3,4-dichloro-phenyl)-3-[4-(1H-pyrazol-4-yl)-phenyl]-propylamine;
   and salts, solvates, tautomers and N-oxides thereof.

For the avoidance of doubt, it is to be understood that each general and specific preference, embodiment and example of the group E and/or R⁴ and/or R⁵ may be combined with each general and specific preference, embodiment and example of the groups A and/or R¹ and/or R² and/or R³ and/or any other R group as defined herein, unless the context requires otherwise, and that all such combinations are embraced by this application.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Particular compounds of the invention are as illustrated in the examples below, and include:
3-(4-chloro-3-fluoro-phenyl)-3-[4-(1H-pyrazol-4-yl)-phenyl]-propylamine;
{(R)-3-(3,4-dichloro-phenyl)-3-[4-(1H-pyrazol-4-yl)-phenyl]-propylamine;
4-(3-methoxy-phenoxymethyl)-4-[4-(3-methyl-1H-pyrazol-4-yl)-phenyl]-piperidine;
2-amino-1-[4-(3-methyl-1H-pyrazol-4-yl)-phenyl]-1-phenyl-ethanol;
2-amino-1-(4-chloro-phenyl)-1-[4-(3-methyl-1H-pyrazol-4-yl)-phenyl]-ethanol;
2-amino-1-(4-chloro-phenyl)-1-[4-(3-ethyl-1H-pyrazol-4-yl)-phenyl]-ethanol;
4-{-4-[3-(4-chloro-phenyl)-pyrrolidin-3-yl]-phenyl}-1H-pyrazole formate;
4-{-4-[3-(4-methoxy-phenyl)-azetidin-3-yl]-phenyl}-1H-pyrazole formate;
4-(2,3-dihydro-benzo[1,4]dioxin-6-yl)-4-[4-(1H-pyrazol-4-yl)-phenyl]-piperidine;
4-(3-chloro-phenyl)-4-[4-(1H-pyrazol-4-yl)-phenyl]-piperidine;
4-(4′-methoxy-biphenyl-3-yl)-4-[4-(1H-pyrazol-4-yl)-phenyl]-piperidine dihydrochloride;
4-(4-methoxy-phenyl)-4-[4-(3-methyl-1H-pyrazol-4-yl)-phenyl]-piperidine dihydrochloride;
4-(4-chloro-phenyl)-4-[4-(5-ethyl-1H-pyrazol-4-yl)-phenyl]-piperidine;
4-(4-chloro-phenyl)-4-[4-(5-cyclopropyl-1H-pyrazol-4-yl)-phenyl]-piperidine;
dimethyl-(3-{-4-[4-(1H-pyrazol-4-yl)-phenyl]-piperidin-4-yl}-phenyl)-amine;
4-(2-methoxy-5-pyridin-yl)-4-[4-(1H-pyrazol-4-yl)-phenyl]-piperidine;
2-amino-1-(4-chloro-phenyl)-1-[5-(1H-pyrazol-4-yl)-pyridin-2-yl]-ethanol hydrochloride;
3-amino-1-(4-chloro-phenyl)-1-[4-(1H-pyrazol-4-yl)-phenyl]-propan-1-ol;
2-(3-{4-[4-(3-methyl-1H-pyrazol-4-yl)-phenyl]piperidin-4-ylmethoxy}-phenyl)-benzo oxazole;
3-amino-1-(4-chlorophenyl)-1-[2-fluoro-4-(1H-pyrazol-4-yl)-phenyl]-propan-1-ol-acetic acid salt;
3-amino-1-(4-chloro-3-fluoro-phenyl)-1-[4-(1H-pyrazol-4-yl)-phenyl]-propan-1-ol;
3-amino-1-(3,4,-dichlorophenyl)-1-[4-(1H-pyrazol-4-yl)-phenyl]-propan-1-ol;
(S)-3-amino-1-(4-chloro-phenyl)-1-[4-(1H-pyrazol-4-yl)-phenyl]-propan-1-ol;
(R)-3-amino-1-(4-chloro-phenyl)-1-[4-(1H-pyrazol-4-yl)-phenyl]-propan-1-ol;
3-amino-1-(3-chloro-phenyl)-1-[4-(1H-pyrazol-4-yl)-phenyl]-propan-1-ol;
3-amino-1-[4-(1H-pyrazol-4-yl)-phenyl]-1-(4-trifluoromethoxy-phenyl)-propan-1-ol;
3-amino-1-[4-(1H-pyrazol-4-yl)-phenyl]-1-(4-trifluoromethyl-phenyl)-propan-1-ol;
3-amino-1-(4-chloro-2-fluoro-phenyl)-1-[4-(1H-pyrazol-4-yl)-phenyl]-propan-1-ol;
3-amino-1-(5-chloro-pyridin-2-yl)-1-[4-(1H-pyrazol-4-yl)-phenyl]-propan-1-ol;
3-amino-1-(4-tert-butyl-phenyl)-1-[4-(1H-pyrazol-4-yl)-phenyl]-propan-1-ol;
3-amino-1-(4-chloro-3-morpholin-4-yl-phenyl)-1-[4-(1H-pyrazol-4-yl)-phenyl]-propan-1-ol;
3-amino-1-(5-chloro-thiophen-2-yl)1-[4-(1H-pyrazol-4-yl)-phenyl]-propan-1-ol;
3-amino-1-(4-chloro-phenyl)-1-[6-(1H-pyrazol-4-yl)-pyridin-3-yl]-propan-1-ol;
3-amino-1-(4-chloro-phenyl)-1-[3-fluoro-4-(1H-pyrazol-4-yl)-phenyl]-propan-1-ol;
3-amino-1-(4-chloro-phenyl)-1-[2-methyl-4-(1H-pyrazol-4-yl)-phenyl]-propan-1-ol;
4-amino-1-(4-chloro-phenyl)-1-[4-(1H-pyrazol-4-yl)-phenyl]-butan-1-ol;
1-(4-chloro-phenyl)-3-methylamino-1-[4-(1H-pyrazol-4-yl)-phenyl]-propan-1-ol;
azetidin-3-yl-(4-chlorophenyl)-[4-(1H-pyrazol-4-yl)-phenyl]-methanol;
3-amino-1-(4-chloro-phenyl)-3-methyl-1-[4-(1H-pyrazol-4-yl)-phenyl]-butan-1-ol;
3-amino-1-(4-chloro-phenyl)-1-[4-(3-methyl-1H-pyrazol-4-yl)-phenyl]-propan-1-ol;
3-amino-1-(4-chloro-phenyl)-1-[4-(3-trifluoromethyl-1H-pyrazol-4-yl)-phenyl]-propan-1-ol;
3-(4-chloro-phenyl)-3-methoxy-3-[4-(1H-pyrazol-4-yl)-phenyl]-propylamine;
N*1*-{(4-chloro-phenyl)-[4-(1H-pyrazol-4-yl)-phenyl]-methyl}-ethane-1,2-diamine;
   and salts, free bases, solvates (e.g. hydrates), tautomers and N-oxides thereof.

Preferred compounds include:
3-amino-1-(4-chloro-phenyl)-1-[4-(1H-pyrazol-4-yl)-phenyl]-propan-1-ol;
3-amino-1-(4-chlorophenyl)-1-[2-fluoro-4-(1H-pyrazol-4-yl)-phenyl]-propan-1-ol;
3-amino-1-(4-chloro-3-fluoro-phenyl)-1-[4-(1H-pyrazol-4-yl)-phenyl]-propan-1-ol;
3-amino-1-(3,4,-dichlorophenyl)-1-[4-(1H-pyrazol-4-yl)-phenyl]-propan-1-ol;
   and salts, solvates (e.g. hydrates) and tautomers thereof.

More preferred compounds are (S)-3-amino-1-(4-chloro-phenyl)-1-[4-(1H-pyrazol-4-yl)-phenyl]-propan-1-ol; and
(R)-3-amino-1-(4-chloro-phenyl)-1-[4-(1H-pyrazol-4-yl)-phenyl]-propan-1-ol;
   and salts, solvates (e.g. hydrates) and tautomers thereof.

A particularly preferred compound is (S)-3-amino-1-(4-chloro-phenyl)-1-[4-(1H-pyrazol-4-yl)-phenyl]-propan-1-ol and salts, solvates (e.g. hydrates) and tautomers thereof.

### Salts, Solvates, Tautomers, Isomers, N-Oxides, Esters, Prodrugs and Isotopes

In this section, as in all other sections of this application, unless the context indicates otherwise, references to formula (I) include references to formulae (Ia), (Ib), (II), (IIa), (IIb), (IIc), (III), (IIIa), (IV), (V), (VI), (VII), (VIIa), (VIII), (IX) and all other sub-groups, preferences and examples thereof as defined herein.

Unless otherwise specified, a reference to a particular compound also includes ionic, salt, solvate, and protected forms thereof, for example, as discussed below.

Many compounds of the formula (I) can exist in the foam of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds. As in the preceding sections of this application, all references to formula (I) should be taken to refer also to formulae (Ia), (Ib), (II), (III), (IIIa), (IV), (V), (VI), (VII), (VIIa), (VIII), (IX) and sub-groups thereof unless the context indicates otherwise.

Salt forms may be selected and prepared according to methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. For example, acid addition salts may be prepared by dissolving the free base in an organic solvent in which a given salt form is insoluble or poorly soluble and then adding the required acid in an appropriate solvent so that the salt precipitates out of solution.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic, (−)-L-malic, malonic, (±)-DL-mandelic, methanesulphonic, naphthalenesulphonic (e.g. naphthalene-2-sulphonic), naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, toluenesulphonic (e.g. p-toluenesulphonic), undecylenic and valeric acids, as well as acylated amino acids and cation exchange resins.

One particular group of acid addition salts includes salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

Another group of acid addition salts includes salts formed from acetic, adipic, ascorbic, aspartic, citric, DL-Lactic, fumaric, gluconic, glucuronic, hippuric, hydrochloric, glutamic, DL-malic, methanesulphonic, sebacic, stearic, succinic and tartaric acids.

The compounds of the invention may exist as mono- or di-salts depending upon the pKa of the acid from which the salt is formed. In stronger acids, the basic pyrazole nitrogen, as well as the nitrogen atom in the group NR²R³, may take part in salt formation. For example, where the acid has a pKa of less than about 3 (e.g. an acid such as hydrochloric acid, sulphuric acid or trifluoroacetic acid), the compounds of the invention will typically form salts with 2 molar equivalents of the acid.

If the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

For example, in compounds of the formula (I) the pyrazole group may take either of the following two tautomeric forms A and B.

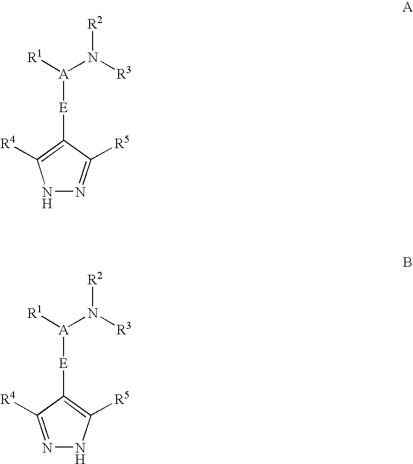

For simplicity, the general formula (I) illustrates form A but the formula is to be taken as embracing both form A and form B.

Where compounds of the formula (I) contain one or more chiral centres, and can exist in the form of two or more optical isomers, references to compounds of the formula (I) include all optical isomeric forms thereof (e.g. enantiomers and diastereoisomers), either as individual optical isomers, or mixtures or two or more optical isomers, unless the context requires otherwise.

For example, the group A can include one or more chiral centres. Thus, when E and R¹ are both attached to the same carbon atom on the linker group A, the said carbon atom is typically chiral and hence the compound of the formula (I) will exist as a pair of enantiomers (or more than one pair of enantiomers where more than one chiral centre is present in the compound).

The optical isomers may be characterised and identified by their optical activity (i.e. as + and − isomers) or they may be characterised in terms of their absolute stereochemistry using the “R and S” nomenclature developed by Calm, Ingold and Prelog, see Advanced Organic Chemistry by Jerry March, 4^{th} Edition, John Wiley & Sons, New York, 1992, pages 109-114, and see also Calm, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

As an alternative to chiral chromatography, optical isomers can be separated by forming diastereoisomeric salts with chiral acids such as (+)-tartaric acid, (−)-pyroglutamic acid, (−)-di-toluloyl-L-tartaric acid, (+)-mandelic acid, (−)-malic acid, and (−)-camphorsulphonic, separating the diastereoisomers by preferential crystallisation, and then dissociating the salts to give the individual enantiomer of the free base.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

Esters such as carboxylic acid esters and acyloxy esters of the compounds of formula (I) bearing a carboxylic acid group or a hydroxyl group are also embraced by Formula (I). In one embodiment of the invention, formula (I) includes within its scope esters of compounds of the formula (I) bearing a carboxylic acid group or a hydroxyl group. In another embodiment of the invention, formula (I) does not include within its scope esters of compounds of the formula (I) bearing a carboxylic acid group or a hydroxyl group. Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇alkyl (e.g., -Me, -Et, -nPr, -iPr, -nBu, -sBu, -iBu, -tBu);
C₁₋₇ aminoalkyl (e.g., aminoethyl; 2-(N,N-diethylamino)ethyl; 2-(4-morpholino)ethyl); and acyloxy-C₁₋₇allyl (e.g., acyloxymethyl; acyloxyethyl; pivaloyloxymethyl; acetoxymethyl; 1-acetoxyethyl ; 1-(1-methoxy-1-methyl)ethyl-carbonyloxyethyl ; 1-(benzoyloxy)ethyl ; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)-carbonyloxyethyl; (4-tetrahydropyranyl)carbonyloxymethyl; and 1-(4-tetrahydropyranyl)-carbonyloxyethyl).

Also, some prodrugs are activated enzymatically to yield the active compound, or a compound which, upon further chemical reaction, yields the active compound (for example, as in antigen-directed enzyme pro-drug therapy (ADEPT), gene-directed enzyme pro-drug therapy (GDEPT) and ligand-directed enzyme pro-drug therapy (LIDEPT). For example, the prodrug may be a sugar derivative or other glycoside conjugate, or may be an amino acid ester derivative.

### Methods for the Preparation of Compounds of the Formula (I)

In this section, as in all other sections of this application, unless the context indicates otherwise, references to formula (I) include references to formulae (Ia), (Ib), (II), (IIa), (IIb), (IIc), (III), (IIIa), (IV), (V), (VI), (VII), (VIIa), (VIII) and (IX) and all other sub-groups, preferences and examples thereof as defined herein.

Compounds of the formula (I) can be prepared by reaction of a compound of the formula (X) with a compound of the formula (XI) or an N-protected derivative thereof:

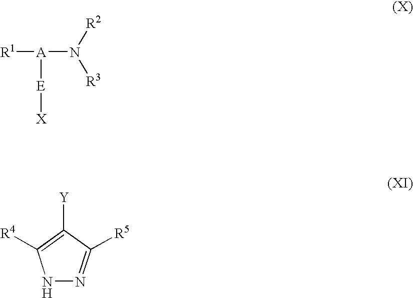

wherein A, E, and R¹ to R⁵ are as hereinbefore defined, one of the groups X and Y is chlorine, bromine or iodine or a trifluoromethanesulphonate (triflate) group, and the other one of the groups X and Y is a boronate residue, for example a boronate ester or boronic acid residue.

The reaction can be carried out under typical Suzuki Coupling conditions in the presence of a palladium catalyst such as bis(tri-t-butylphosphine)palladium and a base (e.g. a carbonate such as potassium carbonate). The reaction may be carried out in an aqueous solvent system, for example aqueous ethanol, and the reaction mixture is typically subjected to heating, for example to a temperature in excess of 100° C.

An illustrative synthetic route involving a Suzuki coupling step is shown in Scheme 1. The starting material for the synthetic route shown in scheme 1 is the halo-substituted aryl- or heteroarylmethyl nitrile (XII) in which X is a chlorine, bromine or iodine atom or a triflate group. The nitrile (XII) is condensed with the aldehyde R¹CHO in the presence of an alkali such as sodium or potassium hydroxide in an aqueous solvent system such as aqueous ethanol. The reaction can be carried out at room temperature.

The resulting substituted acrylonitrile derivative (XIII) is then treated with a reducing agent that will selectively reduce the alkene double bond without reducing the nitrile group. A borohydride such as sodium borohydride may be used for this purpose to give the substituted acetonitrile derivative (XIV). The reduction reaction is typically carried out in a solvent such as ethanol and usually with heating, for example to a temperature up to about 65° C.

The reduced nitrile (XIV) is then coupled with the pyrazole boronate ester (XV) under the Suzuki coupling conditions described above to give a compound of the formula (I) in which A-NR²R³ is a substituted acetonitrile group.

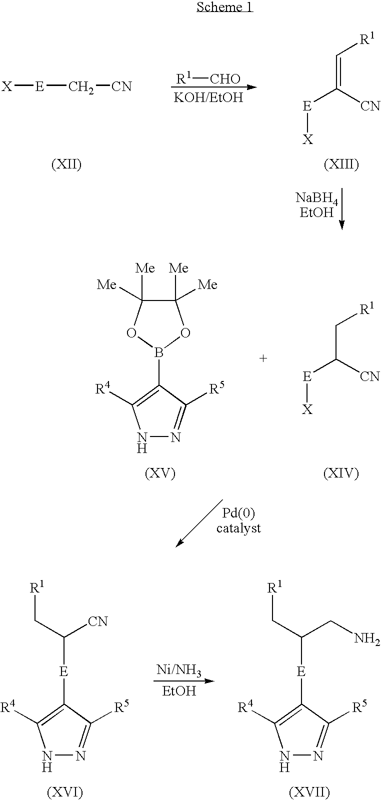

The substituted acetonitrile compound (XVI) may then be reduced to the corresponding amine (XVII) by treatment with a suitable reducing agent such as Raney nickel and ammonia in ethanol.

The synthetic route shown in Scheme 1 gives rise to amino compounds of the formula (I) in which the aryl or heteroaryl group E is attached to the β-position of the group A relative to the amino group. In order to give amino compounds of the formula (I) in which R¹ is attached to the β-position relative to the amino group, the functional groups on the two starting materials in the condensation step can be reversed so that a compound of the formula X-E-CHO wherein X is bromine, chlorine, iodine or a triflate group is condensed with a compound of the formula R¹—CH₂—CN to give a substituted acrylonitrile derivative which is then reduced to the corresponding acetonitrile derivative before coupling with the pyrazole boronate (XV) and reducing the cyano group to an amino group.

Compounds of the formula (I) in which R¹ is attached to the a-position relative to the amino group can be prepared by the sequence of reactions shown in Scheme 2.

In Scheme 2, the starting material is a halo-substituted aryl- or heteroarylmethyl Grignard reagent (XVIII, X=bromine or chlorine) which is reacted with the nitrile R¹—CN in a dry ether such as diethyl ether to give an intermediate imine (not shown) which is reduced to give the amine (XIX) using a reducing agent such as lithium aluminium hydride. The amine (XIX) can be reacted with the boronate ester (XV) under the Suzuki coupling conditions described above to yield the amine (XX).

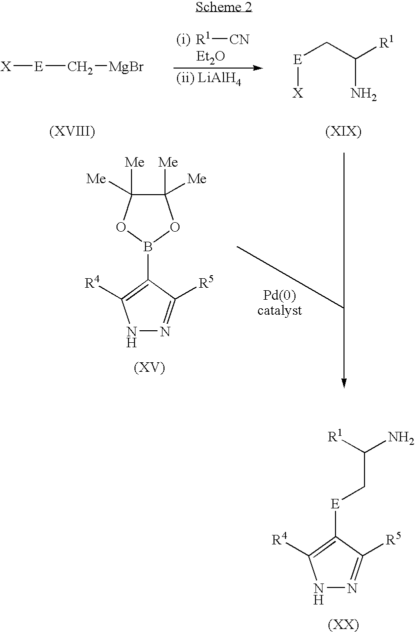

Compounds of the formula (I) can also be prepared from the substituted nitrile compound (XXI):

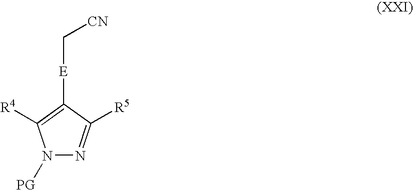

wherein PG is a protecting group such as a tetrahydropyranyl group. The nitrile (XXI) can be condensed with an aldehyde of the formula R¹—(CH₂)ᵣ—CHO, wherein r is 0 or 1, and the resulting substituted acrylonitrile subsequently reduced to the corresponding substituted nitrile under conditions analogous to those set out in Scheme 1 above. The protecting group PG can then be removed by an appropriate method. The nitrile compound may subsequently be reduced to the corresponding amine by the use of a suitable reducing agent as described above.

The nitrile compound (XXI) may also be reacted with a Grignard reagent of the formula R¹—(CH₂)ᵣ—MgBr under standard Grignard reaction conditions followed by deprotection to give an amino compound of the invention which has the structure shown in formula (XXII).

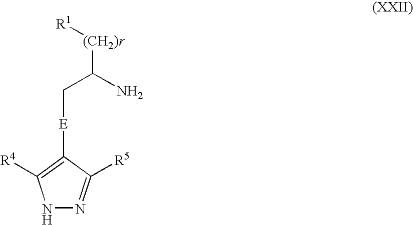

In the preparative procedures outlined above, the coupling of the aryl or heteroaryl group E to the pyrazole is accomplished by reacting a halo-pyrazole or halo-aryl or heteroaryl compound with a boronate ester or boronic acid in the presence of a palladium catalyst and base. Many boronates suitable for use in preparing compounds of the invention are commercially available, for example from Boron Molecular Limited of Noble Park, Australia, or from Combi-Blocks Inc, of San Diego, USA. Where the boronates are not commercially available, they can be prepared by methods known in the art, for example as described in the review article by N. Miyaura and A. Suzuki, Chem. Rev. 1995, 95, 2457. Thus, boronates can be prepared by reacting the corresponding bromo-compound with an alkyl lithium such as butyl lithium and then reacting with a borate ester. The resulting boronate ester derivative can, if desired, be hydrolysed to give the corresponding boronic acid.

Compounds of the formula (XI) wherein Y is B(OH)₂ and wherein a protecting group (such as a dimethylaminosulphonyl group) is attached to the pyrazole 1-nitrogen atom can be prepared from the corresponding compound wherein Y is a bromine atom by reaction with a trialkylborate such as triethylborate in the presence of an alkyl lithium such as methyl lithium. The reaction is typically carried out in a polar non-protic solvent such as tetrahydrofuran and is followed by treatment with an acid to give a boronic acid compound.

Compounds of the formula (I) in which the group A contains a nitrogen atom attached to the group E can be prepared by well known synthetic procedures from compounds of the formula (XXIII) or a protected form thereof. Compounds of the formula (XXIII) can be obtained by a Suzuki coupling reaction of a compound of the formula (XV) (see Scheme 1) with a compound of the formula Br-E-NH₂ such as 4-bromoaniline.

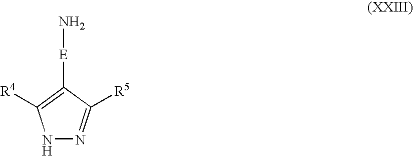

Compounds of the formula (I) in which R¹ and E are connected to the same carbon atom can be prepared as shown in Scheme 3.

In Scheme 3, an aldehyde compound (XXIV) where X is bromine, chlorine, iodine or a triflate group is condensed with ethyl cyanoacetate in the presence of a base to give a cyanoacrylate ester intermediate (XXV). The condensation is typically carried out in the presence of a base, preferably a non-hydroxide such as piperidine, by heating under Dean Stark conditions.

The cyanoacrylate intermediate (XXV) is then reacted with a Grignard reagent R¹MgBr suitable for introducing the group R¹ by Michael addition to the carbon-carbon double bond of the acrylate moiety. The Grignard reaction may be carried out in a polar non-protic solvent such as tetrahydrofuran at a low temperature, for example at around 0° C. The product of the Grignard reaction is the cyano propionic acid ester (XXVI) and this is subjected to hydrolysis and decarboxylation to give the propionic acid derivative (XXVII). The hydrolysis and decarboxylation steps can be effected by heating in an acidic medium, for example a mixture of sulphuric acid and acetic acid.

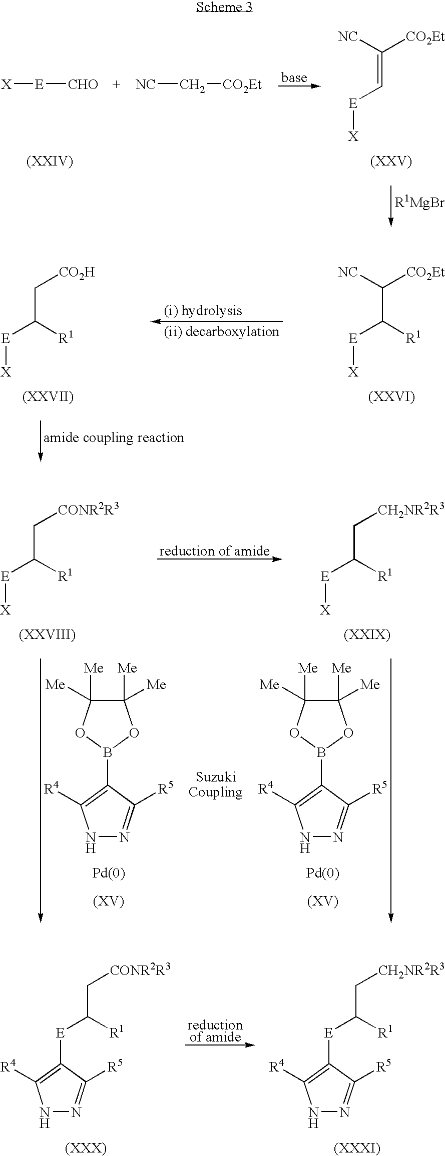

The propionic acid derivative (XXVII) is converted to the amide (XXVIII) by reaction with an amine HNR²R³ under conditions suitable for forming an amide bond. The coupling reaction between the propionic acid derivative (XXVII) and the amine HNR²R³ is preferably carried out in the presence of a reagent of the type commonly used in the formation of peptide linkages. Examples of such reagents include 1,3-dicyclohexylcarbodiimide (DCC) (Sheehan et al, J. Amer. Chem. Soc. 1955, 77, 1067), 1-ethyl-3-(3′-dimethylaminopropyl)-carbodiimide (referred to herein either as EDC or EDAC) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) and phosphonium-based coupling agents such as 1-benzo-triazolyloxytris-(pyrrolidino)phosphonium hexafluorophosphate (PyBOP) (Castro et al, Tetrahedron Letters, 1990, n, 205). Carbodiimide-based coupling agents are advantageously used in combination with 1-hydroxy-7-azabenzotriazole (HOAt) (L. A. Carpino, J. Amer. Chem. Soc., 1993, 115, 4397) or 1-hydroxybenzotriazole (HOBt) (Konig et al, Chem. Ber., 103, 708, 2024-2034). Preferred coupling reagents include EDC (EDAC) and DCC in combination with HOAt or HOBt.

The coupling reaction is typically carried out in a non-aqueous, non-protic solvent such as acetonitrile, dioxan, dimethylsulphoxide, dichloromethane, dimethylformamide or N-methylpyrrolidine, or in an aqueous solvent optionally together with one or more miscible co-solvents. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

Where the amine HNR²R³ is ammonia, the amide coupling reaction can be carried out using 1,1′-carbonyldiimidazole (CDI) to activate the carboxylic acid before addition of the ammonia.

As an alternative, a reactive derivative of the carboxylic acid, e.g. an anhydride or acid chloride, may be used. Reaction with a reactive derivative such an anhydride is typically accomplished by stirring the amine and anhydride at room temperature in the presence of a base such as pyridine.

The amide (XXVIII) can be converted to a compound of the formula (XXX) (which corresponds to a compound of the formula (I) wherein A has an oxo substituent next to the NR²R³ group) by reaction with a boronate (XV) under Suzuki coupling conditions as described above. The amide (XXX) can subsequently be reduced using a hydride reducing agent such as lithium aluminium hydride in the presence of aluminium chloride to give an amine of the formula (XXXI) (which corresponds to a compound of the formula (I) wherein A is CH—CH₂—CH₂-). The reduction reaction is typically carried out in an ether solvent, for example diethyl ether, with heating to the reflux temperature of the solvent.

Rather than reacting the amide (XXVIII) with the boronate (XV), the amide may instead be reduced with lithium aluminium hydride/aluminium chloride, for example in an ether solvent at ambient temperature, to give the amine (XXIX) which is then reacted with the boronate (XV) under the Suzuki coupling conditions described above to give the amine (XXX).

In order to obtain the homologue of the amine (XXIX) containing one fewer methylene group, the carboxylic acid (XXVII) can be converted to the azide by standard methods and subjected to a Curtius rearrangement in the presence of an alcohol such as benzyl alcohol to give a carbamate (see Advanced Organic Chemistry, 4^{th} edition, by Jerry March, John Wiley & sons, 1992, pages 1091-1092). The benzylcarbamate can function as a protecting group for the amine during the subsequent Suzuki coupling step, and the benzyloxycarbonyl moiety in the carbamate group can then be removed by standard methods after the coupling step. Alternatively, the benzylcarbamate group can be treated with a hydride reducing agent such as lithium aluminium hydride to give a compound in which NR²R³ is a methylamino group instead of an amino group.

Intermediate compounds of the formula (X) where the moiety X is a chlorine, bromine or iodine atom and A is a group CH—CH₂— can be prepared by the reductive amination of an aldehyde compound of the formula (XXXII):

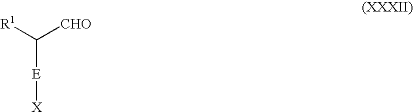

with an amine of the formula HNR²R³ under standard reductive amination conditions, for example in the presence of sodium cyanoborohydride in an alcohol solvent such as methanol or ethanol.

The aldehyde compound (XXXII) can be obtained by oxidation of the corresponding alcohol (XXXIII) using, for example, the Dess-Martin periodinane (see Dess, D. B.; Martin, J. C. J. Org. Soc., 1983, 48, 4155 and Organic Syntheses, Vol. 77, 141).

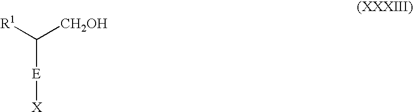

Compounds of the formula (I) where A, N and R² together form a cyclic group can be formed by the Suzuki coupling of a boronate compound of the formula (XV) with a cyclic intermediate of the formula (XXXIV) or an N-protected derivative thereof.

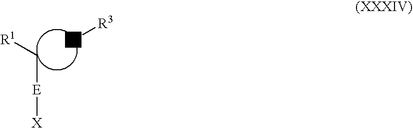

Cyclic intermediates of the formula (XXXIV), where R¹ is an aryl group such as an optionally substituted phenyl group, can be formed by Friedel Crafts alkylation of an aryl compound R¹—H with a compound of the formula (XXXV):

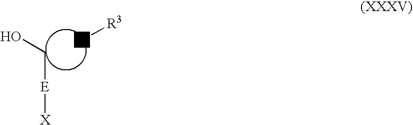

The alkylation is typically carried out in the presence of a Lewis acid such as aluminium chloride at a reduced temperature, for example less than 5° C.

The Friedel Crafts reaction has been found to be of general applicability to the preparation of a range of intermediates of the formula (X). Accordingly, in a general method of making compounds of the formula (X), a compound of the formula (LXX):

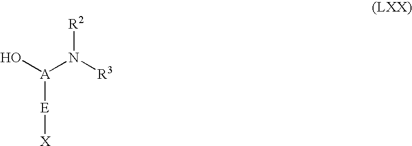

is reacted with a compound of the formula R¹—H under Friedel Crafts alkylation conditions, for example in the presence of an aluminium, halide (e.g. A1Cl₃).

In a further method for the preparation of a compound of the formula (I) wherein the moiety NR²R³ is attached to a CH₂ group of the moiety A, an aldehyde of the formula (XXXVI) can be coupled with an amine of the formula HNR²R³ under reductive amination conditions as described above. In the formulae (XXXVI) and (XXXVII), A′ is the residue of the group A—i.e. the moieties A′ and CH₂ together form the group A. The aldehyde (XXXVII) can be formed by oxidation of the corresponding alcohol using, for example, Dess-Martin periodinane.

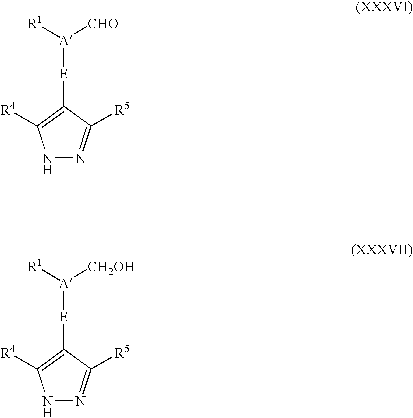

A Friedel Crafts alkylation procedure of the type described above for the synthesis of intermediates of the formula (XXXIV) can also be used to prepare intermediates of the formula (X) wherein X is bromine. An example of such a procedure is shown in Scheme 4.

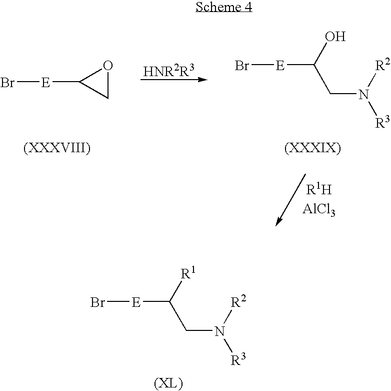

The starting material for the synthetic route shown in Scheme 4 is the epoxide (XXXVII) which can either be obtained commercially or can be made by methods well known to the skilled person, for example by reaction of the aldehyde Br-E-CHO with trimethylsulphonium iodide. The epoxide (XXXVII) is reacted with an amine HNR²R³ under conditions suitable for a ring-opening reaction with the epoxide to give a compound of the formula (XXXIX). The ring opening reaction can be carried out in a polar solvent such as ethanol at room temperature or optionally with mild heating, and typically with a large excess of the amine.

The amine (XXXIX) is then reacted with an aryl compound R¹H, typically a phenyl compound, capable of taking part in a Friedel Crafts alkylation (see for example Advanced Organic Chemistry, by Jerry March, pages 534-542). Thus, the amine of formula (XXXIX) is typically reacted with the aryl compound R¹H in the presence of an aluminium chloride catalyst at or around room temperature. Where the aryl compound R¹H is a liquid, e.g. as in the case of a methoxybenzene (e.g. anisole) or a halobenzene such as chlorobenzene, the aryl compound may serve as the solvent. Otherwise, a less reactive solvent such as nitrobenzene may be used. The Friedel Crafts alkylation of the compound R¹H with the amine (XXXIX) gives a compound of the formula (XL) which corresponds to a compound of the formula (X) wherein X is bromine and A is CHCH₂.

The hydroxy intermediate (XXL) in Scheme 4 can also be used to prepare compounds of the formula (X) in which the carbon atom of the hydrocarbon linker group A adjacent the group R¹ is replaced by an oxygen atom. Thus the compound of formula (XXXIX), or an N-protected derivative thereof (where R² or R³ are hydrogen) can be reacted with a phenolic compound of the formula R¹—OH under Mitsunobu alkylation conditions, e.g. in the presence of diethyl azodicarboxylate and triphenylphosphine. The reaction is typically carried out in a polar non-protic solvent such as tetrahydrofuran at a moderate temperature such as ambient temperature.

A further use of the hydroxy-intermediate (XXXIX) is for the preparation of the corresponding fluoro-compound. Thus, the hydroxy group can be replaced by fluorine by reaction with pyridine:hydrogen fluoride complex (Olah's reagent). The fluorinated intermediate can then be subjected to a Suzuki coupling reaction to give a compound of the formula (I) with a fluorinated hydrocarbon group A. A fluorinated compound of the formula (I) could alternatively be prepared by first coupling the hydroxy intermediate (XXXIX), or a protected form thereof, with a pyrazole boronic acid or boronate under Suzuki conditions and then replacing the hydroxy group in the resulting compound of formula (I) with fluorine using pyridine: hydrogen fluoride complex.

Compounds of the formula (I) in which the moiety:

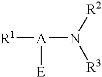

is a group:

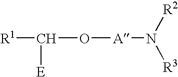

where A″ is the hydrocarbon residue of the group A, can be prepared by the sequence of reactions shown in Scheme 5.

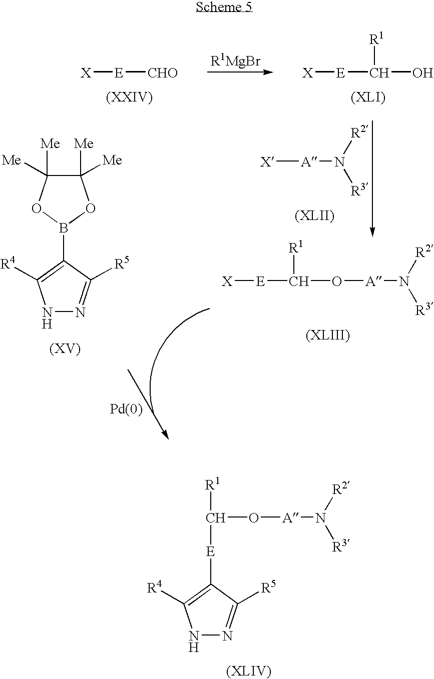

As shown in Scheme 5, the aldehyde (XXIV) is reacted with a Grignard reagent R¹MgBr under standard Grignard conditions to give the secondary alcohol (XLI). The secondary alcohol can then be reacted with a compound of the formula (XLII) in which R^{2′} and R^{3′} represent the groups R² and R³ or an amine-protecting group, A″ is the residue of the group A, and X′ represents a hydroxy group or a leaving group.

The amine protecting group can be, for example, a phthalolyl group in which case NR^{2′}R^{3′} is a phthalimido group.

When X′ is a hydroxy group, the reaction between compound (XLI) and (XLII) can take the form of an toluene sulphonic acid catalysed condensation reaction. Alternatively, when X′ is a leaving group such as halogen, the alcohol (XLI) can first be treated with a strong base such as sodium hydride to form the alcoholate which then reacts with the compound (XLII).

The resulting compound of the formula (XLIII) is then subjected to a Suzuki coupling reaction with the pyrazole boronate reagent (XV) under typical Suzuki coupling conditions of the type described above to give a compound of the formula (XLIV). The protecting group can then be removed from the protected amine group NR^{2′}R^{3′} to give a compound of the formula (I).

Compounds of the formula (I) in which the moiety:

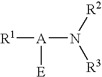

is a group:

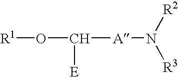

where A″ is the hydrocarbon residue of the group A, can be prepared by the sequence of reactions shown in Scheme 6.

The starting material in Scheme 6 is the chloroacyl compound (XLV) which can be prepared by literature methods (e.g. the method described in J. Med. Chem., 2004, 47, 3924-3926) or methods analogous thereto. Compound (XLV) is converted into the secondary alcohol (XLVI) by reduction with a hydride reducing agent such as sodium borohydride in a polar solvent such as water/tetrahydrofuran.

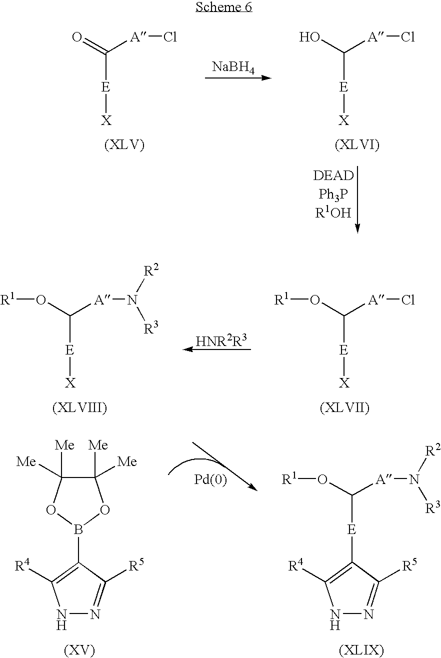

The secondary alcohol (XLVI) can then be reacted with a phenolic compound of the formula R¹—OH under Mitsunobu alkylation conditions, e.g. in the presence of diethyl azodicarboxylate and triphenylphosphine, as described above, to give the aryl ether compound (XLVII).

The chorine atom in the aryl ether compound (XLVII) is then displaced by reaction with an amine HNR²R³ to give a compound of the formula (XLVIII). The nucleophilic displacement reaction may be carried out by heating the amine with the aryl ether in a polar solvent such as an alcohol at an elevated temperature, for example approximately 100° C. The heating may advantageously be achieved using a microwave heater. The resulting amine (XLVIII) can then be subjected to a Suzuki coupling procedure with a boronate of the formula (XV) as described above to give the compound (XLIX).

In a variation on the reaction sequence shown in Scheme 6, the secondary alcohol (XLVI) can be subjected to a nucleophilic displacement reaction with an amine HNR²R³ before introducing the group R¹ by means of the Mitsunobu ether-forming reaction.

Compounds of the formula (X) in which the moiety:

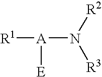

is a group:

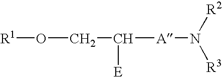

where A″ is the hydrocarbon residue of the group A, can be prepared by the reaction of a compound of the formula (LXX):

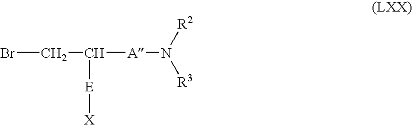

or a protected form thereof with a compound of the formula R¹OH (preferably a phenolic compound) in the presence of a base. When R¹OH is a phenolic compound, an alkali metal carbonate such as caesium carbonate can be used. The reaction is typically carried out with heating in a high boiling polar solvent such as dimethylformamide.

The bromo-compounds of the formula (LXX) can be prepared from the corresponding hydroxy compound by reaction with a brominating reagent such as carbon tetrabromide and triphenylphosphine. The hydroxy compounds can be obtained by reduction of a carboxylic acid of the formula (LXXI):

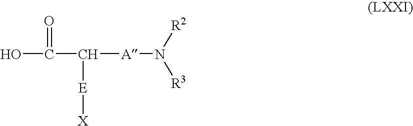

using a borane-containing reducing agent such as borane-tetrahydrofuran complex.

Another route to compounds of the formula (I) in which E and R¹ are attached to the same carbon atom in the group A is illustrated in Scheme 7.

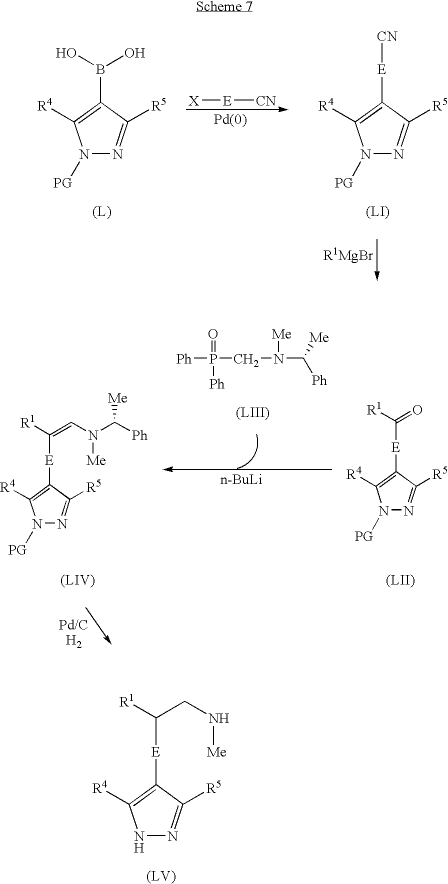

In Scheme 7, an N-protected pyrazolyl boronic acid (L) is reacted under Suzuki coupling conditions with the cyano compound X-E-CN in which X is typically a halogen such as bromine or chlorine. The protecting group PG at the 1-position of the pyrazole ring may be, for example, a triphenylmethyl (trityl) group. The boronic acid (L) can be prepared using the method described in EP 1382603 or methods analogous thereto.

The resulting nitrile (LI) may then be reacted with a Grignard reagent R¹—MgBr to introduce the group R¹ and form the ketone (LII). The ketone (LII) is converted to the enamine (LIV) by reaction with the diphenylphosphinoylmethylamine (LIII) in the presence of a strong base such as an alkyl lithium, particularly butyl lithium.

The enamine (LIV) is then subjected to hydrogenation over a palladium on charcoal catalyst to reduce the double bond of the enamine and remove the 1-phenethyl group. Where the protecting group PG is a trityl group, hydrogenation also removes the trityl group, thereby yielding a compound of the formula (LV).

Alternatively, the enamine (LTV) can be reduced with a hydride reducing agent under the conditions described in Tetrahedron: Asymmetry 14 (2003) 1309-1316 and subjected to a chiral separation. Removal of the protecting 2-phenethyl group and the protecting group PG then gives an optically active form of the compound of formula (LV).

Intermediates of the formula (X) wherein A and R² link to form a ring containing an oxygen atom can be prepared by the general method illustrated in Scheme 8.

In Scheme 8, a ketone (LVI) is reacted with trimethylsulphonium iodide to form the epoxide (LVII). The reaction is typically carried out in the presence of a hydride base such as sodium hydride in a polar solvent such as dimethylsulphoxide.

The epoxide (LVII) is subjected to a ring opening reaction with ethanolamine in the presence of a non-interfering base such as triethylamine in a polar solvent such as an alcohol (e.g. isopropanol), usually with mild heating (e.g. up to approximately 50° C. The resulting secondary alcohol is then cyclised to form the morpholine ring by treatment with concentrated sulphuric acid in a solvent such as ethanolic dichloromethane.

The morpholine intermediate (LIX) can then reacted with the boronate (XV) under Suzuki coupling conditions to give the compound of formula (LX), which corresponds to a compound of the formula (I) in which A-NR²R³ forms a morpholine group.

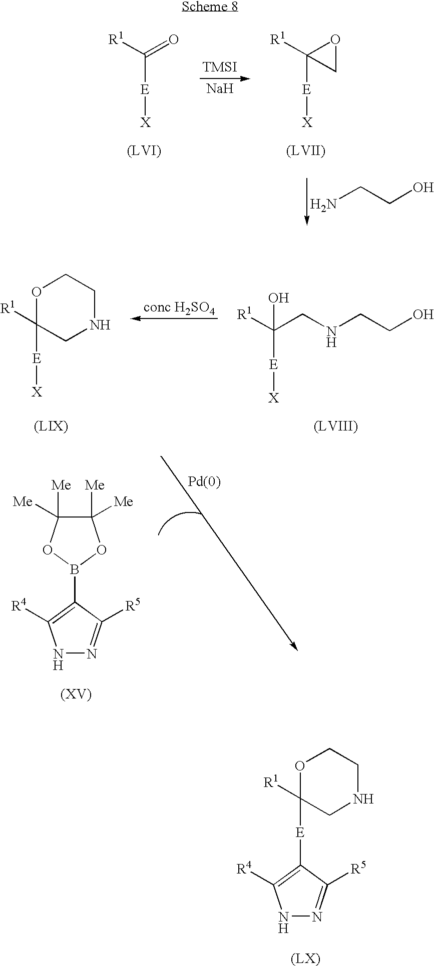

Instead of reacting the epoxide (LVII) with ethanolamine, it may instead be reacted with mono- or dialkylamines thereby providing a route to compounds containing the moiety:

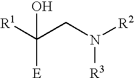

Compounds wherein R² and R³ are both hydrogen can be prepared by reacting the epoxide (LVII) with potassium phthalimide in a polar solvent such as DMSO. During the Suzuki coupling step, the phthalimide group may undergo partial hydrolysis to give the corresponding phthalamic acid which can be cleaved using hydrazine to give the amino group NH₂. Alternatively, the phthalamic acid can be recyclised to the phthalimide using a standard amide-forming reagent and the phthaloyl group then removed using hydrazine to give the amine.

As shown in Scheme 8A, the benzophenone (LVI) can also be reacted with the anion generated by treatment of acetonitrile with an alkyl lithium reagent to give the hydroxy-nitrile compound (LXXV). A subsequent Suzuki coupling, under the standard conditions, followed by reduction of the nitrile (LXXVI) with lithium aluminium hydride, permits the synthesis of compounds of the formula (LXXVII)

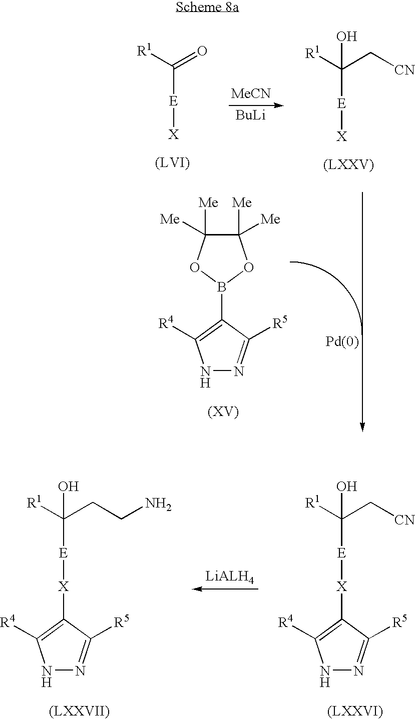

Ketones of the formula (LVI) can be obtained commercially or prepared according to methods well known to the skilled person. For example, they can be formed by the reaction of a compound of the formula Br-E-CN with a Grignard reagent R¹—MgBr suitable for introducing the group R¹. The reaction can be carried out under standard Grignard reaction conditions.

In a variation on the reaction sequence set out in Scheme 8a, the hydroxy-nitrile (LXXV) can be alkylated to give the corresponding C₁₋₄alkoxy or substituted C₁₋₄ alkoxy compound. The alkylation can be carried out using known methods, e.g. by reaction with a base such as sodium hydride to form a hydroxylate anion followed by reaction with an alkyl halide or substituted alkyl halide. The alkoxy compound can then be reacted with the pyrazolyl boronate (XV) under Suzuki coupling conditions and the nitrile group reduced to give the alkoxy analogue of (LXXVII).

In another variation on the reaction sequence set out in Scheme 8a, the hydroxy-nitrile (LXXV) can be reacted with two equivalents of methyl magnesium bromide under Grignard reaction conditions to give a compound of the formula (LXXXXV):

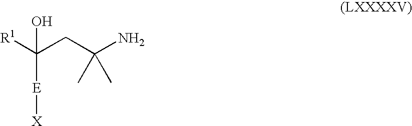

The gem-dimethyl-substituted amine (LXXXXV) can then be reacted with the pyrazolyl-borane (XV) to give a compound of the formula (I)

The homologues of compounds of the formula (LXXVII) in which the groups R¹, E and OH are linked to the 1-position of a 4-aminobutyl group rather than a 3-aminopropyl group can be prepared according to the method described in Tetrahedron, 1989, 2183 by lithiation of Cl—CH₂CH₂CH₂NH(CO)Ph using tert-butyl lithium and then reacting the lithiated intermediate with the ketone (LVI) to give a compound of the formula (LXXXIII):

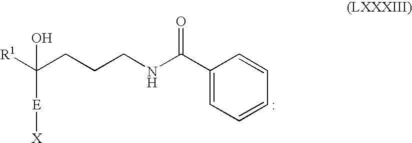

The benzoylamino compound (LXXXIII) is then subjected to basic hydrolysis (for example using lithium hydroxide) to remove the benzoyl group and give the amine: (LXXXIX):

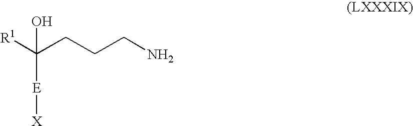

The amine (LXXXIX), X=Br, is then reacted with the pyrazolyl-boronate (XV) under the Suzuki coupling conditions described below in Scheme 8b to introduce the pyrazole group and give a compound of the formula (I).

Analogues of compounds of the formula (LXXVII) wherein an alkylamino group is present in place of the NH₂ group, can be prepared by the sequence of reactions shown in Scheme 8b,

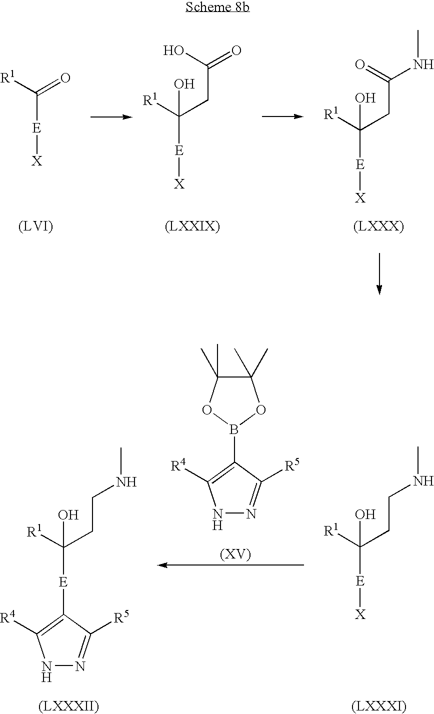

In the reaction sequence shown in Scheme 8b, the ketone (LVI) is converted to the hydroxy-acid (LXXIX) by following the method described in J. Amer. Chem. Soc., 1950, 1522. The hydroxy-acid is then converted to the corresponding amide by reaction with methylamine (or another amine) using the amide coupling conditions described above. Thus, for example, the amide coupling reaction can be brought about using a mixture of 1-hydroxybenztriazole and 1-(3-dimethylamisiopropyl)-ethylcarbodiimide. The resulting amide (LXXX) is then reduced to the amine (LXXXI) using a reducing agent such as lithium aluminium hydride. The amine (LXXXI), in which X is typically a halogen such as bromine, can then be reacted with the pyrazolyl borane (XV) under standard Suzuki coupling conditions as described above to give the product (LXXXII).

A further synthetic route to compounds of the formula (I) wherein A and NR²R³ combine to form a cyclic group is illustrated in Scheme 9.

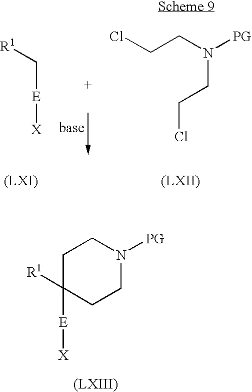

In Scheme 9, the starting material (LXI) is typically a di-aryl/heteroaryl methane in which one or both of the aryl/heteroaryl groups is capable of stabilising or facilitating formation of an anion formed on the methylene group between E and R¹. For example, R¹ may advantageously be a pyridine group. The starting material (LXI) is reacted with the N-protected bis-2-chloroethylamine (LXII) in the presence of a non-interfering strong base such as sodium hexamethyldisilazide in a polar solvent such as tetrahydrofuran at a reduced temperature (e.g. around 0° C.) to give the N-protected cyclic intermediate (LXIII). The protecting group can be any standard amine-protecting group such as a Boc group. Following cyclisation, the intermediate (LXIII) is coupled to a boronate of the formula (XV) under Suzuki coupling conditions and then deprotected to give the compound of the formula (I).

Compounds of the formula (I) in which the moiety:

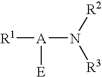

is a group:

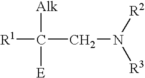

wherein “Alk” is a small alkyl group such as methyl or ethyl can be formed by the synthetic route illustrated in Scheme 10.

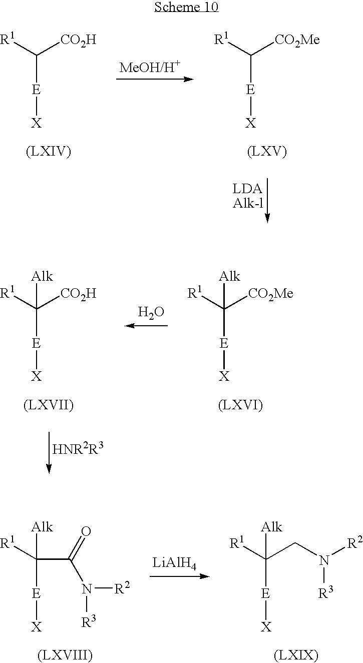

In Scheme 10, a carboxylic acid of the formula (LXIV) is esterified by treatment with methanol in the presence of an acid catalyst such as hydrochloric acid. The ester (LXV) is then reacted with a strong base such as lithium diisopropylamide (LDA) and an alkyl iodide such as methyl iodide at reduced temperature (e.g. between 0° C. and −78° C.). The branched ester (LXVI) is then hydrolysed to the acid (LXVII) and coupled with an amine HNR²R³ under standard amide forming conditions of the type described above. The amide (LXVIII) can then be reduced to the amine (LXIX) using lithium aluminium hydride, and the amine (LXIX) is then reacted with a pyrazole boronate or boronic acid under Suzuki coupling conditions to give a compound of the formula (I).

Compounds of the formula (I) in which the moiety:

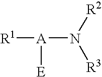

is a group:

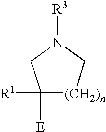

wherein n is 0, 1 or 2 can be prepared by the synthetic route set out in Scheme 11.

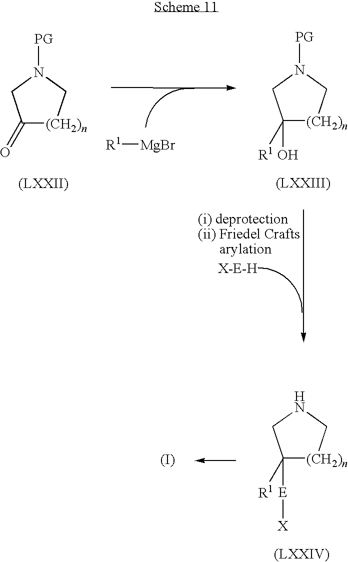

In Scheme 11, an N-protected oxo-piperidine (n=2), oxo-pyrrolidine (n=1), oxo-azetidine (n=0) is reacted with a Grignard reagent suitable for introducing a group R¹ to give a hydroxy compound of the formula (LXIII). The protecting group (e.g. a Boc group) can then be removed and the deprotected compound of formula LXXIII) used in a Friedel Crafts acylation reaction with a compound X-E-H where E is an aromatic group and X is a halogen atom. The Friedel Crafts reaction is carried out in the presence of aluminium chloride, typically in a non-polar solvent such as dichloromethane. The resulting compound of the formula (LXXIV) is then reacted with a compound of the formula (XI) wherein Y is a boronate ester or boronic acid group under the Suzuki coupling conditions described above.

Compounds of the formula (II) above, wherein R¹⁸ and R¹⁹ are both hydrogen and R²⁴ and R^{2a} link to form an azetidine group, can be formed by the sequence of reactions illustrated in Scheme 12.

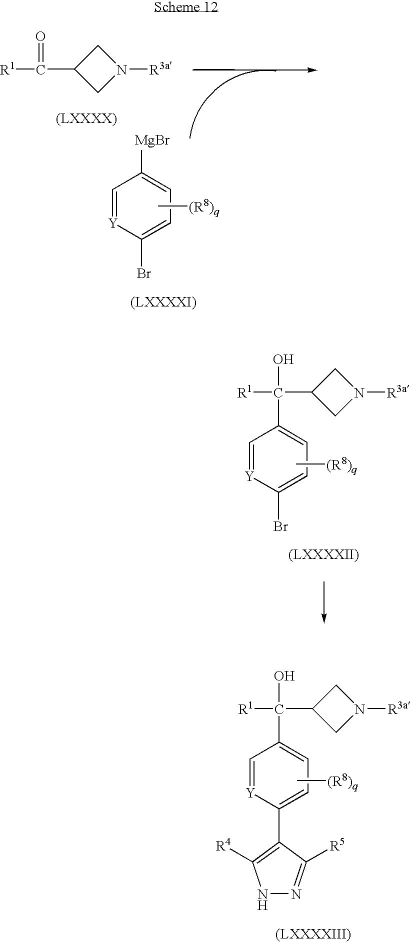

The ketone (LXXXX) can be prepared according to the methods described in WO 2005/026113 or methods analogous thereto. In formula (LXXXX), the group R^{3a′} can either be a protecting group such as a Boc group, or it can be one of the non-hydrogen groups falling under the definition of the group R^{3a}. The ketone (LXXXX) is reacted with the Grignard reagent (LXXXXI) under standard Grignard conditions to give the hydroxy compound (MOOCH) which is subsequently reacted with the pyrazolyl-boronate (XV) under Suzuki coupling conditions to give the compound of formula (II). By using starting materials that are homologous to the azetidine ketone (LXXXX), compounds wherein R²⁴ and R^{2a} link to form a pyrrolidine or piperidine group may be prepared.

Compounds of the formula (I) in which the moiety:

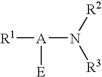

is a group:

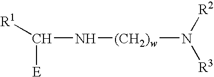

can be prepared by the sequence of reactions shown in Scheme 13.

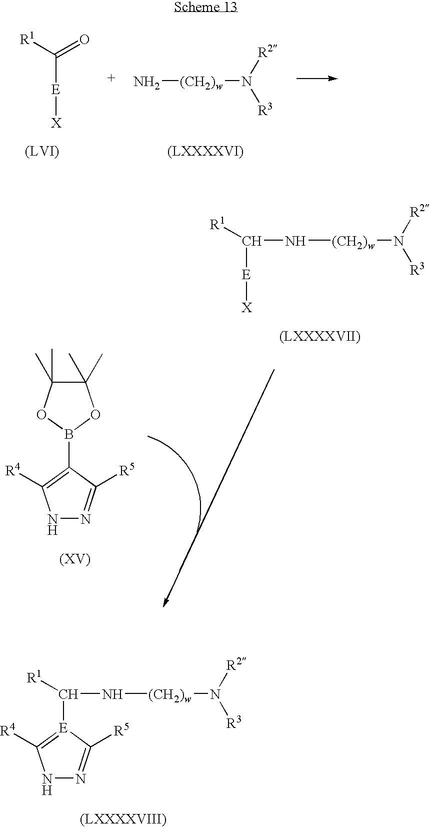

In Scheme 13, the ketone (LVI) is reacted with the amine (LXXXXVI) under reductive amination conditions, for example using sodium cyanoborohydride as the reducing agent. In the amine (LXXXXVI), the group R^{2″} can be either a protecting group (such as a boc group) or a moiety other than hydrogen and selected from the definition of R² above. The intermediate (LXXXXVII) is then coupled with the pyrazolyl boronate (XV) under standard Suzuki coupling conditions to give the compound (LXXXXVIII). When R^{2″} is a protecting group such as boc, this can then be removed under standard conditions to give the target compound.

Once formed, many compounds of the formula (I) can be converted into other compounds of the formula (I) using standard functional group interconversions. For example, compounds of the formula (I) in which the NR²R³ forms part of a nitrile group can be reduced to the corresponding amine. Compounds in which NR²R³ is an NH₂ group can be converted to the corresponding alkylamine by reductive alkylation, or to a cyclic group. Compounds wherein R¹ contains a halogen atom such as chlorine or bromine can be used to introduce an aryl or heteroaryl group substituent into the R¹ group by means of a Suzuki coupling reaction. Further examples of interconversions of one compound of the formula (I) to another compound of the formula (I) can be found in the examples below. Additional examples of functional group interconversions and reagents and conditions for carrying out such conversions can be found in, for example, Advanced Organic Chemistry, by Jerry March, 4^{th} edition, 119, Wiley Interscience, New York, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8).

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

A hydroxy group may be protected, for example, as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2-(phenylsulphonyl)ethyloxy amide (—NH-Psec). Other protecting groups for amines, such as cyclic amines and heterocyclic N—H groups, include toluenesulphonyl (tosyl) and methanesulphonyl (mesyl) groups and benzyl groups such as a para-methoxybenzyl (PMB) group. A carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₄alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃).

The 1(H) position of the pyrazole group in the compounds of the formula (I) or its precursors can be protected by a variety of groups, the protecting group being selected according to the nature of the reaction conditions to which the group is exposed. Examples of protecting groups for the pyrazole N—H include tetrahydropyranyl, benzyl and 4-methoxybenzyl groups.

Many of the chemical intermediates described above are novel and such novel intermediates form a further aspect of the invention.

### Pharmaceutical Formulations

While it is possible for the active compound to be administered alone, it is preferable to present it as a pharmaceutical composition (e.g. formulation) comprising at least one active compound of the invention together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Pharmaceutical compositions containing compounds of the formula (I) can be formulated in accordance with known techniques, see for example, Remington's Pharmaceutical Sciences, Mack Publishing Company, Easton, Pa., USA.

Accordingly, in a further aspect, the invention provides compounds of the formula (I) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Pharmaceutical formulations adapted for parenteral administration include aqueous and non-aqueous sterile injection solutions which may contain anti-oxidants, buffers, bacteriostats, co-solvents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

Liposomes are closed spherical vesicles composed of outer lipid bilayer membranes and an inner aqueous core and with an overall diameter of <100 μm. Depending on the level of hydrophobicity, moderately hydrophobic drugs can be solubilized by liposomes if the drug becomes encapsulated or intercalated within the liposome. Hydrophobic drugs can also be solubilized by liposomes if the drug molecule becomes an integral part of the lipid bilayer membrane, and in this case, the hydrophobic drug is dissolved in the lipid portion of the lipid bilayer.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

The pharmaceutical formulation can be prepared by lyophilising a compound of formula (I), or sub-groups thereof. Lyophilisation refers to the procedure of freeze-drying a composition. Freeze-drying and lyophilisation are therefore used herein as synonyms.

Extemporaneous injection solutions and suspensions may be prepared from sterile powders, granules and tablets.

Pharmaceutical compositions of the present invention for parenteral injection can also comprise pharmaceutically acceptable sterile aqueous or non-aqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In one preferred embodiment of the invention, the pharmaceutical composition is in a form suitable for i.v. administration, for example by injection or infusion. For intravenous administration, the solution can be dosed as is, or can be injected into an infusion bag (containing a pharmaceutically acceptable excipient, such as 0.9% saline or 5% dextrose), before administration.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated, but typically have a coating, for example a protective film coating (e.g. a wax or varnish) or a release controlling coating. The coating (e.g. a Eudragit TM type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

The compounds of the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

This invention also provides solid dosage forms comprising the solid solution described above. Solid dosage forms include tablets, capsules and chewable tablets. Known excipients can be blended with the solid solution to provide the desired dosage form. For example, a capsule can contain the solid solution blended with (a) a disintegrant and a lubricant, or (b) a disintegrant, a lubricant and a surfactant. A tablet can contain the solid solution blended with at least one disintegrant, a lubricant, a surfactant, and a glidant. The chewable tablet can contain the solid solution blended with a bulking agent, a lubricant, and if desired an additional sweetening agent (such as an artificial sweetener), and suitable flavours.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use include ointments, creams, sprays, patches, gels, liquid drops and inserts (for example intraocular inserts). Such compositions can be formulated in accordance with known methods.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the formula (I) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within this range, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 miligrams to 1 gram, of active compound.

The active compound will be administered to a patient in need thereof (for example a human or animal patient) in an amount sufficient to achieve the desired therapeutic effect.

### Protein Kinase Inhibitory Activity

The activity of the compounds of the invention as inhibitors of protein kinase A and protein kinase B can be measured using the assays set forth in the examples below and the level of activity exhibited by a given compound can be defined in terms of the 1050 value. Preferred compounds of the present invention are compounds having an IC50 value of less than 1 μM, more preferably less than 0.1 μM, against protein kinase B.

It has also been found that many compounds of the formula (I) have low hERG activity and a good separation between PHB/PKA activity and hERG activity.

Preferred compounds of the formula (I) have mean IC₅₀ values against hERG that are greater than 30 times, or greater than 40 times, or greater than 50 times the IC₅₀ values of the compounds in cellular proliferation assays. Preferred compounds of the formula (I) have mean IC₅₀ values against hERG that are greater than 5 μM, more particularly greater than 10 μM, and more preferably greater than 15 μM. Some compounds of the invention have mean IC₅₀ values against hERG that are greater than 50 μM.

### Therapeutic Uses

### Prevention or Treatment of Proliferative Disorders

The compounds of the formula (I) are inhibitors of protein kinase A and protein kinase B. As such, they are expected to be useful in providing a means of preventing the growth of or inducing apoptosis of neoplasias. It is therefore anticipated that the compounds will prove useful in treating or preventing proliferative disorders such as cancers. In particular tumours with deletions or inactivating mutations in PTEN or loss of PTEN expression or rearrangements in the (T-cell lymphocyte) TCL-1 gene may be particularly sensitive to PKB inhibitors. Tumours which have other abnormalities leading to an upregulated PKB pathway signal may also be particularly sensitive to inhibitors of PKB. Examples of such abnormalities include but are not limited to overexpression of one or more PI3K subunits, over-expression of one or more PKB isoforms, or mutations in PI3K, PDK1, or PKB which lead to an increase in the basal activity of the enzyme in question, or upregulation or overexpression or mutational activation of a growth factor receptor such as a growth factor selected from the epidermal growth factor receptor (EGFR), fibroblast growth factor receptor (FGFR), platelet derived growth factor receptor (PDGFR), insulin-like growth factor 1 receptor (IGF-1R) and vascular endothelial growth factor receptor (VEGFR) families.

It is also envisaged that the compounds of the invention will be useful in treating other conditions which result from disorders in proliferation or survival such as viral infections, and neurodegenerative diseases for example. PKB plays an important role in maintaining the survival of immune cells during an immune response and therefore PKB inhibitors could be particularly beneficial in immune disorders including autoimmune conditions.

Therefore, PKB inhibitors could be useful in the treatment of diseases in which there is a disorder of proliferation, apoptosis or differentiation.

PKB inhibitors may also be useful in diseases resulting from insulin resistance and insensitivity, and the disruption of glucose, energy and fat storage such as metabolic disease and obesity.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, endometrium, thyroid, prostate, or skin, for example squamous cell carcinoma; a hematopoietic tumour of lymphoid lineage, for example leukaemia, acute lymphocytic leukaemia, B-cell lymphoma, T-cell lymphoma, Hodgkin's lymphoma, non-Hodgkin's lymphoma, hairy cell lymphoma, or Burkett's lymphoma; a hematopoietic tumour of myeloid lineage, for example acute and chronic myelogenous leukaemias, myelodysplastic syndrome, or promyelocytic leukaemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xenoderoma pigmentosum; keratoctanthoma; thyroid follicular cancer; or Kaposi's sarcoma.

Thus, in the pharmaceutical compositions, uses or methods of this invention for treating a disease or condition comprising abnormal cell growth, the disease or condition comprising abnormal cell growth in one embodiment is a cancer.

Particular subsets of cancers include breast cancer, ovarian cancer, colon cancer, prostate cancer, oesophageal cancer, squamous cancer and non-small cell lung carcinomas.

A further subset of cancers includes breast cancer, ovarian cancer, prostate cancer, endometrial cancer and glioma.

It is also possible that some protein kinase B inhibitors can be used in combination with other anticancer agents. For example, it may be beneficial to combine of an inhibitor that induces apoptosis with another agent which acts via a different mechanism to regulate cell growth thus treating two of the characteristic features of cancer development. Examples of such combinations are set out below.

### Immune Disorders

Immune disorders for which PKA and PKB inhibitors may be beneficial include but are not limited to autoimmune conditions and chronic inflammatory diseases, for example systemic lupus erythematosus, autoimmune mediated glomerulonephritis, rheumatoid arthritis, psoriasis, inflammatory bowel disease, and autoimmune diabetes mellitus, Eczema hypersensitivity reactions, asthma, COPD, rhinitis, and upper respiratory tract disease.

### Other Therapeutic Uses

PKB plays a role in apoptosis, proliferation, differentiation and therefore PKB inhibitors could also be useful in the treatment of the following diseases other than cancer and those associated with immune dysfunction; viral infections, for example herpes virus, pox virus, Epstein-Barr virus, Sindbis virus, adenovirus, HIV, HPV, HCV and HCMV; prevention of AIDS development in HIV-infected individuals; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, degenerative diseases of the musculoskeletal system, for example, osteoporosis and arthritis, aspirin-sensitive rhinosinusitis, cystic fibrosis, multiple sclerosis, kidney diseases.

### Advantages of Compounds of the Invention

It is envisaged that compounds of the formula (I) and sub-groups thereof as defined herein have advantages over prior art compounds.

In particular, the compounds of formulae (II), (IIa), (IIb) and (IIc) have advantages over prior art compounds.

Potentially the compounds of the invention have physiochemical properties suitable for oral exposure.

Compounds of the formula (I) should exhibit improved oral bioavailability relative to prior art compounds. Oral bioavailability can be defined as the ratio (F) of the plasma exposure of a compound when dosed by the oral route to the plasma exposure of the compound when dosed by the intravenous (i.v.) route, expressed as a percentage.

Compounds having an oral bioavailability (F value) of greater than 30%, more preferably greater than 40%, are particularly advantageous in that they may be administered orally rather than, or as well as, by parenteral administration.

Furthermore, it is envisaged that compounds of the invention are both more potent and more selective in their activities against different kinases, and demonstrate enhanced selectivity for and potency against PKB and PKB kinases in particular.

It is also envisaged that compounds of the invention are advantageous over prior art compounds in that they have different susceptibilities to P450 enzymes and in that they exhibit improvements with regard to drug metabolism and pharmacokinetic properties.

Furthermore, it is considered that compounds of the invention should exhibit reduced dosage requirements.

Compounds of the invention are advantageous in that they have improved thermodynamic solubilities, thereby leading potentially to an improved dose:solubility ratio and reduced development risk.

It is further envisaged that compounds of the invention also demonstrate improved cell activity in proliferation and clonogenic assays thereby indicating improved anti-cancer activity.

Compounds of the invention are potentially less toxic than prior art compounds.

For example, it is envisaged that compounds of formulae (II), (IIa), (IIb) and (IIc) in particular will have reduced, negligible or no BERG ion channel blocking activity.

### Methods of Treatment

It is envisaged that the compounds of the formula (I) and sub-groups thereof as defined herein will be useful in the prophylaxis or treatment of a range of disease states or conditions mediated by protein kinase A and/or protein kinase B. Examples of such disease states and conditions are set out above.

The compounds are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a pulsatile or continuous manner.

A typical daily dose of the compound of formula (I) can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound of the formula (I) can be administered on a daily basis or on a repeat basis every 2, or 3, or 4, or 5, or 6, or 7, or 10 or 14, or 21, or 28 days for example.

The compounds of the invention may be administered orally in a range of doses, for example 1 to 1500 mg, 2 to 800 mg, or 5 to 500 mg, e.g. 2 to 200 mg or 10 to 1000 mg, particular examples of doses including 10, 20, 50 and 80 mg. The compound may be administered once or more than once each day. The compound can be administered continuously (i.e. taken every day without a break for the duration of the treatment regimen). Alternatively, the compound can be administered intermittently, i.e. taken continuously for a given period such as a week, then discontinued for a period such as a week and then taken continuously for another period such as a week and so on throughout the duration of the treatment regimen. Examples of treatment regimens involving intermittent administration include regimens wherein administration is in cycles of one week on, one week off; or two weeks on, one week off; or three weeks on, one week off; or two weeks on, two weeks off; or four weeks on two weeks off; or one week on three weeks off—for one or more cycles, e.g. 2, 3, 4, 5, 6, 7, 8, 9 or 10 or more cycles.

In one particular dosing schedule, a patient will be given an infusion of a compound of the formula (I) for periods of one hour daily for up to ten days in particular up to five days for one week, and the treatment repeated at a desired interval such as two to four weeks, in particular every three weeks.

More particularly, a patient may be given an infusion of a compound of the formula (I) for periods of one hour daily for 5 days and the treatment repeated every three weeks.

In another particular dosing schedule, a patient is given an infusion over 30 minutes to 1 hour followed by maintenance infusions of variable duration, for example 1 to 5 hours, e.g. 3 hours.

In a further particular dosing schedule, a patient is given a continuous infusion for a period of 12 hours to 5 days, an in particular a continuous infusion of 24 hours to 72 hours.

Ultimately, however, the quantity of compound administered and the type of composition used will be commensurate with the nature of the disease or physiological condition being treated and will be at the discretion of the physician.

The compounds as defined herein can be administered as the sole therapeutic agent or they can be administered in combination therapy with one of more other compounds for treatment of a particular disease state, for example a neoplastic disease such as a cancer as hereinbefore defined. Examples of other therapeutic agents or treatments that may be administered together (whether concurrently or at different time intervals) with the compounds of the formula (I) include but are not limited to:
Topoisomerase I inhibitors
Antimetabolites
Tubulin targeting agents
DNA binder and topoisomerase II inhibitors
Alkylating Agents
Monoclonal Antibodies.
Anti-Hormones
Signal Transduction Inhibitors
Proteasome Inhibitors
DNA methyl transferases
Cytokines and retinoids
Chromatin targeted therapies
Radiotherapy, and,
Other therapeutic or prophylactic agents; for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF). Also included are agents that inhibit bone resorption such as bisphosphonate agents e.g. zoledronate, pamidronate and ibandronate, agents that suppress inflammatory responses (such as dexamethazone, prednisone, and prednisolone) and agents used to reduce blood levels of growth hormone and IGF-I in acromegaly patients such as synthetic forms of the brain hormone somatostatin, which includes octreotide acetate which is a long-acting octapeptide with pharmacologic properties mimicking those of the natural hormone somatostatin. Further included are agents such as leucovorin, which is used as an antidote to drugs that decrease levels of folic acid, or folinic acid it self and agents such as megestrol acetate which can be used for the treatment of side-effects including oedema and thromoembolic episodes.

Each of the compounds present in the combinations of the invention may be given in individually varying dose schedules and via different routes.

Where the compound of the formula (I) is administered in combination therapy with one, two, three, four or more other therapeutic agents (preferably one or two, more preferably one), the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through his or her common general knowledge the dosing regimes and combination therapies to use.

### Methods of Diagnosis

Prior to administration of a compound of the formula (I), a patient may be screened to determine whether a disease or condition from which the patient is or may be suffering is one which would be susceptible to treatment with a compound having activity against protein kinase A and/or protein kinase B.

For example, a biological sample taken from a patient may be analysed to determine whether a condition or disease, such as cancer, that the patient is or may be suffering from is one which is characterised by a genetic abnormality or abnormal protein expression which leads to up-regulation of PKA and/or PKB or to sensitisation of a pathway to normal PKA and/or PKB activity, or to upregulation of a signal transduction component upstream of PKA and/or PKB such as, in the case of PKB, PI3K, GF receptor and PDK 1 & 2.

Alternatively, a biological sample taken from a patient may be analysed for loss of a negative regulator or suppressor of the PKB pathway such as PTEN. In the present context, the term “loss” embraces the deletion of a gene encoding the regulator or suppressor, the truncation of the gene (for example by mutation), the truncation of the transcribed product of the gene, or the inactivation of the transcribed product (e.g. by point mutation) or sequestration by another gene product.

The term up-regulation includes elevated expression or over-expression, including gene amplification (i.e. multiple gene copies) and increased expression by a transcriptional effect, and hyperactivity and activation, including activation by mutations. Thus, the patient may be subjected to a diagnostic test to detect a marker characteristic of up-regulation of PKA and/or PKB. The term diagnosis includes screening. By marker we include genetic markers including, for example, the measurement of DNA composition to identify mutations of PKA and/or PKB. The term marker also includes markers which are characteristic of up regulation of PKA and/or PKB, including enzyme activity, enzyme levels, enzyme state (e.g. phosphorylated or not) and mRNA levels of the aforementioned proteins.

The above diagnostic tests and screens are typically conducted on a biological sample selected from tumour biopsy samples, blood samples (isolation and enrichment of shed tumour cells), stool biopsies, sputum, chromosome analysis, pleural fluid, peritoneal fluid, or urine.

Identification of an individual carrying a mutation in PKA and/or PKB or a rearrangement of TCL-1 or loss of PTEN expression may mean that the patient would be particularly suitable for treatment with a PKA and/or PKB inhibitor. Tumours may preferentially be screened for presence of a PKA and/or PKB variant prior to treatment. The screening process will typically involve direct sequencing, oligonucleotide microarray analysis, or a mutant specific antibody.

Methods of identification and analysis of mutations and up-regulation of proteins are known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtitre plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of upregulation of PKB, or detection of PKB variants could be applicable in the present case.

Therefore all of these techniques could also be used to identify tumours particularly suitable for treatment with PKA and/or PKB inhibitors.

For example, as stated above, PKB beta has been found to be upregulated in 10-40% of ovarian and pancreatic cancers (Bellacosa et al 1995, Int. J. Cancer 64, 280-285; Cheng et al 1996, PNAS 93, 3636-3641; Yuan et al 2000, Oncogene 19, 2324-2330). Therefore it is envisaged that PKB inhibitors, and in particular inhibitors of PKB beta, may be used to treat ovarian and pancreatic cancers.

PKB alpha is amplified in human gastric, prostate and breast cancer (Staal 1987, PNAS 84, 5034-5037; Sun et al 2001, Am. J. Pathol. 159, 431-437). Therefore it is envisaged that PKB inhibitors, and in particular inhibitors of PKB alpha, may be used to treat human gastric, prostate and breast cancer.

Increased PKB gamma activity has been observed in steroid independent breast and prostate cell lines (Nakatani et al 1999, J. Biol. Chem. 274, 21528-21532). Therefore it is envisaged that PKB inhibitors, and in particular inhibitors of PKB gamma, may be used to treat steroid independent breast and prostate cancers.

### Experimental

The invention will now be illustrated, but not limited, by reference to the specific embodiments described in the following procedures and examples.

The starting materials for each of the procedures described below are commercially available unless otherwise specified.

In the examples, the compounds prepared were characterised by liquid chromatography, mass spectroscopy and ¹H nuclear magnetic resonance spectroscopy using the systems and operating conditions set out below.

Proton magnetic resonance (¹H NMR) spectra were recorded on a Bruker AV400 instrument) operating at 400.13 MHz, in Me-d₃-OD at 27 C, unless otherwise stated and are reported as follows: chemical shift δ/ppm (number of protons, multiplicity where s=singlet, d=doublet, t=triplet, q=quartet, m=multiplet, br=broad). The residual protic solvent MeOH (δ_{H}=3.31 ppm) was used as the internal reference.

For the mass spectra, where chlorine is present, the mass quoted for the compound is for ³⁵Cl.

In each of the examples, where the compounds are isolated or formed as the free base, they can be converted into a salt form such as an acetic acid or hydrochloric acid salt. Conversely, where the compounds are isolated or formed as a salt, the salt can be converted into the corresponding free base by methods well known to the skilled person, and then optionally converted to another salt.

A number of liquid chromatography systems were used and these are described below.

### Platform System

HPLC System: Waters 2795
Mass Spec Detector: Micromass Platform LC
PDA Detector: Waters 2996 PDA

### Acidic Analytical Conditions 1:

Eluent A: H₂O (0.1% Formic Acid)
Eluent B: CH₃CN (0.1% Formic Acid)
Gradient: 5-95% eluent B over 3.5 minutes
Flow: 1.5 ml/min
Column: Phenomenex Synergi 4μ, Max-RP 80A, 50×4.6 mm

### Acidic Analytical Conditions 2:

Eluent A: H₂O (0.1% Formic Acid)
Eluent B: CH₃CN (0.1% Formic Acid)
Gradient: 5-95% eluent B over 3.5 minutes
Flow: 0.8 ml/min
Column: Phenomenex Synergi 4p, Max-RP 80A, 50×2.0 mm

### Acidic Analytical Conditions 3:

Eluent A: H₂O (0.1% Formic Acid)
Eluent B: CH₃CN (0.1% Formic Acid)
Gradient: 5-95% eluent B over 15 minutes
Flow: 0.4 ml/min
Column: Phenomenex Synergi 4p. Max-RP 80A, 50×2.0 mm

### Basic Analytical Conditions 1:

Eluent A: H₂O (10 mM NH₄HCO₃ buffer adjusted to pH=9.5 with NH₄OH)
Eluent B: CH₃CN
Gradient: 05-95% eluent B over 3.5 minutes
Flow: 1.5 ml/min
Column: Waters XTerra MS C₁₈ 5 μm 4.6×50 mm

### Basic Analytical Conditions 2:

Eluent A: H₂O (10 mM NH₄HCO₃ buffer adjusted to pH=9.5 with NH₄OH)
Eluent B: CH₃CN
Gradient: 05-95% eluent B over 3.5 minutes
Flow: 0.8 ml/min
Column: Thermo Hypersil-Keystone BetaBasic-18 5 μm, 50×2.1 mm

### Basic Analytical Conditions 3:

Eluent A: H₂O (10 mM NH₄HCO₃ buffer adjusted to pH=9.5 with NH₄OH)
Eluent B: CH₃CN
Gradient: 05-95% eluent B over 3.5 minutes
Flow: 0.8 ml/min
Column: Phenomenex Luna C18(2) 5 μm, 50×2.0 mm

### Basic Analytical Conditions 4:

Eluent A: H₂O (10 mM NH₄HCO₃ buffer adjusted to pH=9.2 with NH₄OH)
Eluent B: CH₃CN
Gradient: 05-95% eluent B over 15 minutes
Flow: 0.8 ml/min
Column: Phenomenex Luna C18(2) 5 μm, 150×2.0 mm

### Basic Analytical Conditions 5:

Eluent A: H₂O (10 mM NH₄HCO₃ buffer adjusted to pH=9.2 with NH₄OH)
Eluent B: CH₃CN
Gradient: 05-95% eluent B over 3.5 minutes
Flow: 0.8 ml/min
Column: Phenomenex Gemini 5μ 2.0×50 mm

### Lipophilic Analytical Conditions 1:

Eluent A: H₂O (0.1% Formic Acid)
Eluent B: CH₃CN (0.1% Formic Acid)
Gradient: 55-95% eluent B over 3.5 minutes
Flow: 0.8 ml/min
Column: Phenomenex Synergi 4μ MAX-RP 80A, 2.0×50 mm

### Polar Analytical Conditions:

Eluent A: H₂O (0.1% Formic Acid)
Eluent B: CH₃CN (0.1% Formic Acid)
Gradient: 00-50% eluent B over 3 minutes
Flow: 1.5 ml/min
Column: Phenomenex Synergi 4μ Hydro 80A, 50×4.6 mm

### MS Conditions:

Capillary voltage: 3.5 kV or 3.6 kV
Cone voltage: 30 V
Source Temperature: 120° C.
Scan Range: 165-700 amu
Ionisation Mode: Electro Spray Negative, Positive or Positive & Negative

### FractionLynx System

System: Waters FractionLynx (dual analytical/prep)
HPLC Pump: Waters 2525
Injector-Autosampler: Waters 2767
Mass Spec Detector: Waters-Micromass ZQ
PDA Detector: Waters 2996 PDA

### Acidic Analytical Conditions:

Eluent A: H₂O (0.1% Formic Acid)
Eluent B: CH₃CN (0.1% Formic Acid)
Gradient: 5-95% eluent B over 5 minutes
Flow: 2.0 ml/min
Column: Phenomenex Synergi 4μ Max-RP 80A, 50×4.6 mm

### Polar Analytical Conditions:

Eluent A: H₂O (0.1% Formic Acid)
Eluent B: CH₃CN (0.1% Formic Acid)
Gradient: 00-50% eluent B over 5 minutes
Flow: 2.0 ml/min
Column: Phenomenex Synergi 4μ, Max-RP 80A, 50×4.6 mm

### MS Parameters for Acidic and Polar Analytical Conditions:

Capillary voltage: 3.5 kV
Cone voltage: 25 V
Source Temperature: 120° C.
Scan Range: 125-800 amu
Ionisation Mode: ElectroSpray Positive or ElectroSpray Positive & Negative

### Chiral Analytical Conditions:

Eluent: MeOH+0.1% NH₄/TFA
Flow: 1.2 ml/min
Total time: 16.00 min
Inj. Volume: 10 μL
Sample conc.: 2 mg/ml
Column: Astec, Chirobiotic V; 250×4.6 mm

Mass spectrometer was taken off-line.

### Agilent System

HPLC System: Agilent 1100 series
Mass Spec Detector: Agilent LC/MSD VL
Multi Wavelength Detector: Agilent 1100 series MWD
Software: HP Chemstation

### Chiral Analytical Conditions:

Eluent: MeOH+0.2% NH₄/AcOH at room Temperature
Flow: 2.0 ml/min
Total time: 8.5 min
Inj. Volume: 20 uL
Sample Conc: 2 mg/ml
Column: Astec, Chirobiotic V; 250×4.6 mm

### Chiral Analytical Conditions 2:

Eluent: 50:50 MeOH:EtOH+0.1% DEA at room Temperature
Flow: 0.5 ml/min
Total time: 16 min
Inj. Volume: 10 uL
Sample Conc: 1 mg/ml (in mobile phase)
Column: DAICEL Chiralpak AD-H; 250×4.6 mm
Wavelength: 257 nm

### Chiral Preparative Conditions 1:

Eluent: MeOH+0.1% NH₄/TFA at room Temperature
Flow: 6.0 ml/min
Total time: 10 min
Inj. Volume: 100 uL
Sample Conc: 20 mg/ml
Column: Astec, Chirobiotic V; 250×10 mm

### Chiral Preparative Conditions 2:

Eluent: MeOH+0.2% NH₄/AcOH at room Temperature
Flow: 20.0 ml/min
Total time: 19 min
Inj. Volume: 950 uL
Sample Conc: 25 mg/ml
Column: Astec, Chirobiotic V2; 250×21.2 mm

### Chiral Preparative Conditions 3:

Eluent: 50:50 MeOH:EtOH+0.1% DEA at room Temperature
Flow: 9 ml/min
Total time: 16 min
Inj. Volume: 220 uL
Sample Conc: 35 mg/ml (in mobile phase)
Column: DAICEL Chiralpak AD-H; 250×20 mm
Wavelength: 257 nm

### MS Conditions (Just Analytical Method):

Capillary voltage: 3000 V
Fragmentor: 150
Gain: 1.00
Drying gas: 12.0 L/min
Drying gas T: 350° C.
Nebulizer pressure: 35 (psig)
Scan Range: 125-800 amu
Ionisation Mode ElectroSpray Positive

In the examples below, the following key is used to identify the LCMS conditions used:
PS-A Platform System acidic analytical conditions 1
PS-A2 Platform System acidic analytical conditions 2
PS-A3 Platform System acidic analytical conditions 3
PS-B Platform System basic analytical conditions 1
PS-B2 Platform System basic analytical conditions 2
PS-B3 Platform System basic analytical conditions 3
PS-B4 Platform System basic analytical conditions 4
PS-B5 Platform System basic analytical conditions 5
PS-L Platform System lipophilic analytical conditions 1
PS-P Platform System polar analytical conditions
FL-A FractionLynx System acidic analytical conditions
FL-P FractionLynx System polar analytical conditions
FL-C FractionLynx System chiral analytical conditions
AG-CA Agilent System chiral analytical conditions
AG-CA2 Agilent System chiral analytical conditions 2
AG-CP1 Agilent System chiral preparative conditions 1
AG-CP2 Agilent System chiral preparative conditions 2
AG-CP3 Agilent System chiral preparative conditions 3

### General Synthetic Procedures

### General Procedure 1

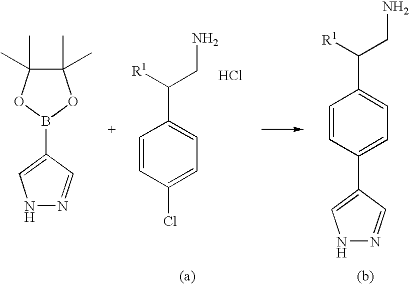

To a suspension of the 2-substituted 2-(4-chlorophenyl)ethylamine hydrochloride (a) (0.5 mmol, 1.0 equiv.) (Array PPA-Q02-1) in toluene (0.8 ml) is added bis(tri-t-butylphosphine)palladium (0) (3 mg, 1 mol %) (Strem) and the mixture is purged with nitrogen. A suspension of 4-(4,4,5,5-tetramethyl-1,3,2-dioxaborolan-2-yl)-1H-pyrazole (107 mg, 0.55 mmol, 1.1 equiv.) (Aldrich 52, 505-7) in ethanol (0.8 ml) is added followed by potassium carbonate (415 mg, 3.0 mmol, 6 equiv.) in water (2.5 ml). The mixture is purged with nitrogen and sealed. The reaction mixture is heated in a CEM Explorer microwave to 135° C. for 15 minutes using 50 watts power. The solvents are removed and the residue is partitioned between ethyl acetate and 2N NaOH. The aqueous layer is extracted with ethyl acetate and the combined organic layers are washed with brine, dried (MgSO₄) and concentrated under reduced pressure. The crude reaction mixture is purified by column chromatography (SiO₂), eluting with a mixture of dichloromethane (90 ml): methanol (18 ml): acetic acid (3 ml): H₂O (2 ml) to afford the product (b).

### General Procedure 2

### 2A

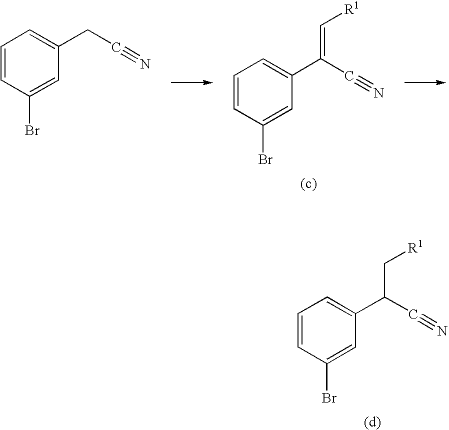

A solution of 40% KOH (2.83 g in 5.0 ml of H₂O) in ethanol (13 ml) is added to a solution of an aryl or heteroaryl aldehyde R¹—CHO (28.05 mmol) and 3-bromophenylacetonitrile (25.50 mmol) in ethanol (9 ml). The reaction mixture is then stirred at room temperature for 2 hours and the precipitate is collected by suction filtration and washed with cold ethanol. The crude product (c) is then dissolved in ethanol (35 ml) and heated to 65° C. Sodium borohydride (459 mg, 12.14 mmol) is added in portions and the reaction mixture is maintained at this temperature for a further 2 hours. Upon cooling, water (10 ml) is added and the solvent is removed under reduced pressure. The residue is partitioned between water (100 ml) and ethyl acetate (100 ml). The organic layer is separated, dried (MgSO₄), filtered and concentrated to afford the desired product (d), which may be used without purification.

### 2B

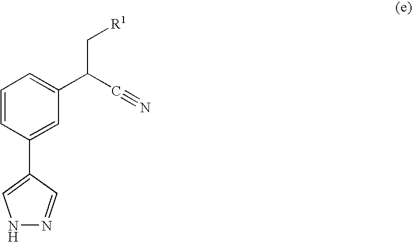

3-Substituted 2-(3-Bromo-phenyl)-propionitrile (d) is reacted with 4-(4,4,5,5-tetramethyl-1,3,2-dioxaborolan-2-yl)-1H-pyrazole following the procedure set out in Example 1 to give the desired product (e).

### General Procedure 3

### 3A

(J. Med. Chem., 1983, 26, 935-947)

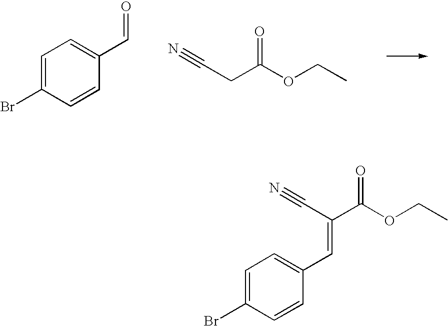

To 4-bromobenzaldehyde (3 g, 16.21 mmol) and ethyl cyanoacetate (1.9 ml, 17.84 mmol) in toluene is added piperidine (27 μl) and the reaction mixture is refluxed for 1 hour with a Dean-Stark separator. The solvent is removed under reduced pressure, the residue triturated with warm ethyl acetate, filtered to yield the desired product as a yellow solid (4.03 g, 89% yield). LC/MS: (PS-A2) Rₜ 3.44.

### 3B

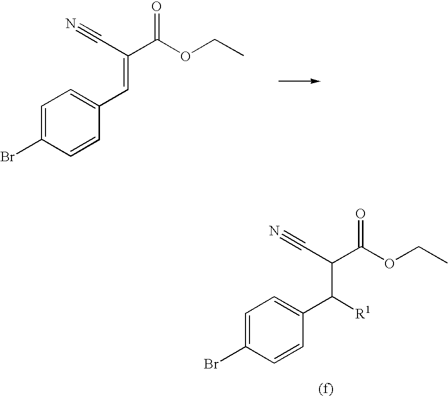

A solution of 3-(4-bromo-phenyl)-2-cyano-acrylic acid ethyl ester (1.5 g, 5.36 mmol) in dry toluene (12 ml) was added dropwise to an arylmagnesium bromide (0.5 M solution in tetrahydrofuran, (6.96 ml, 6.96 mmol) at 0° C. The reaction mixture is heated to 85° C. for 3 hours, poured onto ice, acidified with 1N HCl and extracted with ethyl acetate. The organic layer is separated, dried (MgSO₄), filtered and concentrated, the crude product is purified over flash silica chromatography eluting with petroleum ether to ethyl acetate/petroleum ether (5:95) to afford the desired product (f).

### 3C

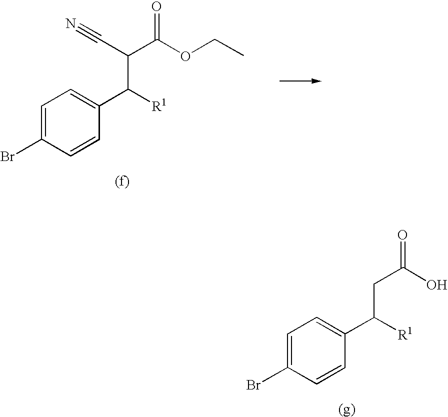

A mixture of ester (f) (4.87 mmol), acetic acid (10 ml), concentrated sulphuric acid (5 ml) and water (5 ml) is refluxed for 2 hours. The reaction mixture is poured into iced water and extracted with ethyl acetate. The organic layer is separated, dried (MgSO₄), filtered and concentrated, the crude product is purified over flash silica chromatography eluting with ethyl acetate/petroleum:ether (1:1) to afford the desired carboxylic acid product (g).

### 3D

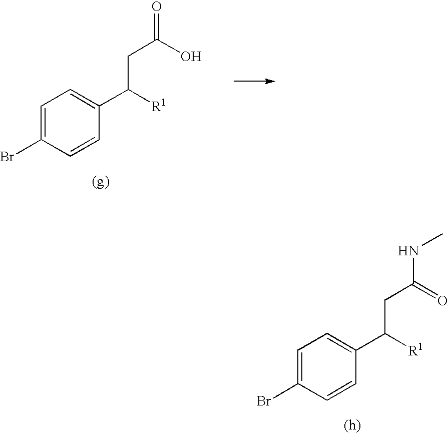

A mixture of carboxylic acid (g) (0.74 mmol) and 1-hydroxybenztriazole (0.88 mmol) in dichloromethane (3 ml) is stirred for 15 minutes before addition of methylamine (40% solution in water, 0.11 μl, 1.47 mmol) and 1-(3-dimethylaminopropyl)-ethylcarbodiimide hydrochloride (0.17 g, 0.88 mmol). The reaction mixture is stirred for 16 hours, solvent removed under reduced pressure and the residue partitioned between ethyl acetate and 1N HCl. The organic layer is separated, washed with saturated sodium hydrogen carbonate, brine, dried (MgSO₄), filtered and concentrated to yield the compound (h) which can be used in the next step without further purification.

### 3E

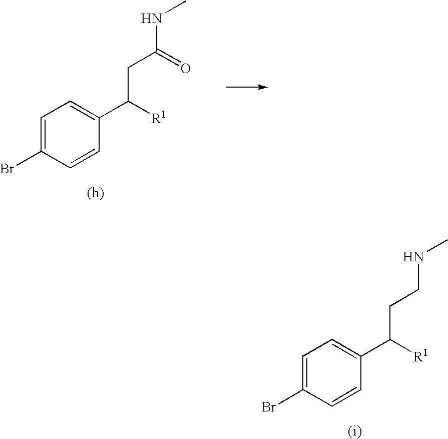

Under a nitrogen atmosphere, the crude amide (h) is cooled to 0° C. and lithium aluminum hydride (0.075 g, 1.97 mmol) and diethyl ether (3 ml) are added. With cooling, aluminum chloride (0.23 g, 1.69 mmol) is dissolved in diethyl ether (2 ml) and added. The reaction mixture is stirred for 16 hours, quenched with addition of water, basified (2N NaOH) and extracted with ethyl acetate. The organic layer is separated, dried (MgSO₄), filtered and concentrated, the crude product is purified over Phenomenex_Strata_SCX column chromatography eluting with methanol followed by 2N ammonia in methanol to afford the desired amine product (i).

### 3F

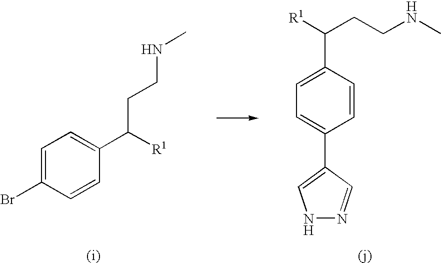

The methyl-amine (j) is reacted with 4-(4,4,5,5-tetramethyl-1,3,2-dioxaborolan-2-yl)-1H-pyrazole following the procedure set out in General Procedure 1 to give the pyrazole compound V).

### General Procedure 4

### 4A

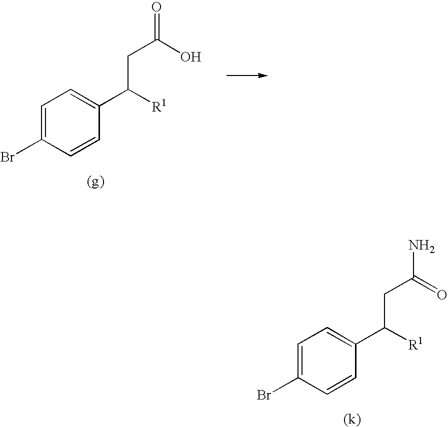

A solution of carboxylic acid (g) (see General Procedure 3C) (0.74 mmol) and 1,1′-carbonyldiimidazole (0.24 g, 1.47 mmol) in dichloromethane is stirred for 45 minutes before the addition of ammonia (2M solution in methanol, 3.68 ml, 7.36 mmol). The reaction mixture is stirred for 2 hours, the solvent is removed under reduced pressure and the residue is purified over flash silica chromatography eluting with ethyl acetate/petroleum ether (1:4) to afford the carboxamide compound (k).

### 4B

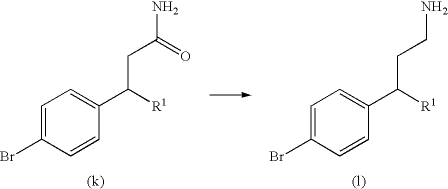

By following the procedure described in General Procedure 3E but substituting amide (k) for amide (h), the primary amine compound (1) is obtained.

### 4C

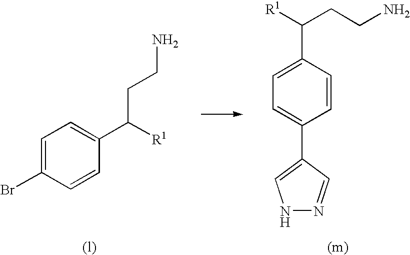

Amine (1) is reacted with 4-(4,4,5,5-tetramethyl-1,3,2-dioxaborolan-2-yl)-1H-pyrazole following the procedure set out in General Procedure 1 to give the pyrazole compound (m).

### General Procedure 5

### 5A. 1-(4-Bromo-phenyl)-2-methylamino-ethanol

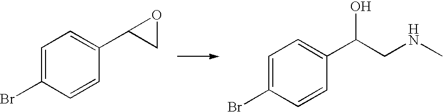

A solution of 2-(4-bromophenyl)-oxirane (0.5 g, 2.51 mmol) in methylamine (6.6 ml, 33% by volume in ethanol, 25.12 mmol) is stirred at room temperature under an atmosphere of nitrogen. After 18 hours the solvent is removed in vacuo and the residue is purified over flash silica eluting with dichloromethane: methanol: acetic acid:water (120:15:3:2) to afford the desired compound as the acetic acid salt. Further purification over a Phenomenex_Strata_SCX column eluting with methanol followed by 2N ammonia in methanol gives the desired product. LC/MS: (PS-B3) Rₜ 2.52 [M+H]⁺ 230.

### 5B

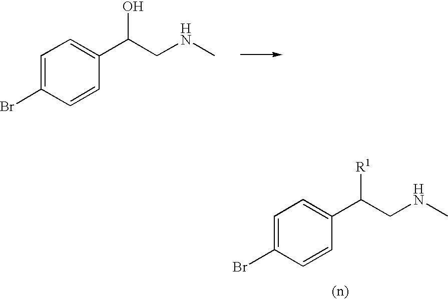

Aluminium chloride (278 mg, 2.087 mmol) is added portionwise to a stirred solution of 1-(4-bromo-phenyl)-2-methylamino-ethanol (160 mg, 0.696 mmol) in chlorobenzene (or other aryl compound R1-H) (3 ml) and the reaction mixture stirred at room temperature for 17 hours. Water (2 ml) is added dropwise and the reaction mixture is then partitioned between dichloromethane (100 ml) and saturated NaHCO₃ (30 ml). The organic layer was dried (MgSO₄), filtered and concentrated under reduced pressure. The crude product is then purified by Phenomenex_Strata_SCX column chromatography eluting with methanol followed by 2N ammonia in methanol to afford the desired amine product (n).

### 5C

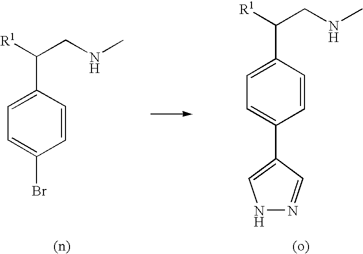

A solution of amine (n) (13.716 mmol), 4-(4,4,5,5-tetramethyl-1,3,2-dioxaborolan-2-yl)-1H-pyrazole (5.3 g, 27.431 mmol) and K₃PO₄ (10.19 g, 48.00 mmol) in ethanol (7.5 ml), methanol (11.5 ml), toluene (7.5 ml) and water (11.5 ml) is purged with nitrogen for 2 minutes. Bis(tri-t-butylphosphine)palladium (0) (175 mg, 2.5 mol %) is then added and the reaction mixture purged with nitrogen for a further 2 minutes. The mixture is then heated to 80° C., under nitrogen for a period of 17 hours. The solvents are removed and the residue is partitioned between ethyl acetate and 2N NaOH. The aqueous layer is extracted with ethyl acetate and the combined organic layers are washed with brine, dried (MgSO₄) and concentrated under reduced pressure. The crude reaction mixture is purified by column chromatography (SiO₂), eluting with dichloromethane: methanol: acetic acid:water (90:18:3:2) to afford the pyrazole compound (o).

### General Procedure 6

### 6A

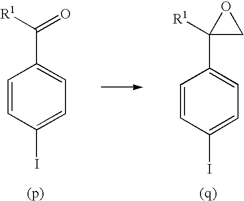

Sodium hydride (60% dispersion in oil, 128 mg, 3.2 mmol) is placed under N₂ then DMSO (5 ml) is added. Trimethylsulphonium iodide (0.66 g, 3.2 mmol) is added as a solid after 15 minutes, followed after a further 30 minutes by carbonyl compound (p) (which may be either a ketone or an aldehyde). The mixture is stirred at room temperature for 24 hours then diluted with ethyl acetate and washed with 1:2 water/brine, water and brine (×2). The organic phase is dried (MgSO₄), filtered and concentrated to give the oxirane compound (q) which can be used without further purification.

### 6B

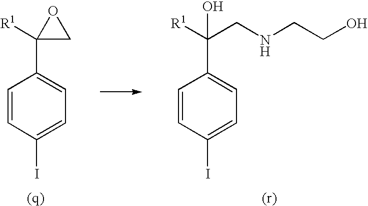

A solution of oxirane (q) (1.68 mmol), ethanolamine (0.5 ml, 8.3 mmol) and triethylamine (0.5 ml, 3.6 mmol) in iso-propanol (5 ml) is maintained at 50° C. for 72 hours then concentrated in vacuo. The residue is taken up in ethyl acetate and washed with saturated potassium carbonate solution/water (1:9). The aqueous phase can be extracted a second time with ethyl acetate, and then the combined extracts are washed with brine, dried (MgSO₄), filtered and concentrated to give the ethanolamine compound (r).

### General Procedure 7

### 7A

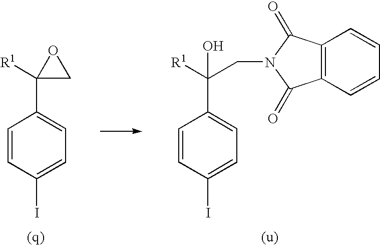

A mixture of the oxirane (q) (see General Procedure 6A) (1.60 mmol) and potassium phthalimide (340 mg, 1.84 mmol) in THF (5 ml) and DMSO (2 ml) is heated at 100° C. for 20 hours. The mixture is concentrated in vacuo, diluted with ethyl acetate and washed with water and brine (×2), dried (MgSO₄), filtered and concentrated to give a crude product which is purified by column chromatography (SiO₂), eluting with a gradient of ethyl acetate/petrol (2.5% to 100%) then 10% methanol/dichloromethane to give the phthalimide compound (u).

### 7B

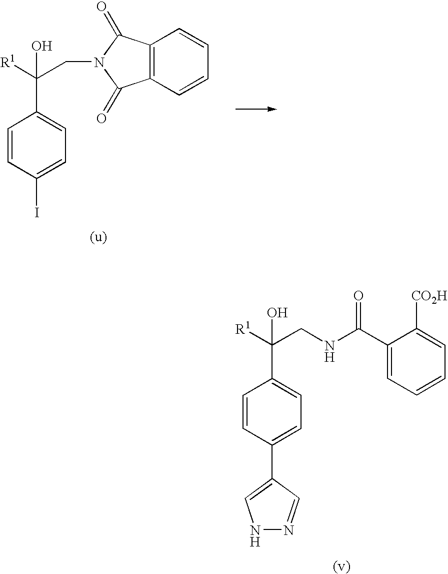

Phthalimide compound (n) is reacted with 4-(4,4,5,5-tetramethyl-1,3,2-dioxaborolan-2-yl)-1H-pyrazole following the procedure set out in General Procedure 1, but using tetrakis(triphenylphosphine) palladium (0) as the catalyst, to obtain the phthalamic acid compound (v).

### 7C

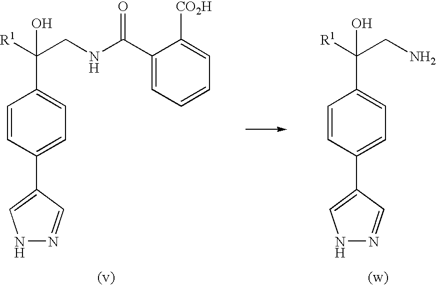

Hydrazine monohydrate (159 μl, 3.28 mmol) is added to a solution of phthalamic acid compound (v) (0.55 mmol) in methanol (6 ml) and the reaction mixture stirred at 80° C. for 16 hours. Upon cooling, the solvent is removed under reduced pressure and the crude product is purified by column chromatography (SiO₂), eluting with dichloromethane: methanol: acetic acid:water (90:18:3:2). Further purification by Phenomenex Strata SCX column chromatography, eluting with methanol followed by 2N ammonia in methanol, affords the hydroxyethylamine product (w) as the free base.

### General Procedure 8

### 8A

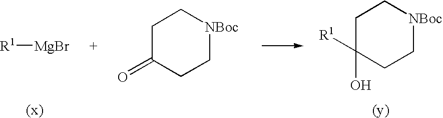

A solution of an aryl-magnesium bromide (x) (7.5 mmol, 0.5 M in THF) is added, under nitrogen, to 4-oxo-piperidine-1-carboxylic acid tert-butyl ester (1.02 g, 5.1 mmol). After 24 hours, saturated ammonium chloride solution is added then the organic solvent is removed in vacuo. The mixture is extracted with ethyl acetate, then this extract is washed with brine, dried (MgSO₄), filtered and concentrated to afford a residue which is purified by column chromatography (SiO₂), eluting with gradient of ethyl acetate/petrol (0% to 20%) to afford the N-protected hydroxypiperidine compound (y) (30%).

### 8B. 4-(4-Bromo-phenyl)-4-(4-chloro-3-fluoro-phenyl)-piperidine

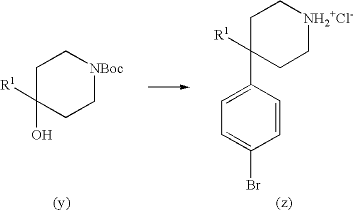

By following the procedure described in General procedure 5B but replacing the aryl compound R¹—H with bromobenzene, the bromophenyl-piperidine compound (z) is obtained.

### 8C

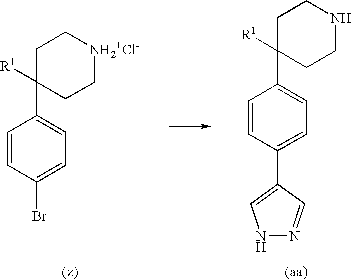

Bromo-phenyl)-piperidine (z) is reacted with 4-(4,4,5,5-tetramethyl-1,3,2-dioxaborolan-2-yl)-1H-pyrazole following the General Procedure 1, but using tetrakis(triphenylphosphine) palladium (0) as the catalyst, to obtain the pyrazole compound (aa).

### General Procedure 9

### 9A

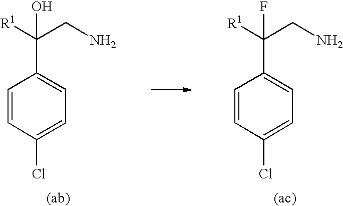

The amino-1,1-bis-aryl-ethanol (ab) (1.04 mmol) is dissolved in pyridine-HF (2 ml) with) cooling. After 24 hours the mixture is diluted into 1N sodium hydroxide solution and extracted with DCM (×3). Each extract is dried (MgSO₄) and filtered before being combined and concentrated to give a residue which is purified by column chromatography (SiO₂), eluting with 0.5% triethylamine in ethyl acetate to afford the fluoro compound (ac).

### 9B. 2-(4-Chloro-phenyl)-2-fluoro-2-[4-(1H-pyrazol-4-yl)-phenyl]-ethylamine

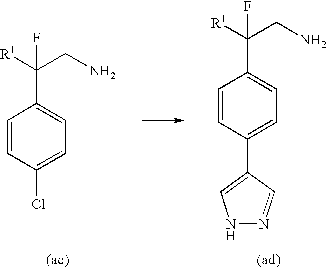

The (4-chloro-phenyl)-2-fluoro-ethylamine (ac) is reacted with 4-(4,4,5,5-tetramethyl-1,3,2-dioxaborolan-2-yl)-1H-pyrazole following the procedure set out in General Procedure 1 except that heating is carried out at 100° C. for 5 minutes using 300 W power in a CEM microwave, to obtain the pyrazole compound (ad).

### EXAMPLES

### Example 1

### 3-(4-Chloro-3-fluoro-phenyl)-3-[4-(1H-pyrazol-4-yl)-phenyl]-propylamine

### 1A. 3-(4-Bromo-phenyl)-2-cyano-acrylic acid ethyl ester

(J. Med. Chem., 1983, 26, 935-947)

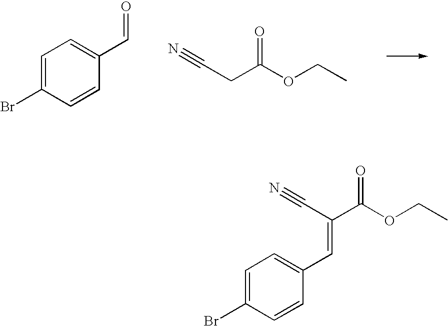

To 4-bromobenzaldehyde (3 g, 16.21 mmol) and ethyl cyanoacetate (1.9 ml, 17.84 mmol) in toluene was added piperidine (0.027 ml) and the reaction mixture was refluxed for 1 hour with a Dean-Stark separator. The solvent was removed under reduced pressure, the residue triturated with warm ethyl acetate, and filtered to yield the desired product as a yellow solid (4.03 g, 89% yield). LC/MS: (PS-A2) Rₜ 3.44.

### 1B. 3-(4-Bromo-phenyl)-3-(4-chloro-3-fluoro-phenyl)-2-cyano-propionic acid ethyl ester

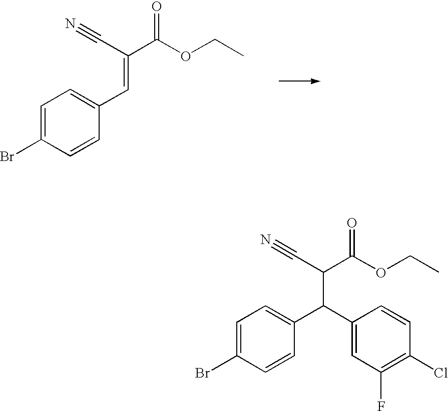

A solution of 3-(4-bromo-phenyl)-2-cyano-acrylic acid ethyl ester (2.1 g, 7.49 mmol) was suspended in dry toluene (15 ml) and cooled to 0° C. 4-Chloro-3-fluorophenylmagnesium bromide (0.5 M solution in tetrahydrofuran, 32.96 ml, 16.48 mmol) was added dropwise, maintaining the temperature at 0° C. The mixture was then heated at 70° C. for 16 hours. The reaction was allowed to cool to room temperature then poured onto ice/ water and acidified with 1N HCl (aq). The aqueous was extracted twice with ethyl acetate and the organics were separated, dried (MgSO₄) and concentrated in vacuo to afford the title compound as a yellow oil (4.29 g, >100% yield). The compound was progressed without further purification. LC/MS: (PS-B3) Rₜ 3.82 [M+H]⁻ 409.93.

### 1C. 3-(4-Bromo-phenyl)-3-(4-chloro-3-fluoro-phenyl)-propionic acid

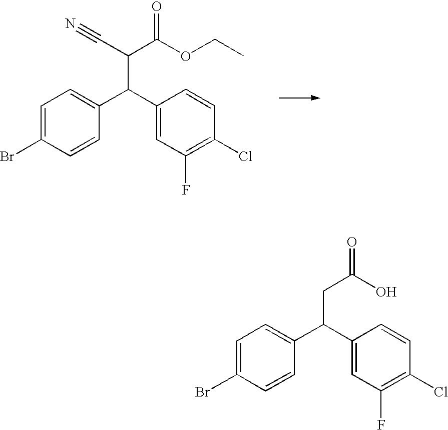

A mixture of 3-(4-bromo-phenyl)-3-(4-chloro-3-fluoro-phenyl)-2-cyano-propionic acid ethyl ester (4.29 g, 10.45 mmol), acetic acid (22.3 ml), concentrated sulphuric acid (11.16 ml) and water (11.16 ml) was refluxed for 2.5 hours. The reaction mixture was allowed to cool to room temperature and was then poured onto ice/ water and extracted twice with ethyl acetate. The organic layer was separated, dried (MgSO₄) and concentrated in vacuo. The crude product was purified by basic ion exchange chromatography to afford the title compound as a yellow solid (1.30 g, 49% yield assuming quantitative yield for previous step). LC/MS: (PS-A2) Rₜ 3.47 [M+H]⁻ 357.00.

### 1D. 3-(4-Bromo-phenyl)-3-(4-chloro-3-fluoro-phenyl)-propionamide

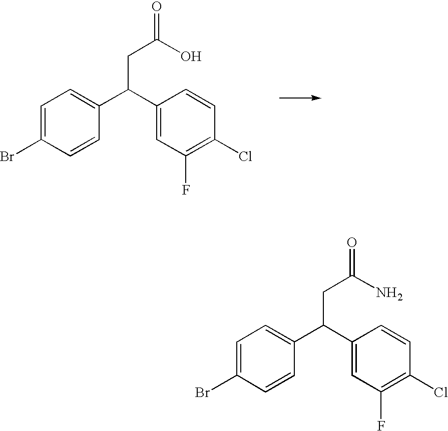

3-(4-Bromo-phenyl)-3-(4-chloro-3-fluoro-phenyl)-propionic acid (0.521 g, 1.46 mmol) was dissolved in thionyl chloride (15 ml) and heated at 50° C. for 1 hour. The reaction mixture was allowed to cool to room temperature and was concentrated in vacuo and azeotroped twice with toluene. Aqueous ammonium hydroxide (38 ml) was added dropwise to the residue and the mixture was stirred at room temperature for 64 hours. The reaction mixture was diluted with brine and extracted twice with ethyl acetate. The organic layer was separated, dried (MgSO₄) and concentrated in vacuo to afford the title compound as a yellow solid (0.45 g, 86% yield). LC/MS: (PS-A2) Rₜ 3.20 [M+H]⁺ 357.99.

### 1E. 3-(4-Chloro-3-fluoro-phenyl)-3-[4-(1H-pyrazol-4-yl)-phenyl]-propionamide

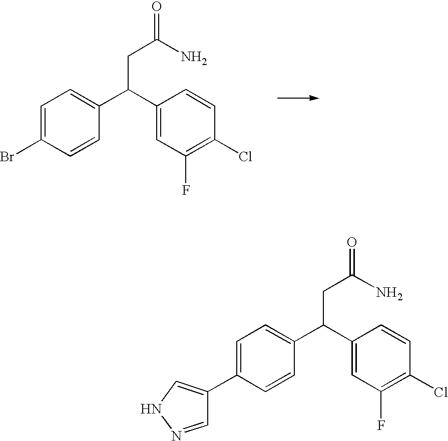

3-(4-Bromo-phenyl)-3-(4-chloro-3-fluoro-phenyl)-propionamide (0.15 g, 0.421 mmol), 4-(4,4,5,5-tetramethyl-1,3,2-dioxaborolan-2-yl)-1H-pyrazole (0.122 g, 0.631 mmol), and potassium phosphate (0.313 g, 1.474 mmol) were combined and suspended in a mixture of methanol, ethanol, toluene and water (1 ml of each). The mixture was degassed with nitrogen and a catalytic amount of tetrakis(triphenyl-phosphine)palladium(0) was added. The mixture was degassed and then irradiated in a CEM Explorer™ microwave at 145° C. for 30 minutes using 100 Watts of power. The reaction was allowed to cool to room temperature and was diluted with 2N sodium hydroxide (aq). The aqueous was extracted twice with ethyl acetate. The organic layer was separated, dried (MgSO₄) and concentrated in vacuo. The residue was purified by flash column chromatography on silica, eluting with methanol/dichloromethane (15:85) to afford the title compound as a white solid (0.097 g, 67% yield). LC/MS: (PS-A2) 2.63 [M+H]⁺ 344.11.

### 1F. 3-(4-Chloro-3-fluoro-phenyl)-3-[4-(1H-pyrazol-4-yl)-phenyl]-propylamine

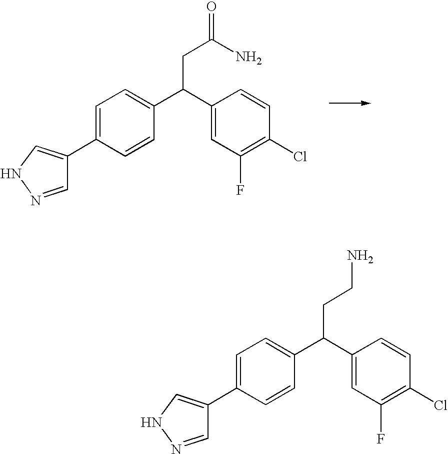

3-(4-Chloro-3-fluoro-phenyl)-3-[4-(1H-pyrazol-4-yl)-phenyl]-propionamide (0.0995 g, 0.289 mmol) was suspended in a mixture of anhydrous diethyl ether (2.89 ml) and anhydrous toluene (1.84 ml). Lithium aluminium hydride powder (0.0615 g, 1.62 mmol) was slowly added followed by a suspension of aluminium (111) chloride (0.108 g, 0.809 mmol) in anhydrous diethyl ether (3 ml). The reaction was heated at 65° C. for 4 hours with stirring. Additional amounts of lithium aluminium hydride (0.0615 g, 1.62 mmol) and aluminium (III) chloride (0.108 g, 0.809 mmol) were added to the reaction and heating was continued at 65° C. for a further 64 hours. The reaction was allowed to cool to room temperature, quenched with water and basified with 2N NaOH (aq). The aqueous was extracted twice with ethyl acetate. The organic layer was separated, dried (MgSO₄) and concentrated in vacuo. The residue was purified by flash column chromatography on silica, eluting with 2N ammonia in methanol/dichloromethane (15:85) followed by preparative HPLC and then basic ion exchange chromatography to afford the title compound as a colourless gum (0.0083 g, 9% yield). LC/MS: (PS-A2) 2.15 [M+H]⁺ 330.12. ¹H NMR (Me-d₃-OD) 2.17-2.31 (2H, m), 2.62 (2H, t), 4.06 (1H, t), 7.13 (114, d), 7.20 (1H, d), 7.29 (2H, d), 7.38 (111, t), 7.53 (2H, d), 7.93 (2H, s).

### Example 2

### {(R)-3-(3,4-Dichloro-phenyl)-3-[4-(1H-pyrazol-4-yl)-phenyl]-propylamine

### 2A. 3-(4-Bromo-phenyl)-2-cyano-acrylic acid ethyl ester

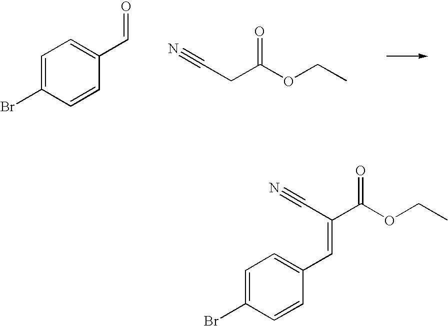

4-Bromobenzaldehyde (13.9 g, 75.13 mmol) and ethyl cyanoacetate (8.79 ml, 82.6 mmol) in toluene was added piperidine (125 μl) and the reaction mixture was refluxed for 2 hour with a Dean-Stark separator. The solvent was removed under reduced pressure, the residue was purified by silica column chromatography eluting with petroleum ether to ethyl acetate/petroleum ether (20:80) to yield the desired product as a white crystalline solid (8.4 g, 40% yield). LC/MS: (acidic_PL01) Rₜ 3.50.

### 2B. 3-(4-Bromo-phenyl)-3-(4-chloro-phenyl)-2-cyano-propionic acid ethyl ester

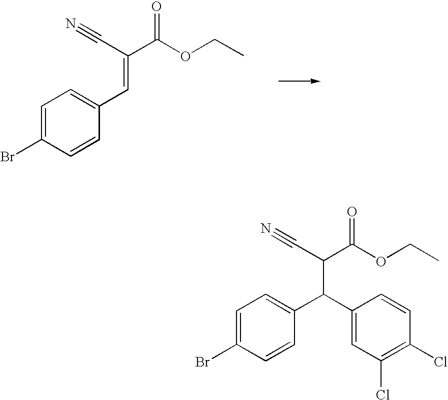

A solution of 3-(4-bromo-phenyl)-2-cyano-acrylic acid ethyl ester (8.4 g, 30.0 mmol) in dry toluene (30 ml) was added dropwise to 3,4-dichlorophenylmagnesium bromide (0.5 M solution in tetrahydrofuran, 120 nil, 60.0 mmol) at 0° C. The reaction mixture was heated to 70° C. for 2 hours, poured onto ice, acidified with 1N HCl and extracted with ethyl acetate. The organic layer was separated, dried (MgSO₄), filtered and concentrated, the crude product was purified over flash silica chromatography eluting with petroleum ether to ethyl acetate/petroleum ether (20:80) to afford the desired product (15.27 g some small impurities). LC/MS: (acidic_PL01) Rₜ 3.97 [M+H]⁻ 425.92.

### 2C. 3-(4-Bromo-phenyl)-3-(4-chloro-phenyl)-propionic acid

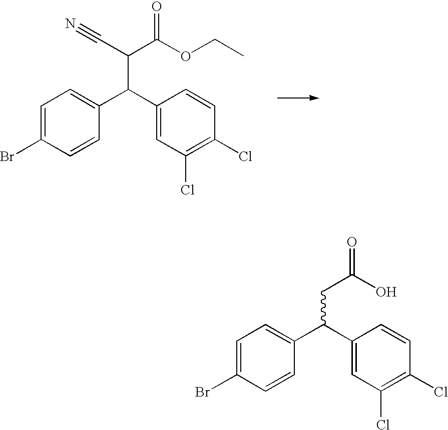

A mixture of 3-(4-bromo-phenyl)-3-(4-chloro-phenyl)-2-cyano-propionic acid ethyl ester (1.91 g, 4.87 mmol), acetic acid (10 ml), concentrated sulphuric acid (5 ml) and water (5 ml) were refluxed for 2 hours. Reaction mixture was poured into iced water and extracted with ethyl acetate. The organic layer was separated, dried (MgSO₄), filtered and concentrated, the crude product was purified over flash silica chromatography eluting with ethyl acetate/petroleum ether (1:1) to afford the desired product (0.82 g, 50% yield). LC/MS: (PS-A2) R₁ 3.39 [M+11]⁻ 338.86.

### 2D. 3-(3,4-Dichloro-phenyl)-3-[4-(1H-pyrazol-4-yl)-phenyl]-propionic acid

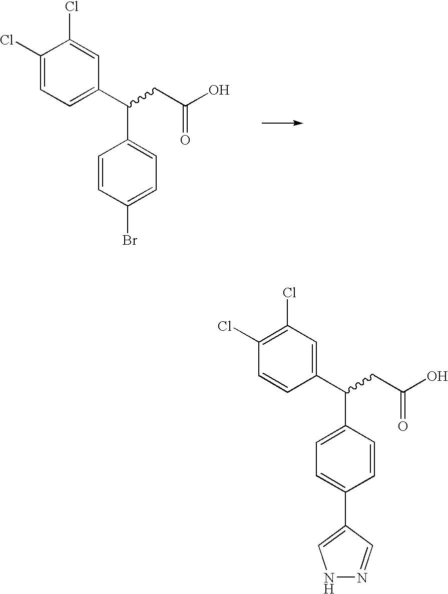

A mixture of 3-(4-Bromo-phenyl)-3-(4-chloro-phenyl)-propionic acid (4.32 g, 11.5 mmol), potassium phosphate (11.03 g, 52.0 mmol) and 4-(4,4,5,5-tetramethyl-1,3,2-dioxaborolan-2-yl)-1H-pyrazole (4.48 g, 23.01 mmol) in ethanol/ methanol/ toluene/ water (15 ml of each solvent) was degassed with nitrogen before addition of tetrakis(triphenylphosphine)palladium (0) (0.67 g, 5 mol %). The reaction was heated at 95° C. for 16 hours, cooled, degassed again with nitrogen more tetrakis(triphenylphosphine)palladium (0) (0.67 g, 5 mol %) added and heated at 95° C. for a further 24 hours. The reaction was partitioned between ethyl acetate and aqueous 5% citric acid. The aqueous layer was extracted with ethyl acetate and the combined organic layers were dried (MgSO₄) and concentrated under reduced pressure. The crude product was purified by silica column chromatography, eluting with dichloromethane to dichloromethane/ methanol/ acetic acid/ H₂O (120:18:3:2) to afford the desired product (2.15 g, 52% yield). LC/MS: (acidic_PL01) Rₜ 2.96 [M+H]⁺ 361.02.

### 2E. (R)-3-(3,4-Dichloro-phenyl)-N-((S)-1-phenyl-ethyl)-3-[4-(1H-pyrazol-4-yl)-phenyl]-propionamide

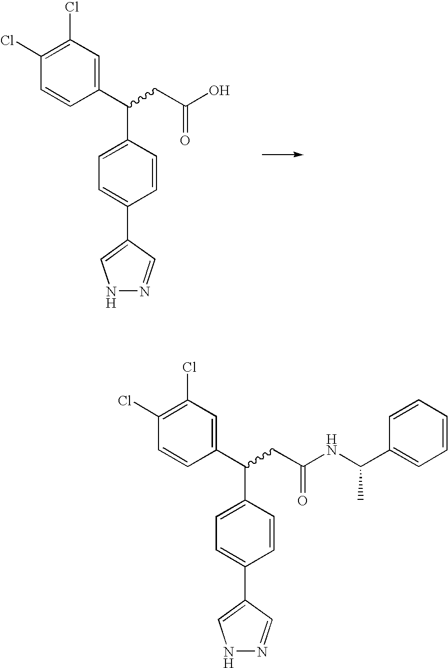

A mixture of 3-(3,4-dichloro-phenyl)-3-[4-(1H-pyrazol-4-yl)-phenyl]-propionic acid (4.22 g, 11.68 mmol) and thionyl chloride (10 ml) was heated at 80° C. for 2 hours. The reaction was allowed to cool then concentrated in vacuo and re-concentrated from toluene. The residue was then dissolved in dichloromethane (10 ml) and added dropwise to a stirred mixture of L-(−)-α-methylbenzylamine (1.78 ml, 14.01 mmol), triethylamine (2.44 ml, 17.52 mmol) and dichloromethane (30 ml) at 0° C. After stirring for 18 hours, the mixture was concentrated in vacuo and partitioned between ethyl acetate and water (aqueous adjusted to pH 8 using saturated sodium bicarbonate), a small amount of methanol was added to aid solubility. The aqueous layer was extracted with ethyl acetate and the combined organic layers were washed with 2N aqueous hydrochloric acid, dried (MgSO₄) and concentrated under reduced pressure. The crude product was purified by silica column chromatography, eluting with ethyl acetate/petroleum ether (65:35) to ethyl acetate (100%), the product was purified again by silica column chromatography eluting with dichloromethane to dichloromethane/methanol (97:3) to afford the desired product (0.61 g, 11% yield). LC/MS: (acidic PL01) Rₜ 3.26 [M+H]⁺ 464.12.

### 2F. (R)-3-(3,4-Dichloro-phenyl)-3-[4-(1H-pyrazol-4-yl)-phenyl]-propionamide

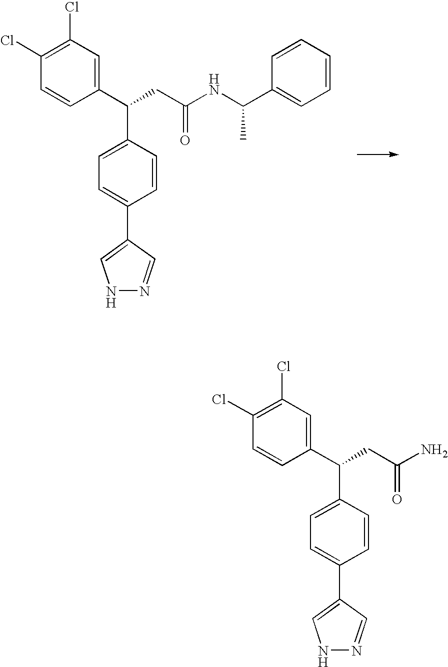

(R)-3-(3,4-Dichloro-phenyl)-N—((S)-1-phenyl-ethyl)-3-[4-(1H-pyrazol-4-yl)-phenyl]-propionamide (0.05 g, 0.108 mmol) was stirred in concentrated sulphuric acid (2 ml) at 40° C. for 1 hour. Reaction mixture was poured into iced water, neutralized with 2N aqueous sodium hydroxide and extracted with ethyl acetate. The organic extracts were combined, dried (MgSO₄) and concentrated in vacuo. The crude product was purified by silica column chromatography eluting with dichloromethane/methanol (90:10) to afford the desired product (0.026 g, 67% yield). LC/MS: (acidic_PL01) Rₜ 2.73 [M+H]⁺ 360.03.

### 2G. (R)-3-(3,4-Dichloro-phenyl)-3-[4-(1H-pyrazol-4-yl)-phenyl]-propylamine

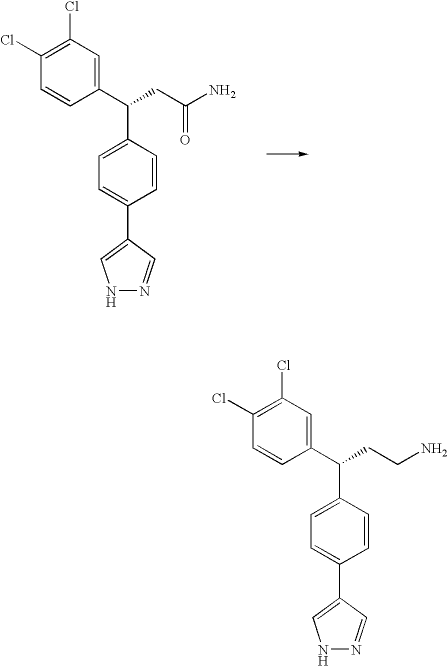

Under a nitrogen atmosphere, (R)-3-(3,4-dichloro-phenyl)-3-[4-(1H-pyrazol-4-yl)-phenyl]-propionamide (0.077 g, 0.214 mmol) suspension in diethyl ether (3 ml) and toluene (0.75 ml) was cooled to 0° C., aluminium chloride (0.071 g, 0.534 mmol) was added followed by lithium aluminum hydride (0.041 g, 1.069 mmol). The reaction mixture was heated at 65° C. for 2 hours, quenched with addition of water, basified (2N NaOH) and extracted with ethyl acetate. The organic layer was separated, dried (MgSO₄) and concentrated in vacuo, the crude product was purified preparative HPLC using formic acid as the modifier. Product partitioned between ethyl acetate and saturated aqueous sodium bicarbonate, organic layer separated, dried (MgSO₄) and concentrated to yield the desired product (0.007 g, 10% yield). LC/MS: (acidic_PL01) Rₜ 2.22 [M+H]⁺ 346.08. ¹H NMR (Me-d₃-OD) 7.80 (2H, s), 7.42 (21-1, d), 7.34 (2H, m), 7.15 (314, m), 3.95 (1H, m), 2.55 (2H, d), 2.15 (2H, m).

### Examples 3 to 76

By following the General Procedures set out above, the compounds of Examples 3 to 76 may be prepared.

### Example 3

### 3-[3-(4-Methyl-piperazin-1-yl)-phenyl]-3-[4-(1H-pyrazol-4-yl)-phenyl]-propylamine

### 3A. 4-[3-(CE)-2-Cyano-2-ethoxycarbonyl-vinyl)-phenyl]-piperazine-1-carboxylic acid tert-butyl ester

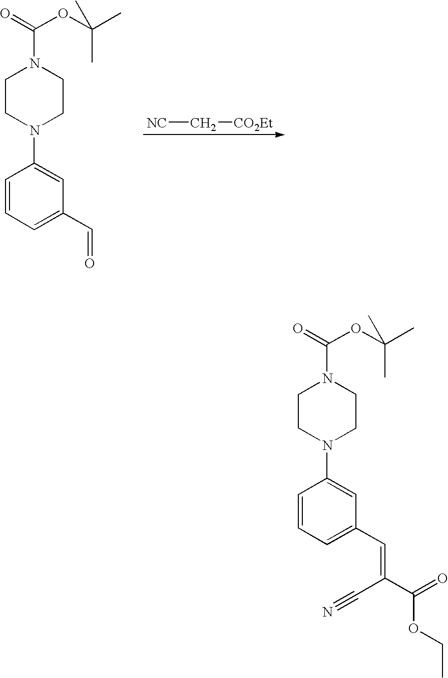

By following General Procedure 3, and replacing 4-bromobenzaldehyde with 4-(3-formyl-phenyl)-piperazine-1-carboxylic acid tert-butyl ester, the title compound can be obtained.

### 3B. (E)-2-Cyano-3-(3-piperazin-1-yl-phenyl)-acrylic acid ethyl ester

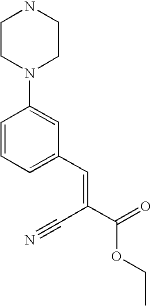

The title compound can be prepared by deprotection of the amine product of Example 3A using trifluoroacetic acid in an aprotic solvent such as dichloromethane (see Protective Groups in Organic Synthesis, Greene and Wuts, Wiley Interscience, 3^{rd} edition).

### 3C. (E)-2-Cyano-3-[3-(4-methyl-piperazin-1-yl)-phenyl]-acrylic acid ethyl ester

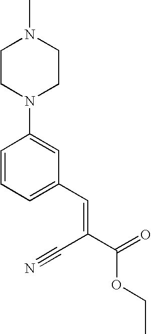

The title compound can be prepared by methylation of the product of Example 3B, (E)-2-cyano-3-(3-piperazin-1-yl-phenyl)-acrylic acid ethyl ester, using iodomethane in a suitable organic solvent (e.g. dichloromethane) in the presence of a base (e.g. triethylamine).

### 3D. 3-[3-(4-Methyl-piperazin-1-yl)-phenyl]-3-[4-(1H-pyrazol-4-yl)-phenyl]-propylamine

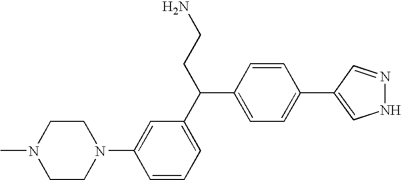

The title compound can be prepared using the method described in General Procedures 3B to 3F, using intermediate 3C in place of stated acrylic ester and methylamine with ammonia in 3D.

### Example 4

### Methyl-{3-[3-(4-methyl-piperazin-1-yl)-phenyl]-3-[4-(1H-pyrazol-4-yl)-phenyl]-propyl}-amine

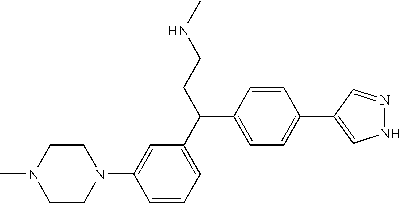

The title compound can be prepared using the method described in General Procedures 3B to 3F, using intermediate 3C in place of the stated acrylic ester.

### Example 5

### Dimethyl-{3-[3-(4-methyl-piperazin-1-yl)-phenyl]-3-[4-(1H-pyrazol-4-yl)-phenyl]-propyl}-amine

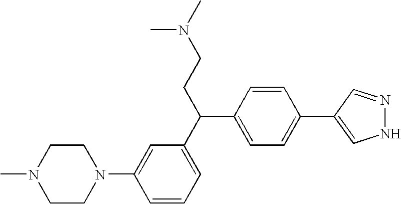

The title compound can be prepared using the methods described in General Procedures 3B to 3F, using intermediate 3C in place of the stated acrylic ester and methylamine with dimethylamine in 3D.

### Example 6

### 3-(4-Chloro-3-morpholin-4-yl-phenyl)-3-[4-(1H-pyrazol-4-yl)-phenyl]-propylamine

### 6A. Ethyl 4-Chloro-3-morpholin-4-yl-benzoate

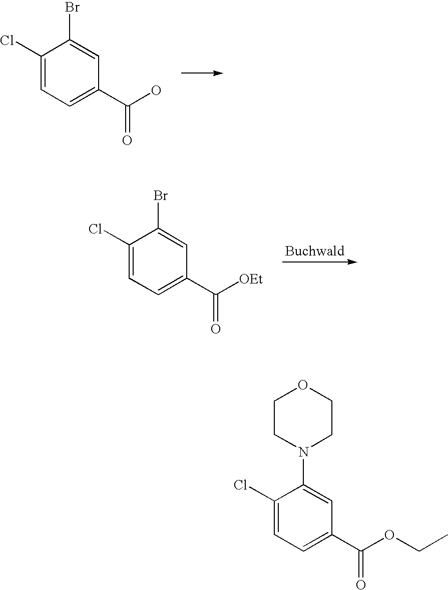

3-Bromo-4-chloro-benzoic acid ethyl ester (obtained by esterification of 3-bromo-4-chloro-benzoic acid) can be aminated according to the method described in Tetrahedron Letters (2001), 42(23), 3917-3919) to obtain the title compound.

### 6B. 4-Chloro-3-morpholin-4-yl-benzaldehyde

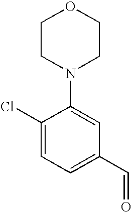

The title compound can be prepared from 4-chloro-3-morpholin-4-yl-benzoic acid ethyl ester by reduction of the ester moiety either directly to the aldehyde or via the alcohol according to standard methods.

### 6C. 3-(4-Chloro-3-morpholin-4-yl-phenyl)-3-[4-(1H-pyrazol-4-yl)-phenyl]-propylamine

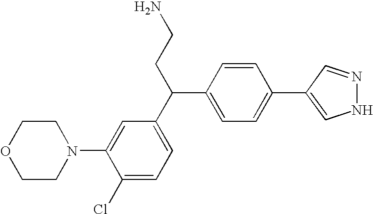

The title compound could be obtained using the methods described in General Procedure 3 where 4-chloro-3-morpholin-4-yl-benzaldehyde replaces 4-bromobenzaldehyde in 3A and ammonia replaces methylamine in 3D.

### Example 7

### {3-(4-Chloro-3-morpholin-4-yl-phenyl)-3-[4-(1H-pyrazol-4-yl)-phenyl]-propyl}-methyl-amine

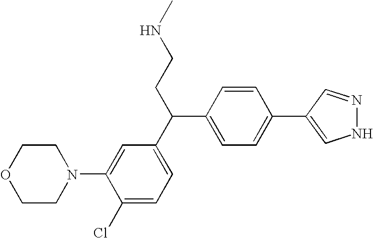

The title compound can be obtained using the methods described in General Procedure 3 where 4-chloro-3-morpholin-4-yl-benzaldehyde replaces 4-bromobenzaldehyde in 3A.

### Example 8

### {3-(4-Chloro-3-morpholin-4-yl-phenyl)-3-[4-(1H-pyrazol-4-yl)-phenyl]-propyl}-dimethyl-amine

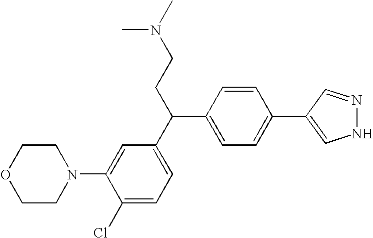

The title compound could be obtained using the method described in General Procedure 3 where 4-chloro-3-morpholin-4-yl-benzaldehyde replaces 4-bromobenzaldehyde in 3A and dimethylamine replaces methylamine in 3D.

### Example 9

### {2-(4-Chloro-3-morpholin-4-yl-phenyl)-2-[4-(1H-pyrazol-4-yl)-phenyl]-ethyl}-methyl-amine

### 9A. 4-(2-Chloro-5-oxiranyl-phenyl)-morpholine

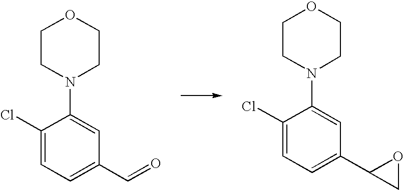

The title compound can be obtained from 4-chloro-3-morpholin-4-yl-benzaldehyde using the method described in General Procedure 6A.

### 9B. {2-(4-Chloro-3-morpholin-4-yl-phenyl)-2-[4-(1H-pyrazol-4-yl)-phenyl]-ethyl}-methyl-amine

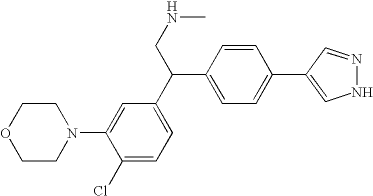

The title compound can be obtained using the method described in General Procedure 5, replacing 2-(4-bromophenyl)-oxirane with 4-(2-chloro-5-oxiranyl-phenyl)-morpholine and using-4-bromobenzene as R¹—H in 5B.

### Example 10

### {2-(4-Chloro-3-morpholin-4-yl-phenyl)-2-[4-(1H-pyrazol-4-yl)-phenyl]-ethyl}-dimethyl-amine

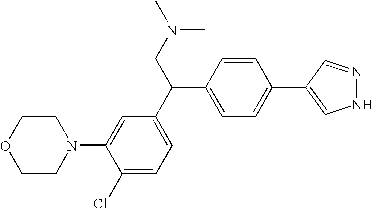

The title compound can be obtained using the methods described in General Procedure 5, replacing 2-(4-bromophenyl)-oxirane with 4-(2-chloro-5-oxiranyl-phenyl)-morpholine and methylamine with dimethylamine and using 4-bromobenzene as R¹—H in 5B.

### Example 11

### 2-(4-Chloro-3-morpholin-4-yl-phenyl)-2-[4-(1H-pyrazol-4-yl)-phenyl]-ethylamine

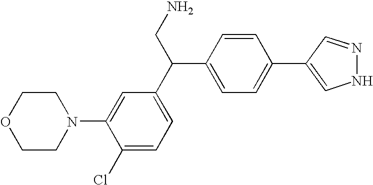

The title compound can be obtained using the methods described in General Procedures 5, replacing 2-(4-bromophenyl)-oxirane with 4-(2-chloro-5-oxiranyl-phenyl)-morpholine and methylamine with ammonia and using 4-bromobenzene in 5B.

### Example 12

### 4-(3-{-4-[4-(1H-Pyrazol-4-yl)-phenyl]-piperidin-4-yl}-phenyl)-morpholine

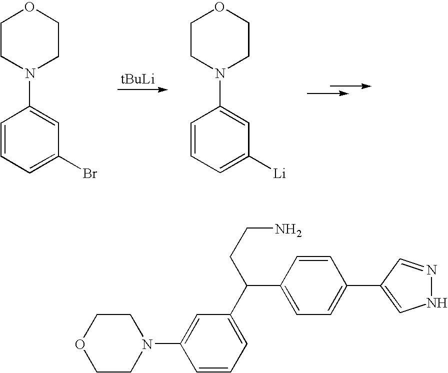

4-(3-lithio-phenyl)-morpholine, obtained according to procedures outlined in WO 2002083642 from 4-(3-bromophenyl)-morpholine, can be used as the aryl-magnesium bromide in General Procedure 3B to give the intermediate biaryl propionic acid ester. This intermediate ester can then be converted to the desired compound according to General Procedures 3C to 3F where ammonia replaces methylamine.

### Example 13

### Methyl-{3-(3-morpholin-4-yl-phenyl)-3-[4-(1H-pyrazol-4-yl)-phenyl]-propyl}-amine

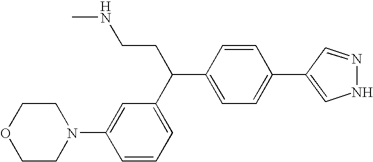

4-(3-lithio-phenyl)-morpholine, obtained according to procedures outlined in WO 2002083642 from 4-(3-bromophenyl)-morpholine, could be used as the aryl-magnesium bromide in General Procedure 3B to give the intermediate biaryl propionic acid ester which can then be converted to the desired compound according to General Procedures 3C to 3F.

### Example 14

### Dimethyl-{3-(3-morpholin-4-yl-phenyl)-3-[4-(1H-pyrazol-4-yl)-phenyl]-propyl}-amine

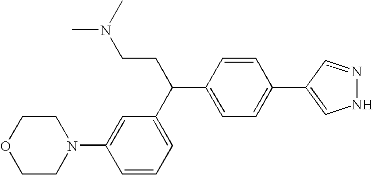

4-(3-lithio-phenyl)-morpholine, obtained according to procedures outlined in WO 2002083642 from 4-(3-bromophenyl)-morpholine, could be used could be used as the aryl-magnesium bromide in General Procedure 3B to give the intermediate biaryl propionic acid ester which can then be converted to the desired compound according to according to General Procedures 3C to 3F where dimethylamine replaces methylamine.

### Example 15

### 3-(3-Morpholin-4-yl-phenyl)-3-[4-(1H-pyrazol-4-yl)-phenyl]-propylamine

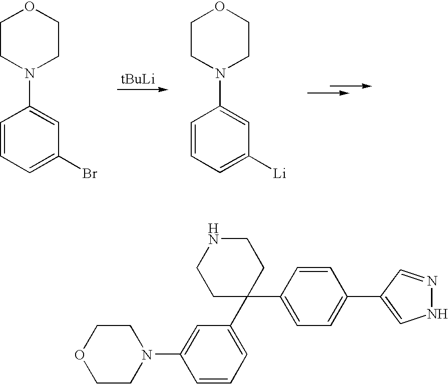

4-(3-Lithio-phenyl)-morpholine, obtained according to procedures described in WO 2002083642 from 4-(3-bromophenyl)-morpholine, can be used as the aryl-magnesium bromide (x) in the methods described in General Procedure 8 to give the title compound.

### Example 16

### 3-(3-Imidazol-1-yl-phenyl)-3-[4-(1H-pyrazol-4-yl)-phenyl]-propylamine

### 16A. 3-Imidazol-1-yl-benzaldehyde

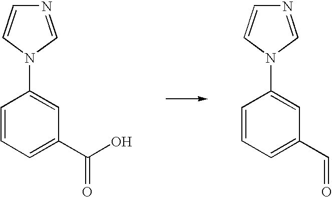

The title compound can be obtained from 3-(1H-imidazol-1-yl)benzoic acid by esterification, reduction and oxidation.

### 16B. 3-(3-Imidazol-1-yl-phenyl)-3-[4-(1H-pyrazol-4-yl)-phenyl]-propylamine

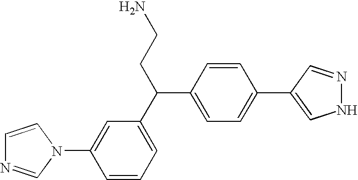

Using the methods described in General Procedure 3, replacing 4-bromobenzaldehyde with 3-imidazol-1-yl-benzaldehyde and methylamine with ammonia, the title compound can be obtained.

### Example 17

### {3-(3-Imidazol-1-yl-phenyl)-3-[4-(1H-pyrazol-4-yl)-phenyl]-propyl}-methyl-amine

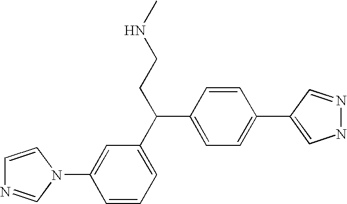

Using the methods described in General Procedure 3, replacing 4-bromobenzaldehyde with 3-imidazol-1-yl-benzaldehyde, the title compound can be obtained.

### Example 18

### {3-(3-Imidazol-1-yl-phenyl)-3-[4-(1H-pyrazol-4-yl)-phenyl]-propyl}-dimethyl-amine

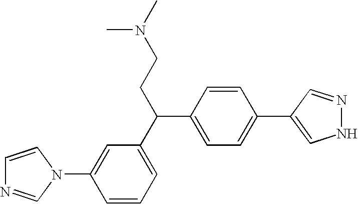

Using the methods described in General Procedure 3, replacing 4-bromobenzaldehyde with 3-imidazol-1-yl-benzaldehyde and methylamine with dimethylamine, the title compound can be obtained.

### Example 19

### {2-(3-Imidazol-1-yl-phenyl)-2-[4-(1H-pyrazol-4-yl)-phenyl]-ethyl}-methyl-amine

### 19A. 1-(3-Oxiranyl-phenyl)-1H-imidazole

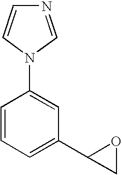

Using the method described in General Procedure 6A, using 3-imidazol-1-yl-benzaldehyde (prepared above) as the ketone (p), the title compound can be obtained.

### 19B. {2-(3-Imidazol-1-yl-phenyl)-2-[4-(1H-pyrazol-4-yl)-phenyl]-ethyl}-methyl-amine

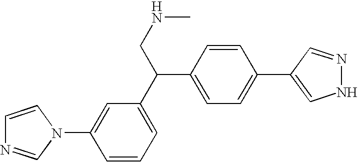

The title compound can be prepared according to the methods described in General Procedure 5 replacing 2-(4-bromophenyl)-oxirane in General Procedure 5A with 1-(3-oxiranyl-phenyl)-1H-imidazole.

### Example 20

### 2-(3-Imidazol-1-yl-phenyl)-2-[4-(1H-pyrazol-4-yl)-phenyl]-ethylamine

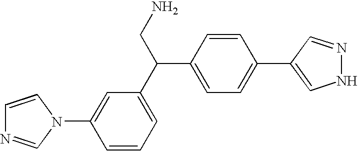

The title compound can be prepared according to the methods described in General Procedure 5 replacing 2-(4-bromophenyl)-oxirane with 1-(3-oxiranyl-phenyl)-1H-imidazole and dimethylamine with ammonia.

### Example 21

### {2-(3-Imidazol-1-yl-phenyl)-2-[4-(1H-pyrazol-4-yl)-phenyl]-ethyl}-dimethyl-amine

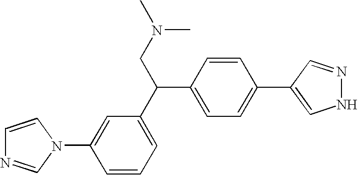

The title compound can be prepared according to the methods described in General Procedure 5 replacing 2-(4-bromophenyl)-oxirane with 1-(3-oxiranyl-phenyl)-1H-imidazole and dimethylamine with dimethylamine.

### Example 22

### 4′-[4-(1H-Pyrazol-4-yl)-phenyl]-1′,2′,3′,4′,5′,6′-hexahydro-[2,4′]bipyridinyl

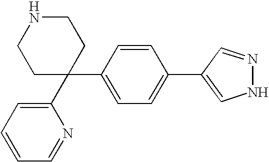

The title compound can be prepared by following General Procedure 8 using pyridine-2-magnesium bromide (prepared from 2-bromopyridine according to the procedures described in Tetrahedron, (2003), 59(43), 8629-8640)) as the aryl-magnesium bromide (x).

### Example 23

### Methyl-{2-[4-(1H-pyrazol-4-yl)-phenyl]-2-pyridin-2-yl-ethyl}-amine

### 23A. 2-Oxiranyl-pyridine

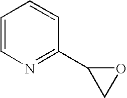

This intermediate can be synthesised from pyridine-2-carboxaldehyde according to the method described in General Procedure 6A or Heterocycles, (1984), 22(11), 2517-22.

### 23B. Methyl-{2-[4-(1H-pyrazol-4-yl)-phenyl]-2-pyridin-2-yl-ethyl}-amine

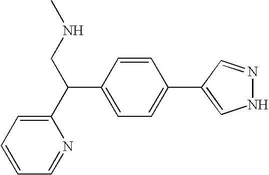

The title compound can be prepared according to the methods described in General Procedure 5, replacing 2-(4-bromophenyl)-oxirane with 2-oxiranyl-pyridine.

### Example 24

### Dimethyl-{2-[4-(1H-pyrazol-4-yl)-phenyl]-2-pyridin-2-yl-ethyl}-amine

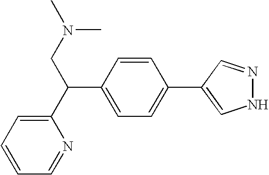

The title compound can be prepared according to the methods described in General Procedure 5, replacing 2-(4-bromophenyl)-oxirane with 2-oxiranyl-pyridine and methylamine with dimethylamine.

### Example 25

### 2-[4-(1H-Pyrazol-4-yl)-phenyl]-2-pyridin-2-yl-ethylamine

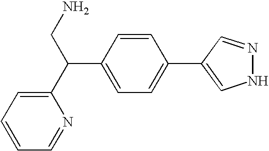

The title compound can be prepared according to the methods described in General Procedure 5, replacing 2-(4-bromophenyl)-oxirane with 2-oxiranyl-pyridine and methylamine with ammonia.

### Example 26

### Methyl-{3-[4-(1H-pyrazol-4-yl)-phenyl]-3-pyridin-3-yl-propyl}-amine

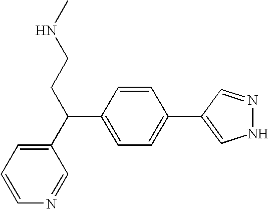

Using the methods described in General Procedure 3, but replacing 4-bromobenzaldehyde with nicotinaldehyde, the title compound can be prepared.

### Example 27

### Dimethyl-{3-[4-(1H-pyrazol-4-yl)-phenyl]-3-pyridin-3-yl-propyl}-amine

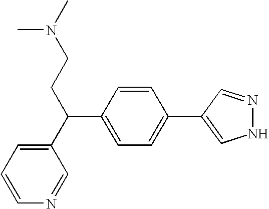

Using the methods described in General Procedure 3, but replacing 4-bromobenzaldehyde with nicotinaldehyde and methylamine with dimethylamine, the title compound can be prepared.

### Example 28

### 3-[4-(1H-Pyrazol-4-yl)-phenyl]-3-pyridin-3-yl-propylamine

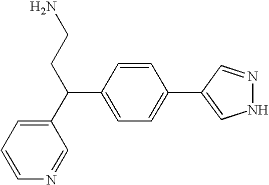

Using the methods described in General Procedure 3, but replacing 4-bromobenzaldehyde with nicotinaldehyde and methylamine with ammonia, the title compound can be prepared.

### Example 29

### Methyl-{3-[4-(1H-pyrazol-4-yl)-phenyl]-3-pyridin-4-yl-propyl}-amine

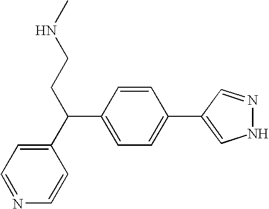

Using the methods described in General Procedure 3, but replacing 4-bromobenzaldehyde with isonicotinaldehyde, the title compound can be prepared.

### Example 30

### Dimethyl-{3-[4-(1H-pyrazol-4-yl)-phenyl]-3-pyridin-4-yl-propyl}-amine

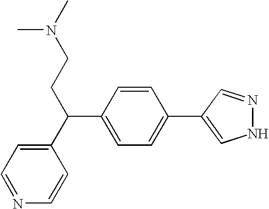

Using the methods described in General Procedure 3, but replacing 4-bromobenzaldehyde with isonicotinaldehyde and methylamine with dimethylamine, the title compound can be prepared.

### Example 31

### 3-[4-(1H-Pyrazol-4-yl)-phenyl]-3-pyridin-4-yl-propylamine

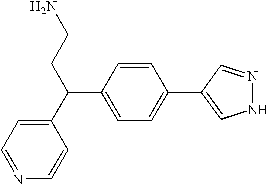

Using the methods described in General Procedure 3, but replacing 4-bromobenzaldehyde with isonicotinaldehyde and methylamine with ammonia, the title compound can be prepared.

### Example 32

### Methyl-{2-[4-(1H-pyrazol-4-yl)-phenyl]-2-pyridin-4-yl-ethyl}-amine

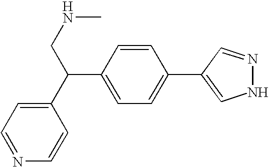

Using the methods described in General Procedures 5B and 5C, replacing 1-(4-bromophenyl)-2-methylaminoethanol with 2-methylamino-1-pyridin-4-yl-ethanol (which can be synthesised according to procedures outlined in WO 2003059911), the title compound can be obtained.

### Example 33

### Methyl-{2-[4-(1H-pyrazol-4-yl)-phenyl]-2-pyrimidin-5-yl-ethyl}-amine

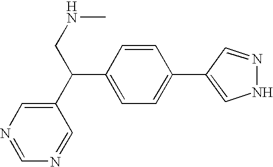

Using the methods described in General Procedure 5, replacing 2-(4-bromophenyl)-oxirane with 5-oxiranyl-pyrimidine (prepared using procedures described in Synthetic Communications (1994), 24(2), 253-6), the title compound can be obtained.

### Example 34

### Dimethyl-{2-[4-(1H-pyrazol-4-yl)-phenyl]-2-pyrimidin-5-yl-ethyl}-amine

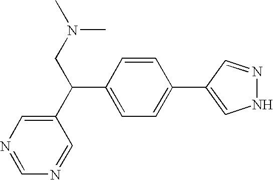

Using the methods described in General Procedure 5, replacing 2-(4-bromophenyl)-oxirane with 5-oxiranyl-pyrimidine (prepared according to procedures described in Synthetic Communications (1994), 24(2), 253-6) and methylamine with dimethylamine, the title compound can be obtained.

### Example 35

### 2-[4-(1H-Pyrazol-4-yl)-phenyl]-2-pyrimidin-5-yl-ethylamine

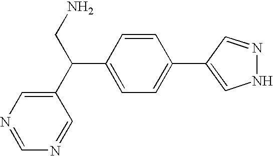

Using the methods described in General Procedure 5, replacing 2-(4-bromophenyl)-oxirane with 5-oxiranyl-pyrimidine (prepared according to procedures described in Synthetic Communications (1994), 24(2), 253-6) and methylamine with ammonia, the title compound can be obtained.

### Example 36

### Methyl-{2-[4-(1H-pyrazol-4-yl)-phenyl]-2-pyrimidin-4-yl-ethyl}-amine

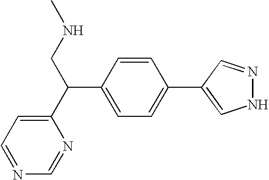

Using the methods described in General Procedure 6A and General Procedure 5, using pyrimidine-4-carboxaldehyde instead of the ketone (p), the title compound can be obtained.

### Example 37

### Dimethyl-{2-[4-(1H-pyrazol-4-yl)-phenyl]-2-pyrimidin-4-yl-ethyl}-amine

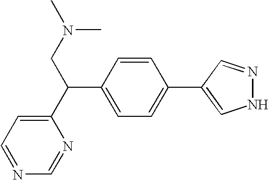

Using the methods described in General Procedure 6A and General Procedure 5, using pyrimidine-4-carboxaldehyde instead of the ketone (p), and replacing methylamine with dimethylamine, the title compound can be obtained.

### Example 38

### 2-[4-(1H-Pyrazol-4-yl)-phenyl]-2-pyrimidin-4-yl-ethylamine

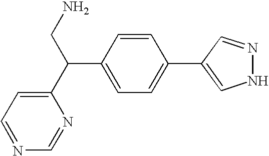

Using the methods described in General Procedure 6A and General Procedure 5, using pyrimidine-4-carboxaldehyde instead of the ketone (p), and replacing methylamine with ammonia, the title compound can be obtained.

### Example 39

### 3-[4-(1H-Pyrazol-4-yl)-phenyl]-3-pyrimidin-4-yl-propylamine

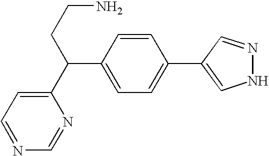

Using the methods described in General Procedure 3, replacing 4-bromobenzaldehyde with pyrimidine-4-carboxaldehyde and methylamine with ammonia, the title compound can be obtained.

### Example 40

### 3-Pyrazin-2-yl-3-[4-(1H-pyrazol-4-yl)-phenyl]-propylamine

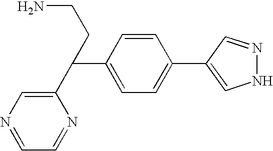

Using the methods described in General Procedure 3, replacing, 4-bromobenzaldehyde with pyrazine-2-carbaldehyde (obtainable according to procedures described in U.S. Pat. No. 4,826,833) and methylamine with ammonia, the title compound can be obtained.

### Example 41

### 3-Furan-2-yl-3-[4-(1H-pyrazol-4-yl)-phenyl]-propylamine

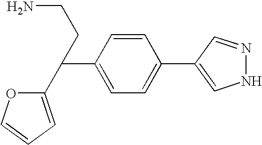

Using the methods described in General Procedure 3, replacing 4-bromobenzaldehyde with furfural and methylamine with ammonia, the title compound can be obtained.

### Example 42

### 3-Furan-3-yl-3-[4-(1H-pyrazol-4-yl)-phenyl]-propylamine

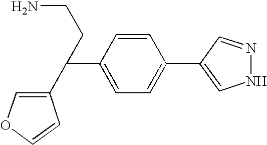

Using the methods described in General Procedure 3, replacing 4-bromobenzaldehyde with 3-furaldehyde and methylamine with ammonia, the title compound can be obtained.

### Example 43

### 3-[4-(1H-Pyrazol-4-yl)-phenyl]-3-thiophen-2-yl-propylamine

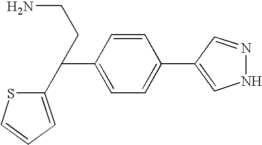

Using the methods described in General Procedure 3, replacing 4-bromobenzaldehyde with thiophene-2-carbaldehyde (obtainable using the procedures described in Bioorganic & Medicinal Chemistry, (2004), 12(5), 1221-1230) and methylamine with ammonia, the title compound can be obtained.

### Example 44

### 4-[4-(1H-Pyrazol-4-yl)-phenyl]-4-thiophen-2-yl-piperidine

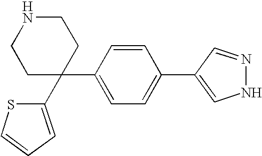

Using the methods described in General Procedure 8, using thiophen-2-yl-magnesium bromide as the aryl-magnesium bromide (x), the title compound can be obtained.

### Example 45

### 3-[4-(1H-Pyrazol-4-yl)-phenyl]-3-thiophen-3-yl-propylamine

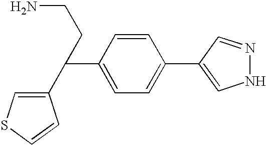

Using the methods described in General Procedure 3, replacing 4-bromobenzaldehyde with thiophene-2-carbaldehyde (obtainable using the procedures described in Synthetic Communications, (1984), 14(1), 1-9), the title compound can be obtained.

### Example 46

### 4-[4-(1H-Pyrazol-4-yl)-phenyl]-4-thiophen-3-yl-piperidine

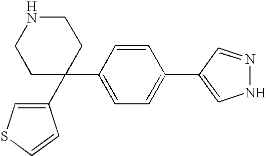

Using the methods described in General Procedure 8, using 3-thienylmagnesium iodide as the aryl-magnesium bromide (x), the title compound can be obtained.

### Example 47

### {3-(3,4-Dichloro-phenyl)-1,1-dimethyl-3-[4-(1H-pyrazol-4-yl)-phenyl]-propyl}-dimethyl-amine

### 47A. 3-(4-Bromo-phenyl)-3-(3,4-dichloro-phenyl)-N,N-dimethyl-propionamide

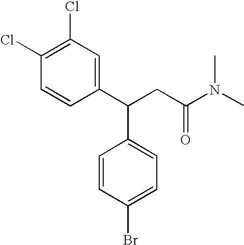

The title compound can be prepared using the methods described in General Procedures 3A to 3D using 3,4-dichlorophenylmagnesium bromide and replacing methylamine with dimethylamine.

### 47B. 3-(3,4-Dichloro-phenyl)-N,N-dimethyl-3-[4-(1H-pyrazol-4-yl)-phenyl]-propionamide

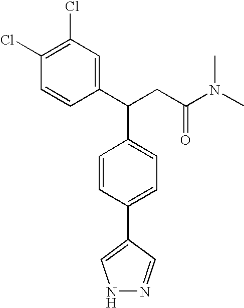

The title compound can be prepared from Example 47A using General Procedure 1.

### 47C. {3-(3,4-Dichloro-phenyl)-1,1-dimethyl-3-[4-(1H-pyrazol-4-yl)-phenyl]-propyl}-dimethylamine

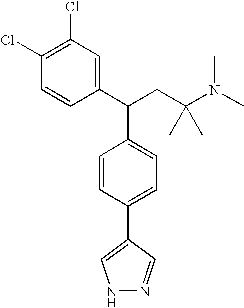

The title compound can be made by treating the product from Example 47B with methylmagnesium bromide in the presence of zirconium tetrachloride (Synlett. 1999 (1), 55-6).

### Example 48

### 3-Amino-1-(3,4-dichloro-phenyl)-1-[4-(1H-pyrazol-4-yl)-phenyl]-propan-1-ol

### 48A. 2-[3-(4-Bromo-phenyl)-3-oxo-propyl]-isoindole-1,3-dione

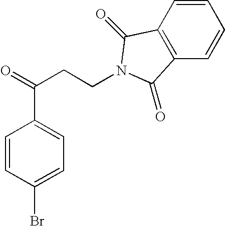

The title compound can be prepared by treating the commercially available 1-(4-bromo-phenyl)-3-chloro-propan-1-one with potassium pthalimide.

### 48B. 2-[3-(4-Bromo-phenyl)-3-(3,4-dichloro-phenyl)-3-hydroxy-propyl]-isoindole-1,3-dione

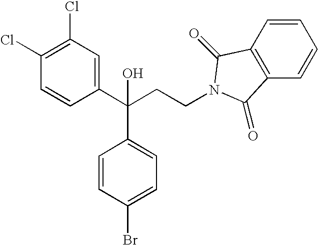

The title compound can be prepared by the addition of 3,4-dichlorophenylmagnesium bromide to the compound prepared in Example 48A.

### 48C. 2-{3-(3,4-Dichloro-phenyl)-3-hydroxy-3-[4-(1H-pyrazol-4-yl)-phenyl]-propyl}-isoindole-1,3-dione

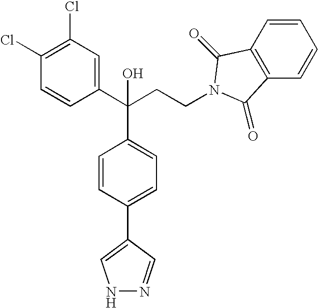

The title compound can be prepared from the compound prepared in Example 48B by following General Procedure 1.

### 48D. 3-Amino-1-(3,4-dichloro-phenyl)-1-[4-(1H-pyrazol-4-yl)-phenyl]-propan-1-ol

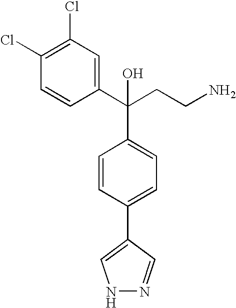

The title compound can be prepared from the compound described in Example 48C using General Procedure 7.

An alternative synthetic route to 3-Amino-1-(3,4,-dichlorophenyl)-1-[4-(1H-pyrazol-4-yl)-phenyl]-propan-1-ol is described in Example 96.

### Example 49

### 3-(3,4-Dichloro-phenyl)-3-fluoro-3-[4-(1H-pyrazol-4-yl)-phenyl]-propylamine

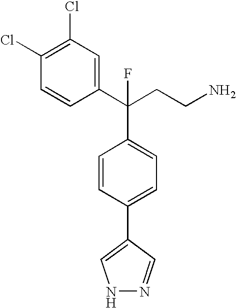

The title compound can be prepared from the product of Example 48 using General Procedure 9.

### Example 50

### 3-(3,4-Dichloro-phenyl)-3-[4-(3-methyl-1H-pyrazol-4-yl)-phenyl]-propylamine

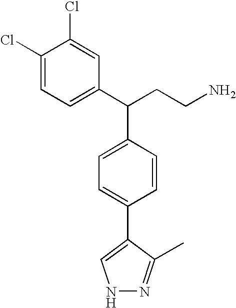

Following the procedure described in General Procedure 4, substituting 3-methyl-1-trityl-1H-pyrazole-4-boronic acid* for 4-(4,4,5,5-tetramethyl-1,3,2-dioxaborolan-2-yl)-1H-pyrazole in Procedure 4C, the title compound can be obtained.

*Prepared as EP1382603A1

### Example 51

### {3-(3,4-Dichloro-phenyl)-3-[4-(3-methyl-1H-pyrazol-4-yl)-phenyl]-propyl}-methyl-amine

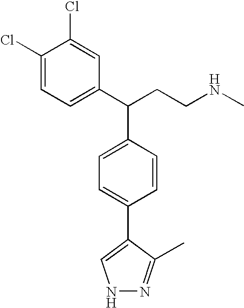

Following the procedure described in General Procedure 4, substituting 3-methyl-1-trityl-1H-pyrazole-4-boronic acid* for 4-(4,4,5,5-tetramethyl-1,3,2-dioxaborolan-2-yl)-1H-pyrazole and methylamine for ammonia, the title compound can be obtained.

*Prepared as EP1382603A1

### Example 52

### {3-(3,4-Dichloro-phenyl)-3-[4-(3-methyl-1H-pyrazol-4-yl)-phenyl]-propyl}-dimethyl-amine

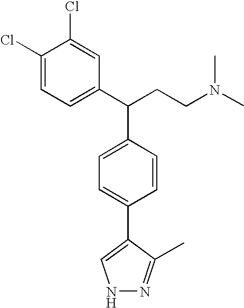

Following the procedure outlined in General Procedure 4, substituting 3-methyl-1-trityl-1H-pyrazole-4-boronic acid* for 4-(4,4,5,5-tetramethyl-1,3,2-dioxaborolan-2-yl)-1H-pyrazole and dimethylamine for methylamine, the title compound can be obtained.

*Prepared as EP1382603A1

### Example 53

### 3-(2-Chloro-phenyl)-3-[4-(1H-pyrazol-4-yl)-phenyl]-propylamine

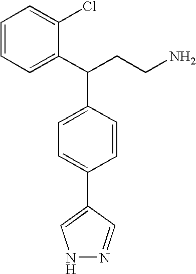

The title compound can be prepared using General Procedure 3 but using 2-chlorophenylmagnesium bromide* as the aryl-magnesium bromide in Procedure 3B, replacing methylamine with ammonia in 3D and using tetrakis(triphenylphosphine) palladium (0) as the catalyst in step 3F.

* 2-chlorophenylmagnesium bromide can be prepared from 1-bromo-2-chloro-benzene as described in the patent PL141742.

### Example 54

### 3-(2-Methoxy-phenyl)-3-[4-(1H-pyrazol-4-yl)-phenyl]-propylamine

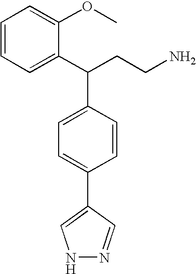

The title compound can be prepared using General Procedure 3 but using 2-methoxyphenylmagnesium bromide in Procedure 3B and replacing methylamine with ammonia in Procedure 3D.

### Example 55

### 3-(2-Fluoro-phenyl)-3-[4-(1H-pyrazol-4-yl)-phenyl]-propylamine

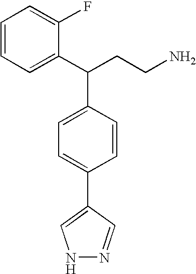

The title compound can be prepared using General Procedure 3 but using 2-fluorophenylmagnesium bromide* as the aryl-magnesium bromide in Procedure 3B, replacing methylamine with ammonia in 3D.

* 2-fluorophenylmagnesium bromide can be prepared from 1-bromo-2-fluoro-benzene as described in the following paper: Journal of Organic Chemistry (1988), 53(25), 5799-5806.

### Example 56

### 3-[4-(1H-Pyrazol-4-yl)-phenyl]-3-(2-trifluoromethyl-phenyl)-propylamine

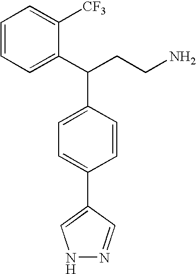

The title compound can be prepared using General Procedure 3 but using 2-(trifluoromethypphenylmagnesium bromide* as the aryl-magnesium bromide in Procedure 3B, replacing methylamine with ammonia in 3D.

* 2-(trifluoromethyl)phenylmagnesium bromide can be prepared from 1-bromo-2-trifluoromethyl-benzene as described in the following paper: Organometallics (1998), 17, 5166-5171.

### Example 57

### 3-(2-tert-Butyl-phenyl)-3-[4-(1H-pyrazol-4-yl)-phenyl]-propylamine

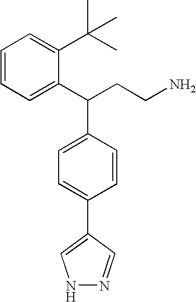

The title compound can be prepared using General Procedure 3 but using 2-(tert-butyl)phenylmagnesium bromide* as the aryl-magnesium bromide in Procedure 3B, replacing methylamine with ammonia in 3D.

* 2-(tert-butyl)phenylmagnesium bromide can be prepared from 1-bromo-2-tert-butylbenzene as described in the Journal of American Chemical Society (1997), 119, 461-465.

### Example 58

### 3-[4-(1H-Pyrazol-4-yl)-phenyl]-3-(2-trifluoromethoxy-phenyl)-propylamine

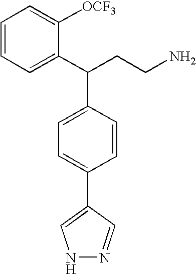

The title compound can be prepared using General Procedure 3 but replacing 4-bromobenzaldehyde with 2-(trifluoromethoxy)benzaldehyde in Procedure 3A and replacing methylamine with ammonia in Procedure 3D.

### Example 59

### 3-(2-Difluoromethoxy-phenyl)-3-[4-(1H-pyrazol-4-yl)-phenyl]-propylamine

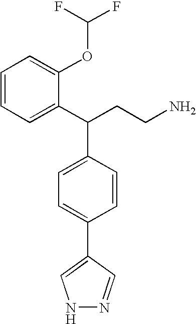

The title compound can be prepared using General Procedure 3 but replacing 4-bromobenzaldehyde with 2-(difluoromethoxy)benzaldehyde in Procedure 3A and replacing methylamine with ammonia in Procedure 3D.

### Example 60

### {2-(2-Chloro-phenyl)-2-[4-(1H-pyrazol-4-yl)-phenyl]-ethyl}-methyl-amine

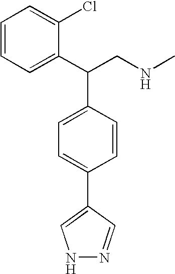

2-(2-Chloro-phenyl)-oxirane (formed according to General Procedure 6A using 2-chlorobenzaldehyde) can be converted to the title compound using General Procedure 5 and using 4-bromobenzene as R¹—H in 5B.

### Example 61

### {2-(2-Methoxy-phenyl)-2-[4-(1H-pyrazol-4-yl)-phenyl]-ethyl}-methyl-amine

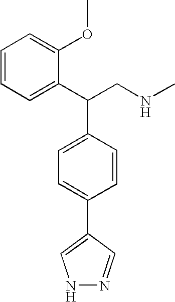

2-(2-Methoxy-phenyl)-oxirane (formed according to General Procedure 6A using 2-methoxybenzaldehyde) can be converted to the title compound using General Procedure 5 and using 4-bromobenzene as R¹—H in 5B.

### Example 62

### {2-(2-Fluoro-phenyl)-2-[4-(1H-pyrazol-4-yl)-phenyl]-ethyl}-methyl-amine

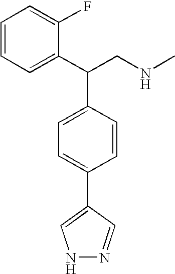

2-(2-Fluoro-phenyl)-oxirane (formed according to General Procedure 6A using 2-fluorobenzaldehyde) can be converted to the title compound using General Procedure 5 and using 4-bromobenzene as R¹—H in 5B.

### Example 63

### Methyl-[2-[4-(1H-pyrazol-4-yl)-phenyl]-2-(2-trifluoromethyl-phenyl)-ethyl]-amine

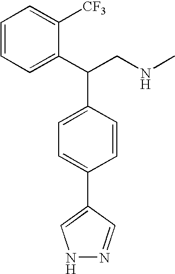

2-(2-Trifluoromethyl-phenyl)-oxirane (formed according to General Procedure 6A using 2-trifluoromethylbenzaldehyde) could be converted to the title compound using General Procedure 5 and using 4-bromobenzene as R¹—H in 5B.

### Example 64

### {2-(2-tert-Butyl-phenyl)-2-[4-(1H-pyrazol-4-yl)-phenyl]-ethyl}-methyl-amine

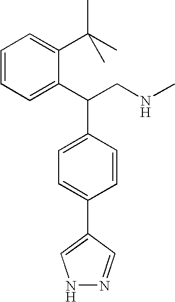

2-(2-tert-butyl-phenyl)-oxirane (formed according to General Procedure 6A using 2-tert-butylbenzaldehyde*) can be converted to the title compound using General Procedure 5 and using 4-bromobenzene as R¹—H in 5B.

[* 2-(tert-butyl)benzaldehyde can be prepared according to the method described in the following paper: Journal of Organic Chemistry (1983), 48(22), 4053-4058.]

### Example 65

### Methyl-[2-[4-(1H-pyrazol-4-yl)-phenyl]-2-(2-trifluoromethoxy-phenyl)-ethyl]-amine

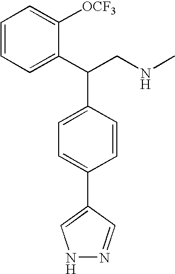

2-(2-Trifluoromethoxy-phenyl)-oxirane (formed according to General Procedure 6A using 2-trifluoromethoxybenzaldehyde) can be converted to the title compound using General Procedure 5 and using 4-bromobenzene as R¹—H in 5B.

### Example 66

### {2-(2-Difluoromethoxy-phenyl)-2-[4-(1H-pyrazol-4-yl)-phenyl]-ethyl}-methyl-amine

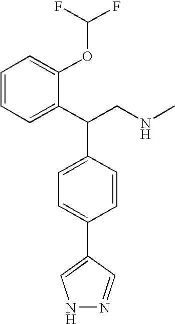

2-(2-Difluoromethoxy-phenyl)-oxirane (formed according to General Procedure 6A using 2-difluoromethoxybenzaldehyde) can be converted to the title compound using General Procedure 5 and using 4-bromobenzene as R¹—H in 5B.

### Example 67

### 4-(2-Chloro-phenyl)-4-[4-(1H-pyrazol-4-yl)-phenyl]-piperidine

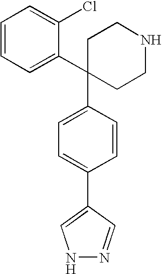

The title compound can be prepared using General Procedure 8 using 2-chlorophenylmagnesium bromide* as the aryl-magnesium bromide (x) in Procedure 8A.

* 2-chlorophenylmagnesium bromide can be prepared from 1-bromo-2-chloro-benzene as described in the patent PL141742.

### Example 68

### 4-(2-Methoxy-phenyl)-4-[4-(1H-pyrazol-4-yl)-phenyl]-piperidine

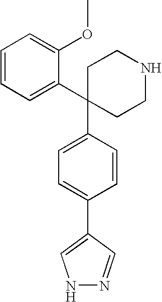

The title compound can be prepared using General Procedure 8 using 2-methoxyphenylmagnesium bromide* as the aryl-magnesium bromide (x) in Procedure 8A.

### Example 69

### 4-(2-Fluoro-phenyl)-4-[4-(1H-pyrazol-4-yl)-phenyl]-piperidine

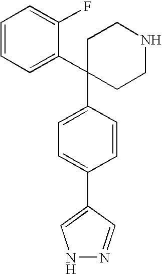

The title compound can be prepared using General Procedure 8 using 2-fluorophenylmagnesium bromide* as the aryl-magnesium bromide (x) in Procedure 8A.

* 2-fluorophenylmagnesium bromide can be prepared from 1-bromo-2-fluoro-benzene as described in the following paper: Journal of Organic Chemistry (1988), 53(25), 5799-5806.

### Example 70

### 4-[4(1H-Pyrazol-4-yl)-phenyl]-4-(2-trifluoromethyl-phenyl)-piperidine

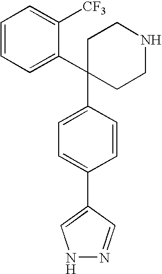

The title compound can be prepared using General Procedure 8 using 2-(trifluoromethypphenylmagnesium bromide* as the aryl-magnesium bromide (x) in Procedure 8A.

* 2-(trifluoromethyl)phenylmagnesium bromide can be prepared from 1-bromo-2-trifluoromethyl-benzene as described in the following paper: Organometallics (1998), 17, 5166-5171.

### Example 71

### 4-(2-tert-Butyl-phenyl)-4-[4-(1H-pyrazol-4-yl)-phenyl]-piperidine

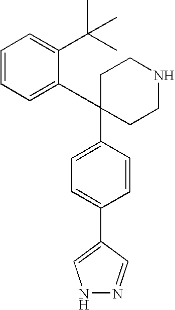

The title compound can be prepared using General Procedure 8 using 2-(tert-butyl)phenylmagnesium bromide* as the aryl-magnesium bromide (x) in Procedure 8A.

* 2-(tert-butyl)phenylmagnesium bromide can be prepared from 1-bromo-2-tert-butylbenzene as described in the Journal of American Chemical Society (1997), 119, 461-465.

### Example 72

### {3-(2,4-Dichloro-phenyl)-3-[4-(1H-pyrazol-4-yl)-phenyl]-propyl}-methyl-amine

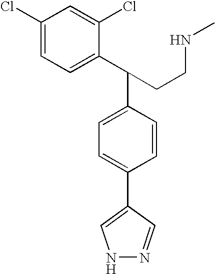

Following General Procedure 3, and using 2,4-dichlorophenylmagnesium bromide as the aryl-magnesium bromide in Procedure 3B, the title compound can be obtained.

### Example 73

### 3-(2,4-Dichloro-phenyl)-3-[4-(1H-pyrazol-4-yl)-phenyl]-propylamine

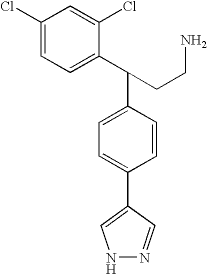

Following General Procedure 3, and using 2,4-dichlorophenylmagnesium bromide as the aryl-magnesium bromide in Procedure 3B, and replacing methylamine with ammonia in Procedure 3D, the title compound can be obtained.

### Example 74

### {3(2,4-Dichloro-phenyl)-3-[4-(1H-pyrazol-4-yl)-phenyl]-propyl}-dimethyl-amine

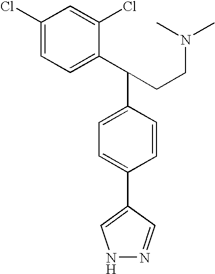

Following General Procedure 3, and using 2,4-dichlorophenylmagnesium bromide as the aryl-magnesium bromide in Procedure 3B, and replacing methylamine with dimethylamine in Procedure 3D, the title compound can be obtained.

### Example 75

### 14-{4-[3-Azetidin-1-yl-1-(3,4-dichloro-phenyl)-propyl]-phenyl}-1H-pyrazole

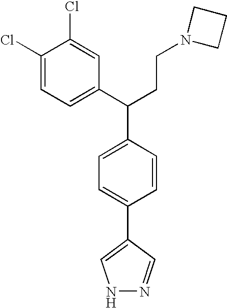

The title compound can be prepared by following General Procedure 3 but substituting methylamine for azetidine and using 3,4-dichlorophenylmagnesium bromide as the aryl-magnesium bromide in Procedure 3B.

### Example 76

### 4-{4-[Azetidin-3-yl-(3,4-dichloro-phenyl)-methyl]-phenyl}-1H-pyrazole

### 76A. 3-[(3,4-Dichloro-phenyl)-hydroxy-methyl]-azetidine-1-carboxylic acid tert-butyl ester

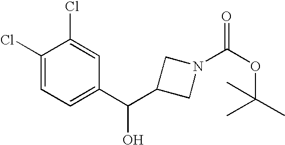

The title compound can be prepared by the addition of 3,4-dichlorophenyl-magnesium bromide to commercially available 3-formyl-azetidine-1-carboxylic acid tert-butyl ester.

### 76B. 3-[(4-Bromo-phenyl)-(3,4-dichloro-phenyl)-methyl]-azetidine

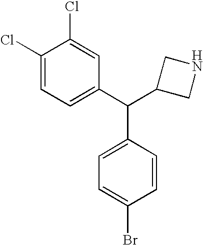

The title compound can be prepared from the product of Example 76A by following General Procedure 5B using bromobenzene as the aryl compound R¹—H.

### 76C. 4-{4-[Azetidin-3-yl-(3,4-dichloro-phenyl)-methyl]-phenyl}-1H-pyrazole

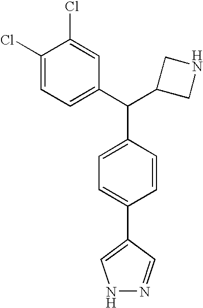

The title compound can be prepared from the product of Example 76B by following General Procedure 1.

### Example 77

### 4-Bromomethyl-4-(4-bromo-phenyl)-piperidine-1-carboxylic acid tert-butyl ester

### 77A. Bis-(2-chloro-ethyl)-carbamic acid tent-butyl ester

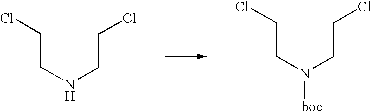

Bis-(2-chloro-ethyl)-carbamic acid tert-butyl ester was made using a method described in J. Chem. Soc., Perkin Trans 1, 2000, p 3444-3450.

### 77B. 4-(4-Bromo-phenyl)-4-cyano-piperidine-1-carboxylic acid tert-butyl ester

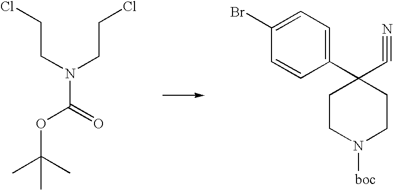

4-(4-Chloro-phenyl)-4-cyano-piperidine-1-carboxylic acid tert-butyl ester was made using a method described in WO2004022539.

### 77C. 4-(4-Bromo-phenyl)-piperidine-4-carboxylic acid

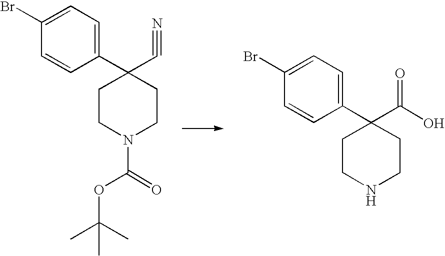

A suspension of 4-(4-bromo-phenyl)-4-cyano-piperidine-1-carboxylic acid tert-butyl ester (13.3 g, 36.4 mmol) in concentrated hydrochloric acid (150 ml) was heated to reflux for 72 hours. The reaction was allowed to cool then was concentrated in vacuo furnishing a brown solid that was used crude in the following protection step (10.2 g, 98%). LC/MS: (PS-A2) Rₜ 1.82 [M+H]⁺ 284

### 77D. 4-(4-Bromo-phenyl)-piperidine-1,4-dicarboxylic acid mono-(9H-fluoren-9-ylmethyl) ester

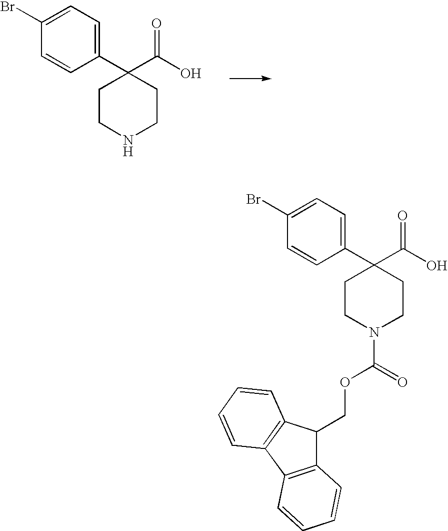

9-Fluorenylmethyl N-succinimidyl carbonate (18.5 g, 54.9 mmol) was added to a solution of 4-(4-Bromo-phenyl)-piperidine-4-carboxylic acid (12 g, 42.2 mmol) in tetrahydrofuran (180 ml), water (100 ml) and aqueous sodium hydroxide (2N, 80 ml) at room temperature. After stirring for 18 hours, the reaction was acidified with hydrochloric acid (2N, 100 ml) and extracted into ethyl acetate (×2). The combined organic liquors were dried (MgSO₄) and concentrated in vacuo to furnish a foam. The material was dissolved in acetonitrile and after standing the precipitate was isolated by suction filtration to furnish the title compound as a white solid (16.7 g, 78%). LC/MS: (PS-A2) Rₜ 3.65 [M+H]⁺ 179

### 77E. 4-(4-Bromo-phenyl)-4-hydroxymethyl-piperidine-1-carboxylic acid 9H-fluoren-9-ylmethyl ester

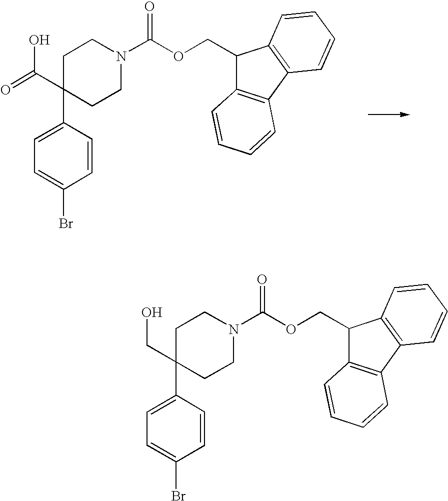

To a solution of 4-(4-bromo-phenyl)-piperidine-1,4-dicarboxylic acid mono-(9H-fluoren-9-ylmethyl) ester (2.02 g, 3.99 mmol) in tetrahydrofuran (10 ml) was added dropwise borane-tetrahydrofuran complex (1M, 12 ml) and the mixture was stirred under nitrogen overnight. The reaction was then quenched by the careful addition of methanol. Once the effervescence had subsided, the reaction was concentrated in vacuo and reconcentrated from methanol (×2). The crude product was purified by silica column chromatography eluting on a gradient 20-50% ethyl acetate/ petroleum ether to furnish the title compound as a white foam (1.64 g, 83%). LC/MS: (PS-A2) Rₜ 3.74 [M+H]⁺ 492

### 77F. 4-Bromomethyl-4-(4-bromo-phenyl)-piperidine-1-carboxylic acid 9H-fluoren-9-ylmethyl ester

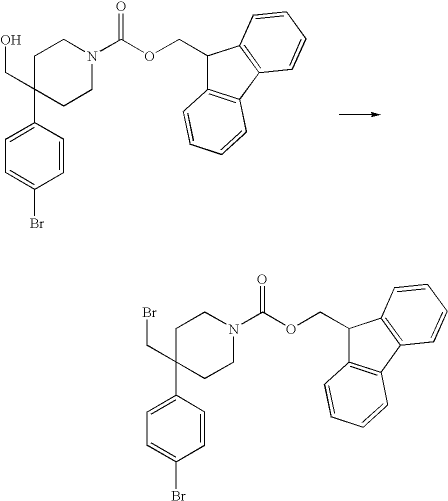

To a solution of 4-(4-Bromo-phenyl)-4-hydroxymethyl-piperidine-1-carboxylic acid 9H-fluoren-9-ylmethyl ester (1.215 g, 2.47 mmol) in pyridine at room temperature was added triphenylphosphine (1.62 g, 6.18 mmol) and carbon tetrabromide (982 mg, 2.96 mmol). The mixture was heated to 60° C. for 1.5 hours. The reaction was then allowed to cool and was quenched with methanol. The reaction was diluted with 2N hydrochloric acid and extracted into dichloromethane (×2). The organic solutions were concentrated in vacuo and purified by silica column chromatography eluting on a gradient 10-30% ethyl acetate/ petroleum ether to furnish the title compound as a colourless oil (1.35 g, 98%). LC/MS: (PS-A2) Rₜ 4.26 [M+H]⁺ none.

### 77G. 4-Bromomethyl-4-(4-bromo-phenyl)-piperidine-1-carboxylic acid tert-butyl ester

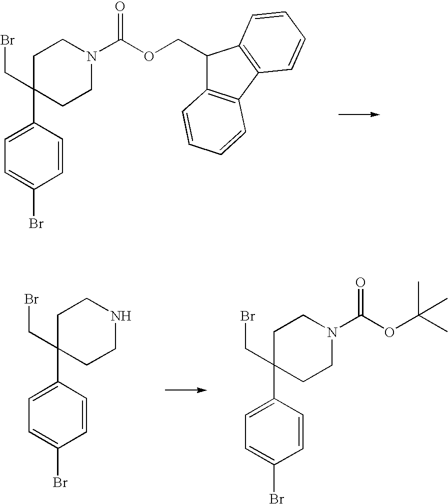

To a solution of 4-bromomethyl-4-(4-bromo-phenyl)-piperidine-1-carboxylic acid 9H-fluoren-9-ylmethyl ester (662 mg, 1.19 mmol) in dichloromethane (1 ml) was added morpholine (10 drops) and 1,8-diazabicyclo[5.4.0]undec-7-ene (DBU, 3 drops). The resultant mixture was stirred for 2 hours. Water was added to the reaction and it was extracted with dichloromethane. The organic layer was separated and concentrated in vacuo then reconcentrated from methanol (×2) to furnish the crude deprotected compound. This material was dissolved in dimethylformamide to which was added triethylamine (216 ul, 1.55 mmol) and di-tert-butyl dicarbonate (312 mg, 1.43 mmol). This mixture was stirred at room temperature for 3.5 hours. Water was added to the reaction and it was extracted with dichloromethane (×3). The organic liquors were concentrated in vacuo then suspended in methanol. After heating and sonication, the solids were removed by filtration and discarded; the solution was purified on a silica column eluting with 0-30% ethyl acetate/petroleum ether to furnish the title compound as a white solid (441 mg, 86%). ¹H NMR (CDCl₃) 7.52 (2H, d), 7.20 (2H, d), 3.79 (2H, m), 3.48 (2H, s), 3.03 (2H, m), 2.22 (2H, m), 1.89 (2H, m), 1.48 (9H, s).

77H. 4-(4-Bromo-phenyl)-4-(3-methoxy-phenoxymethyl)-piperidine-1-carboxylic acid tert-butyl ester

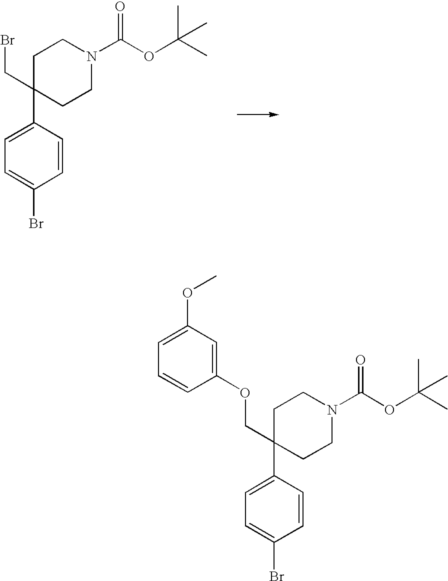

To a mixture of 4-bromomethyl-4-(4-bromo-phenyl)-piperidine-1-carboxylic acid tert-butyl ester (40 mg, 0.09 mmol) and caesium carbonate (59 mg, 0.180 mmol) in dimethylformamide (1 ml) was added 3-methoxyphenol (15 ul, 0.135 mmol) and the reaction was heated to 50° C. for 4 hours, then 100° C. for 18 hours. The reaction was then repeated on 7× scale, heating to 100° C. directly. After 18 hours at this temperature, extra portions of 3-methoxyphenol (100 ul, 0.9 mmol) and caesium carbonate (200 mg, 0.61 mmol) were added and the reaction heated for a further 6 hours. The two reactions were combined, diluted with dichloromethane and washed with aqueous sodium hydroxide solution (2N). The organic liquors were concentrated to remove the majority of residual dimethylformamide then applied to a silica Biotage column, eluting with a gradient 0-20% ethyl acetate/ petrol to furnish the title compound as a colourless oil (347 mg, 100%). LC/MS: (PS-A2) Rₜ 4.19 [M+H]⁺ 476

### 77I. 4-Bromo-3-methyl-pyrazole-1-sulphonic acid dimethylamide

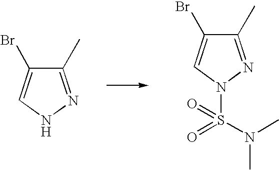

1,4-Diazabicyclo[2.2.2]octane (DABCO, 3.83 g, 34.1 mmol) and dimethylsulphamoylchloride (3.32 ml, 31.0 mmol) were added sequentially to a solution of 4-bromo-3-methylpyrazole (5.0 g, 31.0 mmol) in acetonitrile (50 ml) whilst stirring at room temperature. After 6 hours, the reaction was quenched with water then 2N HCl was added. The solution was extracted twice with ethyl acetate and the organic liquors were washed with brine before drying (MgSO₄) and concentrating in vacuo. The resultant oil was reconcentrated from diethylether twice to afford a white foam (6.7 g, 81%). LC/MS: (PS-A2) Rₜ 2.96 [M+14]⁺ 267

### 77J. 3-Methyl-1-sulphonic acid dimethylamide-pyrazole-4-boronic acid

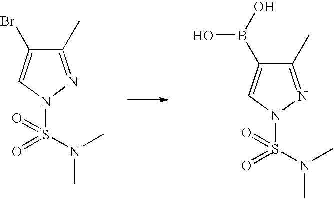

To a solution of 4-bromo-3-methyl-pyrazole-1-sulphonic acid dimethylamide (7.3 g, 27.2 mmol) and triethylborate (6.9 ml, 40.8 mmol) in anhydrous tetrahydrofuran (80 ml) under a nitrogen atmosphere was added dropwise a solution of methyllithium in ether (1.6M, 22 ml, 35.4 mmol) while the temperature was maintained below −55 deg C. The reaction was stirred at this temperature for 20 minutes before allowing the reaction to warm to room temperature. After stirring thus overnight, the reaction was quenched by cautious addition of hydrochloric acid (2N) before extraction of the product into 3 portions of ethyl acetate. The combined organic liquors were washed with brine before drying (MgSO₄) and concentrated in vacuo to furnish an oil. The product was purified on a 40+M silica Biotage column, eluting on a gradient 0-15% methanol/dichloromethane to furnish the title compound as a pale yellow oil (4.2 g, 66%). NMR (Me-d₃-OD) 8.22 (1H, s), 2.90 (6H, s), 2.38 (3H, s).

### 77K. 4-[4-(1-Dimethylsulphamoyl-3-methyl-1H-pyrazol-4-yl)-phenyl]-4-(3-methoxy-phenoxymethyl)-piperidine-1-carboxylic acid tert-butyl ester

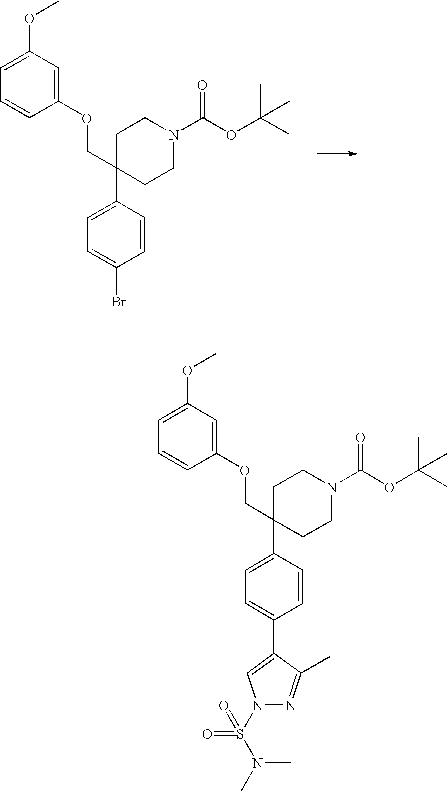

A mixture of 4-(4-bromo-phenyl)-4-(3-methoxy-phenoxymethyl)-piperidine-1-carboxylic acid tert-butyl ester (174 mg, 0.364 mmol), 3-methyl-1-sulphonic acid dimethylamide-pyrazole boronic acid (69 mg, 0.546 mmol), potassium phosphate (271 mg, 1.275 mmol), tetrakis(triphenylphosphine)palladium (0) (13 mg, 0.011 mmol), ethanol (1 ml), toluene (1 ml), methanol (1 ml) and water (1 ml) was heated to 90° C. in a nitrogen atmosphere overnight. The reaction was returned to room temperature, further boronic acid (69 mg, 0.546 mmol) and a new catalyst, bis(tri-tert-butyl)phosphine (13 mg, 0.025 mmol) were added before returning to 90° C. After 3 hours, dichloromethane was added to the reaction and the organic liquors were separated and were washed with dilute sodium bicarbonate solution before concentrating in vacuo and purification via silica Biotage column eluting with a gradient of 20-40% ethyl acetate/ petrol. The title compound was thus produced (63 mg, 30%). LC/MS: (PS-A2) Rₜ 3.99 [M+H]⁺ 585

### 77L. 4-(3-Methoxy-phenoxymethyl)-4-[4-(3-methyl-1H-pyrazol-4-yl)-phenyl]-piperidine

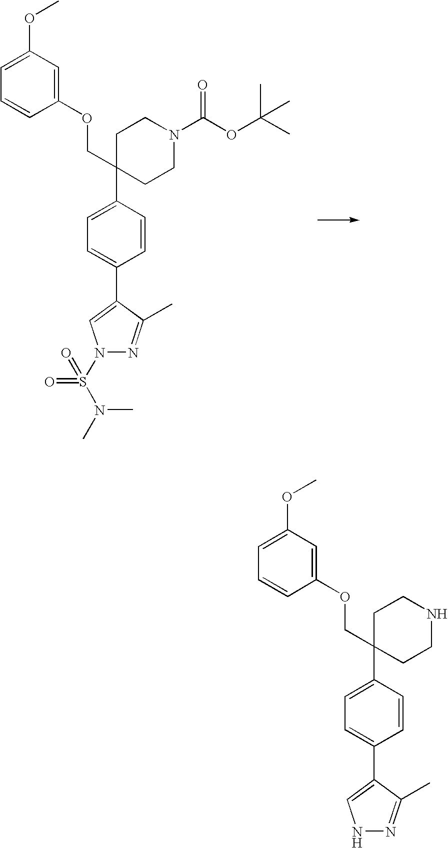

To a solution of 4-[4-(1-dimethylsulphamoyl-3-methyl-1H-pyrazol-4-yl)-phenyl]-4-(3-methoxy-phenoxymethyl)-piperidine-1-carboxylic acid tert-butyl ester (63 mg, 0.108 mmol) in methanol (3 ml) was added concentrated hydrochloric acid (10 drops). The mixture was then heated to reflux for 1 hour then allowed to cool to room temperature. The reaction was basified with saturated sodium bicarbonate solution then extracted into ethyl acetate (×3). The organic liquors were dried (MgSO₄) and concentrated in vacuo. The crude product was purified on silica Biotage column, eluting 60-100% DMAW 90/dichloromethane to furnish the title compound as a pale coloured solid (4 mg, 10%) ¹H NMR (Me-d₃-OD) 7.72 (1H, s), 7.55 (41-1, m), 7.13 (1H, t), 6.51 (2H, d), 6.47 (2H, d), 6.41 (1H, s), 3.95 (2H, s), 3.72 (3H, s), 3.39 (2H, m), 3.02 (2H, m), 2.62 (2H, m), 2.42 (3H, s), 2.35 (214, m), 1.99 (3H, s). LC/MS (PS-A3) Rₜ 7.03 [M-1-11]⁺ 378

### Example 78

### 2-Amino-1-[4-(3-methyl-1H-pyrazol-4-yl)-phenyl]-1-phenyl-ethanol

### 78A. 2-Amino-1-(4-chloro-phenyl)-1-(4-iodo-phenyl)-ethanol

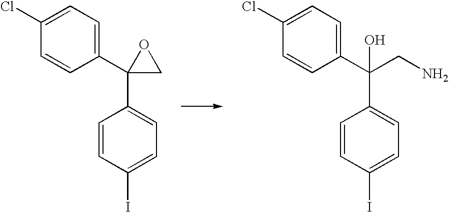

2-(4-Chloro-phenyl)-2-(4-iodo-phenyl)-oxirane* (5 g, 14.04 mmol) was suspended in 2N ammonia in methanol (50 mL) and this mixture was heated at 130° C. in a CEM Explorer microwave for 1 hour 20 minutes. This process was repeated for 3 further batches of 2-(4-chloro-phenyl)-2-(4-iodo-phenyl)-oxirane (5g per batch). All reaction mixtures were then combined and concentrated in vacuo. The residue was triturated with a solvent mixture of 70/ 30 DCM/methanol. The solid was isolated by buchner filtration then dried to afford the title compound as a white solid (9.55 g, 46%). LC/MS: (PS-B5) Rₜ 3.14 [M+H H₂O]⁺ 355.98.

*Accessed according to the procedure outlined in WO 2005/061463

### Example 79

### 78B. 4-[4-(2-Amino-1-hydroxy-1-phenyl-ethyl)-phenyl]-3-methyl-pyrazole-1-sulphonic acid dimethylamide

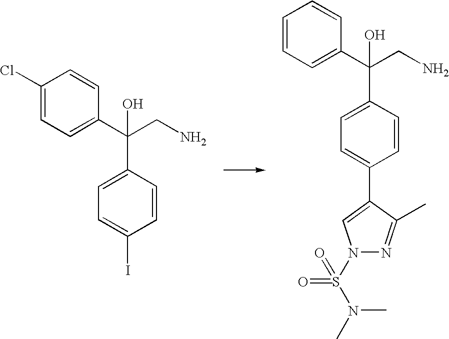

The title compound was prepared according to General Procedure 1, using 3-methyl-1-sulphonic acid dimethylamide-pyrazole boronic acid in place of the stated boronate ester, as a white solid (120 mg, 73%). LC/MS (PS-A2) Rₜ 3.45 [WI-1]⁺ 501

### 78C. 2-Amino-1-[4-(3-methyl-1H-pyrazol-4-yl)-phenyl]-1-phenyl-ethanol

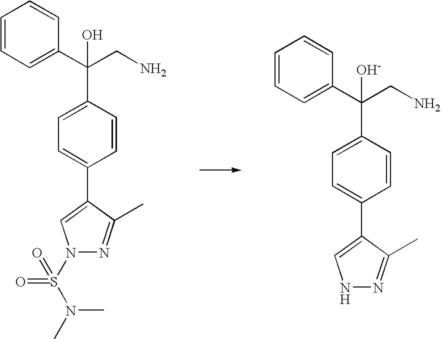

The title compound was prepared according to the procedure outlined in Example 77L. NMR (Me-d₃-OD) 7.70 (1H, s), 7.49 (4H, m), 4.42 (2H, d), 7.38 (2H, t), 7.28 (1H, t), 3.52 (2H, s), 2.40 (31-1, s). LC/MS (PS-B4) Rₜ 5.02 [M+H]⁺ 276.

### Example 79

### 2-Amino-1-(4-chloro-phenyl)-1-[4-(3-methyl-1H-pyrazol-4-yl)-phenyl]-ethanol

### 79A. 4-{4-[2-Amino-1-(4-chloro-phenyl)-1-hydroxy-ethyl]-phenyl}-3-methyl-pyrazole-1-sulphonic acid dimethylamide

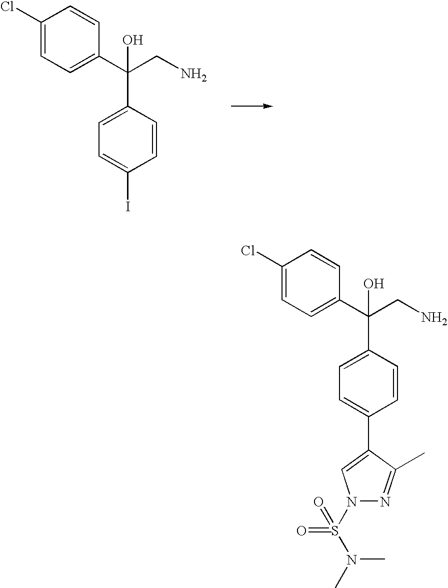

The title compound was prepared according to General Procedure 1 using tetrakis(triphenylphosphine)palladium (0) as catalyst and 3-methyl-1-sulphonic acid dimethylamide-pyrazole boronic acid in place of the stated boronate ester (152 mg, 86%). LC/MS (PS-A2) Rₜ 3.45 [M+H]⁺ 501

### 79B. 2-Amino-1-(4-chloro-phenyl)-1-[4-(3-methyl-1H-pyrazol-4-yl)-phenyl]-ethanol

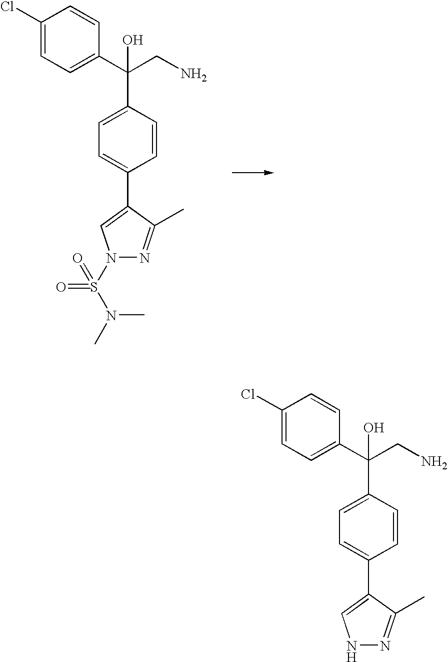

The title compound was prepared according to the procedure described in Example 77L (23 mg, 50% yield). NMR (Me-d₃-OD) 7.69 (1H, s), 7.45 (6H, m), 7.33 (2H, d), 3.42 (2H, d), 2.41 (3H, s). LC/MS (PS-B4) Rₜ 5.95 [M+H]⁺ 328

### Example 80

### 2-Amino-1-(4-chloro-phenyl)-1-[4-(3-ethyl-1H-pyrazol-4-yl)-phenyl]-ethanol

### 80A. Pyrazole-1-sulphonic acid dimethylamide

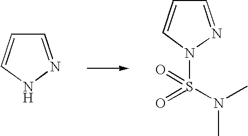

1,4-Diazabicyclo[2.2.2]octane (DABCO, 10.1 g, 90.3 mmol) and dimethylsulphamoylchloride (8.8 ml, 82.9 mmol) were added sequentially to a solution of pyrazole (4.8 g, 81.9 mmol) in acetonitrile (125 ml) whilst stirring at room temperature. After 18 hours, the reaction was concentrated in vacuo and wet with water and ethyl acetate. The organic layer was removed and washed with hydrochloric acid (2N) and then brine before drying (MgSO₄) and concentrating in vacuo to furnish the title compound as a colourless oil (13.1 g, 91%). LC/MS (PS-A2) Rₜ 2.16 [M+H]⁺ 176

### 80B. 3-Ethyl-pyrazole-1-sulphonic acid dimethylamide

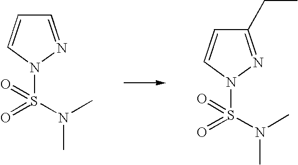

To a solution of pyrazole-1-sulphonic acid dimethylamide (13.1 g, 74.9 mmol) in anhydrous tetrahydrofuran (100 ml) at −78 deg C. under a nitrogen atmosphere was added dropwise a solution of n-butyllithium in hexanes (1.6M, 51 ml, 82.3 mmol). The reaction was stirred thus for 30 minutes then iodoethane (6.6 ml, 82.41=01) was added dropwise. The reaction mixture was allowed to warm to room temperature then stirred thus over the weekend. Water was then added and the solution was extracted with ethyl acetate. The separated organic liquors were washed with brine, dried (MgSO₄) and concentrated to furnish a yellow/ brown liquid (12.6 g, 83% yield). LC/MS (PS-A2) Rₜ 2.78 [M+H]⁺ 204

### 80C. 4-Bromo-3-ethyl-pyrazole-1-sulphonic acid dimethylamide

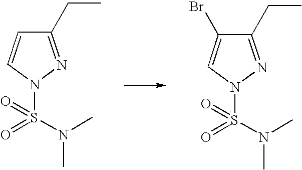

To a solution of 3-ethyl-pyrazole-1-sulphonic acid dimethylamide (12.6 g, 62.1 mmol) in tetrahydrofuran at room temperature was added N-bromosuccinimide (12.7 g, 68.3 mmol). After stirring for 2 hours, water was added and the product extracted into ethyl acetate. The organic layer was separated and washed with brine then dried (MgSO₄) and concentrated in vacuo. The resultant oil was purified on three 40+M silica Biotage columns eluting with 5% ethyl acetate/petrol to furnish the title compound as a pale yellow oil (7.7 g, 44%). LC/MS (PS-A2) Rₜ 3.32 [M+H]⁺ 282

### 80D. 4-Bromo-3-ethyl-pyrazole-1-sulphonic acid dimethylamide

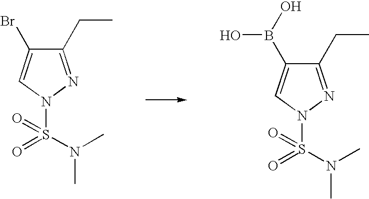

To a solution of 4-bromo-3-ethyl-pyrazole-1-sulphonic acid dimethylamide (2.4 g, 8.5 mmol) and triethylborate (2.17 ml, 12.8 mmol) in anhydrous tetrahydrofuran stirred at −78 deg C. under a nitrogen atmosphere was added a solution of methyllithium in diethylether (1.6M, 6.9 ml, 11.1 mmol). The reaction was allowed to stir warming to room temperature overnight. The reaction was then quenched with hydrochloric acid (2N), stirred for 5 minutes, then extracted into ethyl acetate (×3). The combined organic liquors were washed with brine then dried (MgSO₄) and concentrated in vacuo. The residue was purified on a 40+M silica Biotage column eluting with a gradient 0-15% methanol/ dichloromethane to furnish the product as a pale coloured oil (1.2 g, 57%). LC/MS (PS-A2) Rₜ 2.31 [M+1-1]⁴248.

### 80E. 4-{4-[2-Amino-1-(4-chloro-phenyl)-1-hydroxy-ethyl]-phenyl}-3-ethyl-pyrazole-1-sulphonic acid dimethylamide

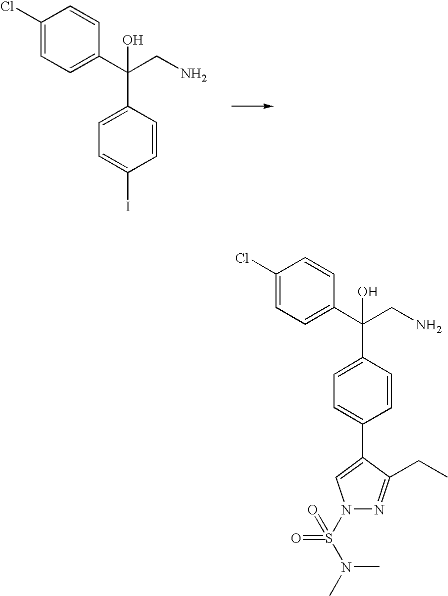

The title compound was prepared according to General Procedure 1 using 2-amino-1-(4-chloro-phenyl)-1-(4-iodo-phenyl)-ethanol as substrate, tetrakis(triphenylphosphine)palladium (0) as catalyst and 4-bromo-3-ethyl-pyrazole-1-sulphonic acid dimethylamide in place of the stated boronate ester (146 mg, 63% yield). LC/MS (PS-A2) Rₜ 3.84 [M+H]⁺ 549

### 80F. 2-Amino-1-(4-chloro-phenyl)-1-[4-(3-ethyl-1H-pyrazol-4-yl)-phenyl]-ethanol

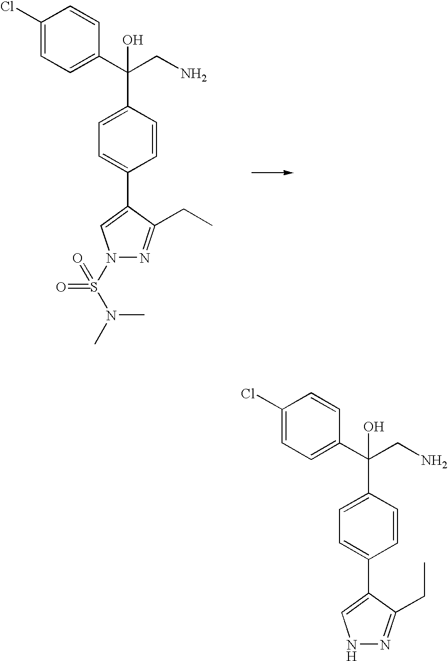

The title compound was prepared according to the procedure outlined in Example 77L (20 mg, 22% yield). ¹H NMR (Me-d₃-OD) 7.63 (111, s), 7.48 (4H, d), 7.41 (2H, d), 7.37 (2H, d), 3.41 (2H, d), 2.82 (214, q), 1.23 (313, t) LC/MS (PS-B4) Rₜ 6.35 [M+H]⁺ 342

### Example 81

### 4-{4-[3-(4-Chloro-phenyl)-pyrrolidin-3-yl]-phenyl}-1H-pyrazole formate

### 81A. 3-(4-Chloro-phenyl)-3-hydroxy-pyrrolidine-1-carboxylic acid tert-butyl ester

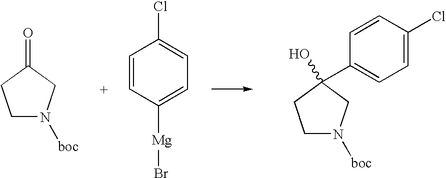

To a solution of 3-oxo-pyrrolidine-1-carboxylic acid tert-butyl ester (1.0 g, 5.40 mmol) in anhydrous tetrahydrofuran (30 ml) stirring at room temperature under nitrogen was added dropwise a solution of 4-chlorophenylmagnesium bromide (1M in ether, 27 ml, 27 mmol). After stirring thus overnight, the reaction was quenched by cautious addition of dilute aqueous ammonium chloride solution followed by addition of ethyl acetate. The mixture was filtered under suction then the aqueous was separated and extracted again with ethyl acetate. The organic liquors were combined, washed with saturated ammonium chloride then brine, dried (MgSO₄) and concentrated in vacuo. The crude product was purified by silica Biotage column, eluting 20-50% ethyl acetate/ petrol to furnish the title compound (935 mg, 58%). LC/MS (PS-A2) Rₜ 3.21 [m+H]⁺ 298

### 81B. 3,3-Bis-(4-chloro-phenyl)-pyrrolidine

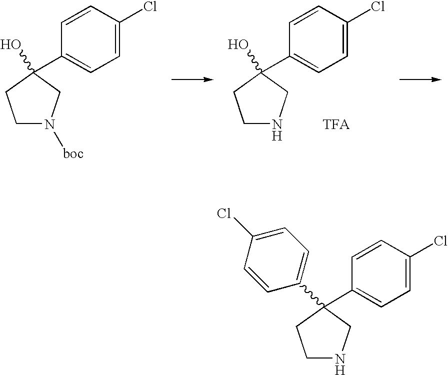

To a solution of 3-(4-chloro-phenyl)-3-hydroxy-pyrrolidine-1-carboxylic acid tert-butyl ester (4.0 g, 13.4 mmol) in dichloromethane (10 ml) stirred at room temperature was added trifluoroacetic acid (2 ml). After stirring for 30 minutes, an additional portion of trifluoroacetic acid (4 ml) was added and the reaction was stirred for 10 minutes then was concentrated in vacuo to furnish the deprotected pyrrolidine salt. This material was used thus in the Friedel Crafts arylation.

The alcohol was suspended in chlorobenzene (10 ml) and aluminium chloride (1.9 g, 14.2 mmol) was added portionwise whilst stirring. After stirring overnight, the reaction was quenched with water then aqueous sodium hydroxide was added to basify (2N, pH˜14). Ethyl acetate was subsequently added and the reaction was filtered under suction. The aqueous layer was separated and extracted with ethyl acetate and the combined organic liquors were washed with aqueous sodium hydroxide solution (2N) then brine, dried (MgSO₄) and concentrated in vacuo. The reaction was then reset using this crude material and the quantities of reagent specified above. After stirring overnight, the reaction was worked up again. The crude material was purified by silica Biotage chromatography eluting 65-85% DMAW 90/dichloromethane to furnish pure (1.8 g, 46% yield) plus impure (2.5 g) fractions. LC/MS (PS-A2) Rₜ 2.32 [M+H]⁺ 292

### 81C. 4-{-4-[3-(4-Chloro-phenyl)-pyrrolidin-3-yl]-phenyl}-1H-pyrazole formate

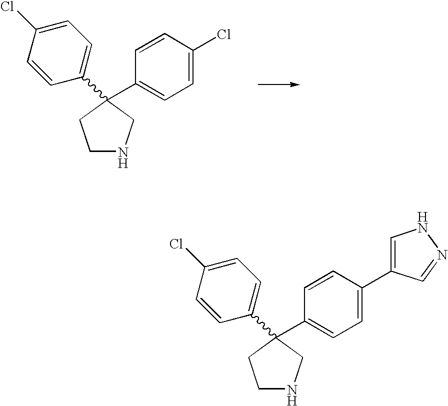

The title compound was prepared according to General Procedure 1 (71 mg, 6% yield). ¹H NMR (Me-d₃-OD) 8.50 (1H, s), 7.95 (214, s), 7.60 (2H, d), 7.39 (6H, m), 4.06 (1H, d), 3.98 (11-1, d), 3.41 (2H, t), 2.82 (2H, m) LC/MS (PS-A3) Rₜ 6.66 [M+H]⁺ 324

### Example 82

### 4-{-4-[3-(4-Methoxy-phenyl)-azetidin-3-yl]-phenyl}-1H-pyrazole formate

### 82A. 1-Benzhydryl-azetidin-3-one

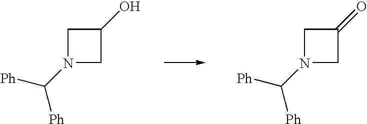

1-benzhydryl-azetidin-3-ol was oxidised to the title compound according to the procedure outlined in J. Heterocyclic Chem. 1994, 31, 271-275.

### 82B. 1-Benzhydryl-3-(4-methoxy-phenyl)-azetidin-3-ol

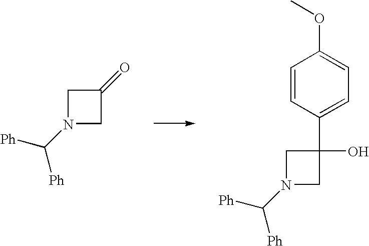

The title compound (with impurity) was accessed using methodology outlined in Example 81A (780 mg, 45% yield). LC/MS (PS-A2) Rₜ 2.30 [M+H]⁺ 346

### 82C. 3-(4-Methoxy-phenyl)-azetidin-3-ol

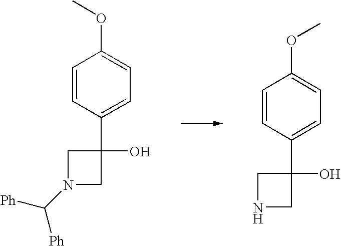

A solution of 1-benzhydryl-3-(4-methoxy-phenyl)-azetidin-3-ol (780 mg, 2.26 mmol) in ethanol (10 ml) was hydrogenated over palladium on carbon (10%, wet, catalytic amount) at room temperature and pressure overnight. The reaction was filtered under suction and the liquors were dried in vacuo. The residue was purified by SCX SPE washing with methanol and eluting the clean product with methanolic ammonia (308 mg, 76% yield). LC/MS (PS-A2) Rₜ 1.70 [M+H]⁺ 180

### 82D. 3-(4-Chloro-phenyl)-3-(4-methoxy-phenyl)-azetidine

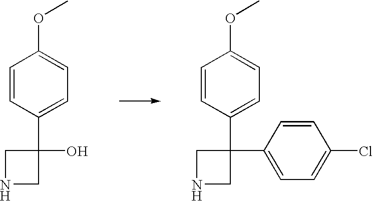

The title compound was prepared using the methods described in Example 81B (258 mg, 55% yield). LC/MS (PS-A2) Rₜ 1.95 [M+H]⁺ 274

### 82E. 4-{-4-[3-(4-Methoxy-phenyl)-azetidin-3-yl]-phenyl}-1H-Pyrazole formate

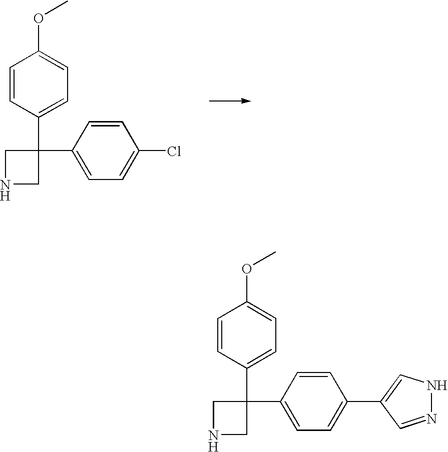

The title compound was obtained by the methodology outlined in General Procedure 1 (14 mg, 4% yield). ¹H NMR (Me-d₃-OD) 8.51 (1H, s), 7.98 (2H, s), 7.63 (2H, d), 7.38 (2H, d), 7.31 (2H, d), 6.98 (2H, d), 4.72 (4H, m), 3.80 (3H, s) LC/MS (PS-A3) Rₜ 6.01 [M+H]⁺ 306

### Example 83

### 4-(2,3-Dihydro-benzo[1,4]dioxin-6-yl)-4-[4-(1H-pyrazol-4-yl)-phenyl]-piperidine

### 83A 4-(4-Chloro-phenyl)-4-(2,3-dihydro-benzo[1,4]dioxin-6-yl)-piperidine

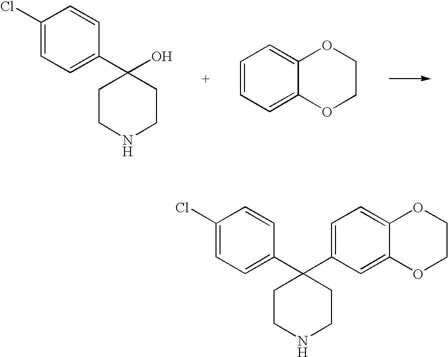

Using the methods described in Example 81B, 2,3-dihydro-benzo[1,4]dioxine and 4-(4-chloro-phenyl)-piperidin-4-ol were reacted to produce the title compound (3.8 g, 68% yield). LC/MS (PS-A2) Rₜ 2.20 [M+H]⁺ 330

### 83B. 4-(2,3-Dihydro-benzo[1,4]dioxin-6-yl)-4-[4-(1H-pyrazol-4-yl)-phenyl]-piperidine

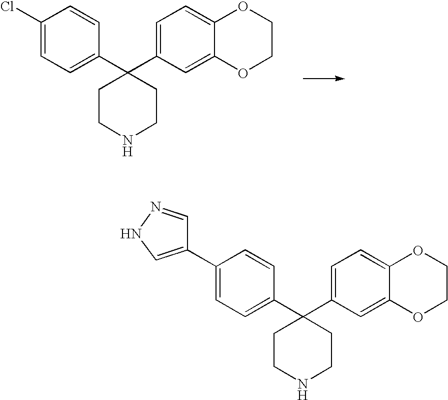

The title compound was obtained by the methods described in General Procedure 1 (78 mg, 0.1% yield). ¹H NMR (Me-d₃-OD) 7.91 (2H, s), 7.52 (2H, d), 7.30 (2H, d), 6.79 (3H, m), 4.21 (4H, s), 3.00 (4H, m), 2.43 (4H, m) LC/MS (PS-B5) Rₜ 2.44 [M+H]⁺ 362

### Example 84

### 4-(3-Chloro-phenyl)-4-[4-(1H-pyrazol-4-yl)-phenyl]-piperidine

### 84A. 4-(3-Chloro-phenyl)-4-hydroxy-piperidine-1-carboxylic acid tert-butyl ester

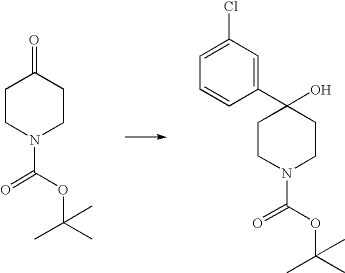

At room temperature a 0.5M solution of 3-chlorophenyl magnesium bromide in THF (50 mL, 25 mmol) was added to solid 4-oxo-piperidine-1-carboxylic acid tert-butyl ester (3.32 g, 16.7 mmol) with stirring under nitrogen. After 22 hours 20 minutes saturated aqueous ammonium chloride solution was added dropwise. The reaction mixture was left to cool to room temperature and then concentrated in vacuo to leave an aqueous slurry. The aqueous was diluted further with saturated aqueous ammonium chloride solution and then extracted twice with ethyl acetate. The organics were combined, washed with brine then dried (MgSO₄) and concentrated in vacuo. The residue was purified by flash column chromatography on silica gel, eluting with a gradient of 100% petroleum ether to 40% ethyl acetate in petroleum ether to afford the title compound as a yellow oil (1.94 g, 37%). LC/MS: (PS-L) Rₜ 2.00 [M+H]⁺ 312.18.

### 84B. 4-(4-Bromo-phenyl)-4-(3-chloro-phenyl)-piperidine hydrochloride

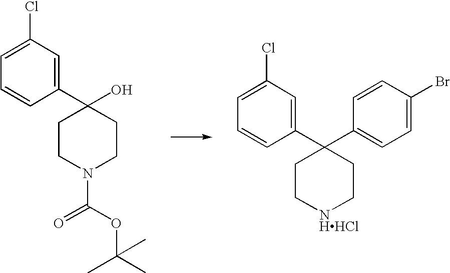

4-(3-Chloro-phenyl)-4-hydroxy-piperidine-1-carboxylic acid tert-butyl ester (0.3 g, 0.962 mmol) and aluminium (III) chloride (0.513 g, 3.85 mmol) were combined as solids and the mixture was cooled to 0° C. Bromobenzene (4 mL) was added and the cooling bath was removed. The reaction mixture was stirred for 20 hours at room temperature. The reaction mixture was then quenched with ice and evaporated in vacuo. The residue was purified by ion exchange chromatography followed by flash column chromatography on silica gel, eluting with 80/ 20 DCM/2N ammonia in methanol to afford the title compound as a colourless gum (0.215 g, 64%). LC/MS: (PS-B5) Rₜ 3.25 [M+H]⁺ 352.03.

### 84C. 4-(3-Chloro-phenyl)-4-[4-(1H-pyrazol-4-yl)-phenyl]-piperidine

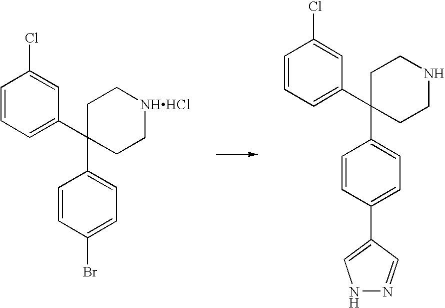

4-(4-bromo-phenyl)-4-(3-chloro-phenyl)-piperidine hydrochloride (0.215 g, 0.555 mmol), 4-(4,4,5,5-tetramethyl-[1,3,2]dioxaborolan-2-yl)-1H-pyrazole (0.238 g, 1.23 mmol) and potassium carbonate (0.455 g, 2.15 mmol) were mixed as solids and dissolved in a solvent mixture of ethanol, methanol, toluene and water (2 mL of each solvent). The solution was degassed with nitrogen and tetrakis(triphenyl-phosphine)palladium(0) (0.035 g, 0.0306 mmol) was added. The mixture was degassed again and then heated at 80° C. for 19 hours 30 minutes. Further batches of potassium carbonate (0.455 g, 2.15 mmol) and 4-(4,4,5,5-tetramethyl-[1,3,2]dioxaborolan-2-yl)-1H-pyrazole (0.238 g, 1.23 mmol) were added and the reaction mixture was degassed with nitrogen. A further batch of tetrakis(triphenyl phosphine)palladium(0) (0.035 g, 0.0306 mmol) was added and the reaction mixture was degassed again. The reaction was heated at 80° C. for a further 24 hours, then was allowed to cool to room temperature. The reaction mixture was concentrated in vacuo to leave the aqueous layer which was diluted with aqueous 2N NaOH solution then extracted three times with ethyl acetate. The organics were combined, dried (MgSO₄) and concentrated in vacuo. The residue was purified by flash column chromatography on silica gel, eluting with a gradient of 95/ 5 DCM/2N ammonia in methanol to 92.5/ 7.5 to 90/ 10 to afford the title compound as a white foam (0.068 g, 33%). LC/MS: (PS-B4) Rₜ 6.32 [M+H]⁺ 338.15. ¹H NMR (Me-d3-OD) 7.93 (2H, d), 7.57-7.49 (2H, m), 7.37-7.22 (5H, m), 7.20-7.13 (1H, m), 2.96-2.82 (4H, m), 2.56-2.30 (4H, m).

### Example 85

### 4-(4′-Methoxy-biphenyl-3-yl)-4-[4-(1H-pyrazol-4-yl)-phenyl]-piperidine dihydrochloride

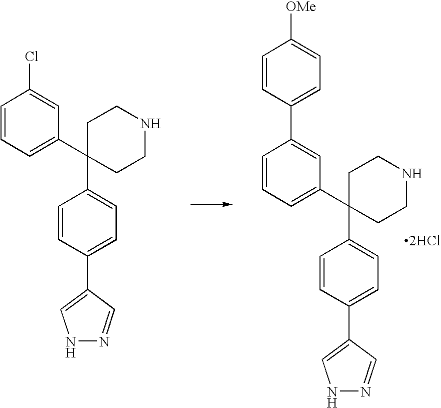

4-(3-chloro-phenyl)-4-[4-(1H-pyrazol-4-yl)-phenyl]-piperidine (0.054 g, 0.16 mmol)*, 2-(4-methoxy-phenyl)-4,4,5,5-tetramethyl-[1,3,2]dioxaborolane (0.075 g, 0.32 mmol) and potassium carbonate (0.119 g, 0.56 mmol) were mixed as solids and dissolved in a solvent mixture of ethanol, methanol, toluene and water (0.5 mL of each solvent). The mixture was degassed with nitrogen and bis(tri-t-butylphosphine)palladium(0) (0.004 g, 0.008 mmol) was added. The mixture was degassed again and then sealed under nitrogen and heated at 135° C. for 30 minutes in a CEM Explorer™ microwave. The reaction mixture was diluted with aqueous 2N NaOH solution then extracted three times with ethyl acetate. The organics were combined, dried (MgSO₄) and concentrated in vacuo. The residue was purified by flash column chromatography on silica gel, eluting with 85/ 15 DCM/2N ammonia in methanol to afford the free base as a colourless gum (0.026 g, 39%). The free base was then dissolved in methanol (5 mL) and a saturated solution of hydrogen chloride in diethyl ether (5 mL) was added. After stirring at room temperature for 16 hours the reaction mixture was evaporated in vacuo to afford the title compound as a white solid (0.030g, 100%). LC/MS: (PS-B4) Rₜ 7.41 [M+H]⁺ 410.25. ¹H NMR (Me-d3-OD) 8.41 (2H, s), 7.68 (2H, d), 7.56-7.47 (5H, m), 7.47-7.40 (2H, m), 7.37-7.32 (111, m), 7.00 (2H, d), 3.84 (3H, s), 3.32-3.25 (4H, m), 2.85-2.73 (4H, m).

* Refer to Example 84 for preparation of 4-(3-Chloro-phenyl)-4-[4-(1H-pyrazol-4-yl)-phenyl]-piperidine

### Example 86

### 4-(4-Methoxy-phenyl)-4-[4-(3-methyl-1H-pyrazol-4-yl)-phenyl]piperidine dihydrochloride

### 86A. 4-(4-Bromo-phenyl)-4-(4-methoxy-phenyl)-piperidine hydrochloride

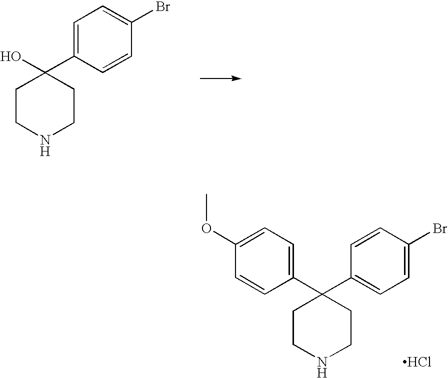

4-(4-bromo-phenyl)-piperidin-4-ol (4.7 g, 18=01) was converted to 4-(4-bromo-phenyl)-4-(4-methoxy-phenyl)-piperidine hydrochloride using the same method as detailed in Example 8B except that methoxybenzene (20 mL) was used instead of bromobenzene. The reaction mixture was quenched with ice and methyl tert-butyl ether was added with stirring. The precipitate was filtered in vacuo, washing with water followed by methyl tert-butyl ether. The precipitate was then dried to afford the title compound as an off-white solid (6.76 g, 98%). LC/MS: (PS-B5) Rₜ 3.07 [M+H]⁺ 346.

### 86B. 3-methyl-1-trityl-1H-4-pyrazolylboronic acid

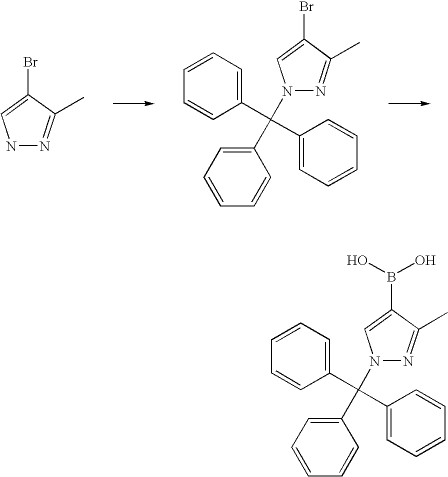

3-methyl-1-trityl-1H-4-pyrazolylboronic acid can be made by the method described in EP1 3 82603A

### 86C. 4-(4-Methoxy-phenyl)-4-[4-(3-methyl-1H-pyrazol-4-yl)-phenyl]-piperidine

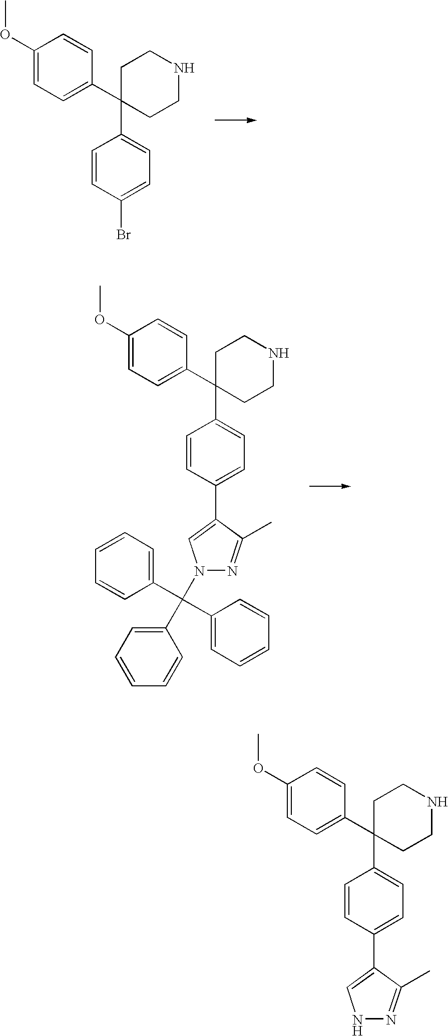

4-(4-Bromo-phenyl)-4-(4-methoxy-phenyl)-piperidine hydrochloride (0.494 g, 1.29 mmol) was converted to 4-(4-methoxy-phenyl)-4-[4-(3-methy 1-1-trityl-1H-pyrazol-4-yl)-phenyl]-piperidine using the same method as detailed in Example 8C except that 3-methyl-1-trityl-1H-4-pyrazolylboronic acid (0.952 g, 2.59 mmol) was used instead of 4-(4,4,5,5-tetramethyl-[1,3,2]dioxaborolan-2-yl)-1H-pyrazole and bis(tri-t-butylphosphine)palladium(0) was used instead of tetrakis(triphenyl-phosphine)palladium(0). Heating was continued for 17 hours only then the reaction was worked up using the same method as detailed in example 5C. The product was purified by flash column chromatography on silica gel, eluting with 90/ 10 DCM/2N ammonia in methanol to afford the trityl-protected product (0.660g, 87%). The product was then dissolved in methanol (10 mL) and aqueous 2N HCl solution (5 mL) was added with stirring. After 1 hour 45 minutes the reaction mixture was evaporated in vacuo to remove the organic solvent. The residue was diluted with aqueous 2N NaOH solution and extracted twice with ethyl acetate. The organics were combined, dried (MgSO₄) and concentrated in vacuo. The residue was purified by flash column chromatography on silica gel, eluting with 80/ 20 DCM/2N ammonia in methanol to afford the title compound as a glassy colourless solid (0.356 g, 92%). LC/MS: (PS-B4) Rₜ 5.65 [M+H]⁺ 348.23.

### 86D. 4-(4-Methoxy-phenyl)-4-[4-(3-methyl-1H-pyrazol-4-yl)-phenyl]-piperidine dihydrochloride

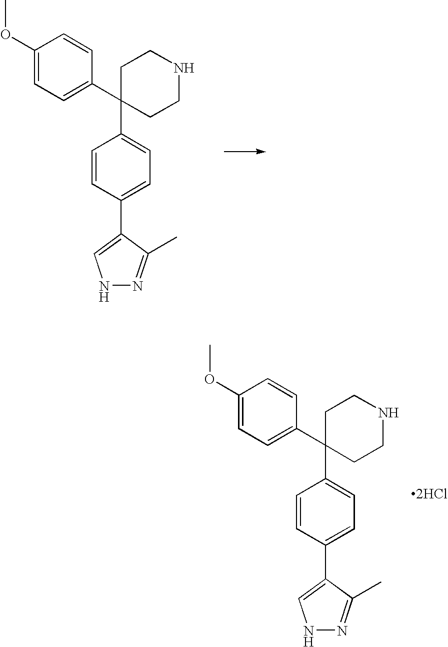

4-(4-Methoxy-phenyl)-4-[4-(3-methyl-1H-pyrazol-4-yl)-phenyl]-piperidine (0.345 g, 0.99 mmol) was converted to 4-(4-methoxy-phenyl)-4-[4-(3-methyl-1H-pyrazol-4-yl)-phenyl]-piperidine dihydrochloride using the same method as detailed in Example 85 except that after the reaction mixture had been evaporated in vacuo, the residue was re-dissolved in methanol and diethyl ether was added. The resulting precipitate was filtered in vacuo, washing with diethyl ether then dried to afford the title compound as a white solid (0.359 g, 86%). LC/MS: (PS-B4) Rₜ 5.84 [M+H]⁺348.16. ¹H NMR (Me-d3-OD) 7.67 (2H, s), 7.39-7.29 (4H, q), 7.25 (2H, d), 6.85 (2H, d), 3.76 (3H, s), 2.95-2.87 (4H, m), 2.48-2.39 (4H, m), 2.39 (3H, s).

### Example 87

### 4-(4-Chloro-phenyl)-4-[4-(5-ethyl-1H-pyrazol-4-yl)-phenyl]-piperidine

### 87A. 4-(4-Bromo-phenyl)-4-(4-chloro-phenyl)-piperidine hydrochloride

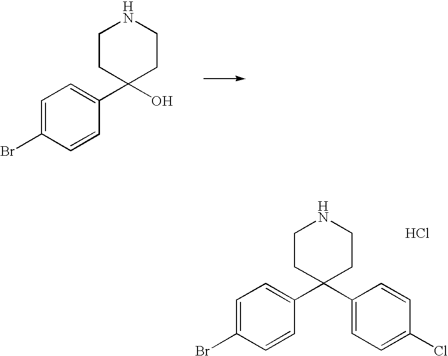

A suspension of 4-(4-bromo-phenyl)-piperidin-4-ol (4.02 g, 15.7 mmol) in chlorobenzene (30 ml) was added dropwise to a suspension of aluminium chloride (7.32 g, 54.9 mmol) in chlorobenzene (10 ml) at 0° C. The reaction mixture was stirred at 0° C. for 2 hours, quenched by addition of ice then methyl t-butyl ether added. After stirring for 1 hour the precipitate was collected by filtration washed with water, methyl t-butyl ether and water to afford the desired compound. LC/MS: (FL-A) Rₜ 2.32 [M+H]⁺ 350

### 87B. 4-(4-Bromo-phenyl)-4-(4-chloro-phenyl)-piperidine-1-carboxylic acid tert-butyl ester

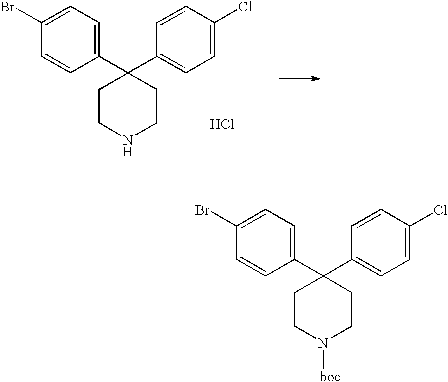

To a suspension of 4-(4-bromo-phenyl)-4-(4-chloro-phenyl)-piperidine (10 g, 25.8 mmol) in dichloromethane (150 ml) was added triethylamine (4.3 ml, 31.0 mmol) and di-tert-butyl dicarbonate (6.2 g, 28.4 mmol). After stirring at room temperature for 72 hours, water was added and the organic layer removed. This was washed with water then saturated sodium chloride solution before drying (MgSO₄) and concentrating in vacuo to furnish the desired compound as a white solid (11.4 g, 98%). LC/MS: (FL-A) Rₜ 4.89 [M−^{t}Bu]⁺ 394

### 87C. 4-(4-Chloro-phenyl)-4-[4-(4,4,5,5-tetramethyl-[1,3,2]dioxaborolan-2-yl)-phenyl]-piperidine-1-carboxylic acid tert-butyl ester

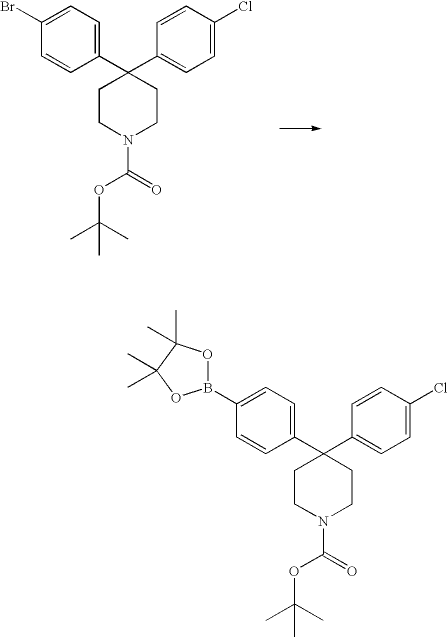

A mixture of 4-(4-bromo-phenyl)-4-(4-chloro-phenyl)-piperidine-1-carboxylic acid tert-butyl ester (5.0 g, 11.1 mmol), bis(pinacolato)diboron (2.8 g, 11.1 mmol), potassium acetate (3.3 g, 33.3 mmol) and [1,1v-bis(diphenylphosphino)ferrocene]dichloro palladium(II) (406 mg, 0.55 mmol) was heated to 80 deg C. under nitrogen for 2.5 hours. The reaction was then allowed to cool, diluted with ethyl acetate then filtered under suction. The solid was triturated with ethyl acetate to furnish the desired compound as a beige solid (3.56 g, 64% yield). ¹H NMR (Me-d3-OD) 7.73 (2H, d), 7.25 (4H, m), 7.13 (2H, d), 3.52 (2H, m), 3.38 (2H, m), 2.40 (2H, m), 2.30 (2H, m), 1.42 (9H, s), 1.32 (12H, s)

### 87D. 4-(4-Chloro-phenyl)-4-[4-(1-dimethylsulphamoyl-5-ethyl-1H-pyrazol-4-yl)-phenyl]-piperidine-1-carboxylic acid tert-butyl ester

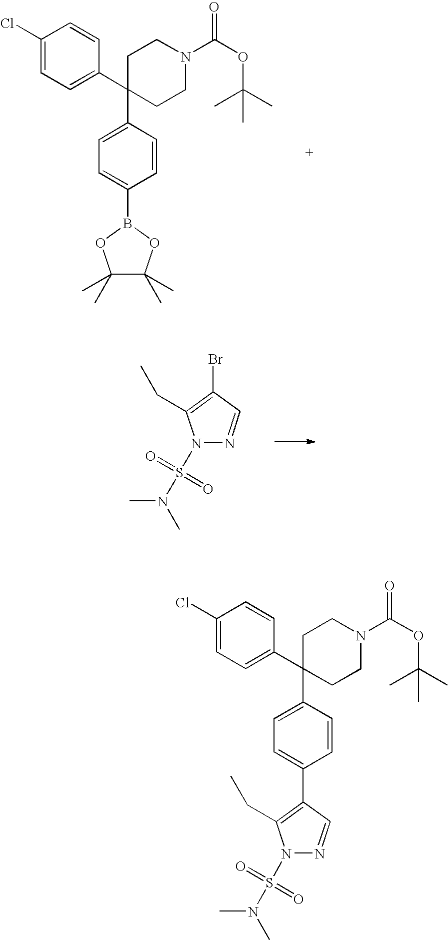

A solution of 4-Bromo-3-ethyl-pyrazole-1-sulphonic acid dimethylamide (03 g, 1.06 mmol), 4-(4-Chloro-phenyl)-4-[4-(4,4,5,5-tetramethyl-[1,3,2]dioxaborolan-2-yl)-phenyl]-piperidine-1-carboxylic acid tert-butyl ester (0.53 g, 1.06 mmol) and K₃PO₄ (0.56 g, 2.66 mmol) in ethanol (3.5 ml), methanol (3.5 ml), toluene (3.5 ml) and water (3.5 ml) is purged with nitrogen for 2 minutes. Tetrakis(triphenylphosphine) palladium (0) (5 mol %) is then added and the reaction mixture purged with nitrogen for a further 2 minutes. The mixture is then heated to 80° C., under nitrogen for a period of 17 hours. The reaction mixture is cooled and partitioned between dichloromethane and water, the organic layer is collected through a phase separator cartridge, concentrated. The crude product was purified by column chromatography (SiO₂), eluting with a gradient 0-40% ethyl acetate/petrol to afford the product (0.59 g, 96% yield). LC/MS (PS-A2) Rₜ 4.30 [M+H]⁺ 573.22.

### 87E. 4-(4-Chloro-phenyl)-4-[4-(5-ethyl-1H-pyrazol-4-yl)-phenyl]-piperidine

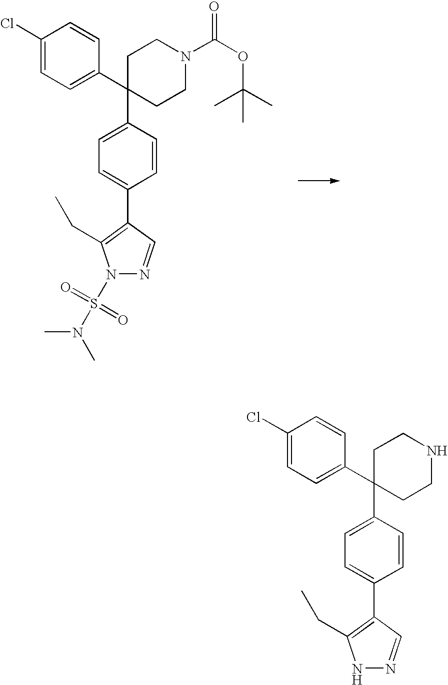

The title compound was prepared according to the procedure outlined in Example 77L (121 mg, 32% yield). ¹H NMR (Me-d₃-OD) 8.44 (1H, s), 7.53 (1H, s), 7.38 (8H, m), 3.10 (4H, m), 2.71 (2H, q), 2.59 (2H, m), 2.52 (2H, m), 1.12 (3H, t). LC/MS (PS-B5) Rₜ 2.96 [M+H]⁺ 366.

### Example 88

### 4-(4-Chloro-phenyl)-4-[4-(5-cyclopropyl-1H-pyrazol-4-yl)-phenyl]-piperidine

### 88A. 5-Iodo-pyrazole-1-sulphonic acid dimethylamide

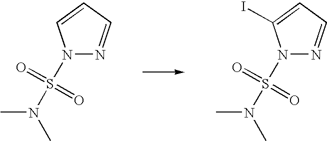

Using the methods described in Example 80B, pyrazole-1-sulphonic acid dimethylamide and 1-chloro-2-iodoethane were reacted to produce the title compound (1.68 g, 89% yield). LC/MS (PS-A2) Rₜ 2.76 [M+H]⁺ 302.

### 88B. 5-Cyclopropyl-pyrazole-1-sulphonic acid dimethylamide

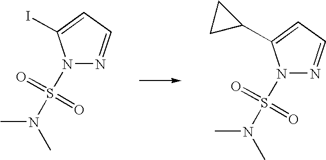

### (Tetrahedron Letters, 43 (2003), 6987-6990)

To a solution of 5-Iodo-pyrazole-1-sulphonic acid dimethylamide (0.5 g, 1.66 mmol), cyclopropylboronic acid (0.19 g, 2.16 mmol), potassium phosphate (1.23 g, 5.81 mmol) and tricyclohexylphosphine (0.047 g, 0.17 mmol) in toluene (7 ml) and water (0.35 ml) under a nitrogen atmosphere was added palladium acetate (19 mg, 0.08 mmol). The mixture was heated to 100° C. for 2 hours and then cooled to room temperature. Water was added and the mixture extracted with ethyl acetate, the combined extracts were dried (MgSO₄) and concentrated in vacuo. The crude product was purified by column chromatography (SiO₂), eluting with a gradient 0-30% ethyl acetate/petrol to afford the product (0.27 g, 76% yield). LC/MS (PS-A2) Rₜ 2.73 [M+H]⁺ 216.08.

### 88C. 4-Bromo-5-cyclopropyl-pyrazole-1-sulphonic acid dimethylamide

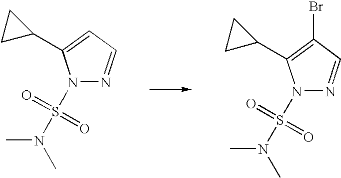

Using the method described in Example 80C₁₋₅-cyclopropyl-pyrazole-1-sulphonic acid dimethylamide was reacted to produce the title compound, crude material used without further purification (0.4 g). LC/MS (PS-A2) Rₜ 3.30 [M+H]⁺ 296.

### 88D. 4-(4-Chloro-phenyl)-4-[4-(5-cyclopropyl-1-dimethylsulphamoyl-1H-pyrazol-4-yl)-phenyl]-piperidine-1-carboxylic acid tert-butyl ester

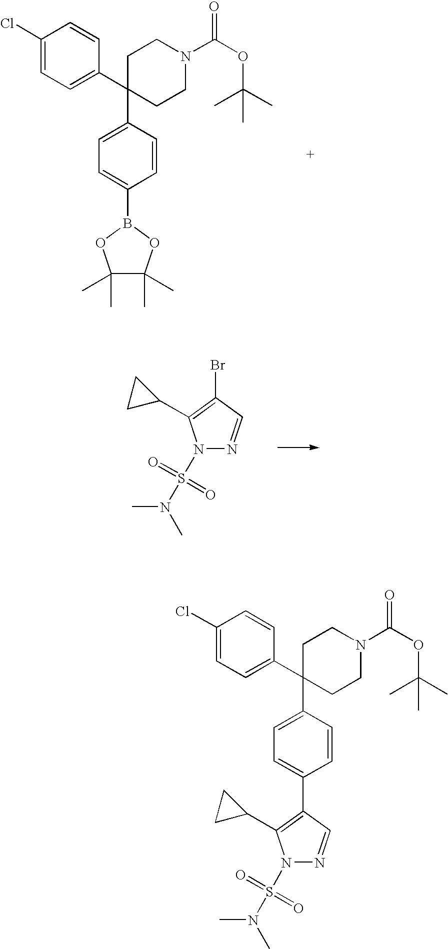

Using the method described in Example 87D, 4-(4-chloro-phenyl)-4-[4-(4,4,5,5-tetramethyl-[1,3,2]dioxaborolan-2-yl)-phenyl]-piperidine-1-carboxylic acid tert-butyl ester and 4-Bromo-5-cyclopropyl-pyrazole-1-sulphonic acid dimethylamide were reacted to produce the title compound (0.32 g, 39% yield). LC/MS (PS-A2) Rₜ 3.30 [M+H]⁺ 296.

### 88E. 4-(4-Chloro-phenyl)-4-[4-(5-cyclopropyl-1H-pyrazol-4-yl)-phenyl]-piperidine

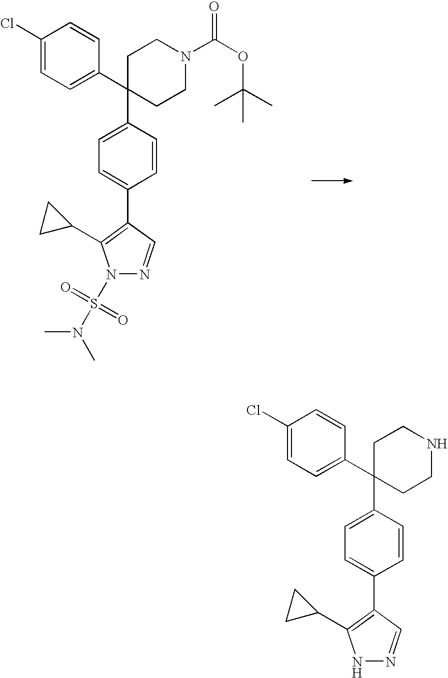

The title compound was prepared according to the procedure described in Example 77L (26 mg, 13% yield). ¹H NMR (Me-d₃-OD) 7.67 (1H, s), 7.55 (2H, d), 7.32 (6H, m), 2.92 (4H, m), 2.48 (4H, m), 2.00 (1H, m), 0.98 (214, m), 0.81 (2H, m). LC/MS (PS-B5) Rₜ 2.79 [M+H]⁺ 378.

### Example 89

### Dimethyl-(3-{4-[4-(1H-pyrazol-4-yl)-phenyl]-piperidin-4-yl}-phenyl)-amine

### 89A. 4-(3-Dimethylamino-phenyl)-4-hydroxy-piperidine-1-carboxylic acid tert-butyl ester

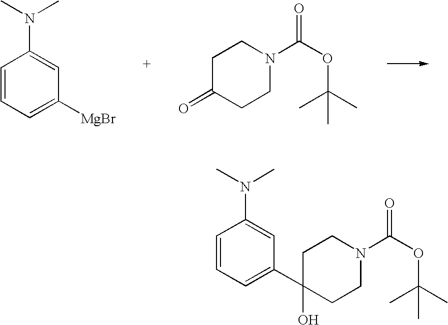

Using the method described in Example 81A, 4-oxo-piperidine-1-carboxylic acid tert-butyl ester and 3-N-dimethylphenylmagnesium bromide were reacted to produce the title compound (3.88 g, 73% yield). LC/MS (PS-A2) Rₜ 2.16 [M+H]⁺ 321.

### 89B. {3-[4-(4-Chloro-phenyl)-piperidin-4-yl]-phenyl}-dimethyl-amine

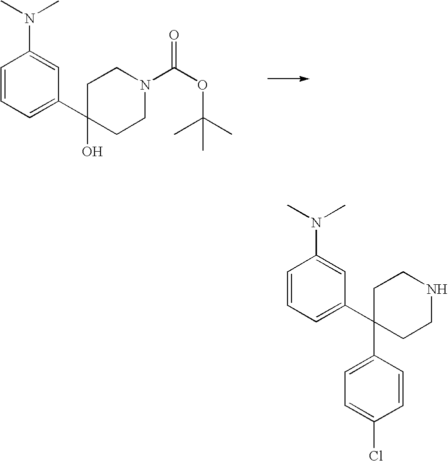

Using the methodology outlined in Example 87A 4-(3-dimethylamino-phenyl)-4-hydroxy-piperidine-1-carboxylic acid tert-butyl ester and chlorobenzene were reacted to produce the title compound (0.07 g, 15% yield). LC/MS (PS-A2) Rₜ 1.95 [M+11]⁺ 315.

### 89C. Dimethyl-(3-{4-[4-(1H-pyrazol-4-yl)-phenyl]-piperidin-4-yl}-phenyl)-amine

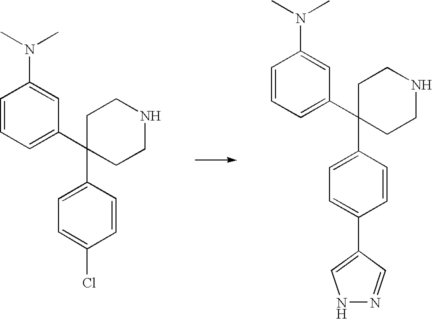

The title compound was prepared according to General Procedure 1 (40 mg, 49% yield). ¹H NMR (Me-d₃-OD) 7.93 (2H, s), 7.56 (2H, d), 7.38 (2H, d), 7.20 (111, t), 6.76 (1H, d), 6.68 (2H, m), 3.25 (4H, m), 2.89 (611, s), 2.68 (4H, m), 1.95 (61-1, s). LC/MS (PS-B5) Rₜ 2.66 [M+H]⁺ 347.

### Example 90

### 4-(2-Methoxy-5-pyridin-yl)-4-[4-(1H-pyrazol-4-yl)-phenyl]-piperidine

### 90A. 4-(2-Methoxy-5-pyridin-yl)-4-hydroxy-piperidine-1-carboxylic acid tert-butyl ester

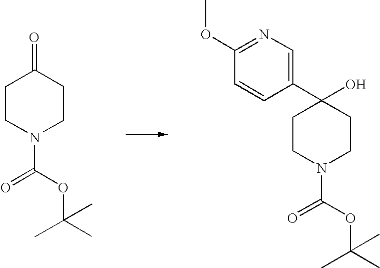

n-Butylithium (2.5 M in hexanes) (14.30 mL, 35.7 mmol) was added dropwise to a stirred solution of 2-methoxy-5-bromopyridine (4.66 mL, 36.1 mmol) in THF at −78° C. After 5 min. a solution of 4-oxo-piperidine-1-carboxylic acid tert-butyl ester (5.98 g, 30.0 mmol), in THF (40 mL), was added dropwise. The mixture was allowed to warm to 0° C. and then quenched by addition of saturated ammonium chloride solution. The aqueous mixture was extracted with AcOEt (×2). The combined organic fraction was washed with brine, dried (MgSO₄) and concentrated in vacuo. The residue was purified by flash column chromatography on silica gel, eluting with a gradient of 100% petroleum ether to 40% ethyl acetate in petroleum ether to afford the title compound as a colourless solid (4.10 g, 44%). LC/MS: (PS-A3) Rₜ 2.69 [M+H]⁺ 309.

### 90B 4-(2-Methoxy-5-pyridin-yl)-4-[4-bromo-phenyl]-piperidine

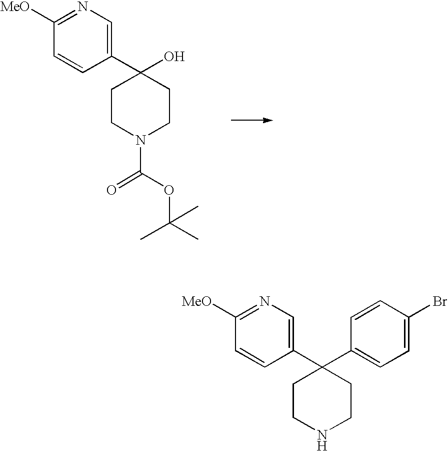

Using the method described in Example 81B, 4-(2-methoxy-5-pyridin-yl)-4-hydroxy-piperidine-1-carboxylic acid tert-butyl ester (1.84 g, 5.99 mmol) and bromobenzene (7 mL) were reacted to produce the title compound (2.5 g). Following aqueous work up and evaporation in vacuo, the product was used in the next step without any further purification.

### 90C 4-(2-Methoxy-5-pyridin-yl)-4-[4-(1H-pyrazol-4-yl)-phenyl]-piperidine

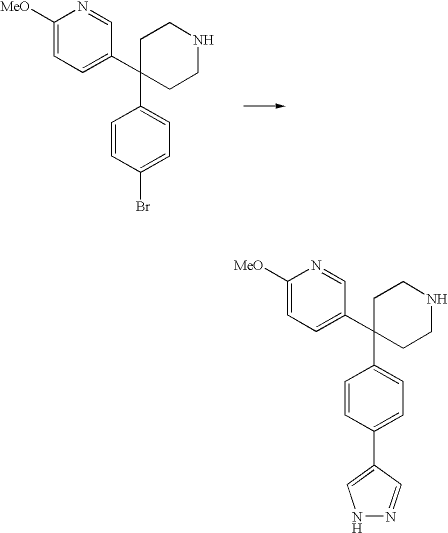

The title compound was prepared according to General Procedure 1, using 4-(2-methoxy-5-pyridin-yl)-4-[4-bromo-phenyl]-piperidine (225 mg). The product was obtained as a colourless solid (11 mg, 10%). LC/MS (PS-B5) Rₜ 2.27 [M+H]⁺ 335. 1H NMR (Me-d3-OD) 7.95 (1H, m), 7.81 (2H, s), 7.46 (111, m), 7.42 (2H, d), 7.22 (2H, d), 6.61 (1H, d), 3.75 (3H, s), 2.84 (4H, m), 2.40 (2H, m), 2.30 (2H, m)

### Example 91

### 2-Amino-1-(4-chloro-phenyl)-[5-(1H-pyrazol-4-yl)-pyridin-2-yl]-ethanol hydrochloride

### 91A. (4-Chloro-phenyl)-(5-bromo-pyridin-2-yl)-methanone

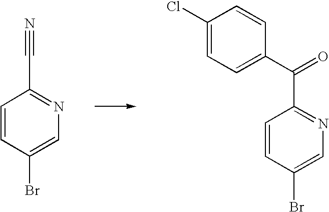

To 4-chlorophenylmagnesium bromide (1M in Et₂O; 50 mL, 50 mmol), in THF at −78° C., was added 5-bromopyridine-2-carbonitrile (9.15 g, 50 mmol), as a solution in Tiff (50 mL). The mixture was allowed to warm to 10° C. at which point 10% aq. HCl (100 mL) was added very cautiously (ice bath used to control temperature). The heterogeneous mixture was stirred overnight and then the product collected by filtration. The solid was then partitioned between AcOEt and brine after which the organic layer was separated and dried (MgSO₄). The solution was concentrated in vacuo and the product recrystallised from AcOEt/hexanes to give the title compound (4.68 g, 32%) as a colourless solid. LC/MS (PS-L) Rₜ 2.27 [M+H]⁺ 296

### 91B. 2-(4-Chloro-phenyl)-2-(5-bromo-pyridin-2-yl)-oxirane

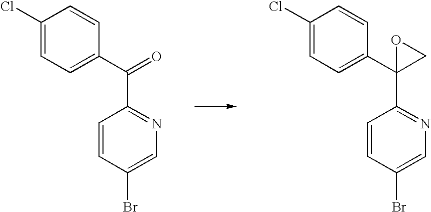

(4-Chloro-phenyl)-(5-bromo-pyridin-2-yl)-methanone (2.31 g, 7.82 mmol) and trimethylsulphonium iodide (1.75 g, 8.61 mmol) were suspended in DMSO (25 mL). NaH (60% dispersion in oil; 350 mg, 8.68 mmol) was then added batchwise and the mixture stirred overnight. The mixture was then partitioned between AcOEt (100 ml) and H₂O:brine (1:1) (100 mL). The organic layer was separated and washed further with H2O (×4), brine and then dried (MgSO₄). The product was obtained as a brown oil (2g, 83%) which was used in the next step without any further purification. LC/MS (PS-L) Rₜ 2.26 [M+H]⁺ 310

### 91C. 2-Amino-1-(4-chloro-phenyl)-1-(5-bromo-pyridin-2-yl)-ethanol

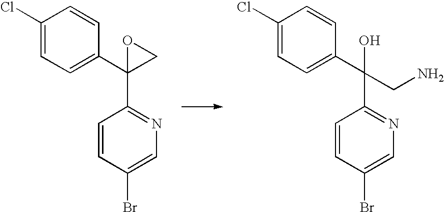

Using the method described in Example 78A, 2-(4-chloro-phenyl)-2-(5-bromo-pyridin-2-yl)-oxirane (2 g, 6.47 mmol) was treated with 2N NH₃ in MeOH to give the title compound (667 mg, 32%) as a yellow oil. LC/MS (PS-B5) Rₜ 2.92 [M−H₂O+H]⁺ 309

### 91D. 2-Amino-1-(4-chloro-phenyl)-1-[5-(1H-pyrazol-4-yl)-pyridin-2-yl]-ethanol hydrochloride

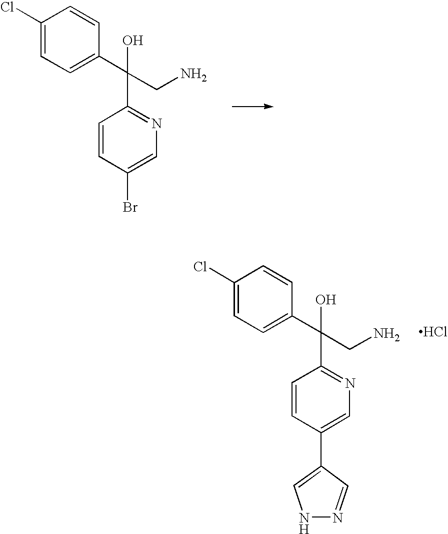

Starting from 2-amino-1-(4-chloro-phenyl)-1-(5-bromo-pyridin-2-yl)-ethanol. The title compound was prepared according to General Procedure 1. The product was obtained as a colourless solid (50 mg). The product was dissolved in MeOH/AcOEt (1:1) and treated with 1 mol. eq. of HCl (4N in dioxane). The solution was evaporated to dryness to give the title compound (40 mg, 13%) as a colourless solid. LC/MS (PS-B5) Rₜ 2.35 [M−H⁺]⁻ 313. 1H NMR (DMSO-d₆) 8.84 (1H, m) 8.17 (2H, s), 8.03 (1H, m), 7.81 (3H, br), 7.57 (1H, d), 7.48 (2H, d), 7.40 (2H, d), 6.82 (1H, br), 3.84 (1H, m), 3.52 (1H, m)

### Example 92

### 3-Amino-1-(4-chloro-phenyl)-1-[4-(1H-pyrazol-4-yl)-phenyl]-propan-1-ol

### 92A 3-(4-Chloro-phenyl)-3-hydroxy-3-(4-iodo-phenyl)-propionitrile

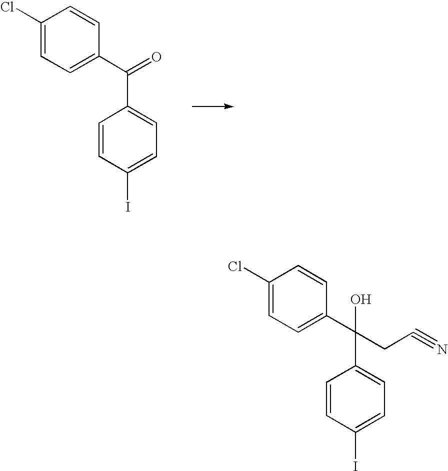

To n-BuLi (2.5M in hexanes) (7.3 mL, 18.25 mmol), in THF (60 mL) at −78° C., was added MeCN (0.95 mL, 18.25 mmol). The mixture was stirred for 30 min at −78° C. followed by addition of a solution of (4-chloro-phenyl)-(4-iodo-phenyl)-methanone (5 g, 14.6 mmol) in THF (60 mL). The mixture was then allowed to warm to r.t. over 30 min. after which saturated aqueous NH₄Cl was added. The organic layer was separated, washed with brine, dried (Na₂SO₄) and then concentrated in vacuo. The title compound was obtained as a yellow oil (7.3 g, 87%). LC/MS (PS-L) Rₜ 2.11, [M−H⁺]⁻ 382.

### 92B. 3-(4-Chloro-phenyl)-3-hydroxy-3-[4-(1H-pyrazol-4-yl)-phenyl]-propionitrile

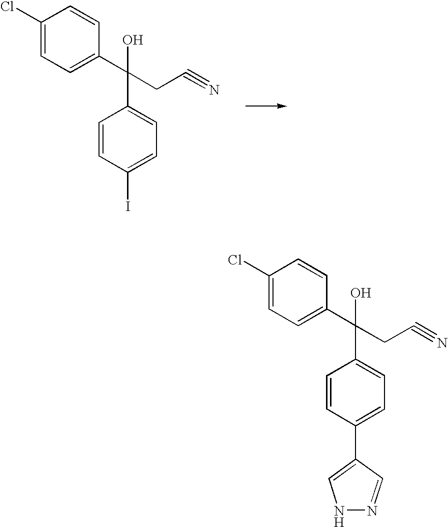

The title compound was prepared according to General Procedure 1. The product was obtained as a colourless solid (1.0 g, 51%). LC/MS (PS-B5) Rₜ 2.70 [M+H]⁺ 324

### 92C. 3-Amino-1-(4-chloro-phenyl)-1-[4-(1H-pyrazol-4-yl)-phenyl]-propan-1-ol

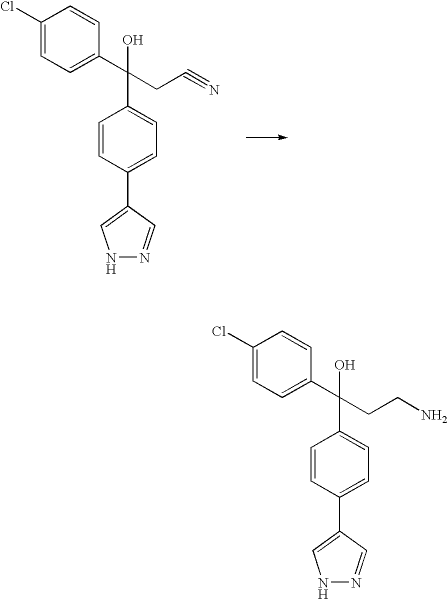

To LiAlH₄ (93 mg, 2.46 mmol), in THF (8 mL) at 0° C., was added 3-(4-chloro-phenyl)-3-hydroxy-3-[4-(1H-pyrazol-4-yl)-phenyl]-propionitrile (400 mg, 1.23 mmol). The mixture was stirred at 0° C. for 30 min. and then r.t for 1 hr. The mixture was then cooled to 0° C. and quenched by successive, careful, addition of H₂O (383 μl) and 10% aq. NaOH (150 μA). After stirring for a further 10 min. the mixture was partitioned between AcOEt and 10% aq. NaOH. The aqueous layer was extracted further with AcOEt (×1). The combined organic fraction was washed with brine, dried (Na₂SO₄) and concentrated in vacuo. The residue was purified by flash column chromatography on silica gel, eluting with CH₂Clᵣ10% MeOH-1% conc. aq. NH₃ to afford the title compound as a colourless solid (100 mg, 25%). LC/MS: (PS-B5) Rₜ 2.58 [M−CH₂NH−H₂O+H]^{F} 281. 1H NMR (DMSO-d₆) 7.97 (2H, br s), 7.47 (411, m), 7.41 (2H, d), 7.32 (2H, d), 3.32 (2H, br), 2.68 (2H, t), 2.32 (2H, m)

### Example 93

### 2-(3-{4-[4-(3-Methyl-1H-pyrazol-4-yl)-phenyl]-piperidin-4-ylmethoxy}-phenyl)-benzooxazole

### 93A. 3-Benzooxazol-2-yl-phenol

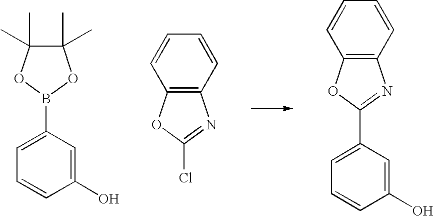

3-(4,4,5,5-tetramethyl-1,3,2-dioxaborolan-2-yl)phenol and 2-chlorobenzooxazole were coupled using the method outlined in Example 77K although in this case the mixture was heated at 100° C. overnight. The title compound was obtained as a red solid (1.0 g, 65% yield). LC/MS (PS-A2) Rₜ 2.86 [M+H]⁺ 212.

### 93B. 2-(3-{4-[4-(3-Methyl-1H-pyrazol-4-yl)-phenyl]-piperidin-4-ylmethoxy}-phenyl)-benzooxazole

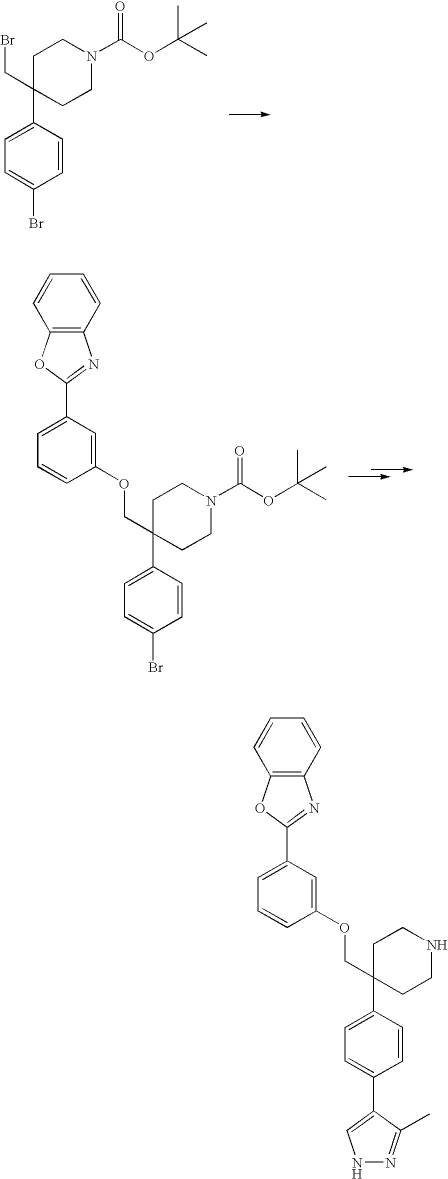

The title compound was obtained according to the methods described in Examples 7711, 77K and 77L, using 3-benzooxazole-2-yl-phenol in place of 3-methoxyphenol. LC/MS (PS-B5) Rₜ 7.78 [M+H]⁺ 465. ¹H NMR (Me-d₃-OD) 8.50 (2H, s), 7.82 (111, d), 7.72 (3H, m), 7.68 (1H, dd), 7.64 (2H, d), 7.59 (2H, d), 7.48 (1H, t), 7.42 (2H, m), 7.13 (111, dd), 4.13 (2H, s), 3.43 (2H, m), 3.09 (2H, m), 2.71 (2H, m), 2.43 (3H, s), 2.40 (2H, m).

### Example 94

### 3-Amino-1-(4-chlorophenyl)-1-[2-fluoro-4-(1H-pyrazol-4-yl)-phenyl]-propan-1-ol-acetic acid salt

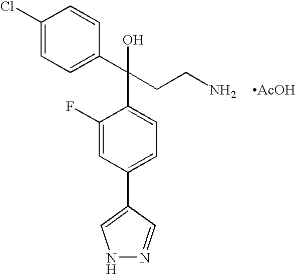

### 94A. (4-Bromo-2-fluoro-phenyl)-(4-chloro-phenyl)-methanone

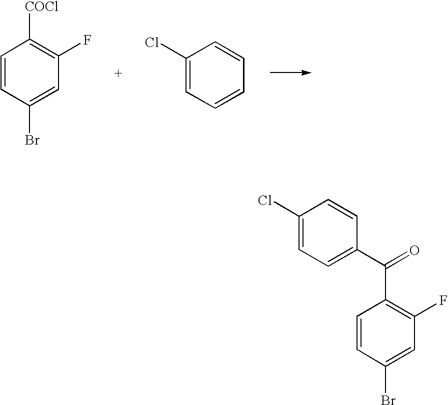

Aluminium trichloride (10.75 g, 80.8 mmol) was added batchwise to a stirred solution of 4-bromo-2-fluoro-benzoyl chloride (12.77 g, 53.9 mmol) in chlorobenzene (75 mL). The solution was heated at 120° C. for 16 h and then allowed to cool. The mixture was poured onto a stirred mixture of crushed ice (500g) and conc. HCl (50 mL). After stirring for 5 min. the organic layer was separated and washed with 5% aqueous NaOH, water and then brine. The solution was dried (MgSO₄) and then evaporated to dryness. Recrystallisation from AcOEt/hexanes afforded the title compound as a colourless crystalline solid (7.67 g, 40%)

### 94B. 3-Amino-1-(4-chloro-phenyl)-1-[2-fluoro-4-(1H-pyrazol-4-yl)-phenyl]-propan-1-ol

The title compound was prepared according to the methods described for Example 92, using (4-bromo-2-fluoro-phenyl)-(4-chlorophenyl)-methanone instead of (4-chloro-phenyl)-(4-iodo-phenyl)-methanone. The product was obtained as a colourless solid. LC/MS: (PS-B5) Rₜ 3.35 [M−H⁺]⁻ 354

### 94C. 3-Amino-1-(4-chloro-phenyl)-1-[2-fluoro-4-(1H-pyrazol-4-yl)-phenyl]-propan-1-ol acetic acid salt

To a stirred solution of 3-amino-1-(4-chloro-phenyl)-1-[2-fluoro-4-(1H-pyrazol-4-yl)-phenyl]-propan-1-ol (121 mg, 0.35 mmol) in THF (2 mL) was added AcOH (22 0.38 mmol). After 10 mins, the resulting precipitate was collected and dried, giving the title compound (46 mg, 33%) as a colourless solid. LC/MS: (PS-B5) Rₜ 2.53 [M−H⁺]⁻ 344. 1H NMR (DMSO-d₆) 8.09 (2H, br), 7.73 (1H, t), 7.45 (1H, d), 7.35 (5H, m), 2.64 (2H, m), 2.34 (2H, m), 1.88 (3H, s)

### Example 95

### 3-Amino-1-(4-chloro-3-fluoro-phenyl)-1-[4-(1H-pyrazol-4-yl)-phenyl]-propan-1-ol

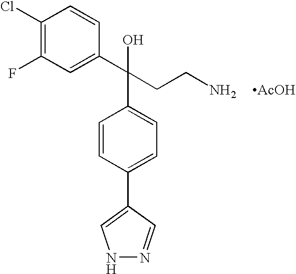

The title compound was prepared according to methods described for Example 94, using 4-chloro-3-fluoro-benzoyl chloride and bromobenzene as starting materials. The product was obtained as a colourless solid. LC/MS: (PS-B5) Rₜ 2.53 [M−H⁺]⁻ 344. 1H NMR (DMSO-d₆) 8.0 (2H, br), 7.46 (6H, m), 7.30 (1H, m), 2.67 (2H, m), 2.37 (2H, m), 1.89 (3H, s)

### Example 96

### 3-Amino-1-(3,4,-dichlorophenyl)-1-[4-(1H-pyrazol-4-yl)-phenyl]-propan-1-ol

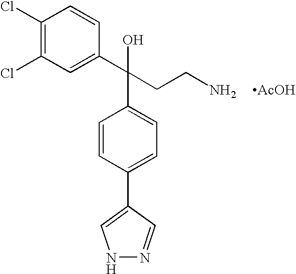

The title compound was prepared according to the methods described for Example 94, using 3,4-dichlorobenzoyl chloride and bromobenzene as starting materials. The product was obtained as a colourless solid. LC/MS: (PS-B5) Rₜ 2.64 [M−H⁺]⁻ 360. 1H NMR (DMSO-d₆) 8.00 (2H, br), 7.68 (1H, m), 7.54 (3H, m), 7.43 (3H, m), 2.69 (2H, m), 2.36 (2H, m), 1.89 (3H, s).

An alternative synthetic route for preparing 3-Amino-1-(3,4,-dichlorophenyl)-1-[4-(1H-pyrazol-4-yl)-phenyl]-propan-1-ol is described in Example 48.

### Example 97

### {2-(3-Chloro-phenoxy)-2-[4-(1H-pyrazol-4-yl)-phenyl]-ethyl}-methyl-amine

### 97A. 1-(4-Bromo-phenyl)-2-methylamino-ethanol

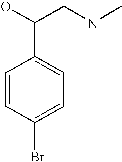

The title compound can be prepared by following General Procedure 5A.

### 97B. [2-(4-Bromo-phenyl)-2-hydroxy-ethyl]-methyl-carbamic acid tert-butyl ester

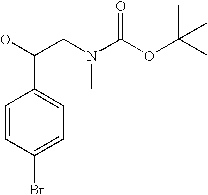

The title compound can be prepared by reacting 1-(4-Bromo-phenyl)-2-methylamino-ethanol with BOC₂O following the procedure outlined in WO05061463A1 Example 48A.

### 97C. [2-(4-Bromo-phenyl)-2-(3-chloro-phenoxy)-ethyl]-methyl-amine

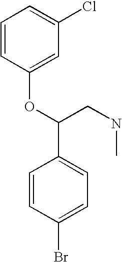

The title compound can be prepared by reacting [2-(4-bromo-phenyl)-2-hydroxy-ethyl]-methyl-carbamic acid tert-butyl ester with 3-chlorophenol following the procedure outlined in WO 2005/061463A1 Example 48B.

### 97D. {2-(3-Chloro-phenoxy)-2-[4-(1H-pyrazol-4-yl)-phenyl]-ethyl}-methyl-amine

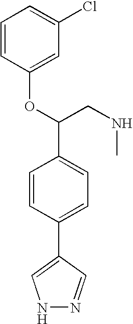

The title compound can be prepared from the compound described in Example 97C by following General Procedure 1, using tetrakis(triphenylphosphine)palladium(0) and heating thermally at 80° C. for 2 hours, instead of bis(tri-t-butylphosphine)palladium(0) and heating using a CEM microwave.

### Example 98

### Methyl-[2-[4-(1H-pyrazol-4-yl)-phenyl]-2-(3-pyridin-2-yl-phenoxy)-ethyl]-amine

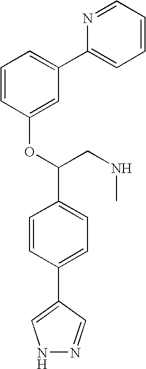

The title compound can be prepared by reacting the compound prepared in Example 97 with 2-(2-pyridyl)-4,4,5,5-tetramethyl-1,3,2-dioxaborolane using General Procedure 1.

### Example 99

### Resolution of racemic 3-amino-1-(4-chloro-phenyl)-1-[4-(1H-pyrazol-4-yl)-phenyl]-propan-1-ol to give Example A: (S)-3-Amino-1-(4-chloro-phenyl)-1-[4-(1H-pyrazol-4-yl)-phenyl]-propan-1-ol and Example B: (R)-3-Amino-1-(4-chloro-phenyl)-1-[4-(1H-pyrazol-4-yl)-phenyl]-propan-1-ol

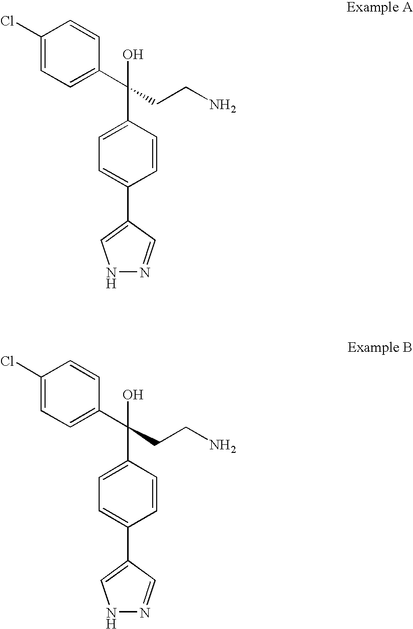

Racemic 3-amino-1-(4-chloro-phenyl)-1-[4-(1H-pyrazol-4-yl)-phenyl]-propan-1-ol (Example 92) (26 mg) was resolved by preparative chiral HPLC (Method AG-CP3). Example A and Example B were isolated as colourless solids (9 mg in each case). LC/MS Example A: (AG-CA2) Rₜ 13.26, ee 98.2%. LC/MS Example B: (AG-CA2) Rₜ 11.79, % ee 97.4. 1H NMR (Me-d₃-OD) 7.83 (2H, m), 7.45 (2H, d), 7.33 (4H, m), 7.20 (2H, d), 2.62 (2H, m), 2.49 (2H, m).

### Example 100

### 3-Amino-1-(3-chloro-phenyl)-1-[4-(1H-pyrazol-4-yl)-phenyl]-propan-1-ol

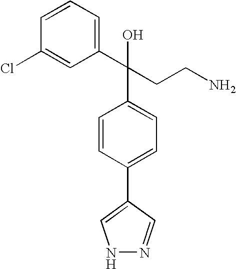

The title compound can be prepared according to methods described for Example 92, using (3-chloro-phenyl)-(4-iodo-phenyl)-methanone as starting material.

### Example 101

### 3-Amino-1-[4-(1H-pyrazol-4-yl)-phenyl]-1-(4-trifluoromethoxy-phenyl)-propan-1-ol

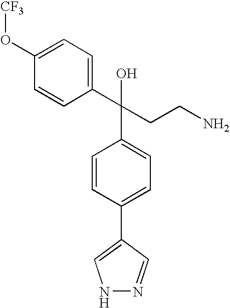

The title compound can be prepared according to methods described for Example 92, using 4-Bromo-4′-(trifluoromethoxy)benzophenone as starting material.

### Example 102

### 3-Amino-1-[4-(1H-pyrazol-4-yl)-phenyl]-1-(4-trifluoromethyl-phenyl)-propan-1-ol

### 102A. (4-Bromo-phenyl)-(4-trifluoromethyl-phenyl)-methanone

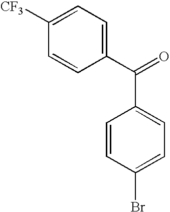

The title compound can be prepared from 4-trifluoromethyl-bromobenzene according to the methods described in Syn. Comm 1998, 28 (6), 1065-1072.

### 102B. 3-Amino-1-[4-(1H-pyrazol-4-yl)-phenyl]-1-(4-trifluoromethyl-phenyl)-propan-1-ol

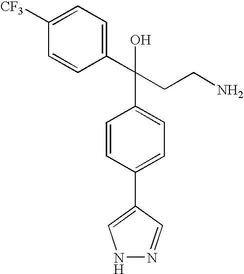

The title compound can be prepared from the product of Example 102A using methods described in Example 92.

### Example 103

### 3-Amino-1-(4-chloro-2-fluoro-phenyl)-1-[4-(1H-pyrazol-4-yl)-phenyl]-propan-1-ol

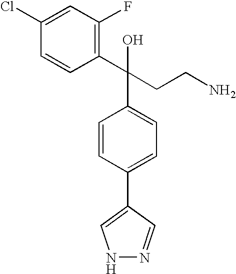

The title compound can be prepared using the methods described in Example 94, but by replacing 4-bromo-2-fluoro-benzoyl chloride with 4-chloro-2-fluorobenzoyl chloride, and chlorobenzene with bromobenzene.

### Example 104

### 3-Amino-1-(5-chloro-pyridin-2-yl)1-[4-(1H-pyrazol-4-yl)-phenyl]-propan-1-ol

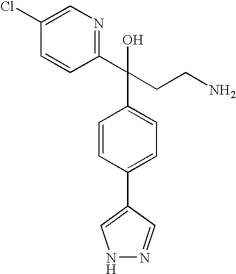

The title compound can be prepared using the methods described in Example 91A, but replacing

4-chlorophenylmagnesium bromide with 4-bromophenyl-magnesium bromide (made freshly in situ), and 5-bromopyridine-2-carbonitrile with 5-chloro-2-cyanopyridine, followed by the methods in Examples 92A-C

### Example 105

### 3-Amino-1-(4-tert-butyl-phenyl)-1-[4-(1H-pyrazol-4-yl)-phenyl]-propan-1-ol

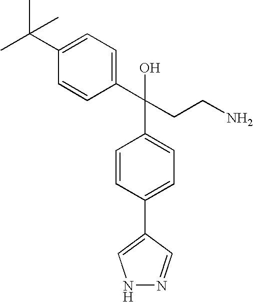

The title compound can be prepared according to methods described for Example 92, using 4-bromo-4′-tert-butyl benzophenone as starting material.

### Example 106

### 3-Amino-1-(4-chloro-3-morpholin-4-yl-phenyl)-1-[4-(1H-pyrazol-4-yl)-phenyl]-propan-1-ol

### 106A. (4-Bromo-phenyl)-(4-chloro-3-morpholin-4-yl-phenyl)-methanol

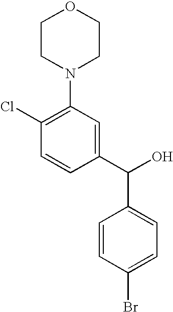

The title compound can be prepared by addition of 4-bromophenylmagnesium bromide (made freshly in situ) to the compound described in example 6B.

### 106B. (4-Bromo-phenyl)-(4-chloro-3-morpholin-4-yl-phenyl)-methanone

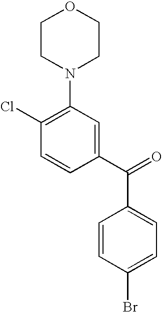

The title compound can be prepared by oxidation of the compound described in example 106A with Manganese dioxide.

### 106C. 3-Amino-1-(4-chloro-3-morpholin-4-yl-phenyl)-1-[4-(1H-pyrazol-4-yl)-phenyl]propan-1-ol

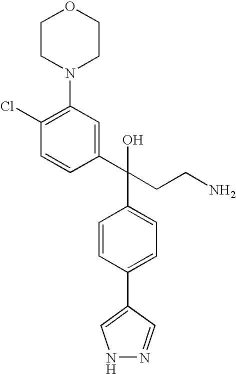

The title compound can be prepared from the compound described in example 106B using the procedures described in Example 92.

### Example 107

### 3-Amino-1-(5-chloro-thiophen-2-yl)1-[4-(1H-pyrazol-4-yl)-phenyl]-propan-1-ol

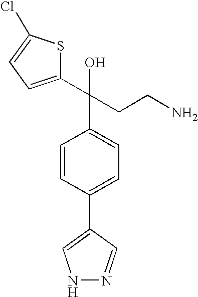

The title compound can be prepared using the method described in Example 91A, but by replacing 4-chlorophenylmagnesium bromide with 5-chloro-2-thienylmagnesium bromide, and 5-bromopyridine-2-carbonitrile with 4-bromobenzonitrile, followed by the methods in Examples 92A-C.

### Example 108

### 3-Amino-1-(4-chloro-phenyl)-1-[6-(1H-pyrazol-4-yl)-pyridin-3-yl]-propan-1-ol

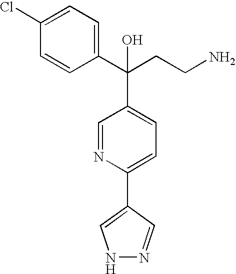

The title compound can be prepared using the methods described in Example 91A, but by replacing 5-bromopyridine-2-carbonitrile with 3-bromo-5-cyanopyridine, followed by the methods in Examples 92A-C

### Example 109

### 3-Amino-1-(4-chloro-phenyl)-1-[3-fluoro-4-(1H-pyrazol-4-yl)-phenyl]-propan-1-ol

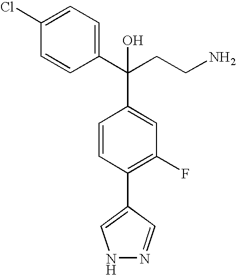

The title compound can be prepared using the methods described in Example 91A, but by replacing 5-bromopyridine-2-carbonitrile with 4-bromo-2-methylbenzonitrile, followed by the methods in Examples 92A-C.

### Example 110

### 3-Amino-1-(4-chloro-phenyl)-1-[2-methyl-4-(1H-pyrazol-4-yl)-phenyl]-propan-1-ol

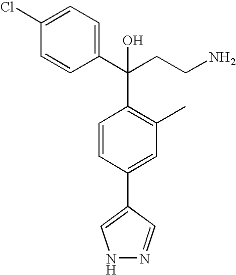

The title compound can be prepared using the methods described in Example 91A, but by replacing 5-bromopyridine-2-carbonitrile with 4-bromo-3-fluorobenzonitrile, followed by the methods in Examples 92A-C.

### Example 111

### 4-Amino-1-(4-chloro-phenyl)-1-[4-(1H-pyrazol-4-yl)-phenyl]-butan-1-ol

### 111A. N-[4-(4-Bromo-phenyl)-4-(4-chloro-phenyl)-4-hydroxy-butyl]-benzamide

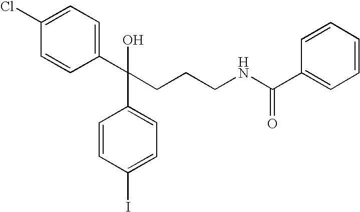

The title compound can be prepared from (4-chloro-phenyl)-(4-iodo-phenyl)-methanone using the method described in Tetrahedron 1989 2183

### 111B. 4-Amino-1-(4-chloro-phenyl)-1-[4-(1H-pyrazol-4-yl)-phenyl]-butan-1-ol

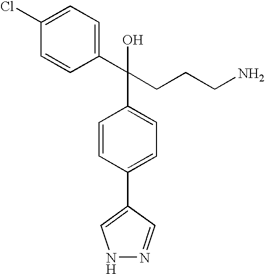

The title compound can be prepared from the compound described in Example 111A using the basic hydrolysis method described in Tetrahedron 1989 2183.

### Example 112

### 1-(4-Chloro-phenyl)-3-methylamino-1-[4-(1H-pyrazol-4-yl)-phenyl]-propan-1-ol

### 112A. 3-(4-Chloro-phenyl)-3-hydroxy-3-(4-iodo-phenyl)-propionic acid

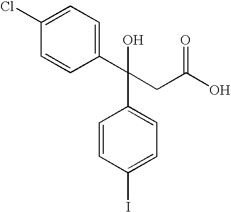

The title compound can be prepared from (4-chloro-phenyl)-(4-iodo-phenyl)-methanone using the method described in J. Am. Chem. Soc. 1950 1522.

### 112B. 1-(4-Chloro-phenyl)-3-methylamino-1-[4-(1H-pyrazol-4-yl)-phenyl]-propan-1-ol

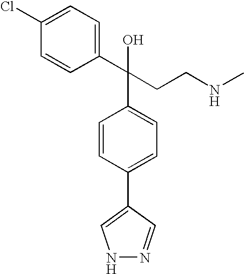

The title compound can be prepared from the compound described in Example 112A using the methods described in General Procedures 3D to 3F.

### Example 113

### Azetidin-3-yl-(4-chlorophenyl)-[4-(1H-pyrazol-4-yl)-phenyl]-methanol

### 113A. 3-(4-Chloro-benzoyl)-azetidine-1-carboxylic acid tert-butyl ester

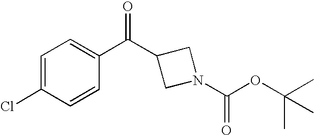

The title compound can be prepared using the method described in international patent application WO 2005/026113

### 113B. 3-[(4-Bromo-phenyl)-(4-chloro-phenyl)-hydroxy-methyl]-azetidine-1-carboxylic acid tert-butyl ester

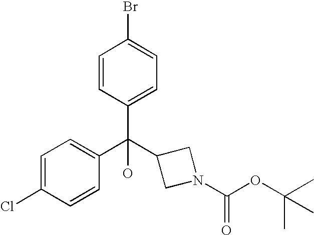

The title compound can be made by the addition of 4-bromophenylmagnesium bromide to the compound described in Example 113A.

### 113C. Azetidin-3-yl-(4-chlorophenyl)-[4-(1H-pyrazol-4-yl)-phenyl]-methanol

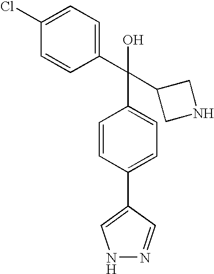

The title compound can be made from the compound described in Example 113B using General Procedure 1.

### Example 114

### 3-Amino-1-(4-chloro-phenyl)-3-methyl-1-[4-(1H-pyrazol-4-yl)-phenyl]-butan-1-ol

### 114A. 3-Amino-1-(4-chloro-phenyl)-1-(4-iodo-phenyl)-3-methyl-butan-1-ol

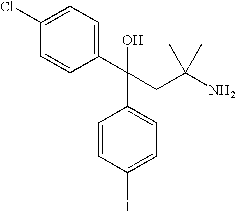

The title compound can be prepared by treatment of the compound described in Example 92B with methylmagnesium bromide.

### 114B. 3-Amino-1-(4-chloro-phenyl)-3-methyl-1-[4-(1H-pyrazol-4-yl)-phenyl]-butan-1-ol

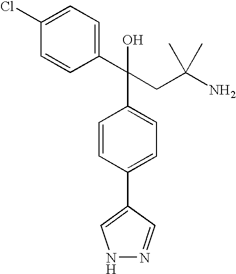

The title compound can be prepared from the compound described in Example 114A using General Procedure 1.

### Example 115

### 3-Amino-1-(4-chloro-phenyl)-1-[4-(3-methyl-1H-pyrazol-4-yl)-phenyl]-propan-1-ol

### 115A. 4-{4-[1-(4-Chloro-phenyl)-2-cyano-1-hydroxy-ethyl]-phenyl}-3-methyl-pyrazole-1-sulphonic acid dimethylamide

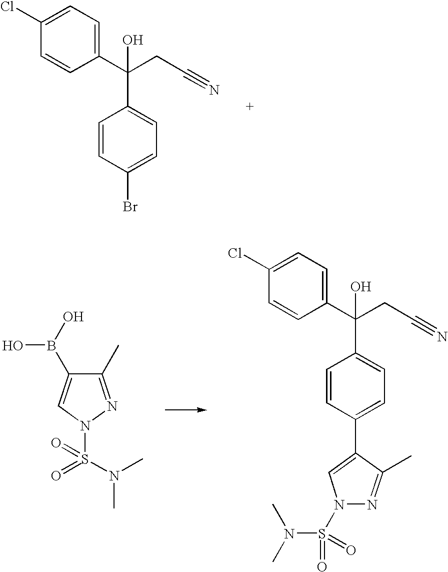

Using the method described in Example 87D, 3-methyl-1-sulphonic acid dimethylamide-pyrazole boronic acid (Example 77J) and 3-(4-chloro-phenyl)-3-hydroxy-3-(4-iodo-phenyl)-propionitrile (Example 92A) would be reacted together to produce the title compound.

### 115B. 3-(4-Chloro-phenyl)-3-hydroxy-3-[4-(3-methyl-1H-pyrazol-4-yl)-phenyl]-propionitrile

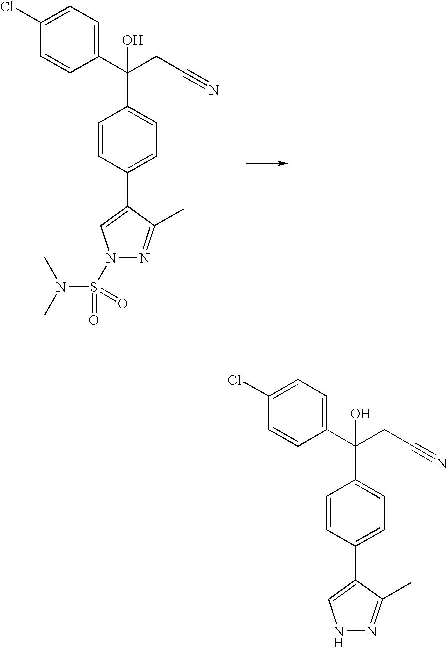

Using 4-{-4-[1-(4-chloro-phenyl)-2-cyano-1-hydroxy-ethyl]-phenyl}-3-methyl-pyrazole-1-sulphonic acid dimethylamide (Example 115A) the title compound could be prepared according to the procedure described in Example 77L.

### 115C. 3-Amino-1-(4-chloro-phenyl)-1-[4-(3-methyl-1H-pyrazol-4-yl)-phenyl]-propan-1-ol

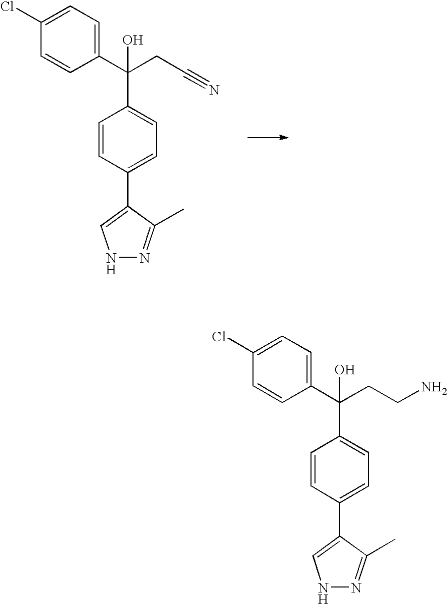

Using 3-(4-chloro-phenyl)-3-hydroxy-3-[4-(3-methyl-1H-pyrazol-4-yl)-phenyl]-propionitrile (Example 115B) the title compound could be prepared according to methods described for Example 92C.

### Example 116

### 3-Amino-1-(4-chloro-phenyl)-1-[4-(3-trifluoromethyl-1H-pyrazol-4-yl)-phenyl]-propan-1-ol

### 116A. 4-Iodo-3-trifluoromethyl-1H-pyrazole

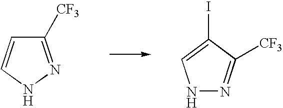

Dissolve 3-trifluoromethyl-1H-pyrazole (5.5 g, 36.7 mmoles, 1 equiv.) in acetonitrile (30 ml). Add ammonium ceric nitrate (11.1 g, 20.2 mmoles, 0.55 equiv.) and iodine (6.2 g, 24.6 mmoles, 0.67 equiv.) to the reaction mixture. Heat the reaction mixture at 75° C. for one hour, cool to ambient temperature and then partitioned between ethyl acetate and a saturated solution of sodium thiosulphate. Wash the organic layer with water and then a saturated brine solution. Dry the organic layer (MgSO₄), filter, and concentrate in vacuo to yield the title compound.

### 116B. 4-Iodo-3-trifluoromethyl-pyrazole-1-sulphonic acid dimethylamide

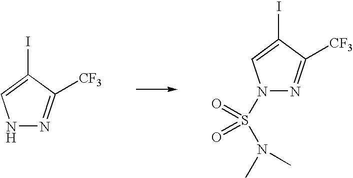

Using 4-iodo-3-trifluoromethyl-1H-pyrazole (Example 116A), the title compound would be prepared according to the procedure described in Example 77I.

### 116C. 3-Trifluoromethyl-1-sulphonic acid dimethylamide-pyrazole-4-boronic acid

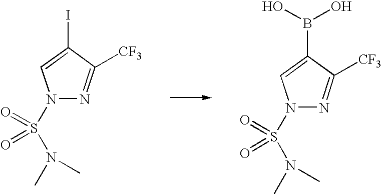

Using 4-iodo-3-trifluoromethyl-pyrazole-1-sulphonic acid dimethylamide (Example 2B), the title compound would be prepared according to the procedure described in Example 77J.

### 116D. 4-{4-[1-(4-chloro-phenyl)-2-cyano-1-hydroxy-ethyl]-phenyl}-3-trifluoromethyl-pyrazole-1-sulphonic acid dimethylamide

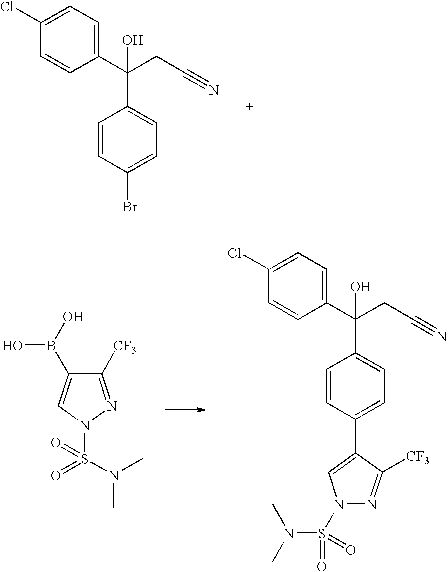

Using the method described in Example 87D, 3-trifluoromethyl-1-sulphonic acid dimethylamide-pyrazole-4-boronic acid (Example 116C) and 3-(4-chloro-phenyl)-3-hydroxy-3-(4-iodo-phenyl)-propionitrile (Example 92A) would be reacted together to produce the title compound.

### 116E. 3-(4-Chloro-phenyl)-3-hydroxy-3-[4-(3-trifluoromethyl-1H-pyrazol-4-yl)-phenyl]-propionitrile

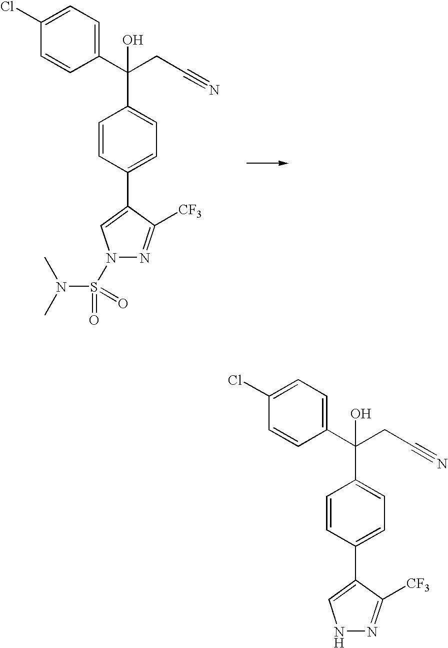

Using 4-{4-[1-(4-chloro-phenyl)-2-cyano-1-hydroxy-ethyl]-phenyl}-3-trifluoromethyl-pyrazole-1-sulphonic acid dimethylamide (Example 116D), the title compound would be prepared according to the procedure described in Example 77L.

### 116F. 3-Amino-1-(4-chloro-phenyl)-1-[4-(3-trifluoromethyl-1H-pyrazol-4-yl)-phenyl]-propan-1-ol

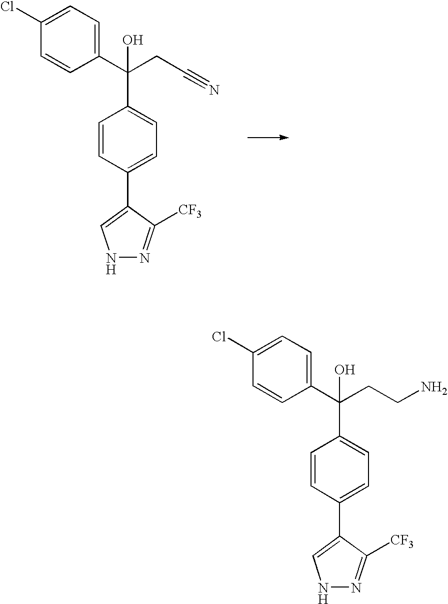

Using 3-(4-chloro-phenyl)-3-hydroxy-3-[4-(3-trifluoromethyl-1H-pyrazol-4-yl)-phenyl]-propionitrile (Example 116E), the title compound would be prepared according to methods described for Example 92C.

### Example 117

### 3-(4-Chloro-phenyl)-3-methoxy-3-[4-(1H-pyrazol-4-yl)-phenyl]-propylamine

### 117A. 3-(4-Chloro-phenyl)-3-hydroxy-3-(4-iodo-phenyl)-propionitrile

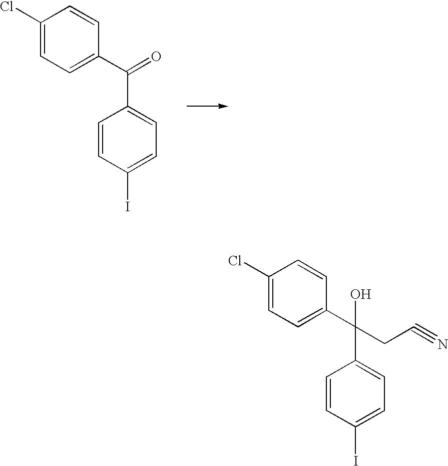

3-(4-Chloro-phenyl)-3-hydroxy-3-(4-iodo-phenyl)-propionitrile can be prepared from (4-chloro-phenyl)-(4-iodo-phenyl)-methanone according to the method described in Example 92A.

### 117B. 3-(4-Chloro-phenyl)-3-(4-iodo-phenyl)-3-methoxy-propionitrile

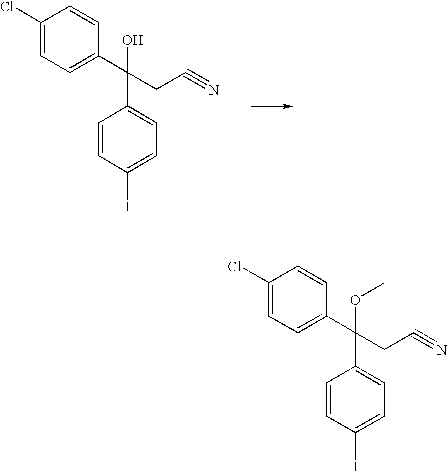

3-(4-Chloro-phenyl)-3-(4-iodo-phenyl)-3-methoxy-propionitrile can be prepared from 3-(4-chloro-phenyl)-3-hydroxy-3-(4-iodo-phenyl)-propionitrile using 1 equivalent of sodium hydride followed by methyl iodide in a suitable anhydrous organic solvent e.g. tetrahydrofuran (see the method described in Journal of Organic Chemistry (2002), 67(22), 7769-7773).

### 117C. 3-(4-Chloro-phenyl)-3-methoxy-3-[4-(1H-pyrazol-4-yl)-phenyl]-propionitrile

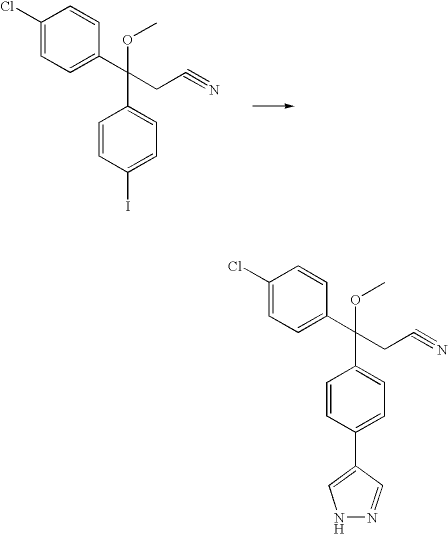

The title compound can be prepared from 3-(4-chloro-phenyl)-3-(4-iodo-phenyl)-3-methoxy-propionitrile according to the General Procedure 1.

### 117D. 3-(4-Chloro-phenyl)-3-methoxy-3-[4-(1H-pyrazol-4-yl)-phenyl]-propylamine

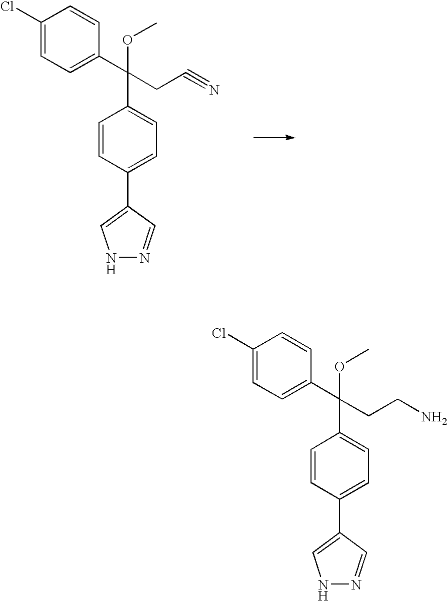

The title compound can be prepared from (4-chloro-phenyl)-3-methoxy-3-[4-(1H-pyrazol-4-yl)-phenyl]-propionitrile according to the method described in Example 92C.

### Example 118

### N*1*-{(4-Chloro-phenyl)-[4-(1H-pyrazol-4-yl)-phenyl]-methyl}-ethane-1,2-diamine

### 118A. (2-{[(4-Chloro-phenyl)-(4-iodo-phenyl)-methyl]-amino}-ethyl)-carbamic acid tert-butyl ester

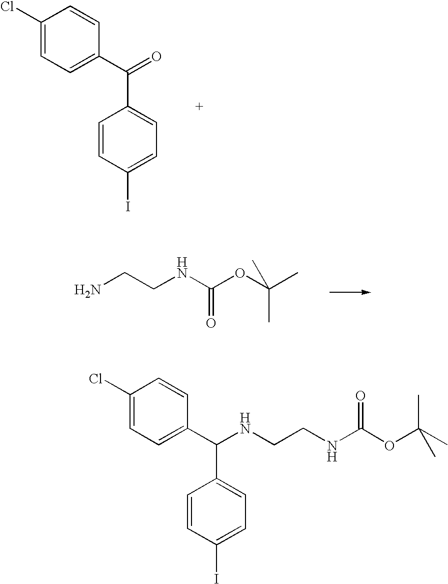

(2-{[(4-Chloro-phenyl)-(4-iodo-phenyl)-methyl]-amino}-ethyl)-carbamic acid tert-butyl ester can be prepared from (4-chloro-phenyl)-(4-iodo-phenyl)-methanone using (2-Amino-ethyl)-carbamic acid tert-butyl ester and sodium cyanoborohydride in a suitable organic solvent e.g. methanol/acetic acid (refer to the method described in Journal of Organic Chemistry (2003), 68(1), 92-103).

### 118B. [2-({(4-Chloro-phenyl)-[4-(1H-pyrazol-4-yl)-phenyl]-methyl}-amino)-ethyl]-carbamic acid tert-butyl ester

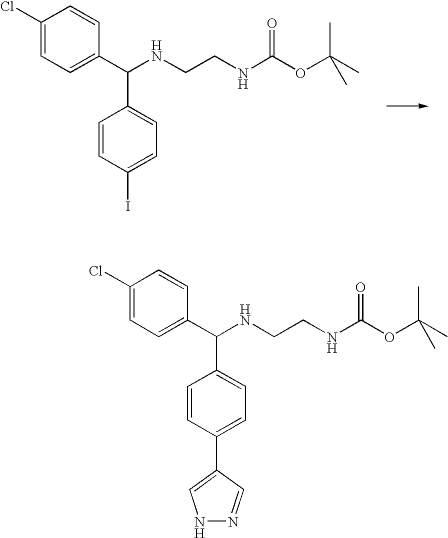

The title compound can be prepared from (2-{[(4-Chloro-phenyl)-(4-iodo-phenyl)-methyl]-amino}-ethyl)-carbamic acid tert-butyl ester according to the General Procedure 1.

### 118C. N*1*-{(4-Chloro-phenyl)-[4-(1H-pyrazol-4-yl)-phenyl]-methyl}-ethane-1,2-diamine

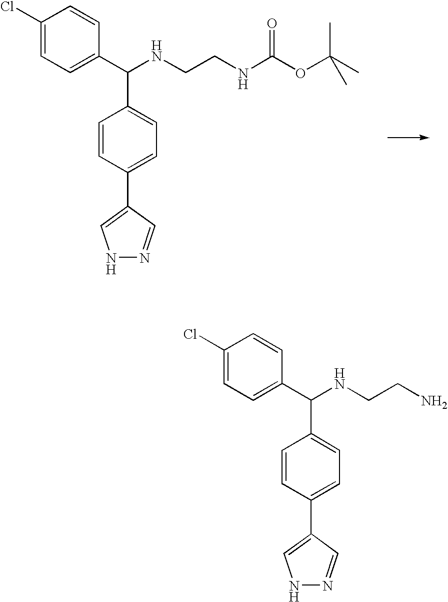

The title compound can be prepared from [2-({(4-chloro-phenyl)-[4-(1H-pyrazol-4-yl)-phenyl]-methyl}-amino)-ethyl]-carbamic acid tert-butyl ester using a saturated solution of HCl in diethyl ether with a co-solvent e.g. methanol.

### Biological Activity

### Example 119

### Measurement of PKA Kinase Inhibitory Activity (IC₅₀)

Compounds of the invention can be tested for PK inhibitory activity using the PKA catalytic domain from Upstate Biotechnology (#14-440) and the 9 residue PKA specific peptide (GRTGRRNSI), also from Upstate Biotechnology (#12-257), as the substrate. A final concentration of 1 nM enzyme is used in a buffer that includes 20 mM MOPS pH 7.2, 40 μM ATP/γ³³P-ATP and 5 μM substrate. Compounds are added in dimethylsulphoxide (DMSO) solution to a final DMSO concentration of 2.5%. The reaction is allowed to proceed for 20 minutes before addition of excess orthophosphoric acid to quench activity. Unincorporated γ³³P-ATP is then separated from phosphorylated proteins on a Millipore MAPH filter plate. The plates are washed, scintillant is added and the plates are then subjected to counting on a Packard Topcount.

The % inhibition of the PKA activity is calculated and plotted in order to determine the concentration of test compound required to inhibit 50% of the PKA activity (IC₅₀).

The compounds of Examples 77, 78, 81, 82, 87, 88 and 89 have IC₅₀ values of less than 1 μM in the above assay whereas the compounds of Examples 1, 79, 80, 83, 84, 85, 86, 90, 91, 92, 94, 95, 96 and 99A have IC₅₀ values of less than 0.1 μM.

### Example 120

### Measurement of PKB Kinase Inhibitory Activity (IC₅₀)

The inhibition of protein kinase B (PKB) activity by compounds can be determined essentially as described by Andjelkovic et al. (Mol. Cell. Biol. 19, 5061-5072 (1999)) but using a fusion protein described as PKB-PIF and described in full by Yang et al (Nature Structural Biology 9, 940-944 (2002)). The protein is purified and activated with PDK1 as described by Yang et al. The peptide AKTide-2T (H-A-R-K-R-E-R-T-Y-S-F-G-H-H-A-OH) obtained from Calbiochem (#123900) is used as a substrate. A final concentration of 0.6 nM enzyme is used in a buffer that includes 20 mM MOPS pH 7.2, 30 μM ATP/γ³³P-ATP and 25 μM substrate. Compounds are added in DMSO solution to a final DMSO concentration of 2.5%. The reaction is allowed to proceed for 20 minutes before addition of excess orthophosphoric acid to quench activity. The reaction mixture is transferred to a phosphocellulose filter plate where the peptide binds and the unused ATP is washed away. After washing, scintillant is added and the incorporated activity measured by scintillation counting.

The % inhibition of the PKB activity is calculated and plotted in order to determine the concentration of test compound required to inhibit 50% of the PKB activity (IC₅₀).

The compounds of Examples 77, 78, 81, 82, 85, 87, 89, 90 and 91 have IC₅₀ values of less than 1 μM in the above assay whereas the compounds of Examples 1, 2, 79, 80, 83, 84, 86, 88, 92, 93, 94, 95, 96, 99A and 99B have IC₅₀ values of less than 0.1 μM.

### Pharmaceutical Formulations

### Example 121

### (i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

### (ii) Capsule Formulation

A capsule formulation is prepared by mixing 100 mg of a compound of the formula (I) with 100 mg lactose and filling the resulting mixture into standard opaque hard gelatin capsules.

### (iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

### (iv) Injectable Formulation II

A parenteral composition for injection is prepared by dissolving in water a compound of the formula (I) (e.g. in salt form) (2 mg/ml) and mannitol (50 mg/ml), sterile filtering the solution and filling into sealable 1 ml vials or ampoules.

### (v) Injectable Formulation III

A formulation for i.v. delivery by injection or infusion can be prepared by dissolving the compound of formula (I) (e.g. in a salt form) in water at 20 mg/ml. The vial is then sealed and sterilised by autoclaving.

### (vi) Injectable Formulation IV

A formulation for i.v. delivery by injection or infusion can be prepared by dissolving the compound of formula (I) (e.g. in a salt form) in water containing a buffer (e.g. 0.2 M acetate pH 4.6) at 20 mg/ml. The vial is then sealed and sterilised by autoclaving.

### (vii) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

### (viii) Lyophilised Formulation

Aliquots of formulated compound of formula (I) are put into 50 ml vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1. A compound of the formula (II): 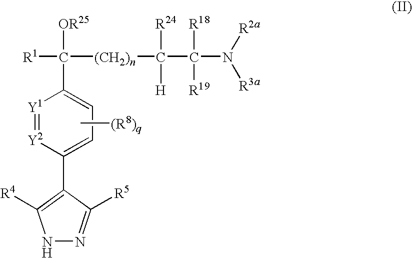
or a salt, solvate, tautomer or N-oxide thereof; wherein
n is 0 or 1;
one of Y¹ and Y² is CH and the other is selected from CH, CR⁸ and N;
q is 0, 1 or 2 provided that q is 0 or 1 when Y¹ or Y² is CR⁸;
R¹ is an aryl or heteroaryl group of 5 to 10 ring members;
R^{2a} and R^{3a} are independently selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy;
or R^{2a} and R^{3a} together with the nitrogen atom to which they are attached form a cyclic group selected from an imidazole group and a saturated monocyclic heterocyclic group having 4-7 ring members and optionally containing a second heteroatom ring member selected from O and N;
R¹⁸ is selected from hydrogen and methyl;
R¹⁹ is selected from hydrogen and methyl;
R²⁴ is hydrogen or R²⁴, R^{2a} and the intervening nitrogen atom and carbon atoms together form an azetidine, pyrrolidine or piperidine ring; and
R²⁵ is selected from hydrogen or a C₁₋₄ alkyl group wherein the C₁₋₄ alkyl group is optionally substituted by hydroxy or amino provided that there are at least two carbon atoms between the hydroxy or amino group and the oxygen atom to which R²⁵ is attached;
R⁴ is selected from hydrogen, halogen, C₁₋₅ saturated hydrocarbyl, C₁₋₅ saturated hydrocarbyloxy, cyano, and CF₃; and
R⁵ is selected from selected from hydrogen, halogen, C₁₋₅ saturated hydrocarbyl, C₁₋₅ saturated hydrocarbyloxy, cyano, CONH₂, CONHR⁹, CF₃, NH₂, NHCOR⁹ or NHCONHR⁹;
R⁸ is selected from hydroxy; halogen; trifluoromethyl; cyano; C₁₋₄ hydrocarbyloxy optionally substituted by C₁₋₂ alkoxy or hydroxy; and C₁₋₄ hydrocarbyl optionally substituted by C₁₋₂ alkoxy or hydroxy;
R⁹ is a group R^{9a} or (CH₂)R^{9a}, wherein R^{9a} is a monocyclic or bicyclic group which may be carbocyclic or heterocyclic;
the carbocyclic group or heterocyclic group R^{9a} being optionally substituted by one or more substituents selected from fluorine, chlorine or a group R¹³;
R¹³ is selected from halogen (other than fluorine or chlorine), hydroxy, trifluoromethyl, cyano, nitro, carboxy, amino, mono- or di-C₁₋₄ hydrocarbylamino; a group R^{a}—R^{b} wherein R^{a} is a bond, O, CO, X¹C(X²), C(X²)X¹, X¹C(X²)X¹, S, SO, SO₂, NR^{c}, SO₂NR^{e} or NR^{o}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; provided that R^{b} is other than hydrogen when R^{a} is a bond;
R^{o} is selected from hydrogen and C₁₋₄ hydrocarbyl; and
X¹ is O, S or Me and X² is ═O, ═S or ═NR^{c}.

2. A compound according to claim 1 wherein R²⁵ is hydrogen, methyl, 2-hydroxyethyl or 2-aminoethyl.

3. A compound according to claim 2 wherein R²⁵ is hydrogen or methyl.

4. A compound according to claim 3 wherein R²⁵ is hydrogen.

5. A compound according to claim 3 wherein R²⁵ is methyl.

6. A compound according to any one of the preceding claims wherein R²⁴ is hydrogen.

7. A compound according to any one of claims 1 to 5 wherein R²⁴, R^{2a} and the intervening nitrogen atom and carbon atoms together form an azetidine, pyrrolidine or piperidine ring.

8. A compound according to claim 7 wherein R²⁴, R^{2a} and the intervening nitrogen atom and carbon atoms together form an azetidine ring.

9. A compound according to any one of the preceding claims wherein n is 0.

10. A compound according to any one of claims 1 to 8 wherein n is 1.

11. A compound according to any one of claims 1 to 10 wherein R¹⁸ is hydrogen.

12. A compound according to any one of claims 1 to 10 wherein R¹⁸ is methyl.

13. A compound according to any one of claims 1 to 12 wherein R¹⁹ is hydrogen.

14. A compound according to any one of claims 1 to 12 wherein R¹⁹ is methyl.

15. A compound according to claim 1 wherein the moiety: 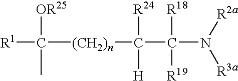
is selected from groups D1 to D5:

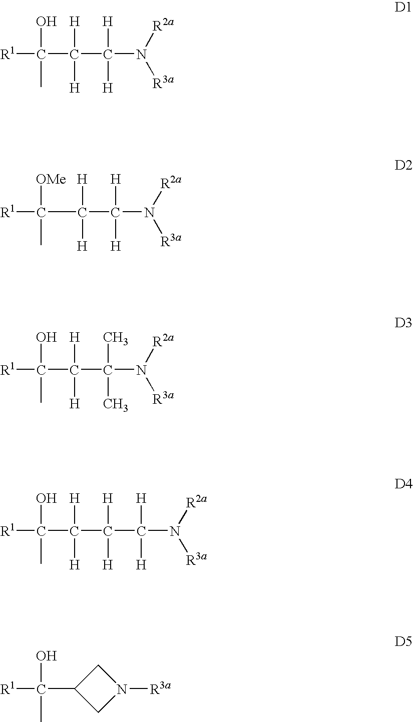

16. A compound according to any one of claims 1 to 15 wherein Y¹ is CH.

17. A compound according to any one of claims 1 to 16 wherein Y² is CH.

18. A compound according to claim 16 wherein Y² is CR⁸ or N.

19. A compound according to claim 18 wherein Y² is N.

20. A compound according to claim 18 wherein Y² is CF.

21. A compound according to any one of claims 1 to 15 wherein Y² is CH and Y¹ is CR⁸ or N.

22. A compound according to claim 21 wherein Y¹ is N.

23. A compound according to claim 21 wherein Y¹ is C-Me or CF.

24. A compound according to claim 21 wherein Y¹ is C-Me.

25. A compound according to claim 1 having the general formula (IIa): 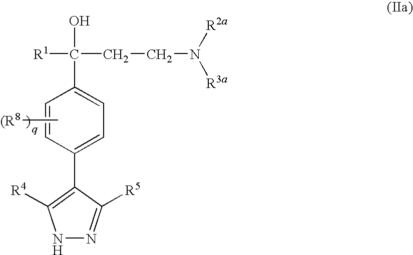
wherein R¹, R^{2a}, R^{3a}, R⁴, R⁵, and R⁸ are each as defined in any one of the preceding claims and q is 0, 1 or 2 (preferably 0 or 1).

26. A compound according to any one of the preceding claims wherein R⁴ is selected from hydrogen, halogen, C₁₋₅ saturated hydrocarbyl, C₁₋₅ saturated hydrocarbyloxy, cyano, and CF₃.

27. A compound according to claim 26 wherein R⁴ is selected from hydrogen, halogen, C₁₋₅ saturated hydrocarbyl, cyano and CF₃.

28. A compound according to claim 27 wherein R⁴ is selected from hydrogen, methyl and trifluoromethyl.

29. A compound according to claim 28 wherein R⁴ is selected from hydrogen and methyl.

30. A compound according to claim 29 wherein R⁴ is hydrogen.

31. A compound according to any one of the preceding claims wherein R⁵ is selected from hydrogen, methyl and cyano.

32. A compound according to claim 31 wherein R⁵ is hydrogen or methyl.

33. A compound according to claim 32 wherein R⁵ is hydrogen.

34. A compound according to any one of the preceding claims wherein e is selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy.

35. A compound according to claim 34 wherein R^{2a} is selected from hydrogen, C₁₋₄ hydrocarbyl and C₁₋₄ acyl.

36. A compound according to claim 35 wherein R^{2a} is selected from hydrogen and methyl.

37. A compound according to claim 36 wherein R^{2a} is hydrogen.

38. A compound according to claim 36 wherein R^{2a} is methyl.

39. A compound according to any one of the preceding claims wherein R^{3a} is selected from hydrogen, C₁₋₄ hydrocarbyl and C₁₋₄ acyl wherein the hydrocarbyl and acyl moieties are optionally substituted by one or more substituents selected from fluorine, hydroxy, amino, methylamino, dimethylamino and methoxy.

40. A compound according to claim 39 wherein R^{3a} is selected from hydrogen, C₁₋₄ hydrocarbyl and C₁₋₄ acyl.

41. A compound according to claim 40 wherein R^{3a} is selected from hydrogen and methyl.

42. A compound according to claim 41 wherein R^{3a} is hydrogen.

43. A compound according to claim 41 wherein R^{3a} is methyl.

44. A compound according to any one of claims 1 to 33 wherein NR^{2a}R^{3a} is an amino group or a methylamino group.

45. A compound according to any one of claims 1 to 34 wherein e and R^{2a} and R^{3a} together with the nitrogen atom to which they are attached form a cyclic group selected from an imidazole group and a saturated monocyclic heterocyclic group having 4-7 ring members and optionally containing a second heteroatom ring member selected from O and N.

46. A compound according to any one of the preceding claims wherein R¹ is a monocyclic aryl or heteroaryl group selected from six membered aryl and heteroaryl groups containing up to 2 nitrogen ring members, and five membered heteroaryl groups containing up to 3 heteroatom ring members selected from O, S and N, and the monocyclic aryl or heteroaryl group is optionally substituted by one or more substituents selected from hydroxy; C₁₋₄ acyloxy; fluorine; chlorine; bromine; trifluoromethyl; cyano; CONH₂; nitro; C₁₋₄ hydrocarbyloxy and C₁₋₄ hydrocarbyl each optionally substituted by C₁₋₂ alkoxy, carboxy or hydroxy; C₁₋₄ acylamino; benzoylamino; pyrrolidino; piperidino; morpholino; piperazine; N-methylpiperazino; pyrrolidinocarbonyl; piperidinocarbonyl; morpholinocarbonyl; piperazinocarbonyl; five and six membered heteroaryl and heteroaryloxy groups containing one or two heteroatoms selected from N, O and S; phenyl; phenyl-C₁₋₄ alkyl; phenyl-C₁₋₄ alkoxy; heteroaryl-C₁₋₄ alkyl; heteroaryl-C₁₋₄ alkoxy and phenoxy, wherein the heteroaryl, heteroaryloxy, phenyl, phenyl-C₁₋₄ alkyl, phenyl-C₁₋₄ alkoxy, heteroaryl-C₁₋₄ alkyl, heteroaryl-C₁₋₄ alkoxy and phenoxy groups are each optionally substituted with 1, 2 or 3 substituents selected from C₁₋₂ acyloxy, fluorine, chlorine, bromine, trifluoromethyl, cyano, CONH₂, C₁₋₂ hydrocarbyloxy and C₁₋₂ hydrocarbyl each optionally substituted by methoxy or hydroxy.

47. A compound according to claim 46 wherein R¹ is an optionally substituted monocyclic aryl or heteroaryl group selected from phenyl, thienyl, furan, pyrimidine and pyridine.

48. A compound according to claim 47 wherein R¹ is an optionally substituted monocyclic aryl or heteroaryl group selected from phenyl, thienyl and pyridine.

49. A compound according to claim 48 wherein R¹ is an optionally substituted phenyl group.

50. A compound according to claim 46 wherein R¹ is a phenyl group optionally substituted by a substituent selected from hydroxy; C₁₋₄ acyloxy; fluorine; chlorine; bromine; trifluoromethyl; cyano; piperidino; morpholino; piperazino; N-methylpiperazino; and C₁₋₄ hydrocarbyloxy and C₁₋₄ hydrocarbyl each optionally substituted by C₁₋₂ alkoxy or hydroxy.

51. A compound according to claim 46 wherein R¹ is a phenyl group substituted by one or two substituents selected from fluorine; chlorine; methyl, methoxy and trifluoromethyl.

52. A compound according to claim 50 wherein R¹ is selected from mono-chlorophenyl (e.g. 4-chlorophenyl or 3-chlorophenyl), trifluoromethylphenyl (e.g. 4-trifluoromethylphenyl), trifluoromethoxyphenyl (e.g. 4-trifluoro-methoxyphenyl), tert-butylphenyl (e.g. 4-tert-butylphenyl), dichlorophenyl (e.g. 3,4-dichlorophenyl), fluoro-chlorophenyl (e.g. 2-fluoro-4-chlorophenyl and 3-fluoro-4-chlorophenyl), and 4-chloro-3-(4-morpholinyl)-phenyl groups.

53. A compound according to claim 52 wherein R¹ is selected from mono-chlorophenyl (e.g. 4-chlorophenyl), dichlorophenyl (e.g. 3,4-dichlorophenyl) and fluoro-chlorophenyl (e.g. 3-fluoro-4-chlorophenyl) groups.

54. A compound according to claim 53 wherein R¹ is a 4-chlorophenyl, 3,4-dichlorophenyl or 3-fluoro-4-chlorophenyl group.

55. A compound according to claim 46 wherein R¹ is a heteroaryl group selected from pyridine and thiophene groups that bear a substituent selected from fluorine, chlorine, C₁₋₄ alkyl (e.g. tert-butyl), C₁₋₄ alkoxy (e.g. methoxy), trifluoromethyl, trifluoromethoxy, difluoromethoxy and 5-6 membered saturated heterocyclic rings containing a nitrogen ring member and optionally a second heteroatom ring member selected from O and N.

56. A compound according to claim 55 wherein R¹ is 5-chloro-2-thienyl or 5-chloro-2-pyridyl groups.

57. A compound according to any one of the preceding claims wherein R⁸ is selected from hydroxy; halogen (e.g. fluorine, chlorine and bromine); trifluoromethyl; cyano; C₁₋₄ alkoxy optionally substituted by C₁₋₂ alkoxy or hydroxy; and C₁₋₄ alkyl optionally substituted by C₁₋₂ alkoxy or hydroxy.

58. A compound according to any one of claims 1 to 56 wherein R⁸ is absent (q=0) or is selected from methyl, fluorine, chlorine, methoxy, trifluoromethyl and cyano.

59. A compound according to claim 58 wherein R⁸ is absent (q=0) or is a methyl group or fluorine atom.

60. A compound according to claim 59 wherein R⁸ is absent (q=0) or is a fluorine atom.

61. A compound according to claim 60 wherein q is 0.

62. A compound according to any one of the preceding claims wherein R⁴ and R⁵ are each selected from hydrogen and saturated C₁₋₄ hydrocarbyl optionally substituted by one or more fluorine atoms.

63. A compound according to claim 1 having the formula (IIb): 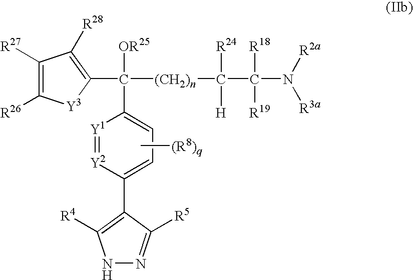
or a salt, solvate, tautomer or N-oxide thereof; wherein
n is 0 or 1;
one of Y¹ and Y² is CH and the other is selected from CH and N;
Y³ is CH═CH, CH═N or S;
q is 0 or 1;
R^{2a} is hydrogen or methyl;
R^{3a} is hydrogen or methyl;
R⁸ is fluorine or methyl;
R¹⁸ is hydrogen or methyl;
R¹⁹ is hydrogen or methyl;
R²⁴ is hydrogen or R²⁴, R^{2a} and the intervening nitrogen atom and carbon atoms together form an azetidine, pyrrolidine or piperidine ring; and
R²⁵ is hydrogen or methyl provided that there are at least two carbon atoms between the hydroxy or amino group and the oxygen atom to which R²⁵ is attached;
one of R⁴ and R⁵ is hydrogen and the other is hydrogen, methyl or trifluoromethyl;
R²⁶ is hydrogen, chlorine, fluorine, trifluoromethyl, difluoromethoxy, trifluoromethoxy, C₁₋₄ alkyl or C₁₋₃ alkoxy;
R²⁷ is hydrogen, chlorine, fluorine, trifluoromethyl, difluoromethoxy, trifluoromethoxy, C₁₋₄ alkyl, C₁₋₃ alkoxy, morpholinyl, pyrrolidinyl, piperidinyl or piperazinyl; and
R²⁸ is hydrogen or fluorine; provided that no more than 2 of R²⁶, R²⁷ and R²⁸ are other than hydrogen.

64. A compound according to claim 63 having the formula (IIc): 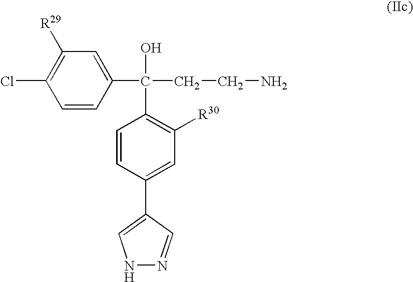
or a salt, solvate or tautomer thereof; wherein R²⁹ is hydrogen, chlorine or fluorine and R³⁰ is hydrogen or fluorine.

65. A compound according to claim 64 wherein R²⁹ is hydrogen and R³⁰ is hydrogen.

66. A compound according to claim 1 which is:
3-amino-1-(4-chloro-phenyl)-1-[4-(1H-pyrazol-4-yl)-phenyl]-propan-1-ol;
3-amino-1-(4-chlorophenyl)-1-[2-fluoro-4-(1H-pyrazol-4-yl)-phenyl]-propan-1-ol;
3-amino-1-(4-chloro-3-fluoro-phenyl)-1-[4-(1H-pyrazol-4-yl)-phenyl]-propan-1-ol;
3-amino-1-(3,4,-dichlorophenyl)-1-[4-(1H-pyrazol-4-yl)-phenyl]-propan-1-ol;
(S)-3-amino-1-(4-chloro-phenyl)-1-[4-(1H-pyrazol-4-yl)-phenyl]-propan-1-ol;
(R)-3-amino-1-(4-chloro-phenyl)-1-[4-(1-pyrazol-4-yl)-phenyl]-propan-1-ol;
3-amino-1-(3-chloro-phenyl)-1-[4-(1H-pyrazol-4-yl)-phenyl]-propan-1-ol;
3-amino-1-[4-(1H-pyrazol-4-yl)-phenyl]-1-(4-trifluoromethoxy-phenyl)-propan-1-ol;
3-amino-1-[4-(1H-pyrazol-4-yl)-phenyl]-1-(4-trifluoromethyl-phenyl)-propan-1-ol;
3-amino-1-(4-chloro-2-fluoro-phenyl)-1-[4-(1H-pyrazol-4-yl)-phenyl]-propan-1-ol;
3-amino-1-(5-chloro-pyridin-2-yl)1-[4-(1H-pyrazol-4-yl)-phenyl]-propan-1-ol;
3-amino-1-(4-tert-butyl-phenyl)-1-[4-(1H-pyrazol-4-yl)-phenyl]-propan-1-ol;
3-amino-1-(4-chloro-3-morpholin-4-yl-phenyl)-1-[4-(1H-pyrazol-4-yl)-phenyl]-propan-1-ol;
3-amino-1-(5-chloro-thiophen-2-yl)1-[4-(1H-pyrazol-4-yl)-phenyl]-propan-1-ol;
3-amino-1-(4-chloro-phenyl)-1-[6-(1H-pyrazol-4-yl)-pyridin-3-yl]-propan-1-ol;
3-amino-1-(4-chloro-phenyl)-1-[3-fluoro-4-(1H-pyrazol-4-yl)-phenyl]-propan-1-ol;
3-amino-1-(4-chloro-phenyl)-1-[2-methyl-4-(1H-pyrazol-4-yl)-phenyl]-propan-1-ol;
4-amino-1-(4-chloro-phenyl)-1-[4-(1H-pyrazol-4-yl)-phenyl]-butan-1-ol;
1-(4-chloro-phenyl)-3-methylamino-1-[4-(1H-pyrazol-4-yl)-phenyl]-propan-1-ol;
azetidin-3-yl-(4-chlorophenyl)-[4-(1H-pyrazol-4-yl)-phenyl]-methanol;
3-amino-1-(4-chloro-phenyl)-3-methyl-1-[4-(1H-pyrazol-4-yl)-phenyl]-butan-1-ol;
3-amino-1-(4-chloro-phenyl)-1-[4-(3-methyl-1H-pyrazol-4-yl)-phenyl]-propan-1-ol;
3-amino-1-(4-chloro-phenyl)-1-[4-(3-trifluoromethyl-1H-pyrazol-4-yl)-phenyl]-propan-1-ol;
3-(4-chloro-phenyl)-3-methoxy-3-[4-(1H-pyrazol-4-yl)-phenyl]-propylamine; or
a salt, solvate, tautomer or N-oxide thereof.

67. A compound according to claim 66 which is:
3-amino-1-(4-chloro-phenyl)-1-[4-(1H-pyrazol-4-yl)-phenyl]-propan-1-ol;
3-amino-1-(4-chlorophenyl)-1-[2-fluoro-4-(1H-pyrazol-4-yl)-phenyl]-propan-1-ol;
3-amino-1-(4-chloro-3-fluoro-phenyl)-1-[4-(1H-pyrazol-4-yl)-phenyl]-propan-1-ol;
3-amino-1-(3,4,-dichlorophenyl)-1-[4-(1H-pyrazol-4-yl)-phenyl]-propan-1-ol;
or a salt, solvate or tautomer thereof.

68. A compound according to claim 67 which is (S)-3-amino-1-(4-chloro-phenyl)-1-[4-(1H-pyrazol-4-yl)-phenyl]-propan-1-ol or a salt, solvate or tautomer thereof.

69. A compound according to claim 67 which is (R)-3-amino-1-(4-chloro-phenyl)-1-[4-(1H-pyrazol-4-yl)-phenyl]-propan-1-ol or a salt, solvate or tautomer thereof.

70. A compound according to any one of the preceding claims wherein, in the moieties 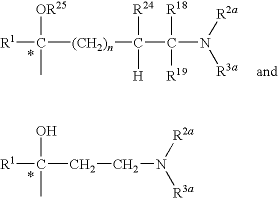
the compounds have the S optical isomeric configuration about the carbon atoms marked with an asterisk.

71. A compound according to any one of the preceding claims in the form of a salt, solvate (such as a hydrate), ester or N-oxide.

72. A compound as defined in any one of claims 1 to 71 for use in the prophylaxis or treatment of a disease state or condition mediated by protein kinase B.

73. The use of a compound as defined in any one of claims 1 to 71 for the manufacture of a medicament for the prophylaxis or treatment of a disease state or condition mediated by protein kinase B.

74. A method for the prophylaxis or treatment of a disease state or condition mediated by protein kinase B, which method comprises administering to a subject in need thereof a compound as defined in any one of claims 1 to 71.

75. A method for treating a disease or condition comprising or arising from abnormal cell growth in a mammal, which method comprises administering to the mammal a compound as defined in any one of claims 1 to 71 in an amount effective in inhibiting abnormal cell growth.

76. A method for treating a disease or condition comprising or arising from abnormal cell growth in a mammal, the method comprising administering to the mammal a compound as defined in any one of claims 1 to 71 in an amount effective to inhibit PKB activity.

77. A method of inhibiting a protein kinase B, which method comprises contacting the kinase with a kinase-inhibiting compound as defined in any one of claims 1 to 71.

78. A method of modulating a cellular process by inhibiting the activity of a protein kinase B using a compound as defined in any one of claims 1 to 71.

79. A method for treating an immune disorder in a mammal, the method comprising administering to the mammal a compound as defined in any one of claims 1 to 71 in an amount effective to inhibit PKB activity.

80. A compound as defined in any one of claims 1 to 71 for use in the prophylaxis or treatment of a disease state or condition mediated by protein kinase A.

81. The use of a compound as defined in any one of claims 1 to 71 for the manufacture of a medicament for the prophylaxis or treatment of a disease state or condition mediated by protein kinase A.

82. The use of a compound of the formula (I) as defined in any one of claims 1 to 71 for the manufacture of a medicament for the prophylaxis or treatment of a disease state or condition arising from abnormal cell growth.

83. The use of a compound of the formula (I) as defined in any one of claims 1 to 71 for the manufacture of a medicament for the prophylaxis or treatment of a disease in which there is a disorder of proliferation, apoptosis or differentiation.

84. A method for the prophylaxis or treatment of a disease state or condition mediated by protein kinase A, which method comprises administering to a subject in need thereof a compound as defined in any one of claims 1 to 71.

85. A method for treating a disease or condition comprising or arising from abnormal cell growth in a mammal, the method comprising administering to the mammal a compound as defined in any one of claims 1 to 71 in an amount effective to inhibit PKA.

86. A method of inhibiting a protein kinase A, which method comprises contacting the kinase with a kinase-inhibiting compound as defined in any one of claims 1 to 71.

87. A method of modulating a cellular process by inhibiting the activity of a protein kinase A using a compound as defined in any one of claims 1 to 71.

88. A method for treating an immune disorder in a mammal, the method comprising administering to the mammal a compound as defined in any one of claims 1 to 71 in an amount effective to inhibit PKA activity.

89. A method of inducing apoptosis in a cancer cell, which method comprises contacting the cancer cell with a compound as defined in any one of claims 1 to 71.

90. A pharmaceutical composition comprising a novel compound as defined in any one of claims 1 to 71 and a pharmaceutically acceptable carrier.

91. A compound as defined in any one of claims 1 to 71 for use in medicine.

92. A process for the preparation of a compound of the formula (II) or (IIa) as defined in any one of claims 1 to 71, wherein R¹⁸, R¹⁹, R^{2a} and R^{3a} are all nitrogen; which process comprises the reduction of a compound of the formula: 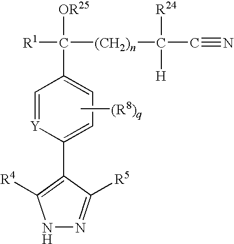
with a reducing agent capable of reducing a nitrile group to an amino group, for example wherein the reducing agent is lithium aluminium hydride.
